SEQUENCE LISTING (333 sequences)

SEQ ID NO: 1 - DNA, 21 bp, Artificial Sequence
  Description of Combined DNA/RNA Molecule Synthetic oligonucleotide
ggcucuuagcaaagucaagtt

SEQ ID NO: 2 - DNA, 21 bp, Artificial Sequence
  Description of Combined DNA/RNA Molecule Synthetic oligonucleotide
cuugacuuugcuaagagcctt

SEQ ID NO: 3 - DNA, 21 bp, Artificial Sequence
  Description of Combined DNA/RNA Molecule Synthetic oligonucleotide
ggaucccauuauuaauggatt

SEQ ID NO: 4 - DNA, 21 bp, Artificial Sequence
  Description of Combined DNA/RNA Molecule Synthetic oligonucleotide
gaucccauuauuaauggaatt

SEQ ID NO: 5 - DNA, 21 bp, Artificial Sequence
  Description of Combined DNA/RNA Molecule Synthetic oligonucleotide
aguuauuuaaaagguguuatt

SEQ ID NO: 6 - DNA, 21 bp, Artificial Sequence
  Description of Combined DNA/RNA Molecule Synthetic oligonucleotide
guuauuuaaaagguguuautt

SEQ ID NO: 7 - DNA, 21 bp, Artificial Sequence
  Description of Combined DNA/RNA Molecule Synthetic oligonucleotide
auuuaaaagguguuaucuctt

SEQ ID NO: 8 - DNA, 21 bp, Artificial Sequence
  Description of Combined DNA/RNA Molecule Synthetic oligonucleotide
uuaaaagguguuaucucuutt

SEQ ID NO: 9 - DNA, 21 bp, Artificial Sequence
  Description of Combined DNA/RNA Molecule Synthetic oligonucleotide
aaguccacuacuagagcautt

SEQ ID NO: 10 - DNA, 21 bp, Artificial Sequence
  Description of Combined DNA/RNA Molecule Synthetic oligonucleotide
aguccacuacuagagcauatt

SEQ ID NO: 11 - DNA, 21 bp, Artificial Sequence
  Description of Combined DNA/RNA Molecule Synthetic oligonucleotide
guccacuacuagagcauautt

SEQ ID NO: 12 - DNA, 21 bp, Artificial Sequence
  Description of Combined DNA/RNA Molecule Synthetic oligonucleotide
uccacuacuagagcauaugtt

SEQ ID NO: 13 - DNA, 21 bp, Artificial Sequence
  Description of Combined DNA/RNA Molecule Synthetic oligonucleotide
gaagagcuauagaaauaagtt

SEQ ID NO: 14 - DNA, 21 bp, Artificial Sequence
  Description of Combined DNA/RNA Molecule Synthetic oligonucleotide
gagcuauagaaauaagugatt

SEQ ID NO: 15 - DNA, 21 bp, Artificial Sequence
  Description of Combined DNA/RNA Molecule Synthetic oligonucleotide
cuauagaaauaagugaugutt

SEQ ID NO: 16 - DNA, 21 bp, Artificial Sequence
  Description of Combined DNA/RNA Molecule Synthetic oligonucleotide
ucaaaacaacacucuugaatt

SEQ ID NO: 17 - DNA, 21 bp, Artificial Sequence
  Description of Combined DNA/RNA Molecule Synthetic oligonucleotide
uagagggauuuauuauguctt

SEQ ID NO: 18 - DNA, 21 bp, Artificial Sequence
  Description of Combined DNA/RNA Molecule Synthetic oligonucleotide
auaaaaggguuuguaaauatt

SEQ ID NO: 19 - DNA, 21 bp, Artificial Sequence
  Description of Combined DNA/RNA Molecule Synthetic oligonucleotide
cucaguguagguagaaugutt

SEQ ID NO: 20 - DNA, 21 bp, Artificial Sequence
  Description of Combined DNA/RNA Molecule Synthetic oligonucleotide
ucaguguagguagaauguutt

SEQ ID NO: 21 - DNA, 21 bp, Artificial Sequence
  Description of Combined DNA/RNA Molecule Synthetic oligonucleotide
caguguagguagaauguuutt

SEQ ID NO: 22 - DNA, 21 bp, Artificial Sequence
  Description of Combined DNA/RNA Molecule Synthetic oligonucleotide
aguguagguagaauguuugtt

SEQ ID NO: 23 - DNA, 21 bp, Artificial Sequence
  Description of Combined DNA/RNA Molecule Synthetic oligonucleotide
guguagguagaauguuugctt

SEQ ID NO: 24 - DNA, 21 bp, Artificial Sequence
  Description of Combined DNA/RNA Molecule Synthetic oligonucleotide
acaagauauggugaucuagtt

SEQ ID NO: 25 - DNA, 21 bp, Artificial Sequence
  Description of Combined DNA/RNA Molecule Synthetic oligonucleotide
agcaaauucaaucaagcautt

SEQ ID NO: 26 - DNA, 21 bp, Artificial Sequence
  Description of Combined DNA/RNA Molecule Synthetic oligonucleotide
gcaaauucaaucaagcauutt

SEQ ID NO: 27 - DNA, 21 bp, Artificial Sequence
  Description of Combined DNA/RNA Molecule Synthetic oligonucleotide
gaugaacaaaguggauuautt

SEQ ID NO: 28 - DNA, 21 bp, Artificial Sequence
  Description of Combined DNA/RNA Molecule Synthetic oligonucleotide
uaauaucucucaaagggaatt

SEQ ID NO: 29 - DNA, 21 bp, Artificial Sequence
  Description of Combined DNA/RNA Molecule Synthetic oligonucleotide
auaucucucaaagggaaautt

SEQ ID NO: 30 - DNA, 21 bp, Artificial Sequence
  Description of Combined DNA/RNA Molecule Synthetic oligonucleotide
uaucucucaaagggaaauutt

SEQ ID NO: 31 - DNA, 21 bp, Artificial Sequence
  Description of Combined DNA/RNA Molecule Synthetic oligonucleotide
caugcucaagcagauuauutt

SEQ ID NO: 32 - DNA, 21 bp, Artificial Sequence
  Description of Combined DNA/RNA Molecule Synthetic oligonucleotide
ugcucaagcagauuauuugtt

SEQ ID NO: 33 - DNA, 21 bp, Artificial Sequence
  Description of Combined DNA/RNA Molecule Synthetic oligonucleotide
uagcauuaaauagccuuaatt

SEQ ID NO: 34 - DNA, 21 bp, Artificial Sequence
  Description of Combined DNA/RNA Molecule Synthetic oligonucleotide
agcauuaaauagccuuaaatt

SEQ ID NO: 35 - DNA, 21 bp, Artificial Sequence
  Description of Combined DNA/RNA Molecule Synthetic oligonucleotide
gcauuaaauagccuuaaautt

SEQ ID NO: 36 - DNA, 21 bp, Artificial Sequence
  Description of Combined DNA/RNA Molecule Synthetic oligonucleotide
cauuaaauagccuuaaauutt

SEQ ID NO: 37 - DNA, 21 bp, Artificial Sequence
  Description of Combined DNA/RNA Molecule Synthetic oligonucleotide
uauuaugcaguuuaauauutt

SEQ ID NO: 38 - DNA, 21 bp, Artificial Sequence
  Description of Combined DNA/RNA Molecule Synthetic oligonucleotide
uuaugcaguuuaauauuuatt

SEQ ID NO: 39 - DNA, 21 bp, Artificial Sequence
  Description of Combined DNA/RNA Molecule Synthetic oligonucleotide
aaaagugcacaacauuauatt

SEQ ID NO: 40 - DNA, 21 bp, Artificial Sequence
  Description of Combined DNA/RNA Molecule Synthetic oligonucleotide
aaagugcacaacauuauactt

SEQ ID NO: 41 - DNA, 21 bp, Artificial Sequence
  Description of Combined DNA/RNA Molecule Synthetic oligonucleotide
auauagaaccuacauaucctt

SEQ ID NO: 42 - DNA, 21 bp, Artificial Sequence
  Description of Combined DNA/RNA Molecule Synthetic oligonucleotide
uauagaaccuacauauccutt

SEQ ID NO: 43 - DNA, 21 bp, Artificial Sequence
  Description of Combined DNA/RNA Molecule Synthetic oligonucleotide
uaagaguuguuuaugaaagtt

SEQ ID NO: 44 - DNA, 21 bp, Artificial Sequence
  Description of Combined DNA/RNA Molecule Synthetic oligonucleotide
acagucaguaguagaccautt

SEQ ID NO: 45 - DNA, 21 bp, Artificial Sequence
  Description of Combined DNA/RNA Molecule Synthetic oligonucleotide
cagucaguaguagaccaugtt

SEQ ID NO: 46 - DNA, 21 bp, Artificial Sequence
  Description of Combined DNA/RNA Molecule Synthetic oligonucleotide
agucaguaguagaccaugutt

SEQ ID NO: 47 - DNA, 21 bp, Artificial Sequence
  Description of Combined DNA/RNA Molecule Synthetic oligonucleotide
gucaguaguagaccaugugtt

SEQ ID NO: 48 - DNA, 21 bp, Artificial Sequence
  Description of Combined DNA/RNA Molecule Synthetic oligonucleotide
ucaguaguagaccaugugatt

SEQ ID NO: 49 - DNA, 21 bp, Artificial Sequence
  Description of Combined DNA/RNA Molecule Synthetic oligonucleotide
caugugaauucccugcauctt

SEQ ID NO: 50 - DNA, 21 bp, Artificial Sequence
  Description of Combined DNA/RNA Molecule Synthetic oligonucleotide
augugaauucccugcaucatt

SEQ ID NO: 51 - DNA, 21 bp, Artificial Sequence
  Description of Combined DNA/RNA Molecule Synthetic oligonucleotide
ugugaauucccugcaucaatt

SEQ ID NO: 52 - DNA, 21 bp, Artificial Sequence
  Description of Combined DNA/RNA Molecule Synthetic oligonucleotide
gugaauucccugcaucaautt

SEQ ID NO: 53 - DNA, 21 bp, Artificial Sequence
  Description of Combined DNA/RNA Molecule Synthetic oligonucleotide
aauucccugcaucaauacctt

SEQ ID NO: 54 - DNA, 21 bp, Artificial Sequence
  Description of Combined DNA/RNA Molecule Synthetic oligonucleotide
gcaucaauaccagcuuauatt

SEQ ID NO: 55 - DNA, 21 bp, Artificial Sequence
  Description of Combined DNA/RNA Molecule Synthetic oligonucleotide
caucaauaccagcuuauagtt

SEQ ID NO: 56 - DNA, 21 bp, Artificial Sequence
  Description of Combined DNA/RNA Molecule Synthetic oligonucleotide
auaccagcuuauagaacaatt

SEQ ID NO: 57 - DNA, 21 bp, Artificial Sequence
  Description of Combined DNA/RNA Molecule Synthetic oligonucleotide
uaccagcuuauagaacaactt

SEQ ID NO: 58 - DNA, 21 bp, Artificial Sequence
  Description of Combined DNA/RNA Molecule Synthetic oligonucleotide
accagcuuauagaacaacatt

SEQ ID NO: 59 - DNA, 21 bp, Artificial Sequence
  Description of Combined DNA/RNA Molecule Synthetic oligonucleotide
ccagcuuauagaacaacaatt

SEQ ID NO: 60 - DNA, 21 bp, Artificial Sequence
  Description of Combined DNA/RNA Molecule Synthetic oligonucleotide
cagcuuauagaacaacaaatt

SEQ ID NO: 61 - DNA, 21 bp, Artificial Sequence
  Description of Combined DNA/RNA Molecule Synthetic oligonucleotide
auagaacaacaaauuaucatt

SEQ ID NO: 62 - DNA, 21 bp, Artificial Sequence
  Description of Combined DNA/RNA Molecule Synthetic oligonucleotide
uauuaacagaaaaguauggtt

SEQ ID NO: 63 - DNA, 21 bp, Artificial Sequence
  Description of Combined DNA/RNA Molecule Synthetic oligonucleotide
ugagauacauuugaugaaatt

SEQ ID NO: 64 - DNA, 21 bp, Artificial Sequence
  Description of Combined DNA/RNA Molecule Synthetic oligonucleotide
gagauacauuugaugaaactt

SEQ ID NO: 65 - DNA, 21 bp, Artificial Sequence
  Description of Combined DNA/RNA Molecule Synthetic oligonucleotide
gauacauuugaugaaaccutt

SEQ ID NO: 66 - DNA, 21 bp, Artificial Sequence
  Description of Combined DNA/RNA Molecule Synthetic oligonucleotide
auacauuugaugaaaccuctt

SEQ ID NO: 67 - DNA, 21 bp, Artificial Sequence
  Description of Combined DNA/RNA Molecule Synthetic oligonucleotide
uacauuugaugaaaccucctt

SEQ ID NO: 68 - DNA, 21 bp, Artificial Sequence
  Description of Combined DNA/RNA Molecule Synthetic oligonucleotide
aagugauacaaaaacagcatt

SEQ ID NO: 69 - DNA, 21 bp, Artificial Sequence
  Description of Combined DNA/RNA Molecule Synthetic oligonucleotide
agugauacaaaaacagcautt

SEQ ID NO: 70 - DNA, 21 bp, Artificial Sequence
  Description of Combined DNA/RNA Molecule Synthetic oligonucleotide
ugauacaaaaacagcauautt

SEQ ID NO: 71 - DNA, 21 bp, Artificial Sequence
  Description of Combined DNA/RNA Molecule Synthetic oligonucleotide
uuuaaguacuaauuuagcutt

SEQ ID NO: 72 - DNA, 21 bp, Artificial Sequence
  Description of Combined DNA/RNA Molecule Synthetic oligonucleotide
uuaaguacuaauuuagcugtt

SEQ ID NO: 73 - DNA, 21 bp, Artificial Sequence
  Description of Combined DNA/RNA Molecule Synthetic oligonucleotide
uaaguacuaauuuagcuggtt

SEQ ID NO: 74 - DNA, 21 bp, Artificial Sequence
  Description of Combined DNA/RNA Molecule Synthetic oligonucleotide
aaguacuaauuuagcuggatt

SEQ ID NO: 75 - DNA, 21 bp, Artificial Sequence
  Description of Combined DNA/RNA Molecule Synthetic oligonucleotide
aguacuaauuuagcuggactt

SEQ ID NO: 76 - DNA, 21 bp, Artificial Sequence
  Description of Combined DNA/RNA Molecule Synthetic oligonucleotide
guacuaauuuagcuggacatt

SEQ ID NO: 77 - DNA, 21 bp, Artificial Sequence
  Description of Combined DNA/RNA Molecule Synthetic oligonucleotide
acuaauuuagcuggacauutt

SEQ ID NO: 78 - DNA, 21 bp, Artificial Sequence
  Description of Combined DNA/RNA Molecule Synthetic oligonucleotide
aauuuagcuggacauuggatt

SEQ ID NO: 79 - DNA, 21 bp, Artificial Sequence
  Description of Combined DNA/RNA Molecule Synthetic oligonucleotide
auuuagcuggacauuggautt

SEQ ID NO: 80 - DNA, 21 bp, Artificial Sequence
  Description of Combined DNA/RNA Molecule Synthetic oligonucleotide
uuagcuggacauuggauuctt

SEQ ID NO: 81 - DNA, 21 bp, Artificial Sequence
  Description of Combined DNA/RNA Molecule Synthetic oligonucleotide
uuuugaaaaagauuggggatt

SEQ ID NO: 82 - DNA, 21 bp, Artificial Sequence
  Description of Combined DNA/RNA Molecule Synthetic oligonucleotide
uuugaaaaagauuggggagtt

SEQ ID NO: 83 - DNA, 21 bp, Artificial Sequence
  Description of Combined DNA/RNA Molecule Synthetic oligonucleotide
ugaaaaagauuggggagagtt

SEQ ID NO: 84 - DNA, 21 bp, Artificial Sequence
  Description of Combined DNA/RNA Molecule Synthetic oligonucleotide
gaaaaagauuggggagaggtt

SEQ ID NO: 85 - DNA, 21 bp, Artificial Sequence
  Description of Combined DNA/RNA Molecule Synthetic oligonucleotide
uaugaacacuucagaucuutt

SEQ ID NO: 86 - DNA, 21 bp, Artificial Sequence
  Description of Combined DNA/RNA Molecule Synthetic oligonucleotide
augaacacuucagaucuuctt

SEQ ID NO: 87 - DNA, 21 bp, Artificial Sequence
  Description of Combined DNA/RNA Molecule Synthetic oligonucleotide
ugcccuuggguuguuaacatt

SEQ ID NO: 88 - DNA, 21 bp, Artificial Sequence
  Description of Combined DNA/RNA Molecule Synthetic oligonucleotide
gcccuuggguuguuaacautt

SEQ ID NO: 89 - DNA, 21 bp, Artificial Sequence
  Description of Combined DNA/RNA Molecule Synthetic oligonucleotide
uauagcauucauaggugaatt

SEQ ID NO: 90 - DNA, 21 bp, Artificial Sequence
  Description of Combined DNA/RNA Molecule Synthetic oligonucleotide
auagcauucauaggugaagtt

SEQ ID NO: 91 - DNA, 21 bp, Artificial Sequence
  Description of Combined DNA/RNA Molecule Synthetic oligonucleotide
uagcauucauaggugaaggtt

SEQ ID NO: 92 - DNA, 21 bp, Artificial Sequence
  Description of Combined DNA/RNA Molecule Synthetic oligonucleotide
auucauaggugaaggagcatt

SEQ ID NO: 93 - DNA, 21 bp, Artificial Sequence
  Description of Combined DNA/RNA Molecule Synthetic oligonucleotide
uugcaaugaucauaguuuatt

SEQ ID NO: 94 - DNA, 21 bp, Artificial Sequence
  Description of Combined DNA/RNA Molecule Synthetic oligonucleotide
ugcaaugaucauaguuuactt

SEQ ID NO: 95 - DNA, 21 bp, Artificial Sequence
  Description of Combined DNA/RNA Molecule Synthetic oligonucleotide
gcaaugaucauaguuuacctt

SEQ ID NO: 96 - DNA, 21 bp, Artificial Sequence
  Description of Combined DNA/RNA Molecule Synthetic oligonucleotide
caaugaucauaguuuaccutt

SEQ ID NO: 97 - DNA, 21 bp, Artificial Sequence
  Description of Combined DNA/RNA Molecule Synthetic oligonucleotide
aaugaucauaguuuaccuatt

SEQ ID NO: 98 - DNA, 21 bp, Artificial Sequence
  Description of Combined DNA/RNA Molecule Synthetic oligonucleotide
augaucauaguuuaccuautt

SEQ ID NO: 99 - DNA, 21 bp, Artificial Sequence
  Description of Combined DNA/RNA Molecule Synthetic oligonucleotide
gaucauaguuuaccuauugtt

SEQ ID NO: 100 - DNA, 21 bp, Artificial Sequence
  Description of Combined DNA/RNA Molecule Synthetic oligonucleotide
aucauaguuuaccuauugatt

SEQ ID NO: 101 - DNA, 21 bp, Artificial Sequence
  Description of Combined DNA/RNA Molecule Synthetic oligonucleotide
ucauaguuuaccuauugagtt

SEQ ID NO: 102 - DNA, 21 bp, Artificial Sequence
  Description of Combined DNA/RNA Molecule Synthetic oligonucleotide
cauaguuuaccuauugagutt

SEQ ID NO: 103 - DNA, 21 bp, Artificial Sequence
  Description of Combined DNA/RNA Molecule Synthetic oligonucleotide
auaguuuaccuauugaguutt

SEQ ID NO: 104 - DNA, 21 bp, Artificial Sequence
  Description of Combined DNA/RNA Molecule Synthetic oligonucleotide
cauuggucuuauuuacauatt

SEQ ID NO: 105 - DNA, 21 bp, Artificial Sequence
  Description of Combined DNA/RNA Molecule Synthetic oligonucleotide
uuggucuuauuuacauauatt

SEQ ID NO: 106 - DNA, 21 bp, Artificial Sequence
  Description of Combined DNA/RNA Molecule Synthetic oligonucleotide
uggucuuauuuacauauaatt

SEQ ID NO: 107 - DNA, 21 bp, Artificial Sequence
  Description of Combined DNA/RNA Molecule Synthetic oligonucleotide
ggucuuauuuacauauaaatt

SEQ ID NO: 108 - DNA, 21 bp, Artificial Sequence
  Description of Combined DNA/RNA Molecule Synthetic oligonucleotide
auaucaugcucaagaugautt

SEQ ID NO: 109 - DNA, 21 bp, Artificial Sequence
  Description of Combined DNA/RNA Molecule Synthetic oligonucleotide
uaucaugcucaagaugauatt

SEQ ID NO: 110 - DNA, 21 bp, Artificial Sequence
  Description of Combined DNA/RNA Molecule Synthetic oligonucleotide
ugauauugauuucaaauuatt

SEQ ID NO: 111 - DNA, 21 bp, Artificial Sequence
  Description of Combined DNA/RNA Molecule Synthetic oligonucleotide
uacuuaguccuuacaauagtt

SEQ ID NO: 112 - DNA, 21 bp, Artificial Sequence
  Description of Combined DNA/RNA Molecule Synthetic oligonucleotide
uuaguccuuacaauagguctt

SEQ ID NO: 113 - DNA, 21 bp, Artificial Sequence
  Description of Combined DNA/RNA Molecule Synthetic oligonucleotide
uaguccuuacaauaggucctt

SEQ ID NO: 114 - DNA, 21 bp, Artificial Sequence
  Description of Combined DNA/RNA Molecule Synthetic oligonucleotide
auauucuauagcuggacgutt

SEQ ID NO: 115 - DNA, 21 bp, Artificial Sequence
  Description of Combined DNA/RNA Molecule Synthetic oligonucleotide
uauucuauagcuggacguatt

SEQ ID NO: 116 - DNA, 21 bp, Artificial Sequence
  Description of Combined DNA/RNA Molecule Synthetic oligonucleotide
auucuauagcuggacguaatt

SEQ ID NO: 117 - DNA, 21 bp, Artificial Sequence
  Description of Combined DNA/RNA Molecule Synthetic oligonucleotide
uccauuaauaaugggaucctt

SEQ ID NO: 118 - DNA, 21 bp, Artificial Sequence
  Description of Combined DNA/RNA Molecule Synthetic oligonucleotide
uuccauuaauaaugggauctt

SEQ ID NO: 119 - DNA, 21 bp, Artificial Sequence
  Description of Combined DNA/RNA Molecule Synthetic oligonucleotide
uaacaccuuuuaaauaacutt

SEQ ID NO: 120 - DNA, 21 bp, Artificial Sequence
  Description of Combined DNA/RNA Molecule Synthetic oligonucleotide
auaacaccuuuuaaauaactt

SEQ ID NO: 121 - DNA, 21 bp, Artificial Sequence
  Description of Combined DNA/RNA Molecule Synthetic oligonucleotide
gagauaacaccuuuuaaautt

SEQ ID NO: 122 - DNA, 21 bp, Artificial Sequence
  Description of Combined DNA/RNA Molecule Synthetic oligonucleotide
aagagauaacaccuuuuaatt

SEQ ID NO: 123 - DNA, 21 bp, Artificial Sequence
  Description of Combined DNA/RNA Molecule Synthetic oligonucleotide
augcucuaguaguggacuutt

SEQ ID NO: 124 - DNA, 21 bp, Artificial Sequence
  Description of Combined DNA/RNA Molecule Synthetic oligonucleotide
uaugcucuaguaguggacutt

SEQ ID NO: 125 - DNA, 21 bp, Artificial Sequence
  Description of Combined DNA/RNA Molecule Synthetic oligonucleotide
auaugcucuaguaguggactt

SEQ ID NO: 126 - DNA, 21 bp, Artificial Sequence
  Description of Combined DNA/RNA Molecule Synthetic oligonucleotide
cauaugcucuaguaguggatt

SEQ ID NO: 127 - DNA, 21 bp, Artificial Sequence
  Description of Combined DNA/RNA Molecule Synthetic oligonucleotide
cuuauuucuauagcucuuctt

SEQ ID NO: 128 - DNA, 21 bp, Artificial Sequence
  Description of Combined DNA/RNA Molecule Synthetic oligonucleotide
ucacuuauuucuauagcuctt

SEQ ID NO: 129 - DNA, 21 bp, Artificial Sequence
  Description of Combined DNA/RNA Molecule Synthetic oligonucleotide
acaucacuuauuucuauagtt

SEQ ID NO: 130 - DNA, 21 bp, Artificial Sequence
  Description of Combined DNA/RNA Molecule Synthetic oligonucleotide
uucaagaguguuguuuugatt

SEQ ID NO: 131 - DNA, 21 bp, Artificial Sequence
  Description of Combined DNA/RNA Molecule Synthetic oligonucleotide
gacauaauaaaucccucuatt

SEQ ID NO: 132 - DNA, 21 bp, Artificial Sequence
  Description of Combined DNA/RNA Molecule Synthetic oligonucleotide
uauuuacaaacccuuuuautt

SEQ ID NO: 133 - DNA, 21 bp, Artificial Sequence
  Description of Combined DNA/RNA Molecule Synthetic oligonucleotide
acauucuaccuacacugagtt

SEQ ID NO: 134 - DNA, 21 bp, Artificial Sequence
  Description of Combined DNA/RNA Molecule Synthetic oligonucleotide
aacauucuaccuacacugatt

SEQ ID NO: 135 - DNA, 21 bp, Artificial Sequence
  Description of Combined DNA/RNA Molecule Synthetic oligonucleotide
aaacauucuaccuacacugtt

SEQ ID NO: 136 - DNA, 21 bp, Artificial Sequence
  Description of Combined DNA/RNA Molecule Synthetic oligonucleotide
caaacauucuaccuacacutt

SEQ ID NO: 137 - DNA, 21 bp, Artificial Sequence
  Description of Combined DNA/RNA Molecule Synthetic oligonucleotide
gcaaacauucuaccuacactt

SEQ ID NO: 138 - DNA, 21 bp, Artificial Sequence
  Description of Combined DNA/RNA Molecule Synthetic oligonucleotide
cuagaucaccauaucuugutt

SEQ ID NO: 139 - DNA, 21 bp, Artificial Sequence
  Description of Combined DNA/RNA Molecule Synthetic oligonucleotide
augcuugauugaauuugcutt

SEQ ID NO: 140 - DNA, 21 bp, Artificial Sequence
  Description of Combined DNA/RNA Molecule Synthetic oligonucleotide
aaugcuugauugaauuugctt

SEQ ID NO: 141 - DNA, 21 bp, Artificial Sequence
  Description of Combined DNA/RNA Molecule Synthetic oligonucleotide
auaauccacuuuguucauctt

SEQ ID NO: 142 - DNA, 21 bp, Artificial Sequence
  Description of Combined DNA/RNA Molecule Synthetic oligonucleotide
uucccuuugagagauauuatt

SEQ ID NO: 143 - DNA, 21 bp, Artificial Sequence
  Description of Combined DNA/RNA Molecule Synthetic oligonucleotide
auuucccuuugagagauautt

SEQ ID NO: 144 - DNA, 21 bp, Artificial Sequence
  Description of Combined DNA/RNA Molecule Synthetic oligonucleotide
aauuucccuuugagagauatt

SEQ ID NO: 145 - DNA, 21 bp, Artificial Sequence
  Description of Combined DNA/RNA Molecule Synthetic oligonucleotide
aauaaucugcuugagcaugtt

SEQ ID NO: 146 - DNA, 21 bp, Artificial Sequence
  Description of Combined DNA/RNA Molecule Synthetic oligonucleotide
caaauaaucugcuugagcatt

SEQ ID NO: 147 - DNA, 21 bp, Artificial Sequence
  Description of Combined DNA/RNA Molecule Synthetic oligonucleotide
uuaaggcuauuuaaugcuatt

SEQ ID NO: 148 - DNA, 21 bp, Artificial Sequence
  Description of Combined DNA/RNA Molecule Synthetic oligonucleotide
uuuaaggcuauuuaaugcutt

SEQ ID NO: 149 - DNA, 21 bp, Artificial Sequence
  Description of Combined DNA/RNA Molecule Synthetic oligonucleotide
auuuaaggcuauuuaaugctt

SEQ ID NO: 150 - DNA, 21 bp, Artificial Sequence
  Description of Combined DNA/RNA Molecule Synthetic oligonucleotide
aauuuaaggcuauuuaaugtt

SEQ ID NO: 151 - DNA, 21 bp, Artificial Sequence
  Description of Combined DNA/RNA Molecule Synthetic oligonucleotide
aauauuaaacugcauaauatt

SEQ ID NO: 152 - DNA, 21 bp, Artificial Sequence
  Description of Combined DNA/RNA Molecule Synthetic oligonucleotide
uaaauauuaaacugcauaatt

SEQ ID NO: 153 - DNA, 21 bp, Artificial Sequence
  Description of Combined DNA/RNA Molecule Synthetic oligonucleotide
uauaauguugugcacuuuutt

SEQ ID NO: 154 - DNA, 21 bp, Artificial Sequence
  Description of Combined DNA/RNA Molecule Synthetic oligonucleotide
guauaauguugugcacuuutt

SEQ ID NO: 155 - DNA, 21 bp, Artificial Sequence
  Description of Combined DNA/RNA Molecule Synthetic oligonucleotide
ggauauguagguucuauautt

SEQ ID NO: 156 - DNA, 21 bp, Artificial Sequence
  Description of Combined DNA/RNA Molecule Synthetic oligonucleotide
aggauauguagguucuauatt

SEQ ID NO: 157 - DNA, 21 bp, Artificial Sequence
  Description of Combined DNA/RNA Molecule Synthetic oligonucleotide
cuuucauaaacaacucuuatt

SEQ ID NO: 158 - DNA, 21 bp, Artificial Sequence
  Description of Combined DNA/RNA Molecule Synthetic oligonucleotide
auggucuacuacugacugutt

SEQ ID NO: 159 - DNA, 21 bp, Artificial Sequence
  Description of Combined DNA/RNA Molecule Synthetic oligonucleotide
cauggucuacuacugacugtt

SEQ ID NO: 160 - DNA, 21 bp, Artificial Sequence
  Description of Combined DNA/RNA Molecule Synthetic oligonucleotide
acauggucuacuacugacutt

SEQ ID NO: 161 - DNA, 21 bp, Artificial Sequence
  Description of Combined DNA/RNA Molecule Synthetic oligonucleotide
cacauggucuacuacugactt

SEQ ID NO: 162 - DNA, 21 bp, Artificial Sequence
  Description of Combined DNA/RNA Molecule Synthetic oligonucleotide
ucacauggucuacuacugatt

SEQ ID NO: 163 - DNA, 21 bp, Artificial Sequence
  Description of Combined DNA/RNA Molecule Synthetic oligonucleotide
gaugcagggaauucacaugtt

SEQ ID NO: 164 - DNA, 21 bp, Artificial Sequence
  Description of Combined DNA/RNA Molecule Synthetic oligonucleotide
ugaugcagggaauucacautt

SEQ ID NO: 165 - DNA, 21 bp, Artificial Sequence
  Description of Combined DNA/RNA Molecule Synthetic oligonucleotide
uugaugcagggaauucacatt

SEQ ID NO: 166 - DNA, 21 bp, Artificial Sequence
  Description of Combined DNA/RNA Molecule Synthetic oligonucleotide
auugaugcagggaauucactt

SEQ ID NO: 167 - DNA, 21 bp, Artificial Sequence
  Description of Combined DNA/RNA Molecule Synthetic oligonucleotide
gguauugaugcagggaauutt

SEQ ID NO: 168 - DNA, 21 bp, Artificial Sequence
  Description of Combined DNA/RNA Molecule Synthetic oligonucleotide
uauaagcugguauugaugctt

SEQ ID NO: 169 - DNA, 21 bp, Artificial Sequence
  Description of Combined DNA/RNA Molecule Synthetic oligonucleotide
cuauaagcugguauugaugtt

SEQ ID NO: 170 - DNA, 21 bp, Artificial Sequence
  Description of Combined DNA/RNA Molecule Synthetic oligonucleotide
uuguucuauaagcugguautt

SEQ ID NO: 171 - DNA, 21 bp, Artificial Sequence
  Description of Combined DNA/RNA Molecule Synthetic oligonucleotide
guuguucuauaagcugguatt

SEQ ID NO: 172 - DNA, 21 bp, Artificial Sequence
  Description of Combined DNA/RNA Molecule Synthetic oligonucleotide
uguuguucuauaagcuggutt

SEQ ID NO: 173 - DNA, 21 bp, Artificial Sequence
  Description of Combined DNA/RNA Molecule Synthetic oligonucleotide
uuguuguucuauaagcuggtt

SEQ ID NO: 174 - DNA, 21 bp, Artificial Sequence
  Description of Combined DNA/RNA Molecule Synthetic oligonucleotide
uuuguuguucuauaagcugtt

SEQ ID NO: 175 - DNA, 21 bp, Artificial Sequence
  Description of Combined DNA/RNA Molecule Synthetic oligonucleotide
ugauaauuuguuguucuautt

SEQ ID NO: 176 - DNA, 21 bp, Artificial Sequence
  Description of Combined DNA/RNA Molecule Synthetic oligonucleotide
ccauacuuuucuguuaauatt

SEQ ID NO: 177 - DNA, 21 bp, Artificial Sequence
  Description of Combined DNA/RNA Molecule Synthetic oligonucleotide
uuucaucaaauguaucucatt

SEQ ID NO: 178 - DNA, 21 bp, Artificial Sequence
  Description of Combined DNA/RNA Molecule Synthetic oligonucleotide
guuucaucaaauguaucuctt

SEQ ID NO: 179 - DNA, 21 bp, Artificial Sequence
  Description of Combined DNA/RNA Molecule Synthetic oligonucleotide
agguuucaucaaauguauctt

SEQ ID NO: 180 - DNA, 21 bp, Artificial Sequence
  Description of Combined DNA/RNA Molecule Synthetic oligonucleotide
gagguuucaucaaauguautt

SEQ ID NO: 181 - DNA, 21 bp, Artificial Sequence
  Description of Combined DNA/RNA Molecule Synthetic oligonucleotide
ggagguuucaucaaauguatt

SEQ ID NO: 182 - DNA, 21 bp, Artificial Sequence
  Description of Combined DNA/RNA Molecule Synthetic oligonucleotide
ugcuguuuuuguaucacuutt

SEQ ID NO: 183 - DNA, 21 bp, Artificial Sequence
  Description of Combined DNA/RNA Molecule Synthetic oligonucleotide
augcuguuuuuguaucacutt

SEQ ID NO: 184 - DNA, 21 bp, Artificial Sequence
  Description of Combined DNA/RNA Molecule Synthetic oligonucleotide
auaugcuguuuuuguaucatt

SEQ ID NO: 185 - DNA, 21 bp, Artificial Sequence
  Description of Combined DNA/RNA Molecule Synthetic oligonucleotide
agcuaaauuaguacuuaaatt

SEQ ID NO: 186 - DNA, 21 bp, Artificial Sequence
  Description of Combined DNA/RNA Molecule Synthetic oligonucleotide
cagcuaaauuaguacuuaatt

SEQ ID NO: 187 - DNA, 21 bp, Artificial Sequence
  Description of Combined DNA/RNA Molecule Synthetic oligonucleotide
ccagcuaaauuaguacuuatt

SEQ ID NO: 188 - DNA, 21 bp, Artificial Sequence
  Description of Combined DNA/RNA Molecule Synthetic oligonucleotide
uccagcuaaauuaguacuutt

SEQ ID NO: 189 - DNA, 21 bp, Artificial Sequence
  Description of Combined DNA/RNA Molecule Synthetic oligonucleotide
guccagcuaaauuaguacutt

SEQ ID NO: 190 - DNA, 21 bp, Artificial Sequence
  Description of Combined DNA/RNA Molecule Synthetic oligonucleotide
uguccagcuaaauuaguactt

SEQ ID NO: 191 - DNA, 21 bp, Artificial Sequence
  Description of Combined DNA/RNA Molecule Synthetic oligonucleotide
aauguccagcuaaauuagutt

SEQ ID NO: 192 - DNA, 21 bp, Artificial Sequence
  Description of Combined DNA/RNA Molecule Synthetic oligonucleotide
uccaauguccagcuaaauutt

SEQ ID NO: 193 - DNA, 21 bp, Artificial Sequence
  Description of Combined DNA/RNA Molecule Synthetic oligonucleotide
auccaauguccagcuaaautt

SEQ ID NO: 194 - DNA, 21 bp, Artificial Sequence
  Description of Combined DNA/RNA Molecule Synthetic oligonucleotide
gaauccaauguccagcuaatt

SEQ ID NO: 195 - DNA, 21 bp, Artificial Sequence
  Description of Combined DNA/RNA Molecule Synthetic oligonucleotide
uccccaaucuuuuucaaaatt

SEQ ID NO: 196 - DNA, 21 bp, Artificial Sequence
  Description of Combined DNA/RNA Molecule Synthetic oligonucleotide
cuccccaaucuuuuucaaatt

SEQ ID NO: 197 - DNA, 21 bp, Artificial Sequence
  Description of Combined DNA/RNA Molecule Synthetic oligonucleotide
cucuccccaaucuuuuucatt

SEQ ID NO: 198 - DNA, 21 bp, Artificial Sequence
  Description of Combined DNA/RNA Molecule Synthetic oligonucleotide
ccucuccccaaucuuuuuctt

SEQ ID NO: 199 - DNA, 21 bp, Artificial Sequence
  Description of Combined DNA/RNA Molecule Synthetic oligonucleotide
aagaucugaaguguucauatt

SEQ ID NO: 200 - DNA, 21 bp, Artificial Sequence
  Description of Combined DNA/RNA Molecule Synthetic oligonucleotide
gaagaucugaaguguucautt

SEQ ID NO: 201 - DNA, 21 bp, Artificial Sequence
  Description of Combined DNA/RNA Molecule Synthetic oligonucleotide
uguuaacaacccaagggcatt

SEQ ID NO: 202 - DNA, 21 bp, Artificial Sequence
  Description of Combined DNA/RNA Molecule Synthetic oligonucleotide
auguuaacaacccaagggctt

SEQ ID NO: 203 - DNA, 21 bp, Artificial Sequence
  Description of Combined DNA/RNA Molecule Synthetic oligonucleotide
uucaccuaugaaugcuauatt

SEQ ID NO: 204 - DNA, 21 bp, Artificial Sequence
  Description of Combined DNA/RNA Molecule Synthetic oligonucleotide
cuucaccuaugaaugcuautt

SEQ ID NO: 205 - DNA, 21 bp, Artificial Sequence
  Description of Combined DNA/RNA Molecule Synthetic oligonucleotide
ccuucaccuaugaaugcuatt

SEQ ID NO: 206 - DNA, 21 bp, Artificial Sequence
  Description of Combined DNA/RNA Molecule Synthetic oligonucleotide
ugcuccuucaccuaugaautt

SEQ ID NO: 207 - DNA, 21 bp, Artificial Sequence
  Description of Combined DNA/RNA Molecule Synthetic oligonucleotide
uaaacuaugaucauugcaatt

SEQ ID NO: 208 - DNA, 21 bp, Artificial Sequence
  Description of Combined DNA/RNA Molecule Synthetic oligonucleotide
guaaacuaugaucauugcatt

SEQ ID NO: 209 - DNA, 21 bp, Artificial Sequence
  Description of Combined DNA/RNA Molecule Synthetic oligonucleotide
gguaaacuaugaucauugctt

SEQ ID NO: 210 - DNA, 21 bp, Artificial Sequence
  Description of Combined DNA/RNA Molecule Synthetic oligonucleotide
agguaaacuaugaucauugtt

SEQ ID NO: 211 - DNA, 21 bp, Artificial Sequence
  Description of Combined DNA/RNA Molecule Synthetic oligonucleotide
uagguaaacuaugaucauutt

SEQ ID NO: 212 - DNA, 21 bp, Artificial Sequence
  Description of Combined DNA/RNA Molecule Synthetic oligonucleotide
auagguaaacuaugaucautt

SEQ ID NO: 213 - DNA, 21 bp, Artificial Sequence
  Description of Combined DNA/RNA Molecule Synthetic oligonucleotide
caauagguaaacuaugauctt

SEQ ID NO: 214 - DNA, 21 bp, Artificial Sequence
  Description of Combined DNA/RNA Molecule Synthetic oligonucleotide
ucaauagguaaacuaugautt

SEQ ID NO: 215 - DNA, 21 bp, Artificial Sequence
  Description of Combined DNA/RNA Molecule Synthetic oligonucleotide
cucaauagguaaacuaugatt

SEQ ID NO: 216 - DNA, 21 bp, Artificial Sequence
  Description of Combined DNA/RNA Molecule Synthetic oligonucleotide
acucaauagguaaacuaugtt

SEQ ID NO: 217 - DNA, 21 bp, Artificial Sequence
  Description of Combined DNA/RNA Molecule Synthetic oligonucleotide
aacucaauagguaaacuautt

SEQ ID NO: 218 - DNA, 21 bp, Artificial Sequence
  Description of Combined DNA/RNA Molecule Synthetic oligonucleotide
uauguaaauaagaccaaugtt

SEQ ID NO: 219 - DNA, 21 bp, Artificial Sequence
  Description of Combined DNA/RNA Molecule Synthetic oligonucleotide
uauauguaaauaagaccaatt

SEQ ID NO: 220 - DNA, 21 bp, Artificial Sequence
  Description of Combined DNA/RNA Molecule Synthetic oligonucleotide
uuauauguaaauaagaccatt

SEQ ID NO: 221 - DNA, 21 bp, Artificial Sequence
  Description of Combined DNA/RNA Molecule Synthetic oligonucleotide
uuuauauguaaauaagacctt

SEQ ID NO: 222 - DNA, 21 bp, Artificial Sequence
  Description of Combined DNA/RNA Molecule Synthetic oligonucleotide
aucaucuugagcaugauautt

SEQ ID NO: 223 - DNA, 21 bp, Artificial Sequence
  Description of Combined DNA/RNA Molecule Synthetic oligonucleotide
uaucaucuugagcaugauatt

SEQ ID NO: 224 - DNA, 21 bp, Artificial Sequence
  Description of Combined DNA/RNA Molecule Synthetic oligonucleotide
uaauuugaaaucaauaucatt

SEQ ID NO: 225 - DNA, 21 bp, Artificial Sequence
  Description of Combined DNA/RNA Molecule Synthetic oligonucleotide
cuauuguaaggacuaaguatt

SEQ ID NO: 226 - DNA, 21 bp, Artificial Sequence
  Description of Combined DNA/RNA Molecule Synthetic oligonucleotide
gaccuauuguaaggacuaatt

SEQ ID NO: 227 - DNA, 21 bp, Artificial Sequence
  Description of Combined DNA/RNA Molecule Synthetic oligonucleotide
ggaccuauuguaaggacuatt

SEQ ID NO: 228 - DNA, 21 bp, Artificial Sequence
  Description of Combined DNA/RNA Molecule Synthetic oligonucleotide
acguccagcuauagaauautt

SEQ ID NO: 229 - DNA, 21 bp, Artificial Sequence
  Description of Combined DNA/RNA Molecule Synthetic oligonucleotide
uacguccagcuauagaauatt

SEQ ID NO: 230 - DNA, 21 bp, Artificial Sequence
  Description of Combined DNA/RNA Molecule Synthetic oligonucleotide
uuacguccagcuauagaautt

SEQ ID NO: 231 - DNA, 21 bp, Artificial Sequence
  Description of Combined DNA/RNA Molecule Synthetic oligonucleotide
aaauuccuagaaucaauaatt

SEQ ID NO: 232 - DNA, 21 bp, Artificial Sequence
  Description of Combined DNA/RNA Molecule Synthetic oligonucleotide
aauuccuagaaucaauaaatt

SEQ ID NO: 233 - DNA, 21 bp, Artificial Sequence
  Description of Combined DNA/RNA Molecule Synthetic oligonucleotide
uuccuagaaucaauaaaggtt

SEQ ID NO: 234 - DNA, 21 bp, Artificial Sequence
  Description of Combined DNA/RNA Molecule Synthetic oligonucleotide
uccuagaaucaauaaagggtt

SEQ ID NO: 235 - DNA, 21 bp, Artificial Sequence
  Description of Combined DNA/RNA Molecule Synthetic oligonucleotide
cuagaaucaauaaagggcatt

SEQ ID NO: 236 - DNA, 21 bp, Artificial Sequence
  Description of Combined DNA/RNA Molecule Synthetic oligonucleotide
acauuugauaacaaugaagtt

SEQ ID NO: 237 - DNA, 21 bp, Artificial Sequence
  Description of Combined DNA/RNA Molecule Synthetic oligonucleotide
cauuugauaacaaugaagatt

SEQ ID NO: 238 - DNA, 21 bp, Artificial Sequence
  Description of Combined DNA/RNA Molecule Synthetic oligonucleotide
auuugauaacaaugaagaatt

SEQ ID NO: 239 - DNA, 21 bp, Artificial Sequence
  Description of Combined DNA/RNA Molecule Synthetic oligonucleotide
uuugauaacaaugaagaagtt

SEQ ID NO: 240 - DNA, 21 bp, Artificial Sequence
  Description of Combined DNA/RNA Molecule Synthetic oligonucleotide
aagugaaauacuaggaaugtt

SEQ ID NO: 241 - DNA, 21 bp, Artificial Sequence
  Description of Combined DNA/RNA Molecule Synthetic oligonucleotide
agugaaauacuaggaaugctt

SEQ ID NO: 242 - DNA, 21 bp, Artificial Sequence
  Description of Combined DNA/RNA Molecule Synthetic oligonucleotide
gugaaauacuaggaaugcutt

SEQ ID NO: 243 - DNA, 21 bp, Artificial Sequence
  Description of Combined DNA/RNA Molecule Synthetic oligonucleotide
ugaaauacuaggaaugcuutt

SEQ ID NO: 244 - DNA, 21 bp, Artificial Sequence
  Description of Combined DNA/RNA Molecule Synthetic oligonucleotide
gaaauacuaggaaugcuuctt

SEQ ID NO: 245 - DNA, 21 bp, Artificial Sequence
  Description of Combined DNA/RNA Molecule Synthetic oligonucleotide
aaauacuaggaaugcuucatt

SEQ ID NO: 246 - DNA, 21 bp, Artificial Sequence
  Description of Combined DNA/RNA Molecule Synthetic oligonucleotide
gaagcauuaaugaccaaugtt

SEQ ID NO: 247 - DNA, 21 bp, Artificial Sequence
  Description of Combined DNA/RNA Molecule Synthetic oligonucleotide
aagcauuaaugaccaaugatt

SEQ ID NO: 248 - DNA, 22 bp, Artificial Sequence
  Description of Combined DNA/RNA Molecule Synthetic oligonucleotide
cgauaauauaacagcaagatst

SEQ ID NO: 249 - DNA, 22 bp, Artificial Sequence
  Description of Combined DNA/RNA Molecule Synthetic oligonucleotide
cgauuauauuacaggaugatst

SEQ ID NO: 250 - DNA, 21 bp, Artificial Sequence
  Description of Combined DNA/RNA Molecule Synthetic oligonucleotide
uuauugauucuaggaauuutt

SEQ ID NO: 251 - DNA, 21 bp, Artificial Sequence
  Description of Combined DNA/RNA Molecule Synthetic oligonucleotide
uuuauugauucuaggaauutt

SEQ ID NO: 252 - DNA, 21 bp, Artificial Sequence
  Description of Combined DNA/RNA Molecule Synthetic oligonucleotide
ccuuuauugauucuaggaatt

SEQ ID NO: 253 - DNA, 21 bp, Artificial Sequence
  Description of Combined DNA/RNA Molecule Synthetic oligonucleotide
cccuuuauugauucuaggatt

SEQ ID NO: 254 - DNA, 21 bp, Artificial Sequence
  Description of Combined DNA/RNA Molecule Synthetic oligonucleotide
ugcccuuuauugauucuagtt

SEQ ID NO: 255 - DNA, 21 bp, Artificial Sequence
  Description of Combined DNA/RNA Molecule Synthetic oligonucleotide
cuucauuguuaucaaaugutt

SEQ ID NO: 256 - DNA, 21 bp, Artificial Sequence
  Description of Combined DNA/RNA Molecule Synthetic oligonucleotide
ucuucauuguuaucaaaugtt

SEQ ID NO: 257 - DNA, 21 bp, Artificial Sequence
  Description of Combined DNA/RNA Molecule Synthetic oligonucleotide
uucuucauuguuaucaaautt

SEQ ID NO: 258 - DNA, 21 bp, Artificial Sequence
  Description of Combined DNA/RNA Molecule Synthetic oligonucleotide
cuucuucauuguuaucaaatt

SEQ ID NO: 259 - DNA, 21 bp, Artificial Sequence
  Description of Combined DNA/RNA Molecule Synthetic oligonucleotide
cauuccuaguauuucacuutt

SEQ ID NO: 260 - DNA, 21 bp, Artificial Sequence
  Description of Combined DNA/RNA Molecule Synthetic oligonucleotide
gcauuccuaguauuucacutt

SEQ ID NO: 261 - DNA, 21 bp, Artificial Sequence
  Description of Combined DNA/RNA Molecule Synthetic oligonucleotide
agcauuccuaguauuucactt

SEQ ID NO: 262 - DNA, 21 bp, Artificial Sequence
  Description of Combined DNA/RNA Molecule Synthetic oligonucleotide
aagcauuccuaguauuucatt

SEQ ID NO: 263 - DNA, 21 bp, Artificial Sequence
  Description of Combined DNA/RNA Molecule Synthetic oligonucleotide
gaagcauuccuaguauuuctt

SEQ ID NO: 264 - DNA, 21 bp, Artificial Sequence
  Description of Combined DNA/RNA Molecule Synthetic oligonucleotide
ugaagcauuccuaguauuutt

SEQ ID NO: 265 - DNA, 21 bp, Artificial Sequence
  Description of Combined DNA/RNA Molecule Synthetic oligonucleotide
cauuggucauuaaugcuuctt

SEQ ID NO: 266 - DNA, 21 bp, Artificial Sequence
  Description of Combined DNA/RNA Molecule Synthetic oligonucleotide
ucauuggucauuaaugcuutt

SEQ ID NO: 267 - DNA, 22 bp, Artificial Sequence
  Description of Combined DNA/RNA Molecule Synthetic oligonucleotide
ucuugcuguuauauuaucgtst

SEQ ID NO: 268 - DNA, 22 bp, Artificial Sequence
  Description of Combined DNA/RNA Molecule Synthetic oligonucleotide
ucauccuguaauauaaucgtst

SEQ ID NO: 269 - DNA, 21 bp, Artificial Sequence
  Description of Combined DNA/RNA Molecule Synthetic oligonucleotide
cucuuagcaaagucaaguutt

SEQ ID NO: 270 - DNA, 21 bp, Artificial Sequence
  Description of Combined DNA/RNA Molecule Synthetic oligonucleotide
cugucauccagcaaauacatt

SEQ ID NO: 271 - DNA, 21 bp, Artificial Sequence
  Description of Combined DNA/RNA Molecule Synthetic oligonucleotide
ugucauccagcaaauacactt

SEQ ID NO: 272 - DNA, 21 bp, Artificial Sequence
  Description of Combined DNA/RNA Molecule Synthetic oligonucleotide
uaauagguauguuauaugctt

SEQ ID NO: 273 - DNA, 21 bp, Artificial Sequence
  Description of Combined DNA/RNA Molecule Synthetic oligonucleotide
auugagauagaaucuagaatt

SEQ ID NO: 274 - DNA, 21 bp, Artificial Sequence
  Description of Combined DNA/RNA Molecule Synthetic oligonucleotide
auucuaccauauauugaactt

SEQ ID NO: 275 - DNA, 21 bp, Artificial Sequence
  Description of Combined DNA/RNA Molecule Synthetic oligonucleotide
uucuaccauauauugaacatt

SEQ ID NO: 276 - DNA, 21 bp, Artificial Sequence
  Description of Combined DNA/RNA Molecule Synthetic oligonucleotide
ucuaccauauauugaacaatt

SEQ ID NO: 277 - DNA, 21 bp, Artificial Sequence
  Description of Combined DNA/RNA Molecule Synthetic oligonucleotide
aacuugacuuugcuaagagtt

SEQ ID NO: 278 - DNA, 21 bp, Artificial Sequence
  Description of Combined DNA/RNA Molecule Synthetic oligonucleotide
uguauuugcuggaugacagtt

SEQ ID NO: 279 - DNA, 21 bp, Artificial Sequence
  Description of Combined DNA/RNA Molecule Synthetic oligonucleotide
guguauuugcuggaugacatt

SEQ ID NO: 280 - DNA, 21 bp, Artificial Sequence
  Description of Combined DNA/RNA Molecule Synthetic oligonucleotide
gcauauaacauaccuauuatt

SEQ ID NO: 281 - DNA, 21 bp, Artificial Sequence
  Description of Combined DNA/RNA Molecule Synthetic oligonucleotide
uucuagauucuaucucaautt

SEQ ID NO: 282 - DNA, 21 bp, Artificial Sequence
  Description of Combined DNA/RNA Molecule Synthetic oligonucleotide
guucaauauaugguagaautt

SEQ ID NO: 283 - DNA, 21 bp, Artificial Sequence
  Description of Combined DNA/RNA Molecule Synthetic oligonucleotide
uguucaauauaugguagaatt

SEQ ID NO: 284 - DNA, 21 bp, Artificial Sequence
  Description of Combined DNA/RNA Molecule Synthetic oligonucleotide
uuguucaauauaugguagatt

SEQ ID NO: 285 - DNA, 20 bp, Artificial Sequence
  Description of Artificial Sequence Synthetic primer
agaaaacttgatgaaagaca

SEQ ID NO: 286 - DNA, 18 bp, Artificial Sequence
  Description of Artificial Sequence Synthetic primer
accataggcattcataaa

SEQ ID NO: 287 - RNA, 21 nt, Artificial Sequence
  Description of Artificial Sequence Synthetic oligonucleotide
gucaucacacugaauaccaau

SEQ ID NO: 288 - RNA, 23 nt, Artificial Sequence
  Description of Artificial Sequence Synthetic oligonucleotide
auugguauucagugugaugacac

SEQ ID NO: 289 - RNA, 25 nt, Artificial Sequence
  Description of Artificial Sequence Synthetic oligonucleotide
cuacacaaaucagcgauuuccaugu

SEQ ID NO: 290 - RNA, 25 nt, Artificial Sequence
  Description of Artificial Sequence Synthetic oligonucleotide
acauggaaaucgcugauuuguguag

SEQ ID NO: 291 - DNA, 21 bp, Artificial Sequence
  Description of Combined DNA/RNA Molecule Synthetic oligonucleotide
ggcucuaagcuaacugaagtt

SEQ ID NO: 292 - DNA, 21 bp, Artificial Sequence
  Description of Combined DNA/RNA Molecule Synthetic oligonucleotide
cuucaguuagcuuagagcctt

SEQ ID NO: 293 - RNA, 44 nt, Artificial Sequence
  Description of Artificial Sequence Synthetic oligonucleotide
cgacuggagcacgaggacacugacauggacugaaggaguagaaa

SEQ ID NO: 294 - DNA, 22 bp, Artificial Sequence
  Description of Artificial Sequence Synthetic primer
ctcaaagctctacatcattatc

SEQ ID NO: 295 - DNA, 23 bp, Artificial Sequence
  Description of Artificial Sequence Synthetic primer
cgactggagcacgaggacactga

SEQ ID NO: 296 - DNA, 28 bp, Artificial Sequence
  Description of Artificial Sequence Synthetic primer
ccactccatttgcttttacatgatatcc

SEQ ID NO: 297 - DNA, 26 bp, Artificial Sequence
  Description of Artificial Sequence Synthetic primer
ggacactgacatggactgaaggagta

SEQ ID NO: 298 - DNA, 29 bp, Artificial Sequence
  Description of Artificial Sequence Synthetic primer
gcttttacatgatatcccgcatctctgag

SEQ ID NO: 299 - DNA, 29 bp, Artificial Sequence
  Description of Artificial Sequence Synthetic primer
ccgcgggttctggcaatgataatctcaac

SEQ ID NO: 300 - DNA, 30 bp, Artificial Sequence
  Description of Artificial Sequence Synthetic primer
gccgcgtgtataattcataaaccttggtag

SEQ ID NO: 301 - DNA, 40 bp, Artificial Sequence
  Description of Artificial Sequence Synthetic primer
ggggccccgcggccgcgcattaatagcaagagttaggaag

SEQ ID NO: 302 - RNA, 19 nt, Artificial Sequence
  Description of Artificial Sequence Synthetic oligonucleotide
ggcucuuagcaaagucaag

SEQ ID NO: 303 - RNA, 19 nt, Artificial Sequence
  Description of Artificial Sequence Synthetic oligonucleotide
ggcucuuagcaaaguuaag

SEQ ID NO: 304 - RNA, 19 nt, Artificial Sequence
  Description of Artificial Sequence Synthetic oligonucleotide
ggcucuuagcaaggucaag

SEQ ID NO: 305 - RNA, 19 nt, Artificial Sequence
  Description of Artificial Sequence Synthetic oligonucleotide
cuugacuuugcuaagagcc

SEQ ID NO: 306 - DNA, 62 bp, Homo sapiens
ttgatctttgttgagtgtatcattcaacttgactttgctaagagccattgttgtatttgccc

SEQ ID NO: 307 - DNA, 62 bp, Homo sapiens
ctgatccttatttaatgtatcatttaacttgactttgctaagagccatcgttgtatttgccc

SEQ ID NO: 308 - DNA, 62 bp, Homo sapiens
  modified_base (51)..(51): a, c, g, t, unknown or other
ttgatctttgttgagtgtatcattcaacttgactttgctaagagccatcgntgtatttgccc

SEQ ID NO: 309 - DNA, 62 bp, Homo sapiens
ttgatctttgttgagtgtatcgttcaacttgactttgctaagagccattgttgtatttgccc

SEQ ID NO: 310 - DNA, 62 bp, Homo sapiens
  modified_base (1)..(1): a, c, g, t, unknown or other
ntgatcnttgttgagtgtatcnttcaacttgactttgctaagagccatngttgtatttgccc

SEQ ID NO: 311 - DNA, 62 bp, Homo sapiens; identical to SEQ ID NO: 309

SEQ ID NO: 312 - DNA, 62 bp, Artificial Sequence; identical to SEQ ID NO: 309
  Description of Artificial Sequence Synthetic oligonucleotide

SEQ ID NO: 313 - RNA, 30 nt, Homo sapiens
caacuugacuuugcuaagagccauuguugu

SEQ ID NO: 314 - RNA, 30 nt, Homo sapiens
caacuugaccuugcuaagagccauuguugu

SEQ ID NO: 315 - RNA, 21 nt, Artificial Sequence
  Description of Artificial Sequence Synthetic oligonucleotide
ggcucuuagcaaagucaaguu

SEQ ID NO: 316 - RNA, 21 nt, Artificial Sequence
  Description of Artificial Sequence Synthetic oligonucleotide
cuugacuuugcuaagagccau

SEQ ID NO: 317 - DNA, 22 bp, Artificial Sequence
  Description of Combined DNA/RNA Molecule Synthetic oligonucleotide
cuucacguuagcuuagagcctt

SEQ ID NO: 318 - DNA, 21 bp, Artificial Sequence
  Description of Combined DNA/RNA Molecule Synthetic oligonucleotide
cuuacgcugaguacuucgatt

SEQ ID NO: 319 - DNA, 21 bp, Artificial Sequence
  Description of Combined DNA/RNA Molecule Synthetic oligonucleotide
ucgaaguacucagcguaagtt

SEQ ID NO: 320 - DNA, 21 bp, Artificial Sequence
  Description of Combined DNA/RNA Molecule Synthetic oligonucleotide
ggcucuuagcaaagucaagtt

SEQ ID NO: 321 - DNA, 21 bp, Artificial Sequence
  Description of Combined DNA/RNA Molecule Synthetic oligonucleotide
ggcucuuagcaaagucaagtt

SEQ ID NO: 322 - DNA, 21 bp, Artificial Sequence
  Description of Combined DNA/RNA Molecule Synthetic oligonucleotide
ggcucuuagcaaagucaagtt

SEQ ID NO: 323 - DNA, 21 bp, Artificial Sequence
  Description of Combined DNA/RNA Molecule Synthetic oligonucleotide
cuugacuuugcuaagagcctt

SEQ ID NO: 324 - DNA, 21 bp, Artificial Sequence
  Description of Combined DNA/RNA Molecule Synthetic oligonucleotide
cuugacuuugcuaagagcctt

SEQ ID NO: 325 - DNA, 21 bp, Artificial Sequence
  Description of Combined DNA/RNA Molecule Synthetic oligonucleotide
cuugacuuugcuaagagcctt

SEQ ID NO: 326 - DNA, 21 bp, Artificial Sequence
  Description of Combined DNA/RNA Molecule Synthetic oligonucleotide
cuugacuuugcuaagagcctt

SEQ ID NO: 327 - RNA, 19 nt, Artificial Sequence
  Description of Artificial Sequence Synthetic oligonucleotide
ggcucuuagcaaagucaag

SEQ ID NO: 328 - RNA, 19 nt, Artificial Sequence
  Description of Artificial Sequence Synthetic oligonucleotide
ggcucuuagcaaagucaag

SEQ ID NO: 329 - RNA, 19 nt, Artificial Sequence
  Description of Artificial Sequence Synthetic oligonucleotide
ggcucuuagcaaagucaag

SEQ ID NO: 330 - RNA, 19 nt, Artificial Sequence
  Description of Artificial Sequence Synthetic oligonucleotide
cuugacuuugcuaagagcc

SEQ ID NO: 331 - RNA, 19 nt, Artificial Sequence
  Description of Artificial Sequence Synthetic oligonucleotide
cuugacuuugcuaagagcc

SEQ ID NO: 332 - RNA, 19 nt, Artificial Sequence
  Description of Artificial Sequence Synthetic oligonucleotide
cuugacuuugcuaagagcc

SEQ ID NO: 333 - RNA, 19 nt, Artificial Sequence
  Description of Artificial Sequence Synthetic oligonucleotide
cuugacuuugcuaagagcc

(19) United States
(12) Patent Application Publication
(10) Pub. No.: US 2010/0168205 A1
(43) Pub. Date: Jul. 1, 2010

(54) Methods and Compositions for Prevention or Treatment of RSV Infection Using Modified Duplex RNA Molecules

(75) Inventors: Rachel Meyers, Newton, MA (US); Rene Alvarez, Boxborough, MA (US); Geoff Cole, Needham, MA (US); Sayda Elbashir, Cambridge, MA (US)

Correspondence Address: ALNYLAM/FENWICK, SILICON VALLEY CENTER, 801 CALIFORNIA STREET, MOUNTAIN VIEW, CA, 94041, US

(73) Assignee: ALNYLAM PHARMACEUTICALS, INC., Cambridge, MA (US)

(21) Appl. No.: 12/605,299

(22) Filed: Oct. 23, 2009

Related U.S. Application Data
(60) Provisional application No. 61/160,679, filed on Mar. 16, 2009.
(60) Provisional application No. 61/108,001, filed on Oct. 23, 2008.

(51) Int. Cl. A61K 31/713; C07H 21/02; A61P 31/14

(52) U.S. Cl. 514/44 A; 536/24.5

(57) ABSTRACT

Methods and compositions are provided for the prevention or treatment of RSV infection in a human. The methods include administering one or more doses of a composition comprising an siRNA. The dose can be formulated for topical or parenteral administration. Topical administration includes administration as a nasal spray, or by inhalation of respirable particles or droplets. The siRNA preferably comprises a sense strand and antisense strand with modified nucleotides.

### RELATED APPLICATIONS

This applications claims the benefit of U.S. provisional application 61/160,679, filed Mar. 16, 2009, and U.S. provisional application 61/108,001, filed Oct. 23, 2008, the disclosure of each of which is hereby incorporated by reference.

### STATEMENT OF GOVERNMENT SUPPORT

This invention was made with government support under U.S. Army Medical Research and Material Command/U.S. Army Medical Research Acquisition Activity Contract #W81XWH-08-1-0001. The U.S. Government has certain rights in the invention.

### SEQUENCE LISTING

The instant application contains a Sequence Listing which has been submitted via EFS-Web and is hereby incorporated by reference in its entirety. Said ASCII copy, created on Oct. 23, 2009, is named 16113US.txt, and is 107 kb in size.

### TECHNICAL FIELD

The invention relates to the field of respiratory syncytial viral (RSV) therapy and compositions and methods for modulating viral replication, and more particularly to the down-regulation of a gene(s) of a respiratory syncytial virus by oligonucleotides via RNA interference which are administered locally to the lungs and nasal passage via inhalation or intranasal administration or systemically via injection or intravenous infusion.

### BACKGROUND

By virtue of its natural function the respiratory tract is exposed to a slew of airborne pathogens that cause a variety of respiratory ailments. Viral infection of the respiratory tract is the most common cause of infantile hospitalization in the developed world with an estimated 91,000 annual admissions in the US at a cost of $300 M. Human respiratory syncytial virus (RSV) and parainfluenza virus (PIV) are two major agents of respiratory illness; together, they infect the upper and lower respiratory tracts, leading to croup, pneumonia and bronchiolitis (Openshaw, P. J. M. Respir. Res. 3 (Suppl 1), S15-S20 (2002), Easton, A. J., et al., Clin. Microbiol. Rev. 17, 390-412 (2004)).

RSV alone infects up to 65% of all babies within the first year of life, and essentially all within the first 2 years. It is a significant cause of morbidity and mortality in the elderly as well. Immunity after RSV infection is neither complete nor lasting, and therefore, repeated infections occur in all age groups. Infants experiencing RSV bronchiolitis are more likely to develop wheezing and asthma later in life. Research for effective treatment and vaccine against RSV has been ongoing for nearly four decades with few successes (Openshaw, P. J. M. Respir. Res. 3 (Suppl 1), S15-S20 (2002), Maggon, K. et al, Rev. Med. Virol. 14, 149-168 (2004)).

Currently, no vaccine is clinically approved for RSV. Strains of RSV also exist for nonhuman animals such as the cattle, goat, pig and sheep, causing loss to agriculture and the dairy and meat industry (Easton, A. J., et al., Clin. Microbiol. Rev. 17, 390-412 (2004)).

Both RSV and PIV contain nonsegmented negative-strand RNA genomes and belong to the Paramyxoviridae family. A number of features of these viruses have contributed to the difficulties of prevention and therapy. The viral genomes mutate at a high rate due to the lack of a replicational proof-reading mechanism of the RNA genomes, presenting a significant challenge in designing a reliable vaccine or antiviral (Sullender, W. M. Clin. Microbiol. Rev. 13, 1-15 (2000)). Promising inhibitors of the RSV fusion protein (F) were abandoned partly because the virus developed resistant mutations that were mapped to the F gene (Razinkov, V., et. al., Antivir. Res. 55, 189-200 (2002), Morton, C. J. et al. Virology 311, 275-288 (2003)). Both viruses associate with cellular proteins, adding to the difficulty of obtaining cell-free viral material for vaccination (Burke, E., et al., Virology 252, 137-148 (1998), Burke, E., et al., J. Virol. 74, 669-675 (2000), Gupta, S., et al., J. Virol. 72, 2655-2662 (1998)). Finally, the immunology of both, and especially that of RSV, is exquisitely complex (Peebles, R. S., Jr., et al., Viral. Immunol. 16, 25-34 (2003), Haynes, L. M., et al., J. Virol. 77, 9831-9844 (2003)). Use of denatured RSV proteins as vaccines leads to “immunopotentiation” or vaccine-enhanced disease (Polack, F. P. et al. J. Exp. Med. 196, 859-865 (2002)). The overall problem is underscored by the recent closure of a number of anti-RSV biopharma programs.

The RSV genome comprises a single strand of negative sense RNA that is 15,222 nucleotides in length and yields eleven major proteins. (Falsey, A. R., and E. E. Walsh, 2000, Clinical Microbiological Reviews 13:371-84.) Two of these proteins, the F (fusion) and G (attachment) glycoproteins, are the major surface proteins and the most important for inducing protective immunity. The SH (small hydrophobic) protein, the M (matrix) protein, and the M2 (22 kDa) protein are associated with the viral envelope but do not induce a protective immune response. The N (major nucleocapsid associated protein), P (phosphoprotein), and L (major polymerase protein) proteins are found associated with virion RNA. The two non-structural proteins, NS1 and NS2, presumably participate in host-virus interaction but are not present in infectious virions.

Human RSV strains have been classified into two major groups, A and B. The G glycoprotein has been shown to be the most divergent among RSV proteins. Variability of the RSV G glycoprotein between and within the two RSV groups is believed to be important to the ability of RSV to cause yearly outbreaks of disease. The G glycoprotein comprises 289-299 amino acids (depending on RSV strain), and has an intracellular, transmembrane, and highly glycosylated stalk structure of 90 kDa, as well as heparin-binding domains. The glycoprotein exists in secreted and membrane-bound forms.

Successful methods of treating RSV infection are currently unavailable (Maggon K and S. Barik, 2004, Reviews in Medical Virology 14:149-68). Infection of the lower respiratory tract with RSV is a self-limiting condition in most cases. No definitive guidelines or criteria exist on how to treat or when to admit or discharge infants and children with the disease. Hypoxia, which can occur in association with RSV infection, can be treated with oxygen via a nasal cannula. Mechanical ventilation for children with respiratory failure, shock, or recurrent apnea can lower mortality. Some physicians prescribe steroids. However, several studies have shown that steroid therapy does not affect the clinical course of infants and children admitted to the hospital with bronchiolitis. Thus corticosteroids, alone or in combination with bronchodilators, may be useless in the management of bronchiolitis in otherwise healthy unventilated patients. In infants and children with underlying cardiopulmonary diseases, such as bronchopulmonary dysphasia and asthma, steroids have also been used.

Ribavirin, a guanosine analogue with antiviral activity, has been used to treat infants and children with RSV bronchiolitis since the mid 1980s, but many studies evaluating its use have shown conflicting results. In most centers, the use of ribavirin is now restricted to immunocompromised patients and to those who are severely ill.

The severity of RSV bronchiolitis has been associated with low serum retinol concentrations, but trials in hospitalized children with RSV bronchiolitis have shown that vitamin A supplementation provides no beneficial effect. Therapeutic trials of 1500 mg/kg intravenous RSV immune globulin or 100 mg/kg inhaled immune globulin for RSV lower-respiratory-tract infection have also failed to show substantial beneficial effects.

In developed countries, the treatment of RSV lower-respiratory-tract infection is generally limited to symptomatic therapy. Antiviral therapy is usually limited to life-threatening situations due to its high cost and to the lack of consensus on efficacy. In developing countries, oxygen is the main therapy (when available), and the only way to lower mortality is through prevention.

RNA interference or “RNAi” is a term initially coined by Fire and co-workers to describe the observation that double-stranded RNA (dsRNA) can block gene expression when it is introduced into worms (Fire et al., Nature 391:806-811, 1998). Short dsRNA directs gene-specific, post-transcriptional silencing in many organisms, including vertebrates, and has provided a new tool for studying gene function. RNAi has been suggested as a method of developing a new class of therapeutic agents. However, to date, these have remained mostly as suggestions with no demonstrate proof that RNAi can be used therapeutically.

Therefore, there is a need for safe and effective vaccines against RSV, especially for infants and children. There is also a need for therapeutic agents and methods for treating RSV infection at all ages and in immuno-compromised individuals. There is also a need for scientific methods to characterize the protective immune response to RSV so that the pathogenesis of the disease can be studied, and screening for therapeutic agents and vaccines can be facilitated.

### SUMMARY

The present invention improves the art by providing methods and compositions effective for modulating or preventing RSV infection using dsRNAs comprising modified nucleotides. Specifically, the present invention advances the art by providing iRNA agents that have been shown to reduce RSV levels in vitro and in vivo, as well as being effective against both major subtypes of RSV, and a showing of therapeutic activity of this class of molecules. More specifically, the present invention comprises dsRNA compositions comprising modified nucleotides that are extremely effective at reducing RSV titer in cells in vitro and in vivo, and possess, in addition, significant beneficial properties including enhanced stability and a reduction in immunomodulatory side effects.

The present invention is based on the in vitro and in vivo demonstration that RSV can be inhibited through intranasal administration or inhalation (e.g., through the mouth) of iRNA agents, as well as by parenteral administration of such agents, and the identification of potent iRNA agents from the P, N and L gene of RSV that can reduce RNA levels with both the A and B subtype of RSV. Based on these findings, the present invention provides specific compositions and methods that are useful in reducing RSV mRNA levels, RSV protein levels and RSV viral titers in a subject, e.g., a mammal, such as a human. It is shown herein that administration of multiple doses of an siRNA agent over a course of days can provide improved results. For example, in a preferred embodiment a preselected amount of siRNA agent results in better inhibition of gene expression when administered as fractional doses over the course of more than one day.

In one aspect, the invention provides for an siRNA composition that comprises a therapeutically effective amount of ALN-RSV01. ALN-RSV01 is also referred to herein as AL-DP-2017 or AD-2017. The structure of ALN-RSV01, along with details about its manufacture are fully described in U.S. Provisional Application No. 61/021,309, filed on Jan. 15, 2008, which is herein incorporated by reference in its entirety, for all purposes, along with U.S. patent application Ser. No. 12/335,467, filed, Dec. 15, 2008; U.S. Pat. No. 7,507,809; and U.S. Pat. No. 7,517,865.

In one embodiment the invention provides for a lyophilized powder. In another embodiment the invention provides for a liquid solution, and in another embodiment a liquid suspension, and in another embodiment a dry powder comprising said amount of ALN-RSV01 or modified ALN-RSV01. In one embodiment, the therapeutically effective amount of ALN-RSV01 or modified ALN-RSV01 is less than or equal to 150 mg of anhydrous oligonucleotide. In another embodiment, the therapeutically effective amount is equal to 150 mg of anhydrous oligonucleotide. In another embodiment, the therapeutically effective amount is equal to 75 mg of anhydrous oligonucleotide. In one embodiment, the liquid solution is formulated to have an osmolality ranging from 200-400 mOsm/kg. In certain embodiments, the liquid solution is a buffered. In certain embodiments, the pH of the liquid solution is between 5 and 8. In other embodiments, the pH of the liquid solution is between 5.6 and 7.6.

In other aspects, the invention provides for a method of preventing or treating RSV infection in a human subject. In one aspect the invention provides such a method of prevention or treating by administering a composition that comprises a therapeutically effective amount of modified ALN-RSV01.

The present invention is based on the in vitro and in vivo demonstration that RSV can be inhibited through intranasal administration of iRNA agents, as well as by parenteral administration of such agents, and the identification of potent iRNA agents from the P, N and L gene of RSV that can reduce RNA levels with both the A and B subtype of RSV. Based on these findings, the present invention provides specific compositions and methods that are useful in reducing RSV mRNA levels, RSV protein levels and RSV viral titers in a subject, e.g., a mammal, such as a human. It is shown herein that administration of multiple doses of an siRNA agent over a course of days can provide improved results. E.g., in a preferred embodiment a preselected amount of siRNA agent results in better inhibition of gene expression when administered as fractional doses over the course of more than one day.

In one aspect, the invention provides for an siRNA composition that includes a therapeutically effective amount of ALN-RSV01. ALN-RSV01 is the same as AL-DP-2017. The structure of AL-DP-2017, along with details about its manufacture are fully described in co-owned U.S. Provisional Application No. 61/021,309 filed on Jan. 15, 2008, which is herein incorporated by reference in its entirety, for all purposes.

In one embodiment, the invention includes a modified double-stranded ribonucleic acid (dsRNA) for inhibiting expression of a Respiratory Syncytial Virus (RSV) gene, wherein the dsRNA includes a modified antisense strand, the antisense strand including a region complementary to a part of the N gene of RSV, wherein the region of complementarity is less than 30 nucleotides in length and the antisense strand includes 15 or more contiguous nucleotides of the sequence CUUGACUUUGCUAAGAGCC (SEQ ID NO: 305), wherein at least three nucleotides in the antisense sequence are modified, and wherein the antisense strand is complementary to at least 15 contiguous nucleotides in the sense strand. In a related embodiment, the sense strand includes the modified nucleotide sequence selected from the group consisting of A-30629 (SEQ ID NO: 322), A-30631 (SEQ ID NO: 320) and A-30633 (SEQ ID NO: 321). In another related embodiment, the sense strand includes 15 or more contiguous nucleotides of GGCUCUUAGCAAAGUCAAG (SEQ ID NO: 302), and wherein at least three nucleotides in the sense strand are modified. In another related embodiment, the antisense strand includes the modified nucleotide sequence selected from the group consisting of A-30653 (SEQ ID NO: 326), A-30648 (SEQ ID NO:323), A-30650 (SEQ ID NO:325), and A-30652 (SEQ ID NO:324). In another related embodiment, the sense strand consists of the modified nucleotide sequence A-30629 (SEQ ID NO: 322). In another related embodiment, the sense strand consists of the modified nucleotide sequence A-30631 (SEQ ID NO: 320). In another related embodiment, the sense strand consists of the modified nucleotide sequence A-30633 (SEQ ID NO: 321). In another related embodiment, the antisense strand consists of the modified nucleotide sequence A-30653 (SEQ ID NO: 326). In another related embodiment, the antisense strand consists of the modified nucleotide sequence A-30648 (SEQ ID NO:323). In another related embodiment, the antisense strand consists of the modified nucleotide sequence A-30652 (SEQ ID NO:324). In another related embodiment, the antisense strand consists of the modified nucleotide sequence A-30653 (SEQ ID NO: 326). In another related embodiment, the antisense strand consists of the modified nucleotide sequence A-30650 (SEQ ID NO:325). In another related embodiment, the antisense strand consists of the modified nucleotide sequence A-30653 (SEQ ID NO: 326). In another related embodiment, the sense strand consists of the modified nucleotide sequence A-30629 and the antisense strand consists of the modified nucleotide sequence A-30653 (SEQ ID NO: 326). In another related embodiment, the sense strand consists of the modified nucleotide sequence A-30629 and the antisense strand consists of the modified nucleotide sequence A-30648 (SEQ ID NO:323).

In another embodiment, a dsRNA's region of complementarity between the antisense strand and the N gene of RSV is 19 nucleotides in length. In a related embodiment, the region of complementarity includes the nucleotide sequence CUUGACUUUGCUAAGAGCC (SEQ ID NO: 305).

In another embodiment, each strand of a dsRNA is 19, 20, 21, 22, 23, or 24 nucleotides in length. In a related embodiment, each strand of a dsRNA is 21 nucleotides in length.

In another embodiment, at least one of the dsRNA modified nucleotides is chosen from the group of: a 2′-O-methyl modified nucleotide, a nucleotide including a 5′-phosphorothioate group, and a terminal nucleotide linked to a cholesteryl derivative or dodecanoic acid bisdecylamide group. In a related embodiment, the modified nucleotide is chosen from the group of: a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base including nucleotide. In another related embodiment, each strand of a dsRNA includes at least one 2′-O-methyl modified pyrimidine nucleotide. In another related embodiment, each strand of a dsRNA includes between three and six 2′-O-methyl modified pyrimidine nucleotides. In another related embodiment, the antisense strand of a dsRNA includes three 2′-O-methyl modified pyrimidine nucleotides. In another related embodiment, antisense strand of a dsRNA includes three 2′-O-methyl modified pyrimidine nucleotides and the sense strand includes four to six 2′-O-methyl modified pyrimidine nucleotides.

In another embodiment, each strand of a dsRNA includes a dTdT overhang. In a related embodiment, a dTdT overhang includes a phosphorothioate linkage. In another related embodiment, a dsRNA antisense strand includes three 2′-O-methyl modified nucleotides and the sense strand includes four to six 2′-O-methyl modified nucleotides, and wherein each strand includes a modified or unmodified dTdT overhang. In another related embodiment, a dTdT overhang on each strand of a dsRNA includes a phosphorothioate linkage.

In another embodiment, a dsRNA can be formulated for intranasal or intrapulmonary delivery. In a related embodiment, the dsRNA is formulated in a buffered saline solution. In another related embodiment, the dsRNA is formulated in a phosphate buffered saline solution. In another related embodiment, the dsRNA reduces viral titer levels by 2-3 orders of magnitude at a dose of 0.1-1.0 mgs/kg, relative to a PBS control group. In another related embodiment, the dsRNA reduces viral titer in vivo in a dose-dependent manner relative to a PBS control group as measured by a plaque assay. In another related embodiment, administration of the dsRNA in vivo results in reduced immunostimulation relative to ALN-RSV01, as measured by TNF-α, IL-6 and IL1-RA ELISA assays on epithelial lining fluid obtained by bronchoalveolar lavage. In another related embodiment, the dsRNA is non-immunostimulatory. In another related embodiment, administration of the dsRNA does not result in immunostimulatory activity in human peripheral blood mononuclear cells (PBMCs) as measured by IFN-alpha ELISA assays. In another related embodiment, administration of the dsRNA results in reduced immunostimulatory activity by at least an order of magnitude in human peripheral blood mononuclear cells (PBMCs) relative to ALN-RSV01, as measured by real-time PCR measurements of TNF-α, IL-6 and IP-10 mRNA. In another related embodiment, administration of the dsRNA results in no immunostimulatory activity in human peripheral blood mononuclear cells (PBMCs) as measured by real-time PCR measurements of G-CSF or IL1-RA mRNA. In another related embodiment, administration of the dsRNA results in 13-26 fold less immunostimulatory activity in human peripheral blood mononuclear cells (PBMCs) relative to ALN-RSV01, as measured by real-time PCR measurements of interferon-inducible cytokine mRNAs selected from the group consisting of: IF127, IFIT1, IFIT2, viperin, OAS3, IL-6, and IP-10 mRNAs. In another related embodiment, administration of the dsRNA results in a 5 fold decrease in IFN-γ mRNA induction in human peripheral blood mononuclear cells (PBMCs) relative to ALN-RSV01, as measured by real-time PCR. In another related embodiment, prophylactic administration of the dsRNA to a subject reduces viral titer as effectively as ALN-RSV01. In another related embodiment, prophylactic administration of the dsRNA to a subject leads to a 500-1000 fold reduction in viral titer in the subject following infection with RSV. In another related embodiment, administration of the dsRNA to an RSV-infected subject results in a 300 to 600 fold reduction in viral titer relative to a control. In another related embodiment, the sense or antisense strand of the dsRNA has a half-life of at least 24 hours in human serum. In another related embodiment, the sense or antisense strand of the dsRNA has a half-life of at least 48 hours in human serum. In another related embodiment, the sense or antisense strand of the dsRNA has a half-life of at least 10 hours in human nasal washes. In another related embodiment, the sense or antisense strand of the dsRNA has a half-life longer than that observed for the corresponding sense or antisense strands of ALN-RSV01 under identical conditions.

In another embodiment, the invention includes a modified dsRNA for inhibiting expression of a Respiratory Syncytial Virus (RSV) gene, wherein the modified dsRNA includes a modified sense strand selected from Table 7, 9, 10, 11, 12, 13 or 15 and a modified antisense strand selected from Table 7, 9, 10, 11, 12, 13 or 15.

In another embodiment, the invention includes a modified dsRNA for inhibiting expression of a Respiratory Synctial Virus (RSV) gene, wherein the dsRNA is selected from Table 7, 9, 10, 11, 12, 13, 15 or 20.

In another embodiment, the invention includes a cell containing any one or more of the above dsRNAs.

In another embodiment, the invention includes a vector including a nucleotide sequence that encodes at least one strand of any one or more of the above dsRNAs. In a related embodiment, the invention includes a cell including one or more of the above vectors.

In another embodiment, the invention includes a pharmaceutical composition for reducing viral titer or retarding viral proliferation in a cell of a subject, wherein the composition includes any one or more of the above dsRNAs and a pharmaceutically acceptable carrier.

In one embodiment the invention provides for a lyophilized powder. In another embodiment the invention provides for a liquid solution, and in another embodiment a liquid suspension, and in another embodiment a dry powder including the amount of ALN-RSV01. In one embodiment, the therapeutically effective amount of ALN-RSV01 is less than or equal to 150 mg of anhydrous oligonucleotide. In another embodiment, the therapeutically effective amount is equal to 150 mg of anhydrous oligonucleotide. In another embodiment, the therapeutically effective amount is equal to 75 mg of anhydrous oligonucleotide. In one embodiment, administration of the therapeutically effective amount to a human subject produces in the subject no significant increase in the subject's white cell count. In another embodiment, administration of the therapeutically effective amount to a human subject produces in the concentration in a subject's inflammatory cytokine(s). In one embodiment those cytokine(s) are one or more of CRP, G-CSF, IL1-RA, or TNF. In one embodiment, the liquid solution is formulated to have an osmolality ranging from 200-400 mOsm/kg. In certain embodiments, the liquid solution is a buffered. In certain embodiments, the pH of the liquid solution is between 5 and 8. In other embodiments, the pH of the liquid solution is between 5.6 and 7.6.

In other aspects, the invention provides for a method of preventing or treating RSV infection in a human subject. In one aspect the invention provides such a method of prevention or treating by administering a composition that includes a therapeutically effective amount of ALN-RSV01. In one aspect the composition is administered intranasally or by inhalation. In one aspect, the composition is administered as an aerosolized liquid. In certain aspects, the aerosolized liquid is a nasal spray. In other embodiments, the aerosolized liquid is produced by a nebulizer. In one embodiment, the composition is administered in a volume of 0.5 ml of aerosolized liquid to each nostril of the human subject. In another embodiment, a plurality of doses are administered. In one embodiment, the administration of multiple doses are one a once-daily dose schedule. In one embodiment, a single dose is administered. In another embodiment the single administered dose has an efficacy equal to that of the same amount of drug present in that single dose, administered in a series of divided doses.

In another embodiment, the invention includes a method of inhibiting RSV replication in a cell of a subject, the method including: (a) contacting the cell with any one or more of the above dsRNAs; and (b) maintaining the cell produced in step (a) for a time sufficient to obtain degradation of an mRNA transcript of an RSV gene, thereby inhibiting replication of the virus in the cell.

In another embodiment, the invention includes a method of reducing RSV titer in a cell of a subject, including administering to the subject a therapeutically effective amount of any one or more of the above dsRNAs. In a related embodiment, the dsRNA is administered to a human subject at about 0.1, 0.2, 0.3, 0.4, 0.5, 0.6, 0.7, 0.8, 0.9, 1.0, 2.5, or 5.0 mg/kg. In another related embodiment, the dsRNA is administered to the human at about 1.0 mg/kg. In another related embodiment, administration is intranasal or intrapulmonary. In another related embodiment, the composition is administered as an aerosol. In another related embodiment, the aerosol is a nasal spray. In another related embodiment, the aerosol is produced by a nebulizer. In another related embodiment, the nebulizer is a PARI eFlow® 30L nebulizer.

In another related embodiment, the method includes administering a plurality of doses of the composition. In a related embodiment, at least one of the plurality of doses is administered once daily. In another related embodiment, the plurality of doses is two or three doses. In another related embodiment, two doses are administered within a single day. In another related embodiment, three doses are administered within a single day. In another related embodiment, the subject is presently infected with RSV when the first of the plurality of doses is administered. In another embodiment, administering reduces RSV protein, RSV mRNA, RSV peak viral load, time to peak RSV viral load, duration of RSV viral shedding, RSV viral AUC, FEV1, BOS or RSV titer in the subject. In another related embodiment, administering of the plurality of doses is by inhalation and delivers a total dose of between 0.6 mg/kg and 5 mg/kg of anhydrous oligonucleotide to the subject.

In another related embodiment, the above method can further include determining a characteristic of RSV infection, wherein the characteristic is selected from RSV mRNA, RSV peak viral load, time to peak RSV viral load, duration of RSV viral shedding, RSV viral AUC, or RSV titer in one or more cells of the subject. In a related embodiment, characteristic of RSV infection is determined by quantitative RT-PCR (qRT-PCR) analysis of a nasal swab sample and/or a sputum sample from the subject. In another related embodiment, administration of the composition to the subject is started within seven days of onset of symptoms of RSV infection, wherein the symptoms include a decrease in FEV₁, fever, new onset rhinorrhea, sore throat, nasal congestion, cough, wheezing, headache, myalgia, chills, or shortness of breath. In another related embodiment, administration of any one or more of the above dsRNAs results in reduced interferon-α production in the subject relative to ALN-RSV01. In another related embodiment, administration of any one or more of the above dsRNAs results in reduced cytokine production in the subject relative to ALN-RSV01. In another related embodiment, administration of any one or more of the above dsRNAs results in reduced induction of inteferon-inducible genes in the subject relative to ALN-RSV01.

In another related embodiment, the subject is a lung transplant recipient. In another related embodiment, the subject is less than 18 years old. In another related embodiment, the subject is less than 12 years old. In another related embodiment, the subject is less than 6 years old.

The details of one or more embodiments of the invention are set forth in the accompanying drawings and the description below. Other features, objects, and advantages of the invention will be apparent from this description, the drawings, and from the claims. This application incorporates all cited references, patents, and patent applications by references in their entirety for all purposes.

### BRIEF DESCRIPTION OF DRAWINGS AND TABLES

FIG. 1: In vitro inhibition of RSV using iRNA agents. iRNA agents provided in Table 1 (a-c) were tested for anti-RSV activity in a plaque formation assay as described in the Examples. Each column (bar) represents an iRNA agent provided in Table 1 (a-c), e.g., column 1 is the first agent in Table 1a, etc. Active iRNA agents were identified.

FIG. 2: In vitro dose response inhibition of RSV using iRNA agents. Examples of active agents from Table 1 were tested for anti-RSV activity in a plaque formation assay as described in the Examples at four concentrations. A dose dependent-response was found with active iRNA agents tested.

FIG. 3: In vitro inhibition of RSV B subtype using iRNA agents. iRNA agents provided in FIG. 2 were tested for anti-RSV activity against subtype B in a plaque formation assay as described in the Examples. Subtype B was inhibited by the iRNA agents tested.

FIG. 4: In vivo inhibition of RSV using iRNA agents. Agents as described in the figure were tested for anti-RSV activity in a mouse model as described in the Examples. The iRNA agents were effective at reducing viral titers in vivo.

FIG. 5: In vivo inhibition of RSV using AL-DP-1730. AL-DP-1730 was tested for dose-dependent activity using the methods provided in the Examples. The agent showed a dose-dependent response.

FIG. 6: In vivo inhibition of RSV using iRNA agents. iRNA agents described in the Figure were tested for anti-RSV activity in vivo as described in the Examples.

FIG. 7: In vivo inhibition of RSV using iRNA agents. iRNA agents described in the Figure were tested for anti-RSV activity in vivo as described in the Examples.

FIG. 8: Sequence analysis of RSV N genes from clinical isolates.

FIG. 9: Sequence analysis of RSV N genes from slower growing clinical RSV isolates showing single base mutation in ALN-RSV01 recognition site for isolate LAP6824.

FIG. 10: Flow chart illustrating manufacturing process for ALN-RSV01 drug substance.

FIG. 11: Illustration of cycle of steps involved in solid-phase synthesis of ALN-RSV01 drug substance.

FIG. 12: Illustration of cleavage and deprotection reactions following solid-phase synthesis of ALN-RSV01 drug substance.

FIGS. 13A and 13B: In vivo inhibition of RSV using iRNA agents delivered via aerosol. iRNA agents described in the Figure were tested for anti-RSV activity in vivo as described in the EXAMPLE

FIG. 14: In vivo protection against RSV infection using iRNA agents. iRNA agents described in the Figure were tested prior to RSV challenge to test for protective activity.

FIG. 15: In vitro activity of nebulized iRNA agent. iRNA agent as described was nebulized and shown to retain activity in an in vitro assay of RSV infection.

FIG. 16: Lung function (FEV1 (L)) 30 minutes post-dose in human subjects after inhalation of siRNA ALN-RSV01 targeting RSV. Dose in mg/kg (assuming average human weight of 70 kg).

FIG. 17: Mean plasma level of siRNA ALN-RSV01 targeting RSV in man vs. non human primates after inhalation.

FIG. 18: White cell count in human subjects after inhalation multi-dosing (once daily for three days with total dose of 0.6 mg/kg) of siRNA ALN-RSV01 targeting RSV.

FIG. 19: RSV in the lung following administration of siRNA ALN-RSV01. RSV instillation was intranasal (10⁶ pfu). Fixed total dose of siRNA was 120 μg. Single administration is indicated by -4 hr, D1, D2, D3; split dose over three days is indicated by D1+D2+D3.

FIG. 20: Structure of ALN-RSV01 duplex. ALN-RSV01 is a synthetic double-stranded RNA (dsRNA) oligonucleotide (SEQ ID NOS: 1 and 2, respectively, in order of appearance) formed by hybridization of two partially complementary single strand RNAs in which the 3′ ends are capped with two thymidine units. Hybridization occurs across 19 ribonucleotide base pairs to yield the ALN-RSV01 molecule. All the phosphodiester functional groups are negatively charged at neutral pH with a sodium ion as the counter ion.

FIG. 21: In vitro IC₅₀ of ALN-RSV01. Vero cells in 24-well plates were transfected with decreasing concentration of ALN-RSV01 followed by infection with 200-300 pfu of RSV/A2. At 5 days post-infection, cells were fixed, immunostained, and counted. Percent activity against PBS was plotted and IC₅₀ measured using XLFit software.

FIG. 22: In vivo activity of ALN-RSV01 following single and multidosing in BALB/c mice. A) ALN-RSV01 in vivo dose response curve. BALB/c mice were intranasally treated with ALN-RSV01 at increasing concentrations (25 μg, 50 μg, or 100 μg), control siRNA AL-DP-1730 or PBS 4 hours prior to infection with 1×10⁶ pfu of RSV/A2. Lungs were harvested and virus quantified by standard immunostaining plaque assay and plotted as log pfu/g lung. B) ALN-RSV01 multidose study. BALB/c mice were intranasally treated with ALN-RSV01 or mismatch siRNA (1730) at either 40 μg, 80 μg, or 120 μg (single dose treatment) or 40 μg (multidose, daily treatment). Lungs were harvested and virus quantified by immunostaining plaque assay on D5. −4, 4 hours prior to infection; D1, day 1 post-infection; D2, day 2 post-infection; D3, day 3 post-infection. C) ALN-RSV01 same day multidose study. BALB/c mice were intranasally treated with ALN-RSV01 or mismatch siRNA (1730) at either 40 μg, 60 μg, 80 μg, or 120 μg for single dose groups at days 1 or 2 post-RSV infection, or 40 μg 2× or 3× daily for multidose groups at day 1 or 2 post-RSV infection. Lungs were harvested and virus quantified by immunostaining plaque assay.

FIG. 23: ALN-RSV01 is a modest stimulatory of IFNα and TNFα in vitro. siRNAs (ALN-RSV01 or mismatch positive controls, 7296 and 5048) were transfected into peripheral blood mononuclear cells and assayed by ELISA for induction of cytokines at 24 hrs post transfection. A) IFNα induction; B) TNFα induction.

FIG. 24: TNFα and IFNα stimulatory mismatched siRNAs do not modulate RSV in vivo. A) siRNAs transfected into peripheral blood mononuclear cells were assayed for TNFα stimulation at 24 hrs post transfection. B) siRNAs transfected into peripheral blood mononuclear cells were assayed for IFNα stimulation at 24 hrs post transfection. C) Lung viral concentrations from mice intranasally dosed with RSV at day 0 and with RSV-specific siRNAs (ALN-RSV01) or mismatch control immunostimulatory siRNAs (2153 and 1730) at 4 hrs pre inoculation. Lung RSV concentrations were measured by immunostaining plaque assay at day 5 post infection.

FIG. 25: Chemically modified ALN-RSV01 (AL-DP-16570) is immunologically silent, while maintaining antiviral activity. A) Chemical composition of modified ALN-RSV01 (AL-DP-16570), lowercase letters indicated 2′OMe modification and HP indicates hydroxyproline (FIG. 25 A discloses SEQ ID NOS: 1 and 2 (when modifications are ignored), in order of appearance). B) AL-DP-16570 or positive control siRNAs AL-DP-5048 and AL-DP-7296 were transfected into peripheral blood mononuclear cells and assayed for IFNα stimulation at 24 hour post-transfection. C) AL-DP-16570 or positive control siRNAs AL-DP-5048 and AL-DP-7296 were transfected into peripheral blood mononuclear cells and assayed for TNFα stimulation at 24 hours post-infection. D) Lung viral concentrations from mice intranasally dosed with RSV at day 0 and with ALN-RSV01, AL-DP-16570, or mismatch control at indicated concentrations, at 4 hrs pre inoculation. Lung RSV concentrations were measured by immunostaining plaque assay at day 5 post infection.

FIG. 26: ALN-RSV01 viral inhibition is mediated by RNAi in vivo. Shown is a schematic representation of the 5′-RACE assay used to demonstrate the generation of site-specific cleavage product. Boxed are the results of sequence analysis of individual clones isolated from per amplification of linker adapted RSV N gene cDNAs generated from an in vivo viral inhibition assay in which mice were inoculated with RSV at day 0 and treated with ALN-RSV01, AL-DP-1730 or PBS at day 3, followed by lung homogenization and evaluation by RACE at day 5 post infection.

FIG. 27: Genotype analysis of RSV primary isolates. Primary isolates were propagated and the RSV G gene was amplified by RT-PCR followed by nucleotide sequence analysis. Phylogenetic analysis of both group A) RSV type A or B) RSV type B was determined by bootstrap datasets and consensus used to produce an extended majority rule phylogenetic tree. Circles indicate isolates analysed at the ALN-RSV01 target site.

FIG. 28: In vitro inhibition of primary RSV isolates by ALN-RSV01. Vero cells in 24-well plates were transfected with decreasing concentrations of ALN-RSV01 followed by infection with 200-300 pfu of RSV primary isolates. At day 5 post-infection, cells were fixed, immunostained, and counted. Percent activity against PBS was plotted.

FIG. 29 is a bar graph illustrating the results of an in vitro TNFα assay comparing modified and unmodified siRNA duplexes.

FIG. 30 depicts the steps in a model for measuring in vivo immuno-stimulation by intranasal dosing of siRNA agents.

FIG. 31 summarizes data from immunostimulatory assays of Bronchioaveolar lavage following administration of ALN-RSV01 and various controls sequences.

FIG. 32 shows the immuno-stimulation attenuating effects of chemical modifications to the ALN-RSV01 siRNA sequence.

FIG. 33: Chemically modified ALN-RSV01 siRNAs stimulate no detectable IFN-a from PBMC in vitro. siRNAs transfected into PBMC were assayed for IFN-α stimulation at 24 hours post transfection. The dashed horizontal line indicates the lower level of assay detection for IFN-α (39 pg/ml). Med., medium alone; DTP; DOTAP alone.

FIG. 34: Data shown are expressed as fold increase above PBMC cultured in medium alone and are from a single donor. <LLOQ; cytokine levels below the lower level of detection. Limits of detection for the cytokines measured: TNF-α, 7-25879 pg/ml; IL-6, 2-8002 pg/ml; IP-10, 38-26230 pg/ml; IFN-γ, 19-26280 pg/ml; G-CSF, 1-5447 pg/ml; IL-ra, 5-79814 pg/ml.

FIG. 35: The Table shows that chemically modified ALN-RSV01 dsRNAs show markedly reduces in vitro induction of IFN-inducible genes in PBMC. PBMC were treated with the indicated siRNAs or control treatments. After 24 hours total RNA was isolated from cells, cDNA amplified, and subjected to RT-PCR analysis for the panel of eight mRNAs shown. Data are expressed as fold change above control PBMC cultured in medium alone and are derived from a single donor. Values represent the mean±STD of duplicate reactions.

FIG. 36: Bar graph showing remaining percentage (averaged values of the three individual NHP BAL experiments) of remaining intact sense strands (left bar in each pair of bars) or antisense strands (right bar in each of bars) after 8 hours incubation.

### DETAILED DESCRIPTION

The instant specification may refer to one or more of the following abbreviations whose meanings are defined in Table 2, below.

TABLE 2
List of Abbreviations
Table 2 - List of Abbreviations

| | |
|---|---|
| A | Adenosine |
| AAS | Atomic Absorption Spectroscopy |
| Ado | Adenosine |
| AE | Adverse Event |
| ALT | Alanine aminotransferase |
| AST | Aspartate aminotransferase |
| AUC | Area under the concentration-time curve |
| AX-HPLC | Anion Exchange High Performance Liquid |
| | Chromatography |
| BMI | Body mass index |
| bpm | Beats per minute |
| BUN | Blood urea nitrogen |
| C | Cytidine |
| Ca | Calcium |
| CBC | Complete blood cell [count] |
| CDER | Center for Drug Evaluation and Research |
| CFR | Code of Federal Regulations |
| CFU | Colony-Forming Units |
| cGMP | Current Good Manufacturing Practices |
| Cl | Chloride |
| COPD | Chronic Obstructive Pulmonary Disease |
| CPG | Controlled Pore Glass |
| CRF | Case Report Form |
| CRO | Contract Research Organization |
| CRP | c-Reactive Protein |
| CTCAE | Common Terminology Criteria for Adverse Events |
| CV | Curriculum Vitae |
| Cyd | Cytidine |
| Da | Daltons |
| DLT | Dose-limiting toxicity (ies) |
| DMT | Dimethoxytrityl |
| dsRNA | Double-stranded ribonucleic acid |
| dT | Thymidine |
| dThd | Thymidine |
| ECG | Electrocardiogram |
| EP | European Pharmacopeia |
| ESI | Electrospray Ionization |
| EU | Endotoxin Units |
| FDA | Food and Drug Administration |
| FLP | Full Length Product |
| FTIR | Fourier Transform Infrared Spectroscopy |
| G | Gram |
| G | Guanosine |
| GC | Gas Chromatography |
| GCP | Good Clinical Practice |
| G-CSF | Granulocyte colony stimulating factor |
| GMP | Good Manufacturing Practices |
| Guo | Guanosine |
| HBsAg | Hepatitis B Surface Antigen |
| Hct | Hematocrit |
| HCV | Hepatitis C Virus |
| HFIP | Hexafluoroisopropanol |
| Hgb | Hemoglobin |
| HIPAA | Health Insurance Portability and Accountability Act |
| HIV | Human Immunodeficiency Virus |
| HPLC | High Performance Liquid Chromatography |
| IB | Investigator's Brochure |
| ICF | Informed Consent Form |
| ICH | International Conference on Harmonization |
| ICP | Inductively Coupled Plasma |
| ICP-MS | Inductively Coupled Plasma Mass Spectrometry |
| ID | Identity |
| IEC | Independent Ethics Committee |
| IL-10 | Interleukin 10 |
| IL-8 | Interleukin 8 |
| IMPD | Investigational Medicinal Product Dossier |
| INR | International Normalized Ratio |
| IRB | Institutional Review Board |
| IRC | Independent Review Committee |
| ITT | Intent to treat |
| K | Potassium |
| kg | Kilogram |
| LAL | Limulus Amebocyte Lysate |
| LC-MS | Liquid Chromatography-Mass Spectrometry |
| LDH | Lactate dehydrogenase |
| LLOQ | Lower Limit of Quantification |
| M | Molar |
| MALDI-TOF | Matrix Assisted Laser Desorption Ionization - |
| | Time of Flight |
| MCH | Mean corpuscular hemoglobin |
| MCHC | Mean corpuscular hemoglobin concentration |
| MCV | Mean corpuscular volume |
| MedDRA | Medical Dictionary for Regulatory Activities |
| mg | Milligram |
| mL | Milliliter |
| mM | Millimolar |
| mRNA | Messenger Ribonucleic Acid |
| MS | Mass Spectrometry |
| MTD | Maximum tolerated dose |
| MW | Molecular Weight |
| N | Full Length Oligonucleotide of the Intermediate |
| | Single Strands |
| Na | Sodium |
| NaCl | Sodium chloride |
| ND | Not Detected |
| NF | National Formulary |
| nm | Nanometer |
| NMR | Nuclear Magnetic Resonance |
| NOAEL | No observed adverse effect level |
| NOEL | No observed effect level |
| NT | Not Tested |
| OTC | Over the counter |
| PBS | Phosphate Buffered Solution |
| PE | Physical examination |
| PES | Polyethersulfone |
| pH | Potential of Hydrogen |
| PI | Principal Investigator |
| PK | Pharmacokinetics |
| PP | Per protocol |
| ppm | Parts Per Million |
| PT | Prothrombin Time |
| PTT | Partial thromboplastin time |
| PVDF | Polyvinylidene Difluoride |
| q.s. | Quantity Sufficient |
| QC | Quality Control |
| RBC | Red Blood Cell |
| RH | Relative Humidity |
| RISC | RNA induced silencing complex |
| RNA | Ribonucleic Acid |
| RNAi | RNA interference |
| RRT | Relative Retention Time |
| RSV | Respiratory Syncytial Virus |
| RTM | RSV Transport Media |
| rt-PCR | Reverse transcriptase polymerase chain reaction |
| SAE | Serious adverse event |
| SAP | Statistical Analysis Plan |
| SAR | Seasonal allergic rhinitis |
| SEC | Size Exclusion Chromatography |
| siRNA | Small Interfering Ribonucleic Acid |
| SOP | Standard Operating Procedure |
| SP | Safety Population |
| TBDMS | Tert-butyldimethylsilyl |
| TCID50 | Tissue culture infective dose producing 50% infection |
| TFF | Tangential Flow Filtration |
| Tm | Duplex Helix to Coil Melting Temperature |
| TNF | Tumor necrosis factor |
| U | Uridine |
| UF | Ultrafiltration |
| UK | United Kingdom |
| Urd | Uridine |
| US | United States |
| USP | United States Pharmacopeia |
| USP/NF | United States Pharmacopeia/National Formulary |
| UV | Ultraviolet |
| w/v | Weight by Volume |
| w/w | Weight by Weight |
| WBC | White Blood Cell |
| WHO | World Health Organization |

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

For ease of exposition the term “nucleotide” or “ribonucleotide” is sometimes used herein in reference to one or more monomeric subunits of an RNA agent. It will be understood that the usage of the term “ribonucleotide” or “nucleotide” herein can, in the case of a modified RNA or nucleotide surrogate, also refer to a modified nucleotide, or surrogate replacement moiety, as further described below, at one or more positions.

An “RNA agent” as used herein, is an unmodified RNA, modified RNA, or nucleoside surrogate, all of which are described herein or are well known in the RNA synthetic art. While numerous modified RNAs and nucleoside surrogates are described, preferred examples include those which have greater resistance to nuclease degradation than do unmodified RNAs. Preferred examples include those that have a 2′ sugar modification, a modification in a single strand overhang, preferably a 3′ single strand overhang, or, particularly if single stranded, a 5′-modification which includes one or more phosphate groups or one or more analogs of a phosphate group.

An “iRNA agent” (abbreviation for “interfering RNA agent”) as used herein, is an RNA agent, which can down-regulate the expression of a target gene, e.g., RSV. While not wishing to be bound by theory, an iRNA agent may act by one or more of a number of mechanisms, including post-transcriptional cleavage of a target mRNA sometimes referred to in the art as RNAi, or pre-transcriptional or pre-translational mechanisms. An iRNA agent can be a double stranded (ds) iRNA agent.

A “ds iRNA agent” (abbreviation for “double stranded iRNA agent”), as used herein, is an iRNA agent which includes more than one, and preferably two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g. by a linker, e.g. a polyethyleneglycol linker, to form but one molecule. At least one strand can include a region which is sufficiently complementary to a target RNA. Such strand is termed the “antisense strand”. A second strand comprised in the dsRNA agent which comprises a region complementary to the antisense strand is termed the “sense strand”. However, a ds iRNA agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

The term “double-stranded RNA” or “dsRNA,” as used herein, refers to a complex of ribonucleic acid molecules, having a duplex structure comprising two anti-parallel and substantially complementary, as defined above, nucleic acid strands. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims

The dsRNA agents of the present invention include molecules which are modified so as to alleviate an immune response in mammalian cells. Thus, the administration of a composition of an iRNA agent (e.g., formulated as described herein) to a mammalian cell can be used to silence expression of an RSV gene while circumventing an immune response.

The isolated iRNA agents described herein, including ds iRNA agents and siRNA agents, can mediate silencing of a gene, e.g., by RNA degradation. For convenience, such RNA is also referred to herein as the RNA to be silenced. Such a gene is also referred to as a target gene. Preferably, the RNA to be silenced is a gene product of an RSV gene, particularly the P, N or L gene product.

As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secret at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

In the anti viral uses of the present invention, silencing of a target gene will result in a reduction in “viral titer” in the cell or in the subject. As used herein, “reduction in viral titer” refers to a decrease in the number of viable virus produced by a cell or found in an organism undergoing the silencing of a viral target gene. Reduction in the cellular amount of virus produced will preferably lead to a decrease in the amount of measurable virus produced in the tissues of a subject undergoing treatment and a reduction in the severity of the symptoms of the viral infection. iRNA agents of the present invention are also referred to as “antiviral iRNA agents”.

As used herein, a “RSV gene” refers to any one of the genes identified in the RSV virus genome (See Falsey, A. R., and E. E. Walsh, 2000, Clinical Microbiological Reviews 13:371-84). These genes are readily known in the art and include the N, P and L genes which are exemplified herein.

As used herein, “target sequence” refers to a contiguous portion of the nucleotide sequence of an mRNA molecule formed during the transcription of a gene from RSV, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. dsRNAs as used herein are also referred to as “siRNAs” (short interfering RNAs).

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

As used herein, a “subject” refers to a mammalian organism undergoing treatment for a disorder mediated by viral expression, such as RSV infection or undergoing treatment prophylactically to prevent viral infection. The subject can be any mammal, such as a primate, cow, horse, mouse, rat, dog, pig, goat. In the preferred embodiment, the subject is a human.

As used herein in the context of RSV infection, the terms “treat,” “treatment,” and the like, refer to relief from or alleviation of any biological or pathological endpoints that 1) is mediated in part by the presence of the virus in the subject and 2) whose outcome can be affected by reducing the level of viral gene products present.

Design and Selection of iRNA Agents

The present invention is based on the demonstration of target gene silencing of a respiratory viral gene in vivo following local administration to the lungs and nasal passage of an iRNA agent either via intranasal administration/inhalation or systemically/parenterally via injection and the resulting treatment of viral infection. The present invention is further extended to the use of iRNA agents to more than one respiratory virus and the treatment of both virus infections with co-administration of two or more iRNA agents.

Based on these results, the invention specifically provides an iRNA agent that can be used in treating viral infection, particularly respiratory viruses and in particular RSV infection, in isolated form and as a pharmaceutical composition described below. Such agents will include a sense strand having at least 15 or more contiguous nucleotides that are complementary to a viral gene and an antisense strand having at least 15 or more contiguous nucleotides that are complementary to the sense strand sequence. Particularly useful are iRNA agents that consist of, consist essentially of or comprise a nucleotide sequence from the P N and L gene of RSV as provided in Table 1 (a-c).

The iRNA agents of the present invention are based on and comprise at least 15 or more contiguous nucleotides from one of the iRNA agents shown to be active in Table 1 (a-c). In such agents, the agent can consist of consist essentially of or comprise the entire sequence provided in the table or can comprise 15 or more contiguous residues provided in Table 1a-c along with additional nucleotides from contiguous regions of the target gene.

An iRNA agent can be rationally designed based on sequence information and desired characteristics and the information provided in Table 1 (a-c). For example, an iRNA agent can be designed according to sequence of the agents provided in the Tables as well as in view of the entire coding sequence of the target gene.

Accordingly, the present invention provides iRNA agents comprising a sense strand and antisense strand each comprising a sequence of at least 15, 16, 17, 18, 19, 20, 21 or 23 nucleotides which is essentially identical to, as defined above, a portion of a gene from a respiratory virus, particularly the P, N or L protein genes of RSV. Exemplified iRNA agents include those that comprise 15 or more contiguous nucleotides from one of the agents provided in Table 1 (a-c).

The antisense strand of an iRNA agent should be equal to or at least, 15, 16 17, 18, 19, 25, 29, 40, or 50 nucleotides in length. It should be equal to or less than 50, 40, or 30, nucleotides in length. Preferred ranges are 15-30, 17 to 25, 19 to 23, and 19 to 21 nucleotides in length. Exemplified iRNA agents include those that comprise 15 or more nucleotides from one of the antisense strands of one of the agents in Table 1 (a-c).

The sense strand of an iRNA agent should be equal to or at least 15, 16 17, 18, 19, 25, 29, 40, or 50 nucleotides in length. It should be equal to or less than 50, 40, or 30 nucleotides in length. Preferred ranges are 15-30, 17 to 25, 19 to 23, and 19 to 21 nucleotides in length. Exemplified iRNA agents include those that comprise 15 or more nucleotides from one of the sense strands of one of the agents in Table 1 (a-c).

The double stranded portion of an iRNA agent should be equal to or at least, 15, 16 17, 18, 19, 20, 21, 22, 23, 24, 25, 29, 40, or 50 nucleotide pairs in length. It should be equal to or less than 50, 40, or 30 nucleotides pairs in length. Preferred ranges are 15-30, 17 to 25, 19 to 23, and 19 to 21 nucleotides pairs in length.

The agents provided in Table 1 (a-c) are 21 nucleotide in length for each strand. The iRNA agents contain a 19 nucleotide double stranded region with a 2 nucleotide overhang on each of the 3′ ends of the agent. These agents can be modified as described herein to obtain equivalent agents comprising at least a portion of these sequences (15 or more contiguous nucleotides) and or modifications to the oligonucleotide bases and linkages.

Generally, the mRNA agents of the instant invention include a region of sufficient complementarity to the viral gene, e.g. the P, N or L protein of RSV, and are of sufficient length in terms of nucleotides, that the iRNA agent, or a fragment thereof, can mediate down regulation of the specific viral gene. The antisense strands of the iRNA agents of the present invention are preferably fully complementary to the mRNA sequences of viral gene, as is herein for the P, L or N proteins of RSV. However, it is not necessary that there be perfect complementarity between the iRNA agent and the target, but the correspondence must be sufficient to enable the iRNA agent, or a cleavage product thereof, to direct sequence specific silencing, e.g., by RNAi cleavage of an RSV mRNA.

Therefore, the iRNA agents of the instant invention include agents comprising a sense strand and antisense strand each comprising a sequence of at least 16, 17 or 18 nucleotides which is essentially identical, as defined below, to one of the sequences of a viral gene, particularly the P, N or L protein of RSV, such as those agent provided in Table 1 (a-c), except that not more than 1, 2 or 3 nucleotides per strand, respectively, have been substituted by other nucleotides (e.g. adenosine replaced by uracil), while essentially retaining the ability to inhibit RSV expression in cultured human cells, as defined below. These agents will therefore possess at least 15 or more nucleotides identical to one of the sequences of a viral gene, particularly the P, L or N protein gene of RSV, but 1, 2 or 3 base mismatches with respect to either the target viral mRNA sequence or between the sense and antisense strand are introduced. Mismatches to the target viral mRNA sequence, particularly in the antisense strand, are most tolerated in the terminal regions and if present are preferably in a terminal region or regions, e.g., within 6, 5, 4, or 3 nucleotides of a 5′ and/or 3′ terminus, most preferably within 6, 5, 4, or 3 nucleotides of the 5′-terminus of the sense strand or the 3′-terminus of the antisense strand. The sense strand need only be sufficiently complementary with the antisense strand to maintain the overall double stranded character of the molecule.

It is preferred that the sense and antisense strands be chosen such that the mRNA agent includes a single strand or unpaired region at one or both ends of the molecule, such as those exemplified in Table 1 (a-c). Thus, an iRNA agent contains sense and antisense strands, preferably paired to contain an overhang, e.g., one or two 5′ or 3′ overhangs but preferably a 3′ overhang of 2-3 nucleotides. Most embodiments will have a 3′ overhang. Preferred siRNA agents will have single-stranded overhangs, preferably 3′ overhangs, of 1 to 4, or preferably 2 or 3 nucleotides, in length, on one or both ends of the iRNA agent. The overhangs can be the result of one strand being longer than the other, or the result of two strands of the same length being staggered. 5′-ends are preferably phosphorylated.

Preferred lengths for the duplexed region is between 15 and 30, most preferably 18, 19, 20, 21, 22, and 23 nucleotides in length, e.g., in the siRNA agent range discussed above. Embodiments in which the two strands of the siRNA agent are linked, e.g., covalently linked are also included. Hairpin, or other single strand structures which provide the required double stranded region, and preferably a 3′ overhang are also within the invention.

Evaluation of Candidate iRNA Agents

A candidate iRNA agent can be evaluated for its ability to down regulate target gene expression. For example, a candidate iRNA agent can be provided, and contacted with a cell, e.g., a human cell, that has been infected with or will be infected with the virus of interest, e.g., a virus containing the target gene. Alternatively, the cell can be transfected with a construct from which a target viral gene is expressed, thus preventing the need for a viral infectivity model. The level of target gene expression prior to and following contact with the candidate iRNA agent can be compared, e.g., on an RNA, protein level or viral titer. If it is determined that the amount of RNA, protein or virus expressed from the target gene is lower following contact with the iRNA agent, then it can be concluded that the mRNA agent down-regulates target gene expression. The level of target viral RNA or viral protein in the cell or viral titer in a cell or tissue can be determined by any method desired. For example, the level of target RNA can be determined by Northern blot analysis, reverse transcription coupled with polymerase chain reaction (RT-PCR), bDNA analysis, or RNAse protection assay. The level of protein can be determined, for example, by Western blot analysis or immuno-fluorescence. Viral titer can be detected through a plaque formation assay.

Stability Testing, Modification, and Retesting of iRNA Agents

A candidate iRNA agent can be evaluated with respect to stability, e.g., its susceptibility to cleavage by an endonuclease or exonuclease, such as when the iRNA agent is introduced into the body of a subject. Methods can be employed to identify sites that are susceptible to modification, particularly cleavage, e.g., cleavage by a component found in the body of a subject.

When sites susceptible to cleavage are identified, a further iRNA agent can be designed and/or synthesized wherein the potential cleavage site is made resistant to cleavage, e.g. by introduction of a 2′-modification on the site of cleavage, e.g. a 2′-O-methyl group. This further iRNA agent can be retested for stability, and this process may be iterated until an iRNA agent is found exhibiting the desired stability.

In Vivo Testing

An iRNA agent identified as being capable of inhibiting viral gene expression can be tested for functionality in vivo in an animal model (e.g., in a mammal, such as in mouse, rat or primate) as shown in the examples. For example, the iRNA agent can be administered to an animal, and the iRNA agent evaluated with respect to its biodistribution, stability, and its ability to inhibit viral, e.g., RSV, gene expression or to reduce viral titer.

The iRNA agent can be administered directly to the target tissue, such as by injection, or the iRNA agent can be administered to the animal model in the same manner that it would be administered to a human. As shown herein, the agent can be preferably administered intranasally or via inhalation as a means of preventing or treating viral infection.

The iRNA agent can also be evaluated for its intracellular distribution. The evaluation can include determining whether the iRNA agent was taken up into the cell. The evaluation can also include determining the stability (e.g., the half-life) of the iRNA agent. Evaluation of an iRNA agent in vivo can be facilitated by use of an iRNA agent conjugated to a traceable marker (e.g., a fluorescent marker such as fluorescein; a radioactive label, such as 35S, 32P, 33P, or 3H; gold particles; or antigen particles for immunohistochemistry) or other suitable detection method.

The iRNA agent can be evaluated with respect to its ability to down regulate viral gene expression. Levels of viral gene expression in vivo can be measured, for example, by in situ hybridization, or by the isolation of RNA from tissue prior to and following exposure to the iRNA agent. Where the animal needs to be sacrificed in order to harvest the tissue, an untreated control animal will serve for comparison. Target viral mRNA can be detected by any desired method, including but not limited to RT-PCR, Northern blot, branched-DNA assay, or RNAse protection assay. Alternatively, or additionally, viral gene expression can be monitored by performing Western blot analysis on tissue extracts treated with the iRNA agent or by ELISA. Viral titer can be determined using a pfu assay.

iRNA Chemistry

Described herein are isolated iRNA agents, e.g., ds RNA agents, that mediate RNAi to inhibit expression of a viral gene, e.g., the P protein of RSV.

Methods for producing and purifying iRNA agents are well known to those of skill in the art of nucleic acid chemistry. In certain embodiments the production methods can include solid phase synthesis using phosphoramidite monomers with commercial nucleic acid synthesizers. See, e.g., “Solid-Phase Synthesis: A Practical Guide,” (Steven A. Kates and Fernando Albericio (eds.), Marcel Dekker, Inc., New York, 2000). In certain embodiments the invention is practiced using processes and reagents for oligonucleotide synthesis and purification as described in co-owned PCT Application No. PCT/US2005/011490 filed Apr. 5, 2005.

RNA agents discussed herein include otherwise unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., (1994) Nucleic Acids Res. 22: 2183-2196. Such rare or unusual RNAs, often termed modified RNAs (apparently because these are typically the result of a post-transcriptional modification) are within the term unmodified RNA, as used herein. Modified RNA as used herein refers to a molecule in which one or more of the components of the nucleic acid, namely sugars, bases, and phosphate moieties, are different from that which occurs in nature, preferably different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of each of the above are discussed herein.

Modifications described herein can be incorporated into any double-stranded RNA and RNA-like molecule described herein, e.g., an iRNA agent. It may be desirable to modify one or both of the antisense and sense strands of an iRNA agent. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. By way of example, a modification may only occur at a 3′ or 5′ terminal position, may only occur in a terminal region, e.g. at a position on a terminal nucleotide or in the last 2, 3, 4, 5, or 10 nucleotides of a strand. A modification may occur in a double strand region, a single strand region, or in both. E.g., a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, the sense and antisense strand will have the same modifications or the same class of modifications, but in other cases the sense and antisense strand will have different modifications, e.g., in some cases it may be desirable to modify only one strand, e.g. the sense strand.

Two prime objectives for the introduction of modifications into iRNA agents is their stabilization towards degradation in biological environments and the improvement of pharmacological properties, e.g., pharmacodynamic properties, which are further discussed below. Other suitable modifications to a sugar, base, or backbone of an iRNA agent are described in co-owned PCT Application No. PCT/US2004/01193, filed Jan. 16, 2004. An iRNA agent can include a non-naturally occurring base, such as the bases described in co-owned PCT Application No. PCT/US2004/011822, filed Apr. 16, 2004. An iRNA agent can include a non-naturally occurring sugar, such as a non-carbohydrate cyclic carrier molecule. Exemplary features of non-naturally occurring sugars for use in iRNA agents are described in co-owned PCT Application No. PCT/US2004/11829 filed Apr. 16, 2003.

An iRNA agent can include an internucleotide linkage (e.g., the chiral phosphorothioate linkage) useful for increasing nuclease resistance. In addition, or in the alternative, an iRNA agent can include a ribose mimic for increased nuclease resistance. Exemplary internucleotide linkages and ribose mimics for increased nuclease resistance are described in co-owned PCT Application No. PCT/US2004/07070 filed on Mar. 8, 2004.

An iRNA agent can include ligand-conjugated monomer subunits and monomers for oligonucleotide synthesis. Exemplary monomers are described in co-owned U.S. application Ser. No. 10/916,185, filed on Aug. 10, 2004.

An iRNA agent can have a ZXY structure, such as is described in co-owned PCT Application

No. PCT/US2004/07070 filed on Mar. 8, 2004.

An iRNA agent can be complexed with an amphipathic moiety. Exemplary amphipathic moieties for use with iRNA agents are described in co-owned PCT Application No. PCT/US2004/07070 filed on Mar. 8, 2004.

In another embodiment, the iRNA agent can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid or protein, e.g., an antibody, that binds to a specified cell type. iRNA agents complexed to a delivery agent are described in co-owned PCT Application No. PCT/US2004/07070 filed on Mar. 8, 2004.

An iRNA agent can have non-canonical pairings, such as between the sense and antisense sequences of the iRNA duplex. Exemplary features of non-canonical iRNA agents are described in co-owned PCT Application No. PCT/US2004/07070 filed on Mar. 8, 2004.

Enhanced Nuclease Resistance

An iRNA agent, e.g., an iRNA agent that targets RSV, can have enhanced resistance to nucleases.

For increased nuclease resistance and/or binding affinity to the target, an iRNA agent, e.g., the sense and/or antisense strands of the iRNA agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)CH₂CH₂OR; “locked” nucleic acids (LNA) in which the 2′ hydroxyl is connected, e.g., by a methylene bridge, to the 4′ carbon of the same ribose sugar; O-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially ds RNA); halo (e.g., fluoro); amino (e.g., NH₂; alkylamino, dialkylamino, heterocyclyl, arylamino, diaryl amino, heteroaryl amino, diheteroaryl amino, or amino acid); NH(CH₂CH₂NH)CH₂CH₂-AMINE (AMINE=NH₂; alkylamino, dialkylamino, heterocyclyl amino, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

Preferred substituents are 2′O-methyl (OMe), 2′-methoxyethyl, 2′-OCH3, 2′-O-allyl, 2′-C-allyl, and 2′-fluoro (2′F). In one aspect, both 2′OMe and 2′F are used as substituents on an iRNA agent.

One way to increase resistance is to identify cleavage sites and modify such sites to inhibit cleavage, as described in co-owned U.S. Application No. 60/559,917, filed on May 4, 2004. For example, the dinucleotides 5′-UA-3′, 5′ UG 3′,5′-CA-3′, 5′ UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The iRNA agent can include at least 2, at least 3, at least 4 or at least 5 of such dinucleotides. In certain embodiments, all the pyrimidines of an iRNA agent carry a 2′-modification, and the iRNA agent therefore has enhanced resistance to endonucleases.

To maximize nuclease resistance, the 2′ modifications can be used in combination with one or more phosphate linker modifications (e.g., phosphorothioate). The so-called “chimeric” oligonucleotides are those that contain two or more different modifications.

The inclusion of furanose sugars in the oligonucleotide backbone can also decrease endonucleolytic cleavage. An iRNA agent can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

An iRNA agent can have increased resistance to nucleases when a duplexed iRNA agent includes a single-stranded nucleotide overhang on at least one end. In preferred embodiments, the nucleotide overhang includes 1 to 4, preferably 2 to 3, unpaired nucleotides. In a preferred embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs. In further embodiments, the nucleotide overhang may have 1 or 2 unpaired nucleotides, and in an exemplary embodiment the nucleotide overhang is 5′-GC-3′. In preferred embodiments, the nucleotide overhang is on the 3′-end of the antisense strand. In one embodiment, the iRNA agent includes the motif 5′-CGC-3′ on the 3′-end of the antisense strand, such that a 2-nt overhang 5′-GC-3′ is formed.

In one aspect, a hydroxy pyrollidine (hp) linker provides exonuclease protection.

Thus, an iRNA agent can include modifications so as to inhibit degradation, e.g., by nucleases, e.g., endonucleases or exonucleases, found in the body of a subject. These monomers are referred to herein as NRMs, or Nuclease Resistance promoting Monomers, the corresponding modifications as NRM modifications. In many cases these modifications will modulate other properties of the iRNA agent as well, e.g., the ability to interact with a protein, e.g., a transport protein, e.g., serum albumin, or a member of the RISC, or the ability of the first and second sequences to form a duplex with one another or to form a duplex with another sequence, e.g., a target molecule.

One or more different NRM modifications can be introduced into an iRNA agent or into a sequence of an iRNA agent. An NRM modification can be used more than once in a sequence or in an iRNA agent.

NRM modifications include some which can be placed only at the terminus and others which can go at any position. Some NRM modifications that can inhibit hybridization are preferably used only in terminal regions, and more preferably not at the cleavage site or in the cleavage region of a sequence which targets a subject sequence or gene, particularly on the antisense strand. They can be used anywhere in a sense strand, provided that sufficient hybridization between the two strands of the ds iRNA agent is maintained. In some embodiments it is desirable to put the NRM at the cleavage site or in the cleavage region of a sense strand, as it can minimize off-target silencing.

In most cases, the NRM modifications will be distributed differently depending on whether they are comprised on a sense or antisense strand. If on an antisense strand, modifications which interfere with or inhibit endonuclease cleavage should not be inserted in the region which is subject to RISC mediated cleavage, e.g., the cleavage site or the cleavage region (as described in Elbashir et al., 2001, Genes and Dev. 15: 188, hereby incorporated by reference). Cleavage of the target occurs about in the middle of a 20 or 21 nt antisense strand, or about 10 or 11 nucleotides upstream of the first nucleotide on the target mRNA which is complementary to the antisense strand. As used herein cleavage site refers to the nucleotides on either side of the site of cleavage, on the target mRNA or on the iRNA agent strand which hybridizes to it. Cleavage region means the nucleotides within 1, 2, or 3 nucleotides of the cleavage site, in either direction.

Such modifications can be introduced into the terminal regions, e.g., at the terminal position or with 2, 3, 4, or 5 positions of the terminus, of a sequence which targets or a sequence which does not target a sequence in the subject.

Tethered Ligands

The properties of an iRNA agent, including its pharmacological properties, can be influenced and tailored, for example, by the introduction of ligands, e.g., tethered ligands.

A wide variety of entities, e.g., ligands, can be tethered to an iRNA agent, e.g., to the carrier of a ligand-conjugated monomer subunit. Examples are described below in the context of a ligand-conjugated monomer subunit but that is only preferred, entities can be coupled at other points to an iRNA agent.

Preferred moieties are ligands, which are coupled, preferably covalently, either directly or indirectly via an intervening tether, to the carrier. In preferred embodiments, the ligand is attached to the carrier via an intervening tether. The ligand or tethered ligand may be present on the ligand-conjugated monomer when the ligand-conjugated monomer is incorporated into the growing strand. In some embodiments, the ligand may be incorporated into a “precursor” ligand-conjugated monomer subunit after a “precursor” ligand-conjugated monomer subunit has been incorporated into the growing strand. For example, a monomer having, e.g., an amino-terminated tether, e.g., TAP-(CH₂)ₙNH₂ may be incorporated into a growing sense or antisense strand. In a subsequent operation, i.e., after incorporation of the precursor monomer subunit into the strand, a ligand having an electrophilic group, e.g., a pentafluorophenyl ester or aldehyde group, can subsequently be attached to the precursor ligand-conjugated monomer by coupling the electrophilic group of the ligand with the terminal nucleophilic group of the precursor ligand-conjugated monomer subunit tether.

In preferred embodiments, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand.

Preferred ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophilic molecules, lipids, lectins, steroids (e.g., uvaol, hecigenin, diosgenin), terpenes (e.g., triterpenes, e.g., sarsasapogenin, Friedelin, epifriedelanol derivatized lithocholic acid), vitamins, carbohydrates(e.g., a dextran, pullulan, chitin, chitosan, inulin, cyclodextrin or hyaluronic acid), proteins, protein binding agents, integrin targeting molecules, polycationics, peptides, polyamines, and peptide mimics.

The ligand may be a naturally occurring or recombinant or synthetic molecule, such as a synthetic polymer, e.g., a synthetic polyamino acid. Examples of polyamino acids include polyamino acid is a polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic moieties, e.g., cationic lipid, cationic porphyrin, quaternary salt of a polyamine, or an alpha helical peptide.

Ligands can also include targeting groups, e.g., a cell or tissue targeting agent, e.g., a thyrotropin, melanotropin, surfactant protein A, Mucin carbohydrate, a glycosylated polyaminoacid, transferrin, bisphosphonate, polyglutamate, polyaspartate, or an RGD peptide or RGD peptide mimetic.

Ligands can be proteins, e.g., glycoproteins, lipoproteins, e.g., low density lipoprotein (LDL), or albumins, e.g., human serum albumin (HSA), or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as cofactors, multivalent lactose, multivalent galactose, N-acetyl-galactosamine, N-acetyl-glucosamine, multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In one aspect, the ligand is a lipid or lipid-based molecule. Such a lipid or lipid-based molecule preferably binds a serum protein, e.g., human serum albumin (HSA). Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another aspect, the ligand is a moiety, e.g., a vitamin or nutrient, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include the B vitamins, e.g., folic acid, B12, riboflavin, biotin, pyridoxal or other vitamins or nutrients taken up by cancer cells.

In another aspect, the ligand is a cell-permeation agent, preferably a helical cell-permeation agent. Preferably, the agent is amphipathic. An exemplary agent is a peptide such as tat or antennapedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

5′-Phosphate Modifications

In preferred embodiments, iRNA agents are 5′ phosphorylated or include a phosphoryl analog at the 5′ prime terminus. 5′-phosphate modifications of the antisense strand include those which are compatible with RISC mediated gene silencing. Suitable modifications include: 5′-monophosphate ((HO)2(O)P-O-5′); 5′-diphosphate ((HO)2(O)P-O-P(HO)(O)-O-5′); 5′-triphosphate ((HO)₂(O)P-O-(HO)(O)P-O-P(HO)(O)-O-5′); 5′-guanosine cap (7-methylated or non-methylated) (7m-G-O-5′-(HO)(O)P-O-(HO)(O)P-O-P(HO)(O)-O-5′); 5′-adenosine cap (Appp), and any modified or unmodified nucleotide cap structure. Other suitable 5′-phosphate modifications will be known to the skilled person.

The sense strand can be modified in order to inactivate the sense strand and prevent formation of an active RISC, thereby potentially reducing off-target effects. This can be accomplished by a modification which prevents 5′-phosphorylation of the sense strand, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage.

Delivery of iRNA Agents to Tissues and Cells

Formulation

The iRNA agents described herein can be formulated for administration to a subject, preferably for administration locally to the lungs and nasal passage (respiratory tissues) via inhalation or intranasal administration, or parenterally, e.g., via injection.

For ease of exposition, the formulations, compositions, and methods in this section are discussed largely with regard to unmodified iRNA agents. It should be understood, however, that these formulations, compositions, and methods can be practiced with other iRNA agents, e.g., modified iRNA agents, and such practice is within the invention.

A formulated iRNA agent composition can assume a variety of states. In some examples, the composition is at least partially crystalline, uniformly crystalline, and/or anhydrous (e.g., less than 80, 50, 30, 20, or 10% water). In another example, the iRNA agent is in an aqueous phase, e.g., in a solution that includes water, this form being the preferred form for administration via inhalation.

The aqueous phase or the crystalline compositions can be incorporated into a delivery vehicle, e.g., a liposome (particularly for the aqueous phase), or a particle (e.g., a microparticle as can be appropriate for a crystalline composition). Generally, the iRNA agent composition is formulated in a manner that is compatible with the intended method of administration.

An iRNA agent preparation can be formulated in combination with another agent, e.g., another therapeutic agent or an agent that stabilizes an iRNA agent, e.g., a protein that complexes with the iRNA agent to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the iRNA agent preparation includes another iRNA agent, e.g., a second iRNA agent that can mediate RNAi with respect to a second gene. Still other preparations can include at least three, five, ten, twenty, fifty, or a hundred or more different iRNA species. In some embodiments, the agents are directed to the same virus but different target sequences. In another embodiment, each iRNA agents is directed to a different virus. As demonstrated in the Example, more than one virus can be inhibited by co-administering two mRNA agents simultaneously, or at closely time intervals, each one directed to one of the viruses being treated.

Treatment Methods and Routes of Delivery

A composition that includes an iRNA agent of the present invention, e.g., an iRNA agent that targets RSV, can be delivered to a subject by a variety of routes. The pharmaceutical compositions of the present invention may be administered in a number of ways depending upon whether local or systemic treatment is desired and upon the area to be treated. Administration may be topical (including intranasal or intrapulmonary), oral or parenteral. Exemplary routes include inhalation, intravenous, nasal, or oral delivery.

In general, the delivery of the iRNA agents of the present invention is done to achieve delivery into the subject to the site of infection. This objective can be achieved through either a local (i.e., topical) administration to the lungs or nasal passage, e.g., into the respiratory tissues via inhalation, nebulization or intranasal administration, or via systemic administration, e.g., parental administration. Parenteral administration includes intravenous drip, subcutaneous, intraperitoneal or intramuscular injection. The preferred means of administering the iRNA agents of the present invention is through direct topical administration to the lungs and/or nasal passage by inhalation of an aerosolized liquid such as a nebulized mist or a nasal spray.

An iRNA agent can be incorporated into pharmaceutical compositions suitable for administration. For example, compositions can include one or more iRNA agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Formulations for inhalation, intranasal, or parenteral administration are well known in the art. Such formulations may include sterile aqueous solutions which may also contain buffers, diluents and other suitable additives, an example being PBS or Dextrose 5% in water. For intravenous use, the total concentration of solutes should be controlled to render the preparation isotonic.

The active compounds disclosed herein are preferably administered to the lung(s) or nasal passage of a subject by any suitable means. Active compounds may be administered by administering an aerosol suspension of respirable particles comprised of the active compound or active compounds, which the subject inhales. The active compound can be aerosolized in a variety of forms, such as, but not limited to, dry powder inhalants, metered dose inhalants, or liquid/liquid suspensions. The respirable particles may be liquid or solid. The particles may optionally contain other therapeutic ingredients such as amiloride, benzamil or phenamil, with the selected compound included in an amount effective to inhibit the reabsorption of water from airway mucous secretions, as described in U.S. Pat. No. 4,501,729.

The particulate pharmaceutical composition may optionally be combined with a carrier to aid in dispersion or transport. A suitable carrier such as a sugar (i.e., dextrose, lactose, sucrose, trehalose, mannitol) may be blended with the active compound or compounds in any suitable ratio (e.g., a 1 to 1 ratio by weight).

In one embodiment, an active compound is topically administered by inhalation. As used in this specification, administration by “inhalation” generally refers to the inspiration of particles comprised of the active compound that are of respirable size, that is, particles of a size sufficiently small to pass through the mouth or nose and larynx upon inhalation and into the bronchi and alveoli of the lungs. In general, particles ranging from about 1 to 10 microns in size (more particularly, less than about microns in size) are respirable and suitable for administration by inhalation.

In another embodiment, an active compound is topically delivered by intranasal administration. As used in this specification, “intranasal” administration refers to administration of a dosage form formulated and delivered to topically treat the nasal epithelium. Particles or droplets used for intranasal administration generally have a diameter that is larger than those used for administration by inhalation. For intranasal administration, a particle size in the range of 10-500 microns is preferred to ensure retention in the nasal cavity. Particles of non-respirable size which are included in the aerosol tend to deposit in the throat and be swallowed, and the quantity of non-respirable particles in the aerosol is preferably minimized.

Liquid pharmaceutical compositions of active compound for producing an aerosol can be prepared by combining the active compound with a suitable vehicle, such as sterile pyrogen free water. In certain embodiments hypertonic saline solutions are used to carry out the present invention. These are preferably sterile, pyrogen-free solutions, comprising from one to fifteen percent (by weight) of a physiologically acceptable salt, and more preferably from three to seven percent by weight of the physiologically acceptable salt.

Aerosols of liquid particles comprising the active compound may be produced by any suitable means, such as with a pressure-driven jet nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation.

Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic, but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate therapeutic aerosol generator. Aerosol generators for administering solid particulate therapeutics to a subject produce particles which are respirable and generate a volume of aerosol containing a predetermined metered dose of a therapeutic at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 μl to 200 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidant and suitable flavoring agents.

Administration can be provided by the subject or by another person, e.g., a caregiver. A caregiver can be any entity involved with providing care to the human: for example, a hospital, hospice, doctor's office, outpatient clinic; a healthcare worker such as a doctor, nurse, or other practitioner; or a spouse or guardian, such as a parent. The medication can be provided in measured doses or in a dispenser which delivers a metered dose.

The term “therapeutically effective amount” is the amount present in the composition that is needed to provide the desired level of drug in the subject to be treated to give the anticipated physiological response. In one embodiment, therapeutically effective amounts of two or more iRNA agents, each one directed to a different respiratory virus, e.g., RSV, and FIV are administered concurrently to a subject.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The term “co-administration” refers to administering to a subject two or more agents, and in particular two or more iRNA agents. The agents can be contained in a single pharmaceutical composition and be administered at the same time, or the agents can be contained in separate formulation and administered serially to a subject. So long as the two agents can be detected in the subject at the same time, the two agents are said to be co-administered.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-beta-cyclodextrin; and polysaccharides, such as raffinose, maltodextrins, dextrans, and the like; alditols, such as mannitol, xylitol, and the like. A preferred group of carbohydrates includes lactose, trehalose, raffinose maltodextrins, and mannitol. Suitable polypeptides include aspartame. Amino acids include alanine and glycine, with glycine being preferred.

Suitable pH adjusters or buffers include organic salts prepared from organic acids and bases, such as sodium citrate, sodium ascorbate, and the like; sodium citrate is preferred.

Dosage. An iRNA agent can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of iRNA agent (e.g., about 4.4×1016 copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of iRNA agent per kg of bodyweight. The unit dose, for example, can be administered by an inhaled dose or nebulization or by injection. In one example, dosage ranges of 0.02-25 mg/kg is used.

Delivery of an mRNA agent directly to the lungs or nasal passage can be at a dosage on the order of about 1 mg to about 150 mg/nasal passage, such as, e.g., 25, 50, 75, 100 or 150 mg/nasal passage.

The dosage can be an amount effective to treat or prevent a disease or disorder.

In one embodiment, the unit dose is administered once a day. In other usage, a unit dose is administered twice the first day and then daily. Alternatively, unit dosing can be less than once a day, e.g., less than every 2, 4, 8 or 30 days. In another embodiment, the unit dose is not administered with a frequency (e.g., not a regular frequency). For example, the unit dose may be administered a single time. Because iRNA agent mediated silencing can persist for several days after administering the iRNA agent composition, in many instances, it is possible to administer the composition with a frequency of less than once per day, or, for some instances, only once for the entire therapeutic regimen.

In general, a suitable dose of dsRNA will be in the range of 0.01 to 200.0 milligrams per kilogram body weight of the recipient per day, generally in the range of 1 to 50 mg per kilogram body weight per day. For example, the dsRNA can be administered at 0.01 mg/kg, 0.05 mg/kg, 0.5 mg/kg, 1 mg/kg, 1.5 mg/kg, 2 mg/kg, 3 mg/kg, 5.0 mg/kg, 10 mg/kg, 20 mg/kg, 30 mg/kg, 40 mg/kg, or 50 mg/kg per single dose. The pharmaceutical composition may be administered once daily, or the dsRNA may be administered as two, three, or more sub-doses at appropriate intervals throughout the day or even using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. In this embodiment, the dosage unit contains a corresponding multiple of the daily dose.

In one embodiment, a subject is administered an initial dose, and one or more maintenance doses of an iRNA agent, e.g., a double-stranded iRNA agent, or siRNA agent, (e.g., a precursor, e.g., a larger iRNA agent which can be processed into an siRNA agent, or a DNA which encodes an iRNA agent, e.g., a double-stranded iRNA agent, or siRNA agent, or precursor thereof). The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 15, 10, 9, 8, 7, 6, 5, 4, 3, 2, 1, 0.5, 0.1, 0.05, 0.01, 0.005, 0.001, or 0.0005 mg per kg of bodyweight per day. The maintenance doses are preferably administered no more than once every 5-14 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

In one embodiment, the iRNA agent pharmaceutical composition includes a plurality of iRNA agent species. The iRNA agent species can have sequences that are non-overlapping and non-adjacent with respect to a naturally occurring target sequence, e.g., a target sequence of the RSV gene. In another embodiment, the plurality of iRNA agent species is specific for different naturally occurring target genes. For example, an iRNA agent that targets the P protein gene of RSV can be present in the same pharmaceutical composition as an mRNA agent that targets a different gene, for example the N protein gene. In another embodiment, the iRNA agents are specific for different viruses, e.g., RSV.

The concentration of the iRNA agent composition is an amount sufficient to be effective in treating or preventing a disorder or to regulate a physiological condition in humans. The concentration or amount of iRNA agent administered will depend on the parameters determined for the agent and the method of administration, e.g., nasal, buccal, or pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The dsRNAs featured in the invention can be administered in combination with other known agents effective in treatment of pathological processes mediated by target gene expression. In any event, the administering physician can adjust the amount and timing of dsRNA administration on the basis of results observed using standard measures of efficacy known in the art or described herein.

The invention is further illustrated by the following examples, which should not be construed as further limiting.

### EXAMPLES

### Example 1

### Designing Antiviral siRNAs Against RSV mRNA

siRNA against RSV P, N and L mRNA were synthesized chemically using know procedures. The siRNA sequences and some inhibition cross-subtype activity and 1050 values as described below are listed in Tables 1a, 1b, and 1c.

TABLE 1a
RSV L gene

| | Whitehead | | | | | |
|---|---|---|---|---|---|---|
| Actual | Start | SEQ ID | | SEQ ID | | RSV L gene |
| start | Pos | NO. | Sense | NO. | Antisense | duplex |
| 3 | 1 | 3 | GGAUCCCAUUAUUAAUGGAdTdT | 117 | UCCAUUAAUAAUGGGAUCCdTdT | AL-DP-2024 |
| 4 | 2 | 4 | GAUCCCAUUAUUAAUGGAAdTdT | 118 | UUCCAUUAAUAAUGGGAUCdTdT | AL-DP-2026 |
| 49 | 47 | 5 | AGUUAUUUAAAAGGUGUUAdTdT | 119 | UAACACCUUUUAAAUAACUdTdT | AL-DP-2116 |
| 50 | 48 | 6 | GUUAUUUAAAAGGUGUUAUdTdT | 120 | AUAACACCUUUUAAAUAACdTdT | AL-DP-2117 |
| 53 | 51 | 7 | AUUUAAAAGGUGUUAUCUCdTdT | 121 | GAGAUAACACCUUUUAAAUdTdT | AL-DP-2118 |
| 55 | 53 | 8 | UUAAAAGGUGUUAUCUCUUdTdT | 122 | AAGAGAUAACACCUUUUAAdTdT | AL-DP-2119 |
| 156 | 154 | 9 | AAGUCCACUACUAGAGCAUdTdT | 123 | AUGCUCUAGUAGUGGACUUdTdT | AL-DP-2027 |
| 157 | 155 | 10 | AGUCCACUACUAGAGCAUAdTdT | 124 | UAUGCUCUAGUAGUGGACUdTdT | AL-DP-2028 |
| 158 | 156 | 11 | GUCCACUACUAGAGCAUAUdTdT | 125 | AUAUGCUCUAGUAGUGGACdTdT | AL-DP-2029 |
| 159 | 157 | 12 | UCCACUACUAGAGCAUAUGdTdT | 126 | CAUAUGCUCUAGUAGUGGAdTdT | AL-DP-2030 |
| 341 | 339 | 13 | GAAGAGCUAUAGAAAUAAGdTdT | 127 | CUUAUUUCUAUAGCUCUUCdTdT | AL-DP-2120 |
| 344 | 342 | 14 | GAGCUAUAGAAAUAAGUGAdTdT | 128 | UCACUUAUUUCUAUAGCUCdTdT | AL-DP-2121 |
| 347 | 345 | 15 | CUAUAGAAAUAAGUGAUGUdTdT | 129 | ACAUCACUUAUUUCUAUAGdTdT | AL-DP-2031 |
| 554 | 552 | 16 | UCAAAACAACACUCUUGAAdTdT | 130 | UUCAAGAGUGUUGUUUUGAdTdT | AL-DP-2122 |
| 1004 | 1002 | 17 | UAGAGGGAUUUAUUAUGUCdTdT | 131 | GACAUAAUAAAUCCCUCUAdTdT | AL-DP-2123 |
| 1408 | 1406 | 18 | AUAAAAGGGUUUGUAAAUAdTdT | 132 | UAUUUACAAACCCUUUUAUdTdT | AL-DP-2124 |
| 1867 | 1865 | 19 | CUCAGUGUAGGUAGAAUGUdTdT | 133 | ACAUUCUACCUACACUGAGdTdT | AL-DP-2032 |
| 1868 | 1866 | 20 | UCAGUGUAGGUAGAAUGUUdTdT | 134 | AACAUUCUACCUACACUGAdTdT | AL-DP-2033 |
| 1869 | 1867 | 21 | CAGUGUAGGUAGAAUGUUUdTdT | 135 | AAACAUUCUACCUACACUGdTdT | AL-DP-2034 |
| 1870 | 1868 | 22 | AGUGUAGGUAGAAUGUUUGdTdT | 136 | CAAACAUUCUACCUACACUdTdT | AL-DP-2112 |
| 1871 | 1869 | 23 | GUGUAGGUAGAAUGUUUGCdTdT | 137 | GCAAACAUUCUACCUACACdTdT | AL-DP-2113 |
| 1978 | 1976 | 24 | ACAAGAUAUGGUGAUCUAGdTdT | 138 | CUAGAUCACCAUAUCUUGUdTdT | AL-DP-2035 |
| 2104 | 2102 | 25 | AGCAAAUUCAAUCAAGCAUdTdT | 139 | AUGCUUGAUUGAAUUUGCUdTdT | AL-DP-2036 |
| 2105 | 2103 | 26 | GCAAAUUCAAUCAAGCAUUdTdT | 140 | AAUGCUUGAUUGAAUUUGCdTdT | AL-DP-2037 |
| 2290 | 2288 | 27 | GAUGAACAAAGUGGAUUAUdTdT | 141 | AUAAUCCACUUUGUUCAUCdTdT | AL-DP-2038 |
| 2384 | 2382 | 28 | UAAUAUCUCUCAAAGGGAAdTdT | 142 | UUCCCUUUGAGAGAUAUUAdTdT | AL-DP-2125 |
| 2386 | 2384 | 29 | AUAUCUCUCAAAGGGAAAUdTdT | 143 | AUUUCCCUUUGAGAGAUAUdTdT | AL-DP-2126 |
| 2387 | 2385 | 30 | UAUCUCUCAAAGGGAAAUUdTdT | 144 | AAUUUCCCUUUGAGAGAUAdTdT | AL-DP-2127 |
| 2485 | 2483 | 31 | CAUGCUCAAGCAGAUUAUUdTdT | 145 | AAUAAUCUGCUUGAGCAUGdTdT | AL-DP-2039 |
| 2487 | 2485 | 32 | UGCUCAAGCAGAUUAUUUGdTdT | 146 | CAAAUAAUCUGCUUGAGCAdTdT | AL-DP-2040 |
| 2507 | 2505 | 33 | UAGCAUUAAAUAGCCUUAAdTdT | 147 | UUAAGGCUAUUUAAUGCUAdTdT | AL-DP-2041 |
| 2508 | 2506 | 34 | AGCAUUAAAUAGCCUUAAAdTdT | 148 | UUUAAGGCUAUUUAAUGCUdTdT | AL-DP-2114 |
| 2509 | 2507 | 35 | GCAUUAAAUAGCCUUAAAUdTdT | 149 | AUUUAAGGCUAUUUAAUGCdTdT | AL-DP-2042 |
| 2510 | 2508 | 36 | CAUUAAAUAGCCUUAAAUUdTdT | 150 | AAUUUAAGGCUAUUUAAUGdTdT | AL-DP-2043 |
| 2765 | 2763 | 37 | UAUUAUGCAGUUUAAUAUUdTdT | 151 | AAUAUUAAACUGCAUAAUAdTdT | AL-DP-2044 |
| 2767 | 2765 | 38 | UUAUGCAGUUUAAUAUUUAdTdT | 152 | UAAAUAUUAAACUGCAUAAdTdT | AL-DP-2045 |
| 3283 | 3281 | 39 | AAAAGUGCACAACAUUAUAdTdT | 153 | UAUAAUGUUGUGCACUUUUdTdT | AL-DP-2128 |
| 3284 | 3282 | 40 | AAAGUGCACAACAUUAUACdTdT | 154 | GUAUAAUGUUGUGCACUUUdTdT | AL-DP-2046 |
| 3338 | 3336 | 41 | AUAUAGAACCUACAUAUCCdTdT | 155 | GGAUAUGUAGGUUCUAUAUdTdT | AL-DP-2047 |
| 3339 | 3337 | 42 | UAUAGAACCUACAUAUCCUdTdT | 156 | AGGAUAUGUAGGUUCUAUAdTdT | AL-DP-2048 |
| 3365 | 3363 | 43 | UAAGAGUUGUUUAUGAAAGdTdT | 157 | CUUUCAUAAACAACUCUUAdTdT | AL-DP-2129 |
| 4021 | 4019 | 44 | ACAGUCAGUAGUAGACCAUdTdT | 158 | AUGGUCUACUACUGACUGUdTdT | AL-DP-2049 |
| 4022 | 4020 | 45 | CAGUCAGUAGUAGACCAUGdTdT | 159 | CAUGGUCUACUACUGACUGdTdT | AL-DP-2050 |
| 4023 | 4021 | 46 | AGUCAGUAGUAGACCAUGUdTdT | 160 | ACAUGGUCUACUACUGACUdTdT | AL-DP-2051 |
| 4024 | 4022 | 47 | GUCAGUAGUAGACCAUGUGdTdT | 161 | CACAUGGUCUACUACUGACdTdT | AL-DP-2052 |
| 4025 | 4023 | 48 | UCAGUAGUAGACCAUGUGAdTdT | 162 | UCACAUGGUCUACUACUGAdTdT | AL-DP-2053 |
| 4037 | 4035 | 49 | CAUGUGAAUUCCCUGCAUCdTdT | 163 | GAUGCAGGGAAUUCACAUGdTdT | AL-DP-2054 |
| 4038 | 4036 | 50 | AUGUGAAUUCCCUGCAUCAdTdT | 164 | UGAUGCAGGGAAUUCACAUdTdT | AL-DP-2055 |
| 4039 | 4037 | 51 | UGUGAAUUCCCUGCAUCAAdTdT | 165 | UUGAUGCAGGGAAUUCACAdTdT | AL-DP-2056 |
| 4040 | 4038 | 52 | GUGAAUUCCCUGCAUCAAUdTdT | 166 | AUUGAUGCAGGGAAUUCACdTdT | AL-DP-2115 |
| 4043 | 4041 | 53 | AAUUCCCUGCAUCAAUACCdTdT | 167 | GGUAUUGAUGCAGGGAAUUdTdT | AL-DP-2057 |
| 4051 | 4049 | 54 | GCAUCAAUACCAGCUUAUAdTdT | 168 | UAUAAGCUGGUAUUGAUGCdTdT | AL-DP-2058 |
| 4052 | 4050 | 55 | CAUCAAUACCAGCUUAUAGdTdT | 169 | CUAUAAGCUGGUAUUGAUGdTdT | AL-DP-2059 |
| 4057 | 4055 | 56 | AUACCAGCUUAUAGAACAAdTdT | 170 | UUGUUCUAUAAGCUGGUAUdTdT | AL-DP-2060 |
| 4058 | 4056 | 57 | UACCAGCUUAUAGAACAACdTdT | 171 | GUUGUUCUAUAAGCUGGUAdTdT | AL-DP-2061 |
| 4059 | 4057 | 58 | ACCAGCUUAUAGAACAACAdTdT | 172 | UGUUGUUCUAUAAGCUGGUdTdT | AL-DP-2062 |
| 4060 | 4058 | 59 | CCAGCUUAUAGAACAACAAdTdT | 173 | UUGUUGUUCUAUAAGCUGGdTdT | AL-DP-2063 |
| 4061 | 4059 | 60 | CAGCUUAUAGAACAACAAAdTdT | 174 | UUUGUUGUUCUAUAAGCUGdTdT | AL-DP-2064 |
| 4067 | 4065 | 61 | AUAGAACAACAAAUUAUCAdTdT | 175 | UGAUAAUUUGUUGUUCUAUdTdT | AL-DP-2065 |
| 4112 | 4110 | 62 | UAUUAACAGAAAAGUAUGGdTdT | 176 | CCAUACUUUUCUGUUAAUAdTdT | AL-DP-2130 |
| 4251 | 4249 | 63 | UGAGAUACAUUUGAUGAAAdTdT | 177 | UUUCAUCAAAUGUAUCUCAdTdT | AL-DP-2066 |
| 4252 | 4250 | 64 | GAGAUACAUUUGAUGAAACdTdT | 178 | GUUUCAUCAAAUGUAUCUCdTdT | AL-DP-2067 |
| 4254 | 4252 | 65 | GAUACAUUUGAUGAAACCUdTdT | 179 | AGGUUUCAUCAAAUGUAUCdTdT | AL-DP-2068 |
| 4255 | 4253 | 66 | AUACAUUUGAUGAAACCUCdTdT | 180 | GAGGUUUCAUCAAAUGUAUdTdT | AL-DP-2069 |
| 4256 | 4254 | 67 | UACAUUUGAUGAAACCUCCdTdT | 181 | GGAGGUUUCAUCAAAUGUAdTdT | AL-DP-2074 |
| 4313 | 4311 | 68 | AAGUGAUACAAAAACAGCAdTdT | 182 | UGCUGUUUUUGUAUCACUUdTdT | AL-DP-2131 |
| 4314 | 4312 | 69 | AGUGAUACAAAAACAGCAUdTdT | 183 | AUGCUGUUUUUGUAUCACUdTdT | AL-DP-2132 |
| 4316 | 4314 | 70 | UGAUACAAAAACAGCAUAUdTdT | 184 | AUAUGCUGUUUUUGUAUCAdTdT | AL-DP-2133 |
| 4473 | 4471 | 71 | UUUAAGUACUAAUUUAGCUdTdT | 185 | AGCUAAAUUAGUACUUAAAdTdT | AL-DP-2075 |
| 4474 | 4472 | 72 | UUAAGUACUAAUUUAGCUGdTdT | 186 | CAGCUAAAUUAGUACUUAAdTdT | AL-DP-2076 |
| 4475 | 4473 | 73 | UAAGUACUAAUUUAGCUGGdTdT | 187 | CCAGCUAAAUUAGUACUUAdTdT | AL-DP-2077 |
| 4476 | 4474 | 74 | AAGUACUAAUUUAGCUGGAdTdT | 188 | UCCAGCUAAAUUAGUACUUdTdT | AL-DP-2078 |
| 4477 | 4475 | 75 | AGUACUAAUUUAGCUGGACdTdT | 189 | GUCCAGCUAAAUUAGUACUdTdT | AL-DP-2079 |
| 4478 | 4476 | 76 | GUACUAAUUUAGCUGGACAdTdT | 190 | UGUCCAGCUAAAUUAGUACdTdT | AL-DP-2080 |
| 4480 | 4478 | 77 | ACUAAUUUAGCUGGACAUUdTdT | 191 | AAUGUCCAGCUAAAUUAGUdTdT | AL-DP-2081 |
| 4483 | 4481 | 78 | AAUUUAGCUGGACAUUGGAdTdT | 192 | UCCAAUGUCCAGCUAAAUUdTdT | AL-DP-2082 |
| 4484 | 4482 | 79 | AUUUAGCUGGACAUUGGAUdTdT | 193 | AUCCAAUGUCCAGCUAAAUdTdT | AL-DP-2083 |
| 4486 | 4484 | 80 | UUAGCUGGACAUUGGAUUCdTdT | 194 | GAAUCCAAUGUCCAGCUAAdTdT | AL-DP-2084 |
| 4539 | 4537 | 81 | UUUUGAAAAAGAUUGGGGAdTdT | 195 | UCCCCAAUCUUUUUCAAAAdTdT | AL-DP-2134 |
| 4540 | 4538 | 82 | UUUGAAAAAGAUUGGGGAGdTdT | 196 | CUCCCCAAUCUUUUUCAAAdTdT | AL-DP-2135 |
| 4542 | 4540 | 83 | UGAAAAAGAUUGGGGAGAGdTdT | 197 | CUCUCCCCAAUCUUUUUCAdTdT | AL-DP-2136 |
| 4543 | 4541 | 84 | GAAAAAGAUUGGGGAGAGGdTdT | 198 | CCUCUCCCCAAUCUUUUUCdTdT | AL-DP-2137 |
| 4671 | 4669 | 85 | UAUGAACACUUCAGAUCUUdTdT | 199 | AAGAUCUGAAGUGUUCAUAdTdT | AL-DP-2085 |
| 4672 | 4670 | 86 | AUGAACACUUCAGAUCUUCdTdT | 200 | GAAGAUCUGAAGUGUUCAUdTdT | AL-DP-2086 |
| 4867 | 4865 | 87 | UGCCCUUGGGUUGUUAACAdTdT | 201 | UGUUAACAACCCAAGGGCAdTdT | AL-DP-2087 |
| 4868 | 4866 | 88 | GCCCUUGGGUUGUUAACAUdTdT | 202 | AUGUUAACAACCCAAGGGCdTdT | AL-DP-2088 |
| 5544 | 5542 | 89 | UAUAGCAUUCAUAGGUGAAdTdT | 203 | UUCACCUAUGAAUGCUAUAdTdT | AL-DP-2089 |
| 5545 | 5543 | 90 | AUAGCAUUCAUAGGUGAAGdTdT | 204 | CUUCACCUAUGAAUGCUAUdTdT | AL-DP-2090 |
| 5546 | 5544 | 91 | UAGCAUUCAUAGGUGAAGGdTdT | 205 | CCUUCACCUAUGAAUGCUAdTdT | AL-DP-2091 |
| 5550 | 5548 | 92 | AUUCAUAGGUGAAGGAGCAdTdT | 206 | UGCUCCUUCACCUAUGAAUdTdT | AL-DP-2092 |
| 5640 | 5638 | 93 | UUGCAAUGAUCAUAGUUUAdTdT | 207 | UAAACUAUGAUCAUUGCAAdTdT | AL-DP-2093 |
| 5641 | 5639 | 94 | UGCAAUGAUCAUAGUUUACdTdT | 208 | GUAAACUAUGAUCAUUGCAdTdT | AL-DP-2094 |
| 5642 | 5640 | 95 | GCAAUGAUCAUAGUUUACCdTdT | 209 | GGUAAACUAUGAUCAUUGCdTdT | AL-DP-2095 |
| 5643 | 5641 | 96 | CAAUGAUCAUAGUUUACCUdTdT | 210 | AGGUAAACUAUGAUCAUUGdTdT | AL-DP-2096 |
| 5644 | 5642 | 97 | AAUGAUCAUAGUUUACCUAdTdT | 211 | UAGGUAAACUAUGAUCAUUdTdT | AL-DP-2097 |
| 5645 | 5643 | 98 | AUGAUCAUAGUUUACCUAUdTdT | 212 | AUAGGUAAACUAUGAUCAUdTdT | AL-DP-2098 |
| 5647 | 5645 | 99 | GAUCAUAGUUUACCUAUUGdTdT | 213 | CAAUAGGUAAACUAUGAUCdTdT | AL-DP-2138 |
| 5648 | 5646 | 100 | AUCAUAGUUUACCUAUUGAdTdT | 214 | UCAAUAGGUAAACUAUGAUdTdT | AL-DP-2139 |
| 5649 | 5647 | 101 | UCAUAGUUUACCUAUUGAGdTdT | 215 | CUCAAUAGGUAAACUAUGAdTdT | AL-DP-2140 |
| 5650 | 5648 | 102 | CAUAGUUUACCUAUUGAGUdTdT | 216 | ACUCAAUAGGUAAACUAUGdTdT | AL-DP-2099 |
| 5651 | 5649 | 103 | AUAGUUUACCUAUUGAGUUdTdT | 217 | AACUCAAUAGGUAAACUAUdTdT | AL-DP-2100 |
| 5752 | 5750 | 104 | CAUUGGUCUUAUUUACAUAdTdT | 218 | UAUGUAAAUAAGACCAAUGdTdT | AL-DP-2101 |
| 5754 | 5752 | 105 | UUGGUCUUAUUUACAUAUAdTdT | 219 | UAUAUGUAAAUAAGACCAAdTdT | AL-DP-2102 |
| 5755 | 5753 | 106 | UGGUCUUAUUUACAUAUAAdTdT | 220 | UUAUAUGUAAAUAAGACCAdTdT | AL-DP-2103 |
| 5756 | 5754 | 107 | GGUCUUAUUUACAUAUAAAdTdT | 221 | UUUAUAUGUAAAUAAGACCdTdT | AL-DP-2141 |
| 5919 | 5917 | 108 | AUAUCAUGCUCAAGAUGAUdTdT | 222 | AUCAUCUUGAGCAUGAUAUdTdT | AL-DP-2142 |
| 5920 | 5918 | 109 | UAUCAUGCUCAAGAUGAUAdTdT | 223 | UAUCAUCUUGAGCAUGAUAdTdT | AL-DP-2104 |
| 5934 | 5932 | 110 | UGAUAUUGAUUUCAAAUUAdTdT | 224 | UAAUUUGAAAUCAAUAUCAdTdT | AL-DP-2105 |
| 6016 | 6014 | 111 | UACUUAGUCCUUACAAUAGdTdT | 225 | CUAUUGUAAGGACUAAGUAdTdT | AL-DP-2106 |
| 6019 | 6017 | 112 | UUAGUCCUUACAAUAGGUCdTdT | 226 | GACCUAUUGUAAGGACUAAdTdT | AL-DP-2107 |
| 6020 | 6018 | 113 | UAGUCCUUACAAUAGGUCCdTdT | 227 | GGACCUAUUGUAAGGACUAdTdT | AL-DP-2108 |
| 6252 | 6250 | 114 | AUAUUCUAUAGCUGGACGUdTdT | 228 | ACGUCCAGCUAUAGAAUAUdTdT | AL-DP-2109 |
| 6253 | 6251 | 115 | UAUUCUAUAGCUGGACGUAdTdT | 229 | UACGUCCAGCUAUAGAAUAdTdT | AL-DP-2110 |
| 6254 | 6252 | 116 | AUUCUAUAGCUGGACGUAAdTdT | 230 | UUACGUCCAGCUAUAGAAUdTdT | AL-DP-2111 |

| | % inh | % inh | | | |
|---|---|---|---|---|---|
| RSV L gene | RSV A2 | RSV A2 | % inh RSV | % inh RSV A2 | |
| duplex | (5 nM) | 500 pM | A2 50 pM | 5 pM | % inh RVS B (5 nM) |
| AL-DP-2038 | 11 | | | | |
| AL-DP-2031 | 15 | | | | |
| AL-DP-2045 | 15 | | | | |
| AL-DP-2050 | 15 | | | | |
| AL-DP-2056 | 16 | | | | |
| AL-DP-2049 | 24 | | | | |
| AL-DP-2026 | 82 | | | | |
| AL-DP-2033 | 84 | | | | |
| AL-DP-2048 | 84 | | | | |
| AL-DP-2027 | 86 | | | | |
| AL-DP-2030 | 86 | | | | |
| AL-DP-2034 | 86 | | | | |
| AL-DP-2058 | 86 | | | | |
| AL-DP-2066 | 86 | | | | |
| AL-DP-2036 | 87 | | | | |
| AL-DP-2039 | 87 | | | | |
| AL-DP-2047 | 87 | | | | |
| AL-DP-2051 | 87 | | | | |
| AL-DP-2040 | 88 | | | | |
| AL-DP-2055 | 88 | | | | |
| AL-DP-2061 | 88 | | | | |
| AL-DP-2029 | 89 | | | | |
| AL-DP-2035 | 89 | | | | |
| AL-DP-2069 | 89 | | | | |
| AL-DP-2028 | 90 | | | | |
| AL-DP-2032 | 90 | | | | |
| AL-DP-2063 | 90 | | | | |
| AL-DP-2037 | 91 | | | | |
| AL-DP-2059 | 91 | | | | |
| AL-DP-2065 | 91 | | | | |
| AL-DP-2024 | 92 | | | | |
| AL-DP-2053 | 92 | 84 | 79 | 76 | 74 |
| AL-DP-2060 | 92 | | | | |
| AL-DP-2067 | 92 | | | | |
| AL-DP-2068 | 93 | | | | |
| AL-DP-2046 | 94 | 94 | 91 | 91 | 93 |
| AL-DP-2057 | 94 | 91 | 86 | 79 | 69 |
| AL-DP-2064 | 94 | 86 | 76 | 67 | 83 |
| AL-DP-2062 | 95 | 79 | 78 | 72 | 94 |
| AL-DP-2041 | 96 | 76 | 73 | 69 | 94 |
| AL-DP-2042 | 96 | 98 | 97 | 97 | 90 |
| AL-DP-2052 | 96 | 84 | 76 | 69 | 87 |
| AL-DP-2043 | 97 | 86 | 79 | 75 | 94 |
| AL-DP-2044 | 97 | 79 | 72 | 67 | 84 |
| AL-DP-2054 | 97 | 79 | 78 | 69 | 96 |

TABLE 1b
RSV P gene

| | | SEQ | | SEQ | | |
|---|---|---|---|---|---|---|
| Actual | | ID | | ID | | RSV P gene |
| start | Start_Pos | NO. | Sense | NO. | Antisense | duplex ID # |
| 55 | 53 | 231 | AAAUUCCUAGAAUCAAUAAdTdT | 250 | UUAUUGAUUCUAGGAAUUUdTdT | AL-DP-2000 |
| 56 | 54 | 232 | AAUUCCUAGAAUCAAUAAAdTdT | 251 | UUUAUUGAUUCUAGGAAUUdTdT | AL-DP-2001 |
| 58 | 56 | 233 | UUCCUAGAAUCAAUAAAGGdTdT | 252 | CCUUUAUUGAUUCUAGGAAdTdT | AL-DP-2002 |
| 59 | 57 | 234 | UCCUAGAAUCAAUAAAGGGdTdT | 253 | CCCUUUAUUGAUUCUAGGAdTdT | AL-DP-2003 |
| 61 | 59 | 235 | CUAGAAUCAAUAAAGGGCAdTdT | 254 | UGCCCUUUAUUGAUUCUAGdTdT | AL-DP-2004 |
| 322 | 320 | 236 | ACAUUUGAUAACAAUGAAGdTdT | 255 | CUUCAUUGUUAUCAAAUGUdTdT | AL-DP-2005 |
| 323 | 321 | 237 | CAUUUGAUAACAAUGAAGAdTdT | 256 | UCUUCAUUGUUAUCAAAUGdTdT | AL-DP-2006 |
| 324 | 322 | 238 | AUUUGAUAACAAUGAAGAAdTdT | 257 | UUCUUCAUUGUUAUCAAAUdTdT | AL-DP-2007 |
| 325 | 323 | 239 | UUUGAUAACAAUGAAGAAGdTdT | 258 | CUUCUUCAUUGUUAUCAAAdTdT | AL-DP-2008 |
| 426 | 424 | 240 | AAGUGAAAUACUAGGAAUGdTdT | 259 | CAUUCCUAGUAUUUCACUUdTdT | AL-DP-2009 |
| 427 | 425 | 241 | AGUGAAAUACUAGGAAUGCdTdT | 260 | GCAUUCCUAGUAUUUCACUdTdT | AL-DP-2010 |
| 428 | 426 | 242 | GUGAAAUACUAGGAAUGCUdTdT | 261 | AGCAUUCCUAGUAUUUCACdTdT | AL-DP-2011 |
| 429 | 427 | 243 | UGAAAUACUAGGAAUGCUUdTdT | 262 | AAGCAUUCCUAGUAUUUCAdTdT | AL-DP-2012 |
| 430 | 428 | 244 | GAAAUACUAGGAAUGCUUCdTdT | 263 | GAAGCAUUCCUAGUAUUUCdTdT | AL-DP-2013 |
| 431 | 429 | 245 | AAAUACUAGGAAUGCUUCAdTdT | 264 | UGAAGCAUUCCUAGUAUUUdTdT | AL-DP-2014 |
| 550 | 548 | 246 | GAAGCAUUAAUGACCAAUGdTdT | 265 | CAUUGGUCAUUAAUGCUUCdTdT | AL-DP-2015 |
| 551 | 549 | 247 | AAGCAUUAAUGACCAAUGAdTdT | 266 | UCAUUGGUCAUUAAUGCUUdTdT | AL-DP-2016 |
| | | 248 | CGAUAAUAUAACAGCAAGAdTsdT | 267 | UCUUGCUGUUAUAUUAUCGdTsdT | AL-DP-1729 |
| | | 249 | CGAUUAUAUUACAGGAUGAdTsdT | 268 | UCAUCCUGUAAUAUAAUCGdTsdT | AL-DP-1730 |

| | % | % inhibition | % inhibition | | % inhibition |
|---|---|---|---|---|---|
| RSV P gene | inhibition | RSV A2 500 | RSV A2 50 | % inhibition | RSV B |
| duplex ID # | (5 nM) | PM | pM | RSV A2 5 pM | (5 nM) |
| AL-DP-2000 | 3 | | | | |
| AL-DP-2001 | 4 | | | | |
| AL-DP-2002 | 7 | | | | |
| AL-DP-2003 | 98 | 93 | 92 | 84 | 97 |
| AL-DP-2004 | 3 | | | | |
| AL-DP-2005 | 7 | | | | |
| AL-DP-2006 | 5 | | | | |
| AL-DP-2007 | 4 | | | | |
| AL-DP-2008 | 7 | | | | |
| AL-DP-2009 | 2 | | | | |
| AL-DP-2010 | 7 | | | | |
| AL-DP-2011 | 4 | | | | |
| AL-DP-2012 | 96 | 77 | 68 | 66 | 92 |
| AL-DP-2013 | 98 | 85 | 76 | 75 | 89 |
| AL-DP-2014 | 98 | 85 | 81 | 68 | 66 |
| AL-DP-2015 | 7 | | | | |
| AL-DP-2016 | 98 | 88 | 82 | 75 | 94 |
| AL-DP-1729 | 90 | | | | |
| AL-DP-1730 | | | | | |

TABLE 1c
RSV N gene

| Actual | SEQ ID | | SEQ ID | | RSV N gene |
|---|---|---|---|---|---|
| start | NO. | Sense | NO. | Antisense | DUPLEX ID # |
| 3 | 1 | GGCUCUUAGCAAAGUCAAGdTdT | 2 | CUUGACUUUGCUAAGAGCCdTdT | AL-DP-2017 |
| | | | | | (ALN-RSV01) |
| 5 | 269 | CUCUUAGCAAAGUCAAGUUdTdT | 277 | AACUUGACUUUGCUAAGAGdTdT | AL-DP-2018 |
| 52 | 270 | CUGUCAUCCAGCAAAUACAdTdT | 278 | UGUAUUUGCUGGAUGACAGdTdT | AL-DP-2019 |
| 53 | 271 | UGUCAUCCAGCAAAUACACdTdT | 279 | GUGUAUUUGCUGGAUGACAdTdT | AL-DP-2020 |
| 191 | 272 | UAAUAGGUAUGUUAUAUGCdTdT | 280 | GCAUAUAACAUACCUAUUAdTdT | AL-DP-2021 |
| 379 | 273 | AUUGAGAUAGAAUCUAGAAdTdT | 281 | UUCUAGAUUCUAUCUCAAUdTdT | AL-DP-2022 |
| 897 | 274 | AUUCUACCAUAUAUUGAACdTdT | 282 | GUUCAAUAUAUGGUAGAAUdTdT | AL-DP-2023 |
| 898 | 275 | UUCUACCAUAUAUUGAACAdTdT | 283 | UGUUCAAUAUAUGGUAGAAdTdT | AL-DP-2024 |
| 899 | 276 | UCUACCAUAUAUUGAACAAdTdT | 284 | UUGUUCAAUAUAUGGUAGAdTdT | AL-DP-2025 |

| | | % inhibition | | % inhibition | |
|---|---|---|---|---|---|
| RSV N gene | % inhibition | RSV A2 500 | % inhibition | RSV A2 | % inhibition |
| Duplex ID # | (5 nM) | pM | RSV A2 50 pM | 5 pM | RSV B (5 nM) |
| AL-DP-2017 | 98 | 86 | 84 | 80 | 93 |
| (ALN-RSV01) | | | | | |
| AL-DP-2018 | 2 | | | | |
| AL-DP-2019 | 5 | | | | |
| AL-DP-2020 | 2 | | | | |
| AL-DP-2021 | 3 | | | | |
| AL-DP-2022 | 98 | 78 | 77 | 75 | 94 |
| AL-DP-2023 | 1 | | | | |
| AL-DP-2024 | 7 | | | | |
| AL-DP-2025 | 96 | 89 | 84 | 77 | 96 |

### Example 2

### In Vitro Assay and Virus Infection

Vero E6 cells were cultured to 80% confluency in DMEM containing 10% heat-inactivated FBS. For siRNA introduction, 4 μl of Transit-TKO was added to 50 μl of serum-free DMEM and incubated at room temperature for 10 minutes. Then, an indicated concentration of siRNA was added to media/TKO reagent respectively and incubated at room temperature for 10 minutes. This mixture was added to 200 μl of DMEM containing 10% FBS and then to a monolayer of cultured cells. The cells were incubated at 37° C., 5% CO₂ for 6 hours. The RNA mixture was removed by gentle washing with 1× Hank's Balanced Salt Solutions (HBSS) and 300 plaque-forming units (pfu) per well of RSV/A2 (MOI=30) was added to wells and adsorbed for 1 hour at 37° C., 5% CO₂. Virus was removed and the cells were washed with 1×HBSS. Cells were overlaid with 1% methylcellulose in DMEM containing 10% FBS media, and incubated for 6 days at 37° C., 5% CO₂. Cells were immunostained for plaques using anti-F protein monoclonal antibody 131-2A.

### Example 3

### siRNA Delivery and Virus Infection In Vivo

Pathogen-free 4 week old female BALB/c mice were purchased from Harlan. Mice were under anesthesia during infection and intranasal instillation (i.n.). Mice were immunized by intranasal instillation with indicated amount of siRNA, either uncomplexed, or complexed with 5 ul Transit TKO. 150 μg of Synagis (monoclonal antibody clone 143-6C, anti-RSV F protein) and Mouse Isotype control (IgG1) were administered intraperitoneal (i.p.) four hours prior to RSV challenge (10⁶ PFU of RSV/A2). Ten mice per group were used. Animal weights were monitored at days 0, 2, 4, and 6 post-infection. Lungs were harvested at day 6 post-infection, and assayed for RSV by immunostaining plaque assay.

### Example 4

### Immunostaining Plaque Assay

24-well plates of Vero E6 cells were cultured to 90% confluency in DMEM containing 10% heat inactivated FBS. Mice lungs were homogenized with hand-held homogenizer in 1 ml sterile Dulbecco's PBS (D-PBS) and 10 fold diluted in serum-free DMEM. Virus containing lung lysate dilutions were plated onto 24 well plates in triplicate and adsorbed for 1 hour at 37° C., 5% CO₂. Wells were overlaid with 1% methylcellulose in DMEM containing 10% FBS. Then, plates were incubated for 6 days at 37° C., 5% CO₂. After 6 days, overlaid media was removed and cells were fixed in acetone:methanol (60:40) for 15 minutes. Cells were blocked with 5% dry Milk/PBS for 1 hour at 37° C. 1:500 dilution of anti-RSV F protein antibody (131-2A) was added to wells and incubated for 2 hours at 37° C. Cells were washed twice in PBS/0.5% Tween 20. 1:500 dilution of goat anti-mouse IgG-Alkaline Phosphatase was added to wells and incubated for 1 hour at 37° C. Cells were washed twice in PBS/0.5% Tween 20. Reaction was developed using Vector's Alkaline Phosphatase substrate kit II (Vector Black), and counterstained with Hematoxylin. Plaques were visualized and counted using an Olympus Inverted microscope.

### Example 5

### Treatment Assay

Mice were challenged with RSV (10⁶ PFU of RSV/A2) by intranasal instillation at day 0 and treated with 50 ug of indicated siRNA, delivered by intranasal instillation, at the indicated times (day 1-4 post viral challenge). 3-5 mice per group were used and viral titers were measured from lung lysates at day 5 post viral challenge, as previously described.

### Example 6

### In Vitro Inhibition of RSV Using iRNA Agents

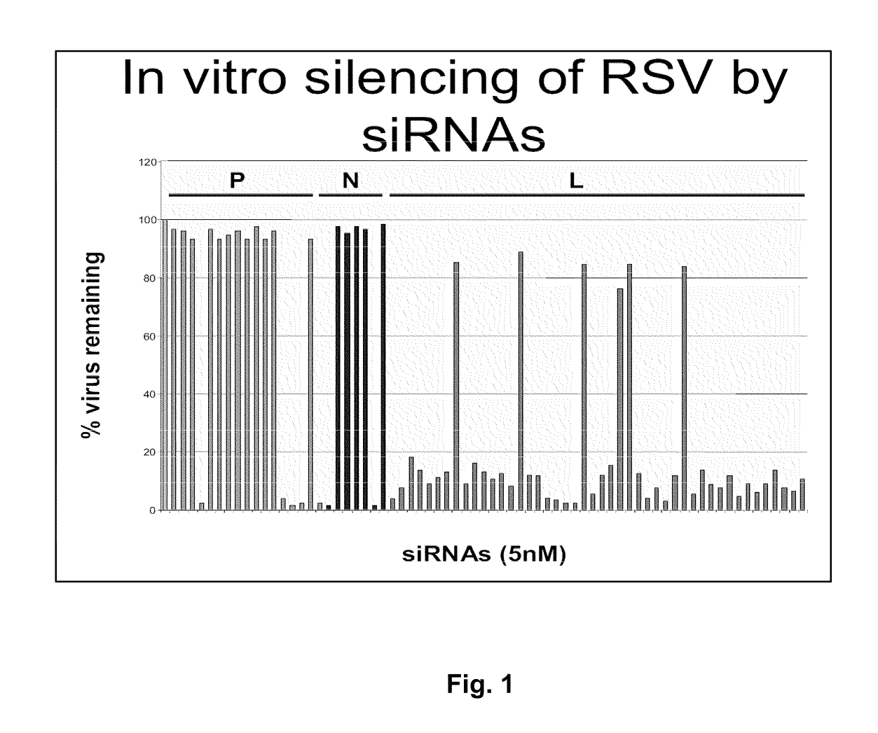

iRNA agents provided in Table 1 (a-c) were tested for anti-RSV activity in a plaque formation assay as described above. Results are shown in FIG. 1. Each column (bar) represents an iRNA agent provided in Table 1 (a-c), e.g., column 1 is the first agent in Table 1a, second column is the second agent and so on. Active iRNA agents were identified by the % of virus remaining. Several agents were identified that showed as much as 90% inhibition. The results are summarized in Table 1 (a-c).

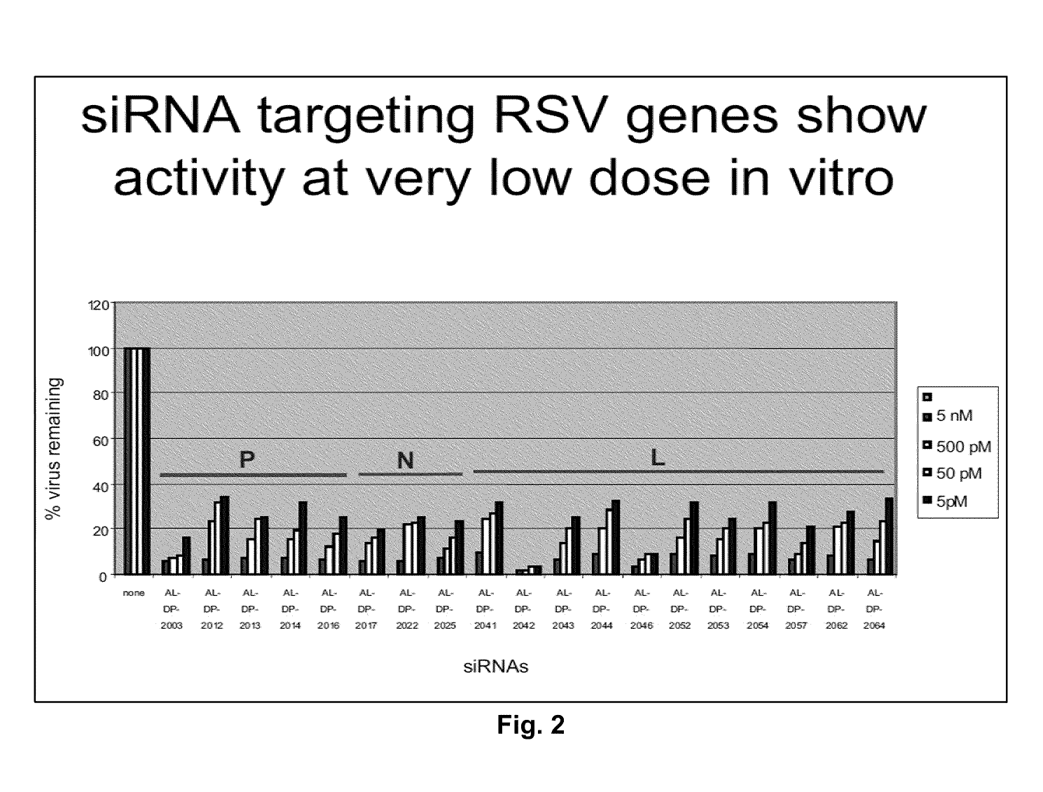

In vitro dose response inhibition of RSV using iRNA agents was determined. Examples of active agents from Table 1 were tested for anti-RSV activity in a plaque formation assay as described above at four concentrations. A dose-dependent response was found with active iRNA agents tested as illustrated in FIG. 2) and summarized in Tables 1(a-c).

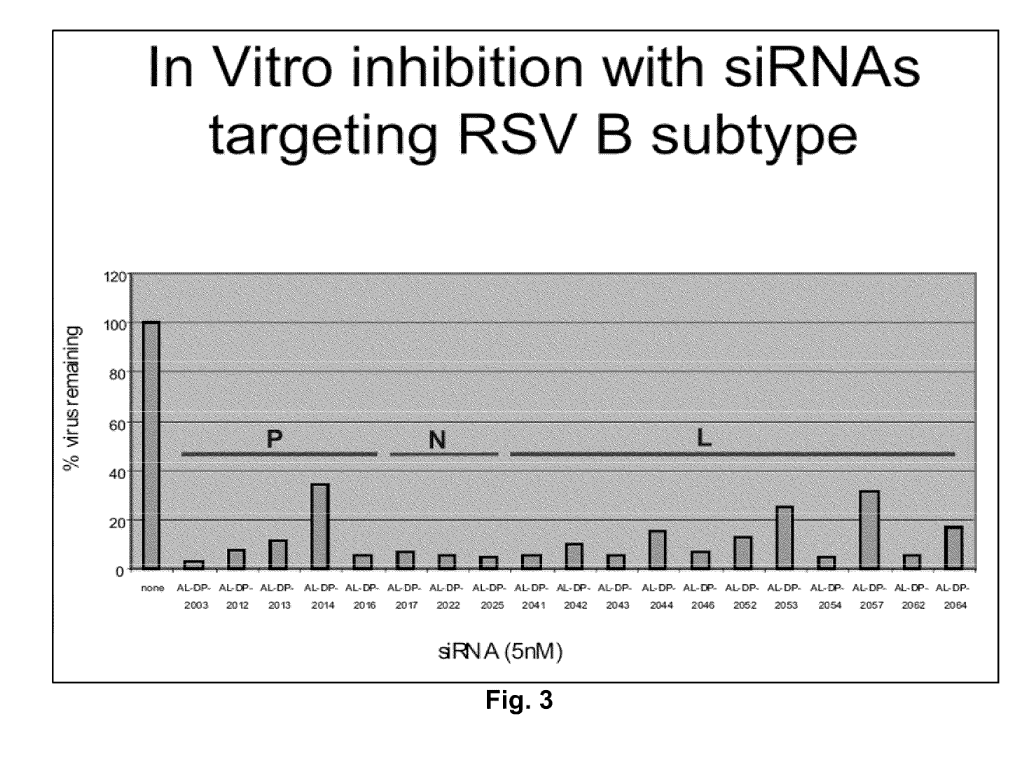

In vitro inhibition of RSV B subtype using iRNA agents was tested as described above. iRNA agents provided in FIG. 2 were tested at 5 nM for anti-RSV activity against subtype B as shown in FIG. 3. RSV subtype B was inhibited by the iRNA agents tested to varying degrees. These results also are summarized in Table 1(a-c).

### Example 7

### In Vivo Inhibition of RSV Using iRNA Agents

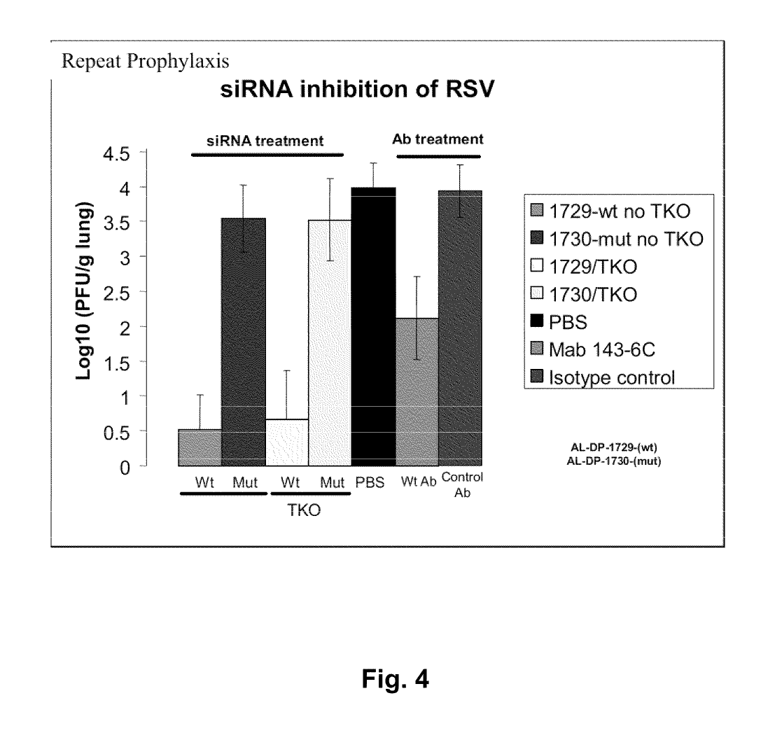

In vivo inhibition of RSV using AL1729 and AL1730 was tested as described above. Agents as described in FIG. 4 were tested for anti-RSV activity in a mouse model. The iRNA agents were effective at reducing viral titers in vivo and more effective than a control antibody (Mab 143-6c, a mouse IgG1 Ab that is approved for RSV treatment).

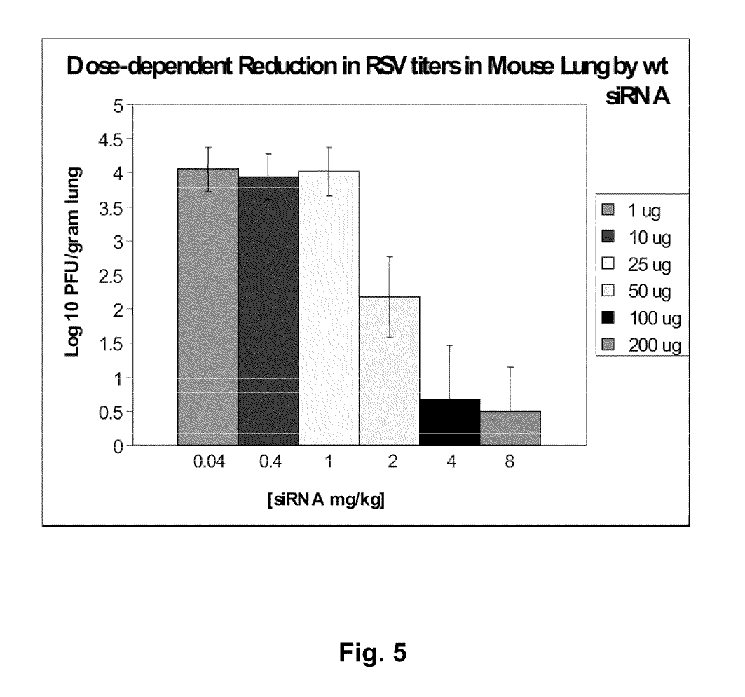

AL1730 was tested for dose-dependent activity using the methods provided above. The agent showed a dose-dependent response as illustrated in FIG. 5.

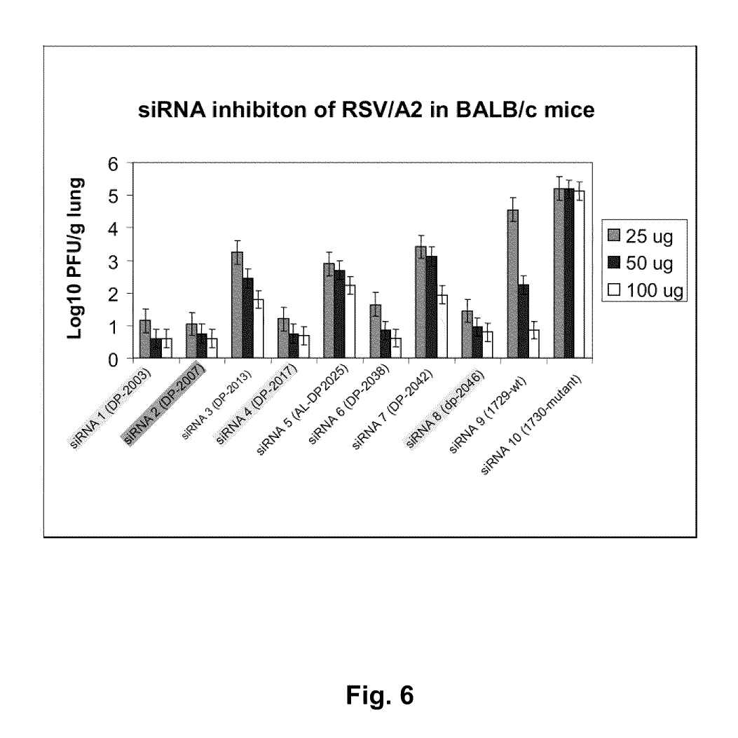

iRNA agents showing in vitro activity were tested for anti-RSV activity in vivo as outlined above. Several agents showed a reduction in viral titers of >4 logs when given prophylactically as illustrated in FIG. 6.

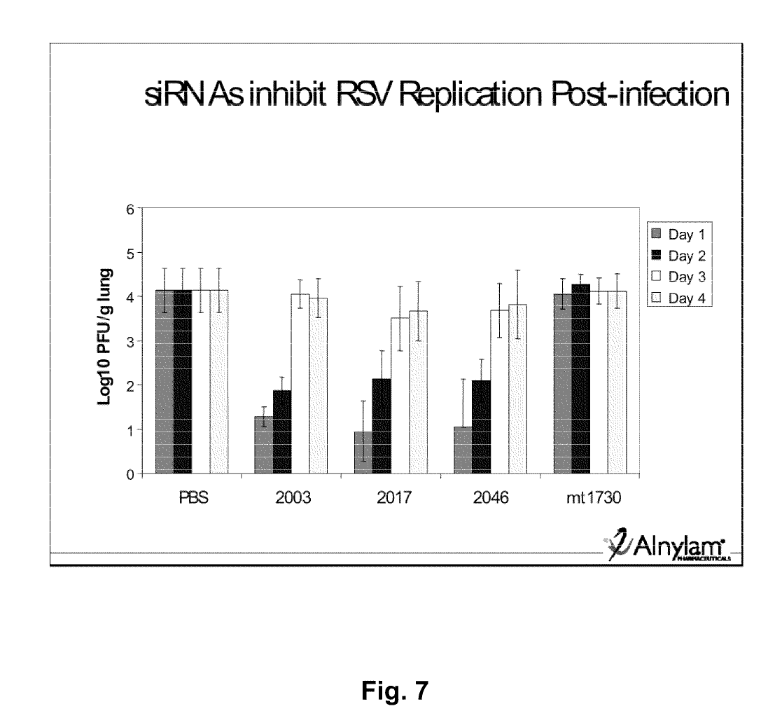

iRNA agents showing in vitro and/or in vivo activity were tested for anti-RSV activity in vivo as in the treatment protocol outlined above. Several agents showed a reduction in viral titers of 2-3 logs as shown in FIG. 7 when given 1-2 days following viral infection.

### Example 8

### Sequence Analysis of Isolates Across Target Sequence

Growth of isolates and RNA isolation: Clinical isolates from RSV infected patients were obtained from Larry Anderson at the CDC in Atlanta Ga. (4 strains) and John DeVincenzo at the University of Tenn., Memphis (15 strains). When these were grown in HEp-2, human epithelial cells (ATCC, Cat# CCL-23) cells, it was noted that the 4 isolates from Georgia were slower growing than the 15 strains from Tennessee; hence, these were processed and analyzed separately. The procedure is briefly described as follows:

Vero E6, monkey kidney epithelial cells (ATCC, Cat# CRL-1586) were grown to 95% confluency and infected with a 1/10 dilution of primary isolates. The virus was absorbed for 1 hour at 37° C., then cells were supplemented with D-MEM and incubated at 37° C. On a daily basis, cells were monitored for cytopathic effect (CPE) by light microscopy. At 90% CPE, the cells were harvested by scraping and pelleted by centrifugation at 3000 rpm for 10 minutes. RNA preparations were performed by standard procedures according to manufacturer's protocol.

Amplification of RSV N gene: Amplification of the RSV N gene fragment containing the ALN-RSV01 recognition site was performed using two step RT-PCR.

First, RNA was reverse transcribed using random hexamers and Superscript III Reverse transcriptase (Invitrogen, Carlsbad, Calif.) at 42° C. for 1 hour, to generate a cDNA library. Next a 1200 nt gene specific fragment was amplified using the forward primer RSV NF: 5′-AGAAAACTTGATGAAAGACA-3′ (SEQ ID NO: 285); and the reverse primer RSV NR: 5′-ACCATAGGCATTCATAAA-3′ (SEQ ID NO: 286) for 35 cycles at 55° C. for 30 sec followed by 68° C. for 1 min, using Platinum Taq polymerase (Invitrogen, Carlsbad, Calif.). PCR products were analyzed by 1% agarose gel electrophoresis.

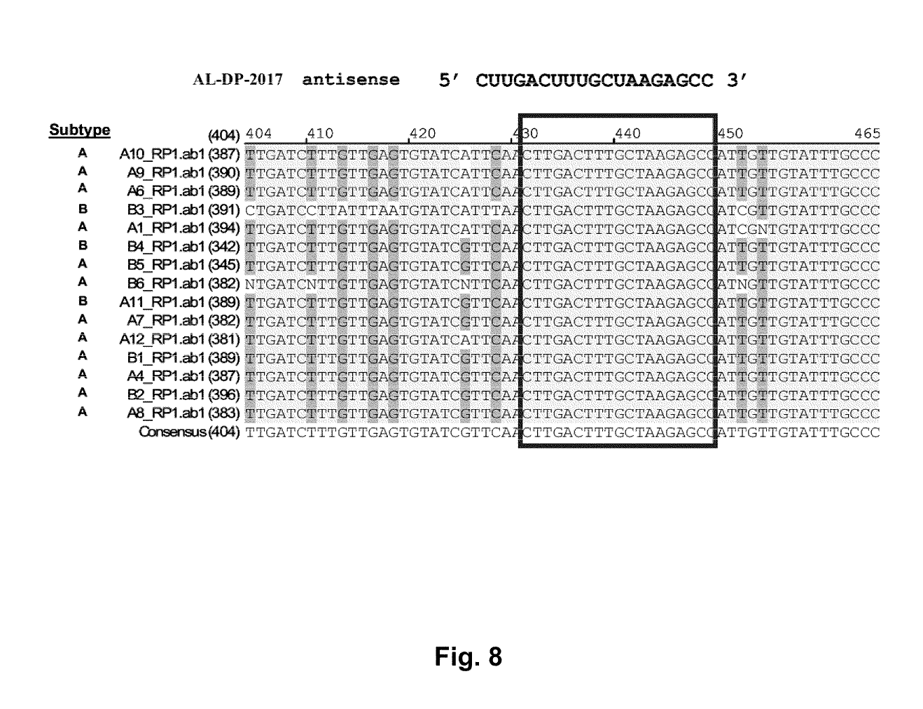

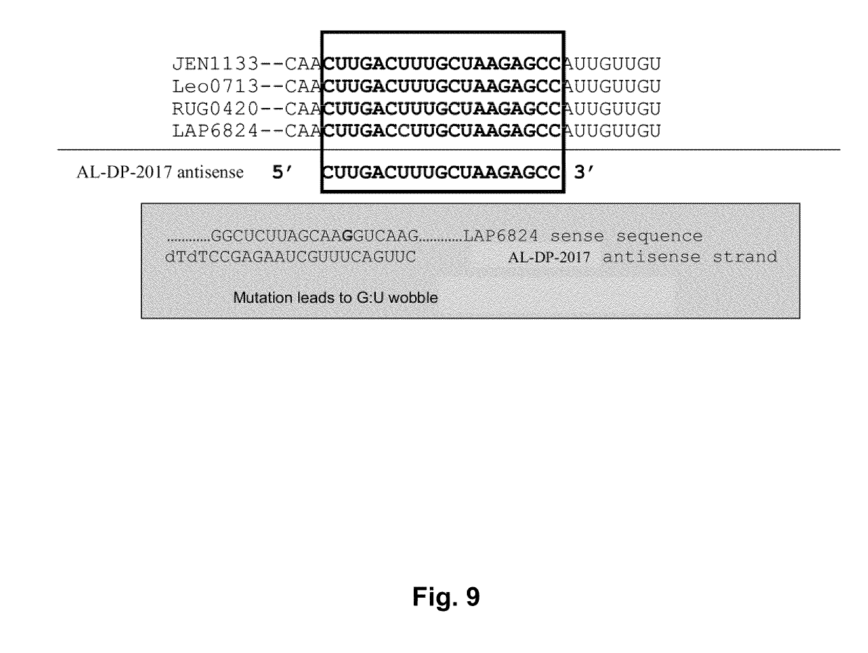

Results: Sequence analysis of the first 15 isolates confirmed that the target site for ALN-RSV01 was completely conserved across every strain. Sequence alignments are provided in FIG. 8. Importantly, this conservation was maintained across the diverse populations, which included isolates from both RSV A and B subtypes. Interestingly, when the 4 slower-growing isolates were analyzed, we observed that one of the 4 (LAP6824) had a single base mutation in the ALN-RSV01 recognition site as shown in the top part of FIG. 9. This mutation changed the coding sequence at position 13 of the RSV N gene in this isolate from an A to a G (FIG. 9, bottom).

Conclusions: From 19 patient isolates, the sequence of the RSV N gene at the target site for ALN-RSV01 has been determined. In 18 of 19 cases (95%), the recognition element for ALN-RSV01 was determined to be 100% conserved. In one of the isolates, there was detected a single base alteration changing the nucleotide at position 13 from an A to a G within the RSV N gene. This alteration creates a single G:U wobble between the antisense strand of ALN-RSV01 and the target sequence as shown in FIG. 9, bottom. Based on an understanding of the hybridization potential of such a G:U wobble, ALN-RSV01 is predicted to be effective in silencing the RSV N gene in this isolate.

### Example 9

### Synthesis and Purification of ALN-RSV01

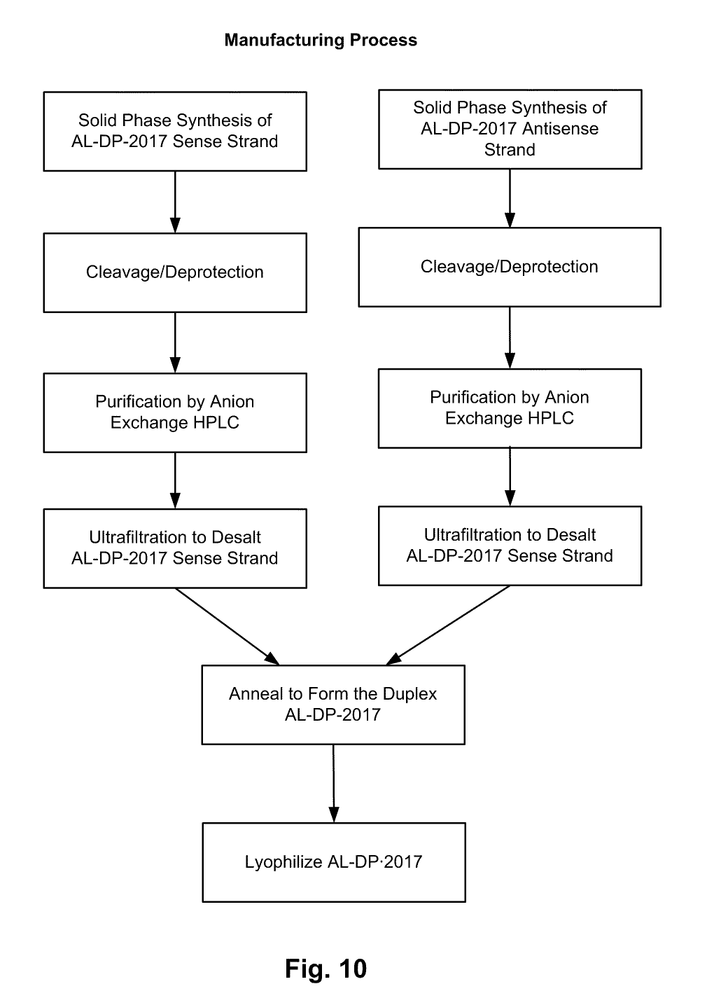

As shown in FIG. 10, the process for manufacturing the ALN-RSV01 drug substance consists of synthesizing the two single strand oligonucleotides (sense and antisense) by conventional solid phase synthesis using 3′-O-(2-cyanoethyl) phosphoramidite chemistry with the 5′-hydroxyls protected with 4,4′-dimethoxytriphenylmethyl (dimethoxytrityl, DMT) groups and tert-butyldimethylsilyl (TBDMS) protection on the 2′-hydroxyls of the ribose nucleosides. The crude single strand oligonucleotides were cleaved from the solid support, de-protected in a two-step process and purified by preparative anion exchange high performance liquid chromatography (AX-HPLC). The two single strands were combined in an equimolar ratio followed by annealing and lyophilization to produce the ALN-RSV01 drug substance.

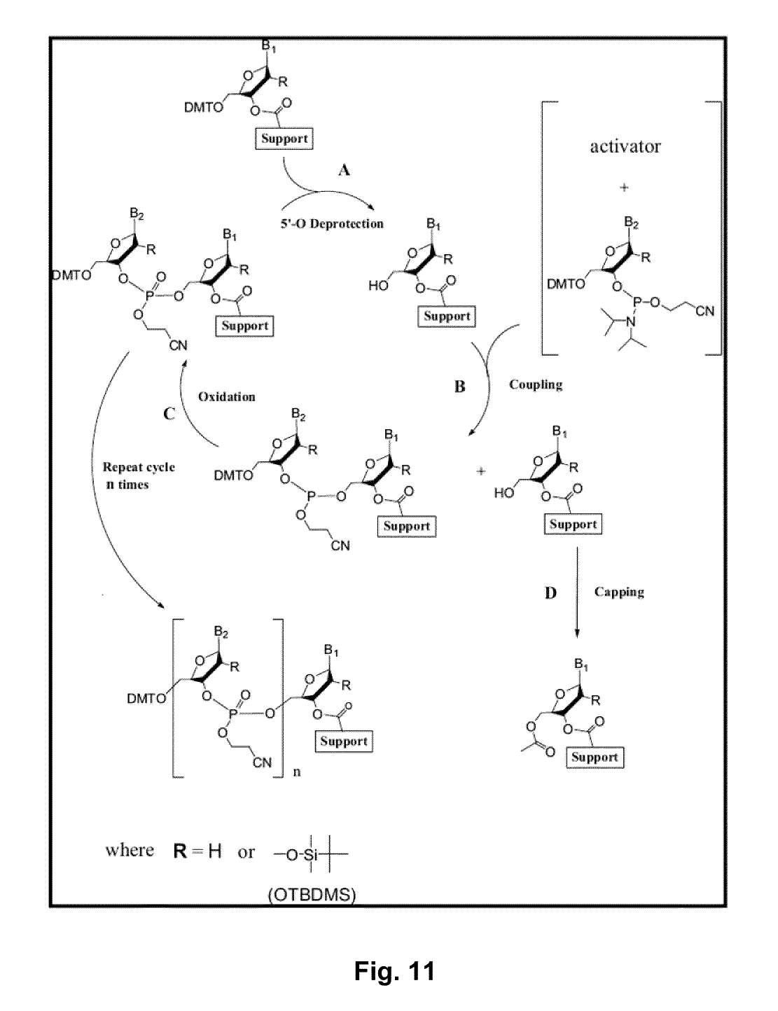

Solid Phase Synthesis: Assembly of an oligonucleotide chain by the phosphoramidite method on a solid support, such as controlled pore glass (CPG) or polystyrene followed the iterative process outlined in FIG. 11. The synthesis of ALN-RSV01 sense and antisense single strand intermediates was carried out on support loaded with 5′-dimethoxytrityl thymidine. Each intermediate was assembled from the 3′ to the 5′ terminus by the addition of protected nucleoside phosphoramidites and an activator. All the reactions took place on the derivatized support in a packed column. Each elongation cycle consisted of four distinct steps.

5′-Hydroxyl Deprotection (Detritylation, FIG. 11 step A): In the beginning of the synthesis the DMT-thymidine support was subjected to removal of the acid labile 4,4′-dimethoxytrityl protecting group from the 5′-hydroxyl. Each cycle of the synthesis thereafter commenced with removal of the corresponding DMT protecting group from the 5′ oxygen atom of the support-bound oligonucleotide (FIG. 11 step A). This was accomplished by treatment with a solution of dichloroacetic acid in toluene. Following detritylation the support-bound material was washed with acetonitrile in preparation for the next reaction.

Coupling (FIG. 11 step B): The elongation of the growing oligonucleotide chain was achieved by reaction of the 5′-hydroxyl group of the support-bound oligonucleotide with an excess of a solution of the protected nucleoside phosphoramidite, in the presence of the activator 5-ethylthio-1H-tetrazole. The amidite required in each step was determined by the oligonucleotide sequence described in Table 1c. This resulted in the formation of a phosphite triester internucleotide linkage. After allowing sufficient time for the coupling reaction to complete, excess phosphoramidite and activator was rinsed from the reactor using acetonitrile.

Oxidation (FIG. 11 step C): The newly created phosphite triester linkage was then oxidized by treatment with a solution of iodine in pyridine in the presence of water. This resulted in the formation of the corresponding phosphotriester bond (FIG. 11 step C). After the oxidation was complete, the excess reagent (iodine in pyridine/water) was removed from the column by rinsing the support with acetonitrile.

Capping (FIG. 11 step D): Although the coupling reaction proceeds in very high yield it is not quite quantitative. A small proportion (typically less than 1%) of the 5′-hydroxy groups, available in any given cycle, fails to couple with the activated phosphoramidite. In order to prevent reaction during subsequent cycles these sites were blocked by using capping reagents (acetic anhydride and N methylimidazole/2,6 lutidine/acetonitrile). As a result 5′-O-acetylated (‘capped’) support-bound oligonucleotide sequences were formed.

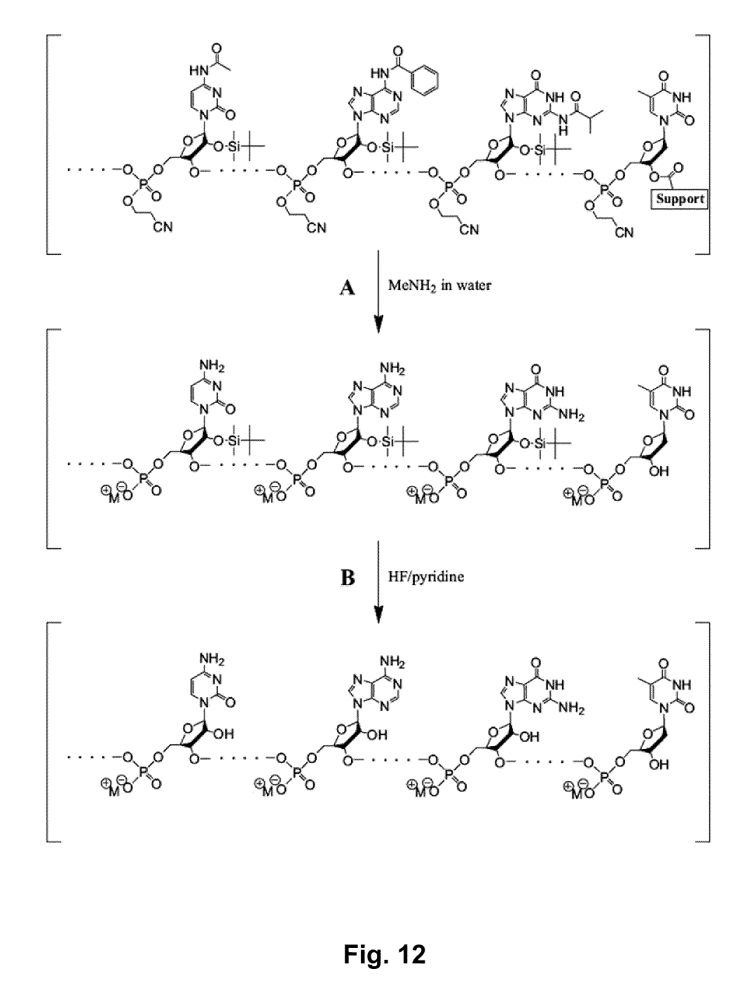

Cleavage and De-protection: Reiteration of this basic four-step cycle using the appropriate protected nucleoside phosphoramidites allowed assembly of the entire protected sequence. The DMT group protecting the hydroxyl at the 5′-terminus of the oligonucleotide chain was removed. The crude oligonucleotide was cleaved from the solid support by aqueous methylamine treatment with concomitant removal of the cyanoethyl phosphate protecting group. The support was removed by filtration and washed with dimethyl sulfoxide. The cleavage solution and wash were combined and held at room temperature or elevated temperatures to complete the deprotection of the exocyclic amino groups (benzoyl, isobutyryl, and acetyl) as shown in FIG. 12 step A. The 2′-O-TBDMS protecting groups were then cleaved using a solution of pyridine-hydrogen fluoride to yield the crude oligonucleotide (FIG. 12 step B). At the completion of the deprotection the solution was diluted with aqueous buffer and subjected to the purification step.

AX-HPLC Purification: Purification of each crude product solution was accomplished by AX-HPLC. A solution of crude product was loaded onto the purification column packed with Source 15Q media. The purification run was performed using sodium phosphate buffered eluents containing approximately 10% acetonitrile. A sodium chloride gradient was used to elute the oligonucleotide from the column. The purification was carried out at elevated temperatures (65-75° C.). The elution profile was monitored by UV absorption spectroscopy. Fractions were collected and pooled. Pools containing product at target purity levels were subjected to the next step in the process. Fractions that did not meet the acceptance criteria were, in some instances, repurified.

Desalting: The oligonucleotide solutions were concentrated using tangential flow filtration (TFF) using a polyethersulfone (PES) membrane cassette with a nominal 1,000 molecular weight cut-off. The retentate from the concentration step was pH adjusted and diafiltered with water to remove salts and solvents used in the AX-HPLC purification. The desalted product solution (retentate) was sometimes further concentrated by TFF before transfer to the next step.

Duplex Formation: The ultrafiltered solutions of the sense and antisense strand were combined in the desired proportions to form an equimolar mixture of the two intermediates. The required amounts of each single strand oligonucleotide were calculated based on UV assay and their molecular weights. To assure better control, the calculated amount of the first strand was mixed with less than the calculated amount of the second strand. AX-HPLC analysis of a sample of that mixture showed a well-resolved peak for the excess of the first strand together with a peak for the duplex. An additional amount of the second strand was added and a sample was analyzed again. This process was repeated until excess of one of the strands is was determined to be <1 area % as judged by the HPLC chromatogram. The solution was then heated and cooled under controlled conditions to anneal the duplex.

Freeze Drying: The duplex solution was filtered through a 0.2-micron filter before loading into disposable single use trays for bulk drying. The filtered product solution was freeze dried using a cycle consisting of three steps: (1) a freeze step, (2) primary drying at 0° C., and (3) secondary drying at 25° C. The result of this process is a lyophilized powder, i.e., a powder produced by the process that includes the steps of freezing a liquid and, drying the frozen liquid product under vacuum to remove by sublimation some or all of the frozen water.

Container Closure System: The lyophilized ALN-RSV01 drug substance was packaged in clean high-density polyethylene bottles with screw closures, labeled and stored in a freezer at −10 to −25° C. until shipment. In some instances, a moisture barrier bag was added to the packaging of the inventory. The selected bag (Model LF4835W from Laminated Films & Packaging) has three layers (white PET, foil, and polyethylene) and is specifically recommended as a barrier for oxygen and moisture.

Drug Finishing: ALN-RSV01 drug substance was delivered to a sterile fill/finish site as a lyophilized powder in sealed containers. Each container held a known weight of ALN-RSV01 drug substance. The bulk weight, the duplex purity, and the water content value were used to calculate the ALN-RSV01 drug substance available for formulation. As the drug substance is hygroscopic, whole containers were allocated for the manufacturing process. The size of the containers used allowed drug allocation to be close to the target lot size. The phosphate buffer solution was prepared to the required composition in a quantity in excess of that required to prepare the target lot size. The pH of the buffer was adjusted to 7.4±0.7. Allocated ALN-RSV01 drug substance, in whole vials, was dissolved into 80% of the target batch volume of phosphate buffer solution. An in-process sample was taken and the potency measured by UV/SEC. Using this assay value the theoretical batch size was calculated to give 100% potency. Using the remaining prepared buffer the lot was brought to this theoretical volume. The pH was monitored and adjusted as needed to 6.6+1.0. The lot was then aseptically filtered through two 0.22 μm sterile filters in series, filled into individual, sterile vials, stoppered, sealed, inspected (100% visual), and labeled. All vials were stored at 2-8° C.

Formulation Development: ALN-RSV01 drug product was formulated to a pH of 6.6 with sodium phosphate buffer. Phosphoric acid and sodium hydroxide were available for pH adjustment as needed. The formulation was near isotonicity, therefore there was no need to use sodium chloride to adjust osmolality. Osmolality of the ALN-RSV01 drug product used for intranasal administration or inhalation preferably ranges between 200-400 mOsm/kg.

Each vial of ALN-RSV01 drug product contains a volume of 0.5 mL. The product was filled into clear Type I glass vials sealed with Teflon-coated butyl rubber stoppers with aluminum flip-off overseals. All vials were maintained at 2-8° C. and were warmed to room temperature prior to use. In some instances, dilutions of drug product were prepared in normal saline by pharmacy staff.

Description and Composition of the Drug Product: ALN-RSV01 drug product was formulated as an aqueous solution in 50 mM phosphate buffer, pH 6.6 at a nominal concentration of 150 mg/mL. The quantitative composition of the ALN-RSV01 drug product is shown in Table 3. The weight shown for formulation reflects pure, anhydrous oligonucleotide. The amount of active ingredient per batch was calculated to account for the “as is” purity as determined by UV, ALN-RSV01 area value by SEC and the moisture content.

TABLE 3
quantitative composition of the ALN-RSV01 drug product

| | Quantity | | |
|---|---|---|---|
| Ingredient | per mL | | Function |
| ALN-RSV01 | 150 | mg | Active |
| | | | Ingredient |
| Dibasic Sodium Phosphate Heptahydrate | 11.42 | mg | Buffer |
| Monobasic Sodium Phosphate | 1.01 | mg | Buffer |
| Monohydrate | | | |
| Phosphoric Acid | q.s. | | pH Adjustment |
| Sodium Hydroxide | q.s | | pH Adjustment |
| Water for Injection | q.s. to 1 | mL | Vehicle |

Stability Studies: ALN-RSV01 (Lot# R01) was evaluated after initial, one, two, three, six and nine months of storage and found to be chemically stable using stability indicating methods such as denaturing AX-HPLC and SEC. Follow on studies confirmed stability of an aqueous buffered solution of the drug substance when stored at 2° C.-8° C. The lyophilized drug substance stored at −20° C. is expected to be at least as stable as the aqueous buffered solution. As used herein, “stable” means resistant to chemical changes that preclude product use in human subjects. Stability can be assessed by measuring stability and purity using methods that include denaturing AX-HPLC and SEC to provide measures of the overall proportion of the drug product comprised of the sense and antisense strands, as well as the fraction of the drug product that is found in duplex form. Other measures of stability include one or more of: tests for pyrogens, analysis of water content, tests of the Tm, i.e., the parameter that addresses the quality of the duplex, and assay values for inhibition of RSV gene expression, drop in viral titre, etc. using, e.g., tests exemplified in the working examples.

Compatibility with BD AccuSpray™ Nasal Spray System: A phase 2a clinical study was conducted by nasal instillation using the commercially available Becton-Dickinson Accuspray™ nasal spray system. Compatibility of ALN-RSV01 drug product was confirmed by evaluating the stability of ALN-RSV01 drug product in contact with the system over a fourteen-day period, both in ambient and refrigerated (2-8° C.) conditions. No degradation was observed upon storage of up to 14 days at 10 and 150 mg/mL, in ambient and refrigerated conditions as measured by appearance, SEC, stability indicating denaturing AX-HPLC, pH, osmolality and UV assay.

### Example 9

### Silencing Data on Isolates

Methods: Vero E6 cells were cultured to 80% confluency in DMEM containing 10% heat-inactivated FBS. For siRNA introduction, 4 μl of Transit-TKO was added to 50 μl of serum-free DMEM and incubated at room temperature for 10 minutes. Then, indicated concentration of siRNA was added to media/TKO reagent respectively and incubated at room temperature for 10 minutes. RNA mixture was added to 200 μl of DMEM containing 10% FBS and then to cell monolayer. Cells were incubated at 37° C., 5% CO₂ for 6 hours. RNA mixture was removed by gentle washing with 1× Hank's Balanced Salt Solutions (HBSS) and 300 plaque-forming units (pfu) per well of RSV/A2 (MOI=30) was added to wells and adsorbed for 1 hour at 37° C., 5% CO₂. Virus was removed and cells were washed with 1×HBSS. Cells were overlaid with 1% methylcellulose in DMEM containing 10% FBS media, and incubated for 6 days at 37° C., 5% CO₂. Cells were immunostained for plaques using anti-F protein monoclonal antibody 131-2A.

Results: Silencing by ALN-RSV01 was seen for all isolates as shown in Table 4.

TABLE 4
silencing of isolates by
ALN-RSV01

| | ALN-RSV01 | 2153 |
|---|---|---|
| | % plaques | % plaques |
| Isolate name | remaining | remaining |
| RSV/A2 | 4.49 | 80.34 |
| RSV/96 | 5.36 | 87.50 |
| RSV/87 | 10.20 | 79.59 |
| RSV/110 | 5.41 | 81.08 |
| RSV/37 | 4.80 | 89.60 |
| RSV/67 | 2.22 | 91.67 |
| RSV/121 | 6.25 | 82.50 |
| RSV/31 | 4.03 | 96.77 |
| RSV/38 | 2.00 | 92.67 |
| RSV/98 | 5.13 | 91.03 |
| RSV/124 | 3.74 | 90.37 |
| RSV/95 | 7.32 | 64.02 |
| RSV/32 | 5.45 | 92.73 |
| RSV/91 | 8.42 | 95.79 |
| RSV/110 | 12.07 | 94.83 |
| RSV/54 | 1.90 | 89.87 |
| RSV/53 | 7.41 | 94.07 |
| RSV/33 | 7.69 | 95.19 |

Conclusion: All clinical isolates tested were specifically inhibited by ALN-RSV01 by greater than 85%. No isolates were significantly inhibited by the mismatch control siRNA 2153.

### Example 10

### Silencing in Plasmid Based Assay

Methods: A 24-well plate was seeded with HeLa S6 cells and grown to 80% confluence. For each well, 1 μg of RSV N-V5 plasmid was mixed with a siRNA (at indicated concentration), in 50 ul OPTI-MEM which then was added to a Lipofectamine 2000 (Invitrogen)-Optimem mixture prepared according to manufacturer's instructions. This mixture was incubated for 20 minutes at room temperature to allow time for complex formation between the siRNA and the Lipofectamine-Optimem components. The complexed mixture was added complex to cells and incubated at 37° C. overnight. The media was removed, cells were washed with phosphate-buffered saline (PBS) and then lysed by the incubation with 50 ul Lysis buffer (RIPA buffer (50 mM Tris-HCl pH 8.0, 150 mM NaCl, 1 mM EDTA, 0.5% Na deoxycholate, 1% NP-40, 0.05% SDS) for 1-2 min. Lysates were analyzed and inhibition of RSV-N protein expression was quantified by measuring the level of RSV-N protein in cell lysates, as detected by Western blotting with an anti-V5 antibody.

Results: Transient plasmid expression was shown to be an effective assay for RNAi agents (Table 5).

TABLE 5
Silencing as measured in a plasmid based assay

| | siRNA | Concentration | Protein % | Activity % |
|---|---|---|---|---|
| 1 | ALN-RSV01 | 10 nM | 0 | 100 |
| 2 | ALN-RSV01 | 1 nM | 0 | 100 |
| 3 | ALN-RSV01 | 100 pM | 0 | 100 |
| 4 | ALN-RSV01 | 10 pM | 11.78 | 88.22 |
| 5 | ALN-RSV01 | 1 pM | 70.63 | 29.37 |
| 6 | ALN-RSV01 | 100 fM | 72.7 | 27.3 |
| 7 | Control | PBS | 100 | 0 |
| 8 | 2153 | 10 nM | 94.54 | 4.5 |

Conclusions: siRNA 2017 (ALN-RSV01) was shown to specifically and dose-dependently inhibit the production of RSV N protein when transiently cotransfected with a plasmid expressing the RSV N gene. Inhibition is not observed using mismatch control siRNA 2153.

### Example 11

### Silencing of RSV Via Aerosol Delivery of siRNA

Method: A 2 mg/ml solution of AL-DP-1729 or AL-DP-1730 was delivered via nebulization using an aerosol device for a total of 60 sec. Viral samples were prepared from lung as described above and measured using an ELISA instead of a plaque assay. The ELISA measures the concentration of the RSV N protein in virus-infected cells obtained from mouse lung lysates.

Methods: Lung lysate was diluted 1:1 with carbonate-bicarbonate buffer (NaHCO₃ pH 9.6) to a working concentration of 6-10 μg/100 μL, added to each test well and incubated at 37° C. for 1 hour or overnight at 4° C. Wells were washed 3× with PBS/0.5% Tween 20 then blocked with 5% dry milk/PBS for 1 hour at 37° C. or overnight at 4° C. Primary antibody (F protein positive control=clone 131-2A; G protein positive control=130-2G; negative control=normal IgG1κ, (BD Pharmingen, cat. #553454, test sera, or hybridoma supernatant) was added to the wells at a final dilution of 1:1000, and incubated at 37° C. for 1 hour or overnight at 4° C. Wells were washed 3× with PBS/0.5% Tween 20. Secondary antibody (Goat Anti-mouse IgG (H+L) whole molecule-alkaline phosphatase conjugated) was added to the wells at a final dilution of 1:1000 (100 μl/well) and incubated at 37° C. for 1 hour or overnight at 4° C. The wells were washed 3× with PBS/0.5% Tween 20, after which time 200 μl of Npp (Sigmafast) substrate (Sigma Aldrich N2770) made according to manufacturer's instructions was added to the wells. This mixture was incubated for 10-15 at 37° C., and absorbances at OD 405/495 were measured.

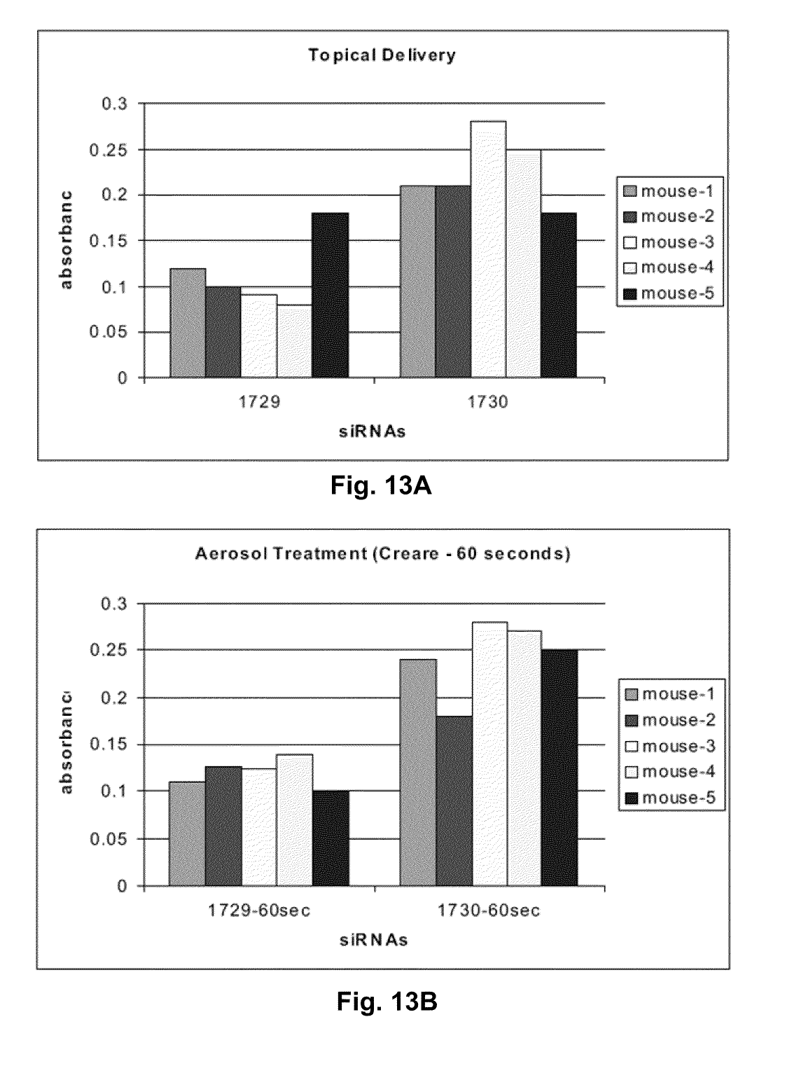

Conclusion: Delivery of RSV specific siRNA decreases the levels of RSV N protein in mouse lungs as compared to the mismatch control siRNA (FIG. 13a-b).

### Example 12

### In Vivo Inhibition at Day −3-Prophylaxis

Method: In vivo prophylaxis was tested using the in vivo method described above except that the siRNA is delivered at different times prior to infection with RSV from 3 days before to 4 hrs before. Results were obtained for AL-DP-1729 (active) and AL-DP-1730 (mismatch control).

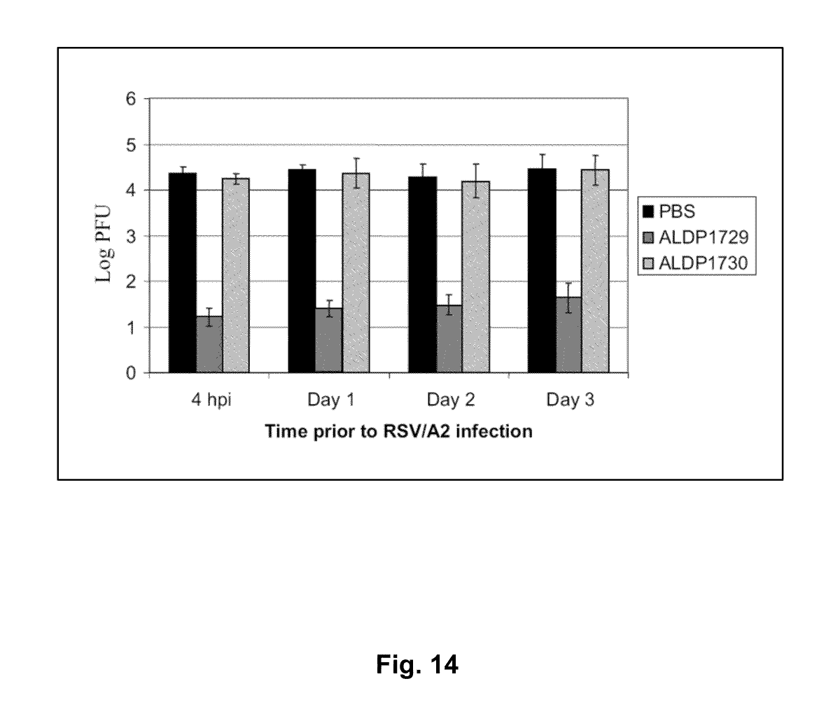

Results: Active siRNA delivered intranasally up to 3 days prior to viral challenge show specific and significant silencing in vivo as shown in FIG. 14.

### Example 13

### Nebulization of ALN-RSV01 with Pari eFlow® Device

Droplet Size and Analytical Integrity

Methods: A 150 mg/ml solution of ALN-RSV01 (in 2 mls of PBS) was filled into the Pari eFlow® electronic device and run until nebulization was completed and all aerosol was collected and allowed to condense in a polypropylene tube. Aliquots of material post nebulization were analyzed to determine geometric droplet size distribution by laser diffraction (Malvern MasterSizerX) under standard conditions. Aliquots of material pre and post nebulization were analyzed to determine analytical integrity by a stability using anion exchange HPLC methodology.

Results: Aerosolized ALN-RSV01 had a Mass Median Diameter (MMD) of 3.1 μm, a Geometric Standard Deviation (GSD) of 1.6 and a total respirable fraction of 85% (i.e., % particles <5 μm) confirming that a 75 mg/ml solution could be aerosolized to yield respirable material with appropriate particle size. Comparison to control samples of ALN-RSV01 formulation which were not nebulized showed matching chromatograms, demonstrating that the oligonucleotide can be nebulized by eFlow® without degradation.

Biological Activity: A 25 mg/ml solution of ALN-RSV01 (in 1 ml of PBS) was prepared, 100 μl was removed (pre-nebulization aliquot) prior to nebulization with the Pari eFlow® electronic device, and 500 μl of the nebulized solution was collected after condensing by passage over an ice bath into a chilled 50 ml conical tube (post-nebulization aliquot). Serial dilutions of both aliquots were tested in our in vitro transfection/infection plaque assay as previously described with the exception that siRNA was complexed with lipofectamine-2000.

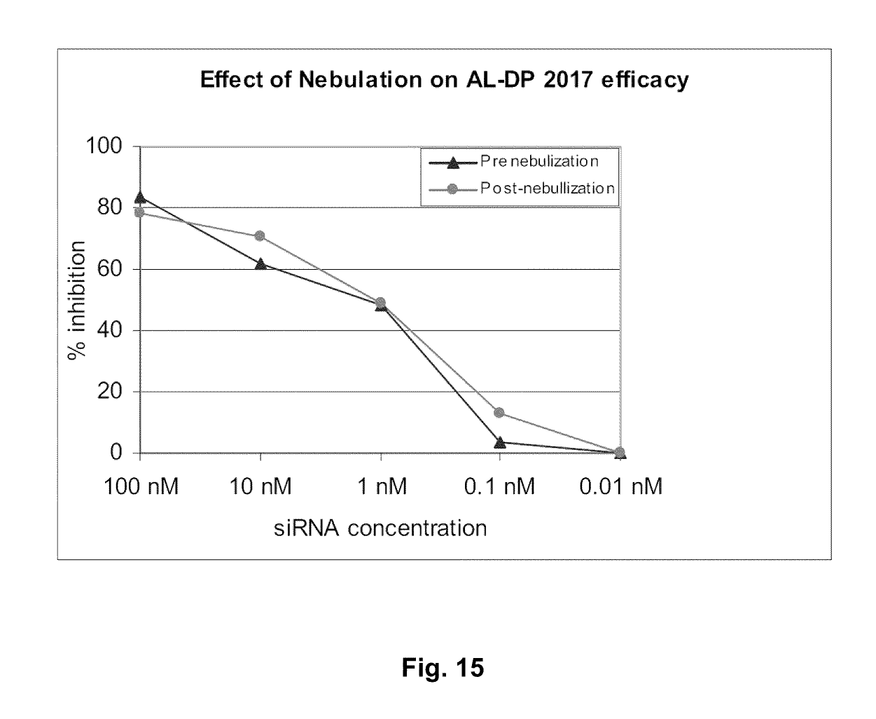

Results: siRNA pre and post nebulization efficiently inhibited RSV viral replication in a Vero cell plaque assay. The degree of inhibition was almost identical between the two samples and showed a dose response leading to >80% silencing at the highest siRNA concentrations confirming that nebulized ALN-RSV01 maintains biological activity. Results are shown in FIG. 15.

### Example 14

### Inhalable siRNAs: ALN-RSV01

To investigate the in vivo effects of aerosolization and delivery by inhalation of siRNAs targeting RSV as well as the pharmacokinetic properties of inhaled siRNAs, a double-blind, randomized, placebo-controlled, evaluation study in human adult subjects was performed. The study measured routine bloods and clinical observations, inflammatory biomarkers, tolerability and plasma pharmacokinetics. As used in this specification “inhalation” refers to administration of a dosage form that is formulated and delivered for topical treatment of the pulmonary epithelium. As described above, an inhalable dosage form comprise particles of respirable size, i.e., particles that are sufficiently small to pass through the mouth or nose and larynx upon inhalation and into the bronchi and alveoli of the lungs.

In the study, ascending doses of aerosolized ALN-RSV01 or placebo were administered once daily by inhalation for 3 consecutive days to 4 cohorts of 12 subjects each with 8 subjects receiving ALN-RSV01 and 4 subjects receiving placebo in each cohort for a total of 48 subjects. ALN-RSV01 maximum solubility concentration in the finished product is 150 mg/mL. Therefore, a 150 mg/ml solution of ALN-RSV01 was diluted to the appropriate concentration and filled into the Pari eFlow® electronic device and run until nebulization was completed.

Blood samples evaluated for pharmacokinetics (PK) included pre dose and post dose at 2, 5, 15, and 30 minutes, 1 hour and 24 hours on Day 0 and post third dose at 2, 5, 15, and 30 minutes, 1 hour and 24 hours after the third dose (13 samples per subject). Urine collection for PK included: pre dose and post third dose at 0-6 hours, 6-12 hours and 12-24 hours.

Plasma ALN-RSV01 concentrations, and derived parameters (Cₚᵣₑ, Cₘₐₓ, tₘₐₓ, t_{1/2}, CL/F, V_{d}/F, AUCₗₐₛₜ) were evaluated for PK.

ALN-RSV01 has previously been evaluated for toxicity by inhalation administration in rats and monkeys at doses as high as 36 mg/kg/day and 30 mg/kg/day, respectively. The highest dose to be administered in the single dose part of the current study was 210 mg/day (or 3 mg/kg, assuming 70 kg body weight). On a mg/kg basis, this dose is approximately 10 fold lower than the doses given previously to rats and monkeys.

The initial doses in this study were 7.0 mg, 21.0 mg and 70.0 mg providing a safety margin of about 300 fold, 100-fold and 30 fold, respectively.

Dose levels for the multiple dose part of the study were 7.0 mg, 21.0 mg, 70.0 mg and 210 mg, given as a daily delivered dose (DD) for three consecutive days.

The highest dose to be administered in the single dose part of the current study was chosen at 210 mg/day (or 3 mg/kg, assuming 70 kg body weight).

Study drug exposure duration in the multiple dose part of the study was chosen to be 3 days, with once daily dosing, based on the intended therapeutic dosing duration which is likely to be short due to the acute nature of RSV infections.

Pulmonary Function Tests

PFT were conducted at screening to identify healthy volunteers with respect to capacities and flow-rates. PFT provides an objective method for assessing the mechanical and functional properties of the lungs and chest wall. PFT measures:
Lung capacities e.g., Slow Vital Capacity (SVC) and Force Vital Capacity (FVC), which provide a measurement of the size of the various compartments within the lung
Volume parameters (e.g., FEV1) and flow-rates (e.g., FEF25-75), which measure maximal flow within the airways

Serial evaluation of pulmonary function after inhalation of ALN-RSV01 or placebo were conducted. Additional PFT testing was conducted on Day 0 at pre-dose (about −30 min) and at 30 min and 2 h, 6 h, and 12 h on Days 1, 1 and 2 at the same time as pre-dose on Day 0.

PFT provides lung capacities and flow-rates. The SVC is the volume of gas slowly inhaled when going from complete expiration to complete inhalation. The FVC is the volume expired when going from complete inhalation to complete exhalation as hard and fast as possible. The FEV1 is the amount expired during the first second of the FVC maneuver. The Forced Expiratory Flow (FEF25-75) is the average expiratory flow over the middle half of the FVC. SVC, FVC, FEV1 and FEF25-75 was measured according the ATS/ERS guidelines. In this study, FEV1 was the main parameter.

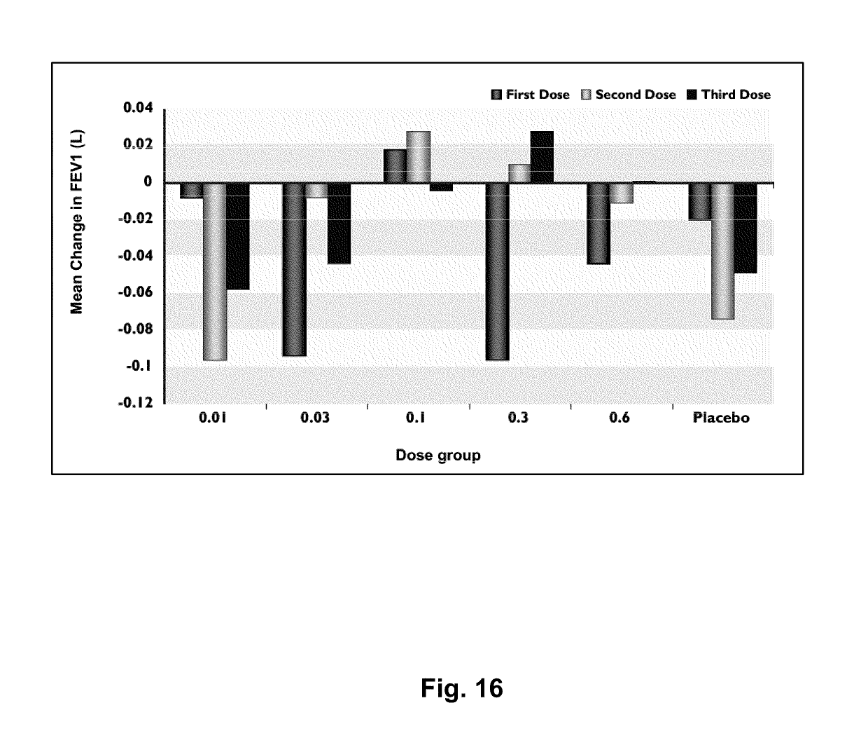

As shown in FIG. 16, no significant change in lung function was seen on aerosol administration of ALN-RSV01.

Plasma

For single dosing, blood samples were collected for the analysis of ALN-RSV01 in plasma at pre dose and post dose (post nebulization) at 2, 5, 15, and 30 minutes, 1 hour and 24 hours on Day 0 (7 samples per volunteer).

For multi-dosing, blood samples were collected for analysis of ALN-RSV01 in plasma at pre-dose and at 2, 5, 15 and 30 min, 1 h, and 24 h post first-dose on Day 0 (post nebulization), and at 2, 5, 15, 30 min, 1 h, and 24 h after the third dose (post dose nebulization of third dose).

Blood samples of 5 mL each were taken via an indwelling intravenous catheter or by direct venipuncture into tubes containing K3EDTA as the anticoagulant. In case of sampling through the intravenous catheter, the first 1 mL of blood was discarded in order to prevent any dilution of blood with heparin used to flush the catheter.

Results

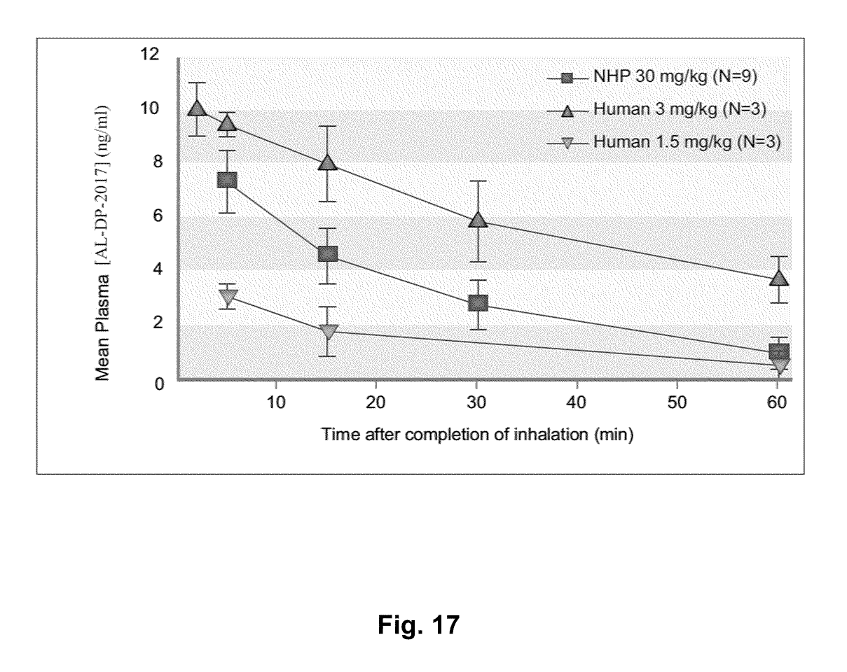

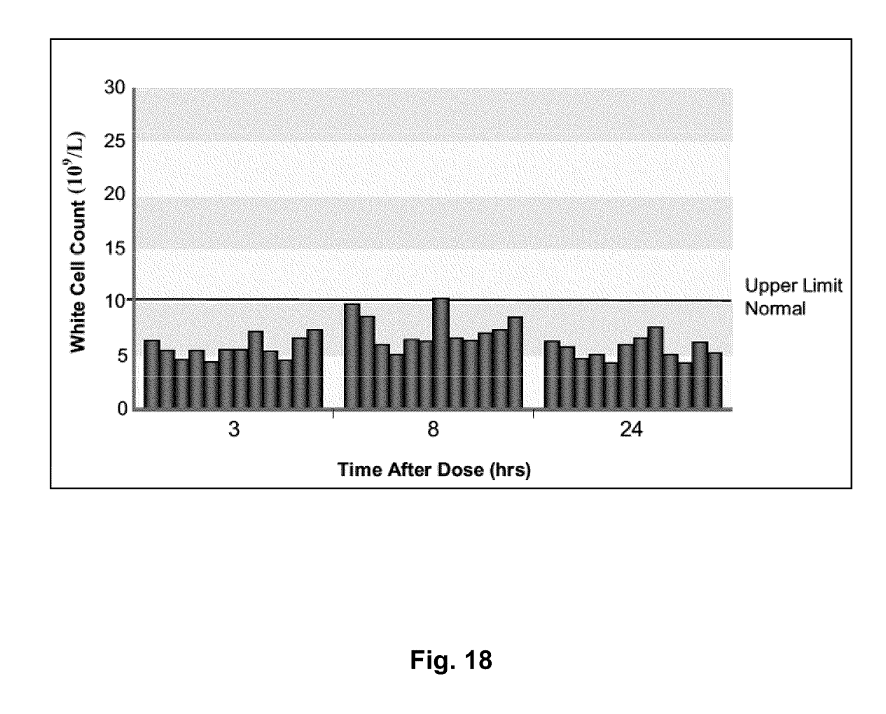

A safe and well tolerated regimen of ALN-RSV01 has been defined for further clinical development. To this end the data show that plasma exposure for a given dose in man is greater than in nonhuman primates. See FIG. 17. While single dose administration at 3 mg/kg equivalent was associated with a greater incidence of a flu-like adverse event (cough, headache, non-cardiac chest pain, pharyngo-laryngeal pain and chills) relative to placebo multi-dose administration of ALN-RSV01 (AL-DP 2017) was safe and well-tolerated when given once daily for 3 days up to 0.6 mg/kg per dose. There was also no evidence of neutrophil leucocytosis after multi dosing of ALN-RSV01 (AL-DP 2017) in the highest dose cohort (0.6 mg/kg). See FIG. 18.

### Example 15

### A Split Dose of ALN-RSV01 Reduced RSV Titer Levels In Vivo

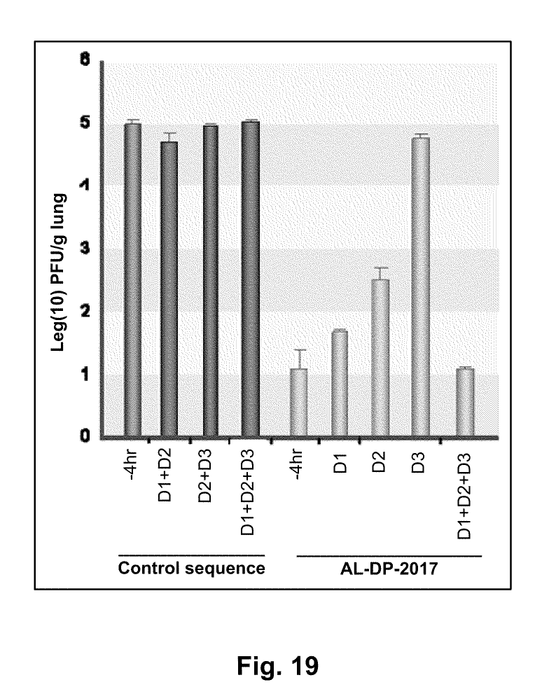

A fixed dose (120 μg) of ALN-RSV01 was administered to rodents intranasally 4 hours prior to RSV instillation (10⁶ pfu at timepoint zero). Mice were then administered 120 μg of ALN-RSV01 intranasally on the first, second or third day following instillation, or in three administrations split equally over days 1, 2, and 3 following instillation. The dose administered over the course of three days maintained the same reduced RSV titer levels in the lung as observed by the single dose of the siRNA on the first day following infection. See FIG. 19.

### Example 16

### Randomized, Double-Blind, Placebo-Controlled, Parallel-Group Study of Safety and Efficacy of Intranasal ALN-RSV01 Administered to Adult Volunteers Experimentally Inoculated with RSV

A study was conducted to assess the safety and tolerability of intranasal ALN-RSV01 versus placebo, administered in a multiple-dose schedule (once daily for 5 days) to healthy male adult volunteers experimentally inoculated with respiratory syncytial virus (RSV). Secondary objectives included determining the impact of ALN-RSV01 on symptoms of RSV infection, RSV infection rate based on measures of viral load, and understanding the potential antiviral activity of ALN-RSV01

Study design: A randomized, placebo-controlled, double-blind, parallel-group, inpatient (quarantine) study was undertaken to assess the safety and efficacy of intranasal ALN-RSV01 administered to healthy male adult volunteers 32 and 8 hours prior to inoculation and daily for 3 days after inoculation with an RSV challenge strain. A total of 5 doses of ALN-RSV01 were administered to each subject. A total of 88 subjects (44 ALN-RSV01 and 44 placebo) were enrolled into the trial in sequential cohorts. Two initial dose cohorts of 8 subjects each (4 ALN-RSV01 and 4 placebo) were enrolled to evaluate initial safety and tolerability of the ALN-RSV01 dose levels (75 mg in Cohort 1 and 150 mg in Cohort 2, given once daily for 5 days) prior to enrolling sequential target-dose cohorts (e.g., Cohorts 3, 4, 5) of approximately 24 subjects each (12 ALN-RSV01 and 12 placebo in a 1:1 ratio, 150 mg given once daily for 5 days), until a total of 88 subjects were exposed to study drug (active or placebo).

Subjects were screened during a 4-month period (initial Screening, Days −120 to 14) and a 2-month period (Screening, Days −60 to −14) with key screening assessments repeated on Day -2 prior to admission to the quarantine unit. Subjects who continued to satisfy eligibility criteria on Day -2 were checked in to a quarantine facility and remained in the facility until discharged on Day 11 (or Day 12/13 if discharge is delayed); all subjects were required to return for a follow-up visit on Day 28.

Viral challenge agent: In all cohorts, RSV inoculum (RSV-A obtained from Viral Antigens Inc, TN, US) was administered at a dose of 5 log 10 PFU one time, 8 hours after the second dose of study drug on Day 0. This was administered as a dose of 0.5 mL/naris (intranasal administration via two 0.25 mL nasal sprays produced by a Becton-Dickinson Accuspray™ nasal spray system) of RSV challenge solution (thawed RSV stock diluted to a final concentration of 5 log 10 PFU/mL in an RSV stabilization medium). The total dose volume per subject was 1.0 mL.

Dosage, route of administration and duration of treatment of investigational drug and control: 0.5 mL of ALN-RSV01 was administered intranasally. In general, in each naris (intranasal administration via two 0.25 mL nasal sprays produced by a Becton-Dickinson Accuspray™ nasal spray system) for a total of 1.0 mL per dose, administered 32 and 8 hours prior to inoculation with RSV and at 16, 40, and 64 hours post-inoculation (total doses=5). The entire contents of the BD Accuspray™ were sprayed into one naris (0.25 mL). With the subject in an upright position with head tilted back, the tip of the sprayer was placed just inside the nostril. The plunger was rapidly depressed until the plunger could not be depressed further, and the entire contents of the BD Accuspray™ was thereby administered. With the subject's head remaining tilted back, the entire contents of a filled BD Accuspray™ was sprayed into the other nostril. This process was repeated with a second set of BD Accuprays™ for a total of two sprays per naris. Subjects were instructed to make every attempt not to allow any study drug to drip out of their nose and the subjects also were instructed to try not to blow their nose for 15 minutes after investigational drug administration. Once the ALN-RSV01 was administered, each sprayer will be disposed of according to standard procedures at the study site. Cohort 1 received 75 mg/dose for 5 doses; cohorts 2 through the final cohort received 150 mg/dose for 5 doses. The same administration volumes and schedules were used for the control group. This group received sterile normal saline (0.9% NaCl).

Endpoints: Safety endpoints evaluated the tolerability of ALN-RSV01 relative to placebo in RSV-exposed individuals, and included the following: Frequency and severity of treatment related adverse events; treatment-related changes in vital signs; treatment-related changes in physical examination (PE) findings; treatment-related changes in nasal examination findings; treatment-related changes in electrocardiogram (ECG) parameters; treatment-related changes in clinical laboratory assessments. Efficacy endpoints were exploratory and included one or more of the following: changes in symptoms of RSV infection; frequency of RSV infection, expressed as the percentage of subjects developing infection after inoculation; assessment of RSV viral load (including one or more of the following measures: peak amount of viral load; time to peak viral load; mean daily viral load; duration of viral shedding; overall viral load (based on the area under the concentration-time curve [AUC]); and serum RSV antibody response. These endpoints were evaluated from one or more of the following: subject-reported signs and symptoms of RSV infection (runny nose, stuffy nose, sneezing, sore throat, earache, malaise, cough, shortness of breath, headache, or muscle and/or joint aches) recorded on the RSV Symptom Diary Card; RSV detected and/or quantified in respiratory secretions (nasal washes) using several virologic detection and quantification methods which could include: (a) RSV rapid antigen detection; (b) non-quantitative culture (spin enhanced); (c) quantitative culture (plaque assay); or (d) quantitative real time reverse transcriptase polymerase chain reaction (rt PCR); directed physical examination results; mucus weight measurements; RSV serology (neutralizing antibody titer); and cytokine panel measures (obtained from nasal wash samples and blood draws). Cytokine panel measures included measures of C-reactive protein (CRP), and the cytokines tumor necrosis factor (TNF), interleukin 1 Ra (IL1-Ra) and granulocyte colony stimulating factor (G CSF).

Nasal wash procedures were at Screening (Day −60 /−14) to familiarize subjects with the procedure, on Day −2 (baseline and for infectious disease screen), and daily on Days 2-11. Nasal wash was not conducted on Days, −1, 0 and 1. After instructing the subjects to place a catheter into his/her nostril, the study nurse or technician injected 5 mL of sterile saline into the catheter lumen, waited 10 seconds and withdrew the fluid; this injection and withdrawal process was repeated twice more for a total of 3 washes, then the nasal wash fluid was dispensed into the sterile collecting container. The procedure was repeated (wash with 5 mL three times) in the other nostril. Nasal wash samples were divided into aliquots and processed as follows: 1 mL chilled on ice and used immediately for the Viral Plaque Assay, shell vial culture, rapid RSV antigen assay, and the infectious disease screen (ID screen only used for subjects developing new symptoms after Day 8; samples taken on Days 9-11/12/13); 1 mL aliquot frozen for cytokine analysis; 1 mL aliquot frozen for PCR Assay; 1 mL frozen for Viral Resistance Protocol assessments; 1 mL aliquot frozen for pharmacodynamic assessment; and remaining 1 mL aliquots was frozen and stored.

Safety Analysis: Primary safety analyses were performed on the Safety population. Where appropriate, demographics, subject disposition, screening and baseline characteristics were summarized for the PP and ITT populations.

Nasal examinations, physical examinations, and vital signs (pulse rate, blood pressure, respiration rate, and oral temperature) were tabulated and summarized by treatment group and study day for the SP. Change from baseline vital signs was calculated and summarized.

Laboratory values were tabulated and summarized by treatment group and study day for the SP, using the appropriate summary statistics. Change from baseline laboratory data for hematology and biochemistry was calculated and summarized. Laboratory values outside of the normal ranges were listed separately, together with comments as to their clinical significance.

Values for inflammatory biomarkers (cytokine panel from blood samples) were tabulated and summarized by treatment group and study day for the SP, using the appropriate summary statistics. Change from baseline for these biomarkers were calculated and summarized.

Treatment emergent adverse events were defined as those AEs not relating to RSV infection that occur after the first dosing of study drug. Treatment-emergent AEs were tabulated and summarized.

Efficacy Analysis: RSV Infection. The number of RSV infected subjects (using each of the virologic detection methods separately), was summarized by treatment group. The duration of time from inoculation to first detection of RSV in all previously published experiences with experimental human RSV infection ranges between 2 and 6 days 21-27. Therefore in this protocol, a subject was defined as infected if they showed their initial presence of RSV in the nasal wash starting from Day 2 through Day 8 (inclusive) after inoculation. Data before Day 2 was excluded from this analysis because it was assumed that any RSV detected in the nasal wash on days prior to study Day 2 are from inoculum itself, and not because the subject was successfully infected. Fisher's exact test was to compare the proportion of infected subjects in each treatment group. If RSV is first detected after Day 8, the subject was not considered infected. This was done to avoid including subjects in the analysis who have experienced (an unlikely) cross-infection, not affiliated with the inoculation.

Clinical Signs and Symptoms. The level of each clinical sign and symptom of RSV infection (runny nose, stuffy nose, sneezing, sore throat, earache, malaise, and headache) were as categorical data, by treatment group and study day. An average daily score and overall average symptom score (post inoculation) was calculated, and summarized as continuous data. The Wilcoxon rank-sum test was used to compare the overall average symptom score between each treatment group. The results of the directed PE was treated in a similar way.

Assessments of RSV Load. Quantitative measurement of RSV in the nasal wash from Day 2 onwards were summarized using the following parameters: duration of viral shedding; peak viral load; time to peak viral load; mean daily viral load; overall viral load (based on AUC). Viral load was calculated as zero if: a) there was no virus detected during the 2-8 day post inoculation time frame or b) if 1st viral detection occurred after the day 8 post inoculation time frame. The Wilcoxon rank-sum test was used to compare each parameter between each treatment group.

Daily mucus weight was summarized by treatment group and study day. An average mucus weight post inoculation was calculated and summarized. The Wilcoxon rank-sum test was used to compare the average mucus weight between each treatment group.

Serum RSV antibody response was summarized as continuous data by treatment group and study day. The change from baseline to end-of-study visit (Day 11) and follow-up visit (Day 28) was calculated and summarized. The Wilcoxon rank-sum test was used to compare the change from baseline values between each treatment group.

Values for cytokines (from nasal wash samples) were tabulated and summarized by treatment group and study day for the SP, using the appropriate summary statistics. Change from baseline for these biomarkers were calculated and summarized.

Results: ALN-RSV01 is shown to be a safe and effective treatment for the prevention or treatment of RSV infection in humans.

### Example 17

### In Vitro Activity of Modified RSV siRNAs Using Plasmid Based Assay (psiCHECKTM-2 Vector-RSV01 Target Reporter Construct)

Materials and Methods

The psiCHECKTM-2 Vector-RSV01 target Reporter, a construction of a Renilla luciferase RSV01 target site reporter plasmid was made. The RSV01 target site was cloned between the stop-codon and the polyA-signal of Renilla-Luciferase. RSV01 on-target assay with psiCheck2 vector was used for the activity screening of the RSV siRNAs and measuring the suppression of Renilla-Luciferase expression in relation to Firefly-Luciferase by Dual-Glo-system (Promega). Renilla luciferase was used for activity screening. Firefly luciferase was used for normalization. HeLa-S3 cells were transfected with psiCheck2 reporter plasmid. The cells were transfected with siRNA 4 h after plasmid transfection. DualGlo luminescence assay was performed after 16 h cell culture. For the Single Dose assay 5 nM & 1 nM siRNA concentrations were used. For the Dose Response assay 3 nM-648 fM concentrations were used. 132 RSV siRNAs with 2′Fluoro modifications or combination of 2′O-Methyl (OMe) and 2′Fluoro modification and 33 RSV siRNAs with only 2′OMe modification no 2′Fluoro, without PS and without UA protection were tested.

Results

Table 6 shows the in vitro activity of 2′Fluoro or combination of 2′OMe and 2′Fluoro modified RSV siRNA. Table 7 shows the sequences of 2′Fluoro or combination of 2′OMe and 2′Fluoro modified RSV siRNA. Table 8 shows the in vitro activity of 2′OMe modified RSV siRNA. Table 9 shows the sequences of 2′OMe modified RSV siRNA.

Activity % refers to the suppression of Rluc expression. Note: 1 means no activity equivalent to 0% activity. 55 of 132 modified compounds show >90% activity and >50% are better than the Rluc specific siRNA.

TABLE 6
in vitro activity of 2′Fluoro or combination of 2′OMe and 2′Fluoro
modified RSV siRNA.

| | | | | | Average |
|---|---|---|---|---|---|
| | Residual | Activity % | Residual | Activity % | IC50 |
| Duplex ID # | 5 nM | 5 nM | 1 nM | 1 nM | (PM) |
| Blank | 1 | 0 | 1.00 | 0.00 | |
| AD8185 | 0.10 | 90 | 0.18 | 82 | |
| (Rluc siRNA) | | | | | |
| AD7298 | 0.94 | 6 | 1.09 | −9 | |
| (βGal siRNA) | | | | | |
| AD-16210 | 0.12 | 88 | 0.17 | 83 | 25 |
| AD-16211 | 0.06 | 94 | 0.18 | 82 | 47 |
| AD-16212 | 0.10 | 90 | 0.13 | 87 | 53 |
| AD-16213 | 0.11 | 89 | 0.30 | 70 | |
| AD-16214 | 0.12 | 88 | 0.25 | 75 | |
| AD-16215 | 0.21 | 79 | 0.30 | 70 | |
| AD-16216 | 0.10 | 90 | 0.15 | 85 | |
| AD-16217 | 0.07 | 93 | 0.18 | 82 | 77 |
| AD-16218 | 0.13 | 87 | 0.11 | 89 | |
| AD-16219 | 0.14 | 86 | 0.49 | 51 | |
| AD-16220 | 0.14 | 86 | 0.51 | 49 | |
| AD-16221 | 0.09 | 91 | 0.30 | 70 | |
| AD-16222 | 0.39 | 61 | 0.85 | 15 | |
| AD-16223 | 0.46 | 54 | 0.73 | 27 | |
| AD-16224 | 0.32 | 68 | 0.86 | 14 | |
| AD-16225 | 0.19 | 81 | 0.57 | 43 | |
| AD-16226 | 0.12 | 88 | 0.55 | 45 | |
| AD-16227 | 0.11 | 89 | 0.26 | 74 | |
| AD-16228 | 0.30 | 70 | 0.77 | 23 | |
| AD-16229 | 0.43 | 57 | 0.73 | 27 | |
| AD-16230 | 0.27 | 73 | 0.63 | 37 | |
| AD-16231 | 0.53 | 47 | 1.16 | −16 | |
| AD-16232 | 0.54 | 46 | 0.97 | 3 | |
| AD-16233 | 0.54 | 46 | 1.12 | −12 | |
| AD-16234 | 0.49 | 51 | 1.09 | −9 | |
| AD-16235 | 0.38 | 62 | 0.84 | 16 | |
| AD-16236 | 0.18 | 82 | 0.58 | 42 | |
| AD-16237 | 0.05 | 95 | 0.20 | 80 | 118 |
| AD-16238 | 0.08 | 92 | 0.15 | 85 | |
| AD-16239 | 0.09 | 91 | 0.18 | 82 | |
| AD-16240 | 0.13 | 87 | 0.26 | 74 | |
| AD-16241 | 0.13 | 87 | 0.23 | 77 | |
| AD-16242 | 0.09 | 91 | 0.22 | 78 | |
| AD-16243 | 0.07 | 93 | 0.12 | 88 | |
| AD-16244 | 0.10 | 90 | 0.22 | 78 | |
| AD-16245 | 0.09 | 91 | 0.17 | 83 | 58 |
| AD-16246 | 0.19 | 81 | 0.62 | 38 | |
| AD-16247 | 0.33 | 67 | 0.53 | 47 | |
| AD-16248 | 0.20 | 80 | 0.56 | 44 | |
| AD-16249 | 0.51 | 49 | 0.88 | 12 | |
| AD-16250 | 0.46 | 54 | 0.84 | 16 | |
| AD-16251 | 0.50 | 50 | 0.87 | 13 | |
| AD-16252 | 0.44 | 56 | 0.86 | 14 | |
| AD-16253 | 0.39 | 61 | 0.82 | 18 | |
| AD-16254 | 0.35 | 65 | 0.57 | 43 | |
| AD-16255 | 0.10 | 90 | 0.23 | 77 | |
| AD-16256 | 0.18 | 82 | 0.20 | 80 | |
| AD-16257 | 0.08 | 92 | 0.15 | 85 | 125 |
| AD-16258 | 0.12 | 88 | 0.39 | 61 | |
| AD-16259 | 0.12 | 88 | 0.29 | 71 | |
| AD-16260 | 0.11 | 89 | 0.38 | 62 | |
| AD-16261 | 0.05 | 95 | 0.15 | 85 | |
| AD-16262 | 0.07 | 93 | 0.14 | 86 | 73 |
| AD-16263 | 0.08 | 92 | 0.16 | 84 | |
| AD-16264 | 0.13 | 87 | 0.50 | 50 | |
| AD-16265 | 0.18 | 82 | 0.52 | 48 | |
| AD-16266 | 0.18 | 82 | 0.41 | 59 | |
| AD-16267 | 0.39 | 61 | 0.82 | 18 | |
| AD-16268 | 0.38 | 62 | 0.93 | 7 | |
| AD-16269 | 0.31 | 69 | 0.94 | 6 | |
| AD-16270 | 0.36 | 64 | 0.78 | 22 | |
| AD-16271 | 0.18 | 82 | 0.57 | 43 | |
| AD-16272 | 0.16 | 84 | 0.43 | 57 | |
| AD-16273 | 0.06 | 94 | 0.29 | 71 | |
| AD-16274 | 0.08 | 92 | 0.37 | 63 | |
| AD-16275 | 0.06 | 94 | 0.20 | 80 | |
| AD-16276 | 0.11 | 89 | 0.36 | 64 | |
| AD-16277 | 0.08 | 92 | 0.20 | 80 | |
| AD-16278 | 0.10 | 90 | 0.28 | 72 | |
| AD-16279 | 0.07 | 93 | 0.24 | 76 | |
| AD-16280 | 0.08 | 92 | 0.28 | 72 | |
| AD-16281 | 0.08 | 92 | 0.17 | 83 | |
| AD-16282 | 0.08 | 92 | 0.29 | 71 | |
| AD-16283 | 0.08 | 92 | 0.31 | 69 | |
| AD-16284 | 0.09 | 91 | 0.26 | 74 | |
| AD-16285 | 0.10 | 90 | 0.28 | 72 | |
| AD-16286 | 0.09 | 91 | 0.26 | 74 | |
| AD-16287 | 0.09 | 91 | 0.23 | 77 | |
| AD-16288 | 0.06 | 94 | 0.25 | 75 | |
| AD-16289 | 0.06 | 94 | 0.25 | 75 | |
| AD-16290 | 0.08 | 92 | 0.21 | 79 | |
| AD-16291 | 0.08 | 92 | 0.37 | 63 | |
| AD-16292 | 0.08 | 92 | 0.34 | 66 | |
| AD-16293 | 0.07 | 93 | 0.34 | 66 | |
| AD-16294 | 0.09 | 91 | 0.30 | 70 | |
| AD-16295 | 0.06 | 94 | 0.25 | 75 | |
| AD-16296 | 0.10 | 90 | 0.33 | 67 | |
| AD-16297 | 0.07 | 93 | 0.27 | 73 | |
| AD-16298 | 0.07 | 93 | 0.27 | 73 | |
| AD-16299 | 0.07 | 93 | 0.29 | 71 | |
| AD-16300 | 0.18 | 82 | 0.73 | 27 | |
| AD-16301 | 0.18 | 82 | 0.33 | 67 | |
| AD-16302 | 0.23 | 77 | 0.55 | 45 | |
| AD-16303 | 0.41 | 59 | 0.90 | 10 | |
| AD-16304 | 0.32 | 68 | 0.97 | 3 | |
| AD-16305 | 0.26 | 74 | 0.80 | 20 | |
| AD-16306 | 0.26 | 74 | 0.79 | 21 | |
| AD-16307 | 0.19 | 81 | 0.72 | 28 | |
| AD-16308 | 0.15 | 85 | 0.43 | 57 | |
| AD-16309 | 0.13 | 87 | 0.82 | 18 | |
| AD-16310 | 0.20 | 80 | 0.75 | 25 | |
| AD-16311 | 0.14 | 86 | 0.56 | 44 | |
| AD-16312 | 0.34 | 66 | 0.89 | 11 | |
| AD-16313 | 0.32 | 68 | 0.93 | 7 | |
| AD-16314 | 0.29 | 71 | 0.99 | 1 | |
| AD-16315 | 0.22 | 78 | 1.35 | −35 | |
| AD-16316 | 0.22 | 78 | 0.82 | 18 | |
| AD-16317 | 0.20 | 80 | 0.81 | 19 | |
| AD-16318 | 0.02 | 98 | 0.15 | 85 | 46 |
| AD-16319 | 0.03 | 97 | 0.15 | 85 | |
| AD-16320 | 0.02 | 98 | 0.14 | 86 | 30 |
| AD-16321 | 0.03 | 97 | 0.21 | 79 | |
| AD-16322 | 0.03 | 97 | 0.24 | 76 | |
| AD-16323 | 0.05 | 95 | 0.65 | 35 | |
| AD-16324 | 0.03 | 97 | 0.07 | 93 | 20 |
| AD-16325 | 0.06 | 94 | 0.15 | 85 | |
| AD-16326 | 0.05 | 95 | 0.18 | 82 | |
| AD-16327 | 0.15 | 85 | 0.22 | 78 | |
| AD-16328 | 0.09 | 91 | 0.11 | 89 | |
| AD-16329 | 0.09 | 91 | 0.13 | 87 | 34 |
| AD-16330 | 0.10 | 90 | 0.11 | 89 | |
| AD-16331 | 0.12 | 88 | 0.18 | 82 | |
| AD-16332 | 0.12 | 88 | 0.12 | 88 | |
| AD-16333 | 0.14 | 86 | 0.24 | 76 | |
| AD-16334 | 0.14 | 86 | 0.16 | 84 | |
| AD-16335 | 0.19 | 81 | 0.48 | 52 | |
| AD-16336 | 0.10 | 90 | 0.11 | 89 | |
| AD-16337 | 0.17 | 83 | 0.22 | 78 | |
| AD-16338 | 0.07 | 93 | 0.09 | 91 | |
| AD-16339 | 0.11 | 89 | 0.18 | 82 | |
| AD-16340 | 0.06 | 94 | 0.08 | 92 | |
| AD-16341 | 0.07 | 93 | 0.07 | 93 | 39 |

TABLE 7
Sequences and modifications of 2′Fluoro or combination of 2′OMe and 2′Fluoro modified RSV siRNA.

| ss-ID # | sense strand (5′--3′) | SEQ ID NO: | as-ID# | Antisense strand (5′--3′) | SEQ ID NO: | Duplex ID | As-chem. |
|---|---|---|---|---|---|---|---|
| A26547 | GGC UCU UAG CAA AGU CAA Guu | 315 | A26556 | CUU GAC UUU GCUf AAG AGC CdTdT-Hp | 2 | AD-16210 | 2′F |
| A26548 | GGC UCU UAG CAA AGU CAA Guu-Hp | 315 | A26556 | CUU GAC UUU GCUf AAG AGC CdTdT-Hp | 2 | AD-16211 | 2′F |
| A26549 | GgC UCU UAG CAA AGU CAA Guu-Hp | 315 | A26556 | CUU GAC UUU GCUf AAG AGC CdTdT-Hp | 2 | AD-16212 | 2′F |
| A26550 | GgC UCU uAG cAA AGU cAA Guu-Hp | 315 | A26556 | CUU GAC UUU GCUf AAG AGC CdTdT-Hp | 2 | AD-16213 | 2′F |
| A26551 | GgC UCU uAG cAA AGU cAA GdTdT-Hp | 1 | A26556 | CUU GAC UUU GCUf AAG AGC CdTdT-Hp | 2 | AD-16214 | 2′F |
| A26552 | GgC UCU uAG cAA AGU cAA G-Hp | 302 | A26556 | CUU GAC UUU GCUf AAG AGC CdTdT-Hp | 2 | AD-16215 | 2′F |
| A26553 | GfgCf uCfu UfaGf cAfa AfgUf cAfa GfdTdT-Hp | 1 | A26556 | CUU GAC UUU GCUf AAG AGC CdTdT-Hp | 2 | AD-16216 | 2′F |
| A26554 | GgC uCu UaG cAa AgU cAa GdTdT-Hp | 1 | A26556 | CUU GAC UUU GCUf AAG AGC CdTdT-Hp | 2 | AD-16217 | 2′F |
| A26555 | GgC uCu UaG cAa AgU cAa G-Hp | 302 | A26556 | CUU GAC UUU GCUf AAG AGC CdTdT-Hp | 2 | AD-16218 | 2′F |
| A26547 | GGC UCU UAG CAA AGU CAA Guu | 315 | A26565 | CuU GAC UUU GCUf AAG AGC cAU-Hp | 316 | AD-16219 | 2′-OMe + 2′F |
| A26548 | GGC UCU UAG CAA AGU CAA Guu-Hp | 315 | A26565 | CuU GAC UUU GCUf AAG AGC cAU-Hp | 316 | AD-16220 | 2′-OMe + 2′F |
| A26549 | GgC UCU UAG CAA AGU CAA Guu-Hp | 315 | A26565 | CuU GAC UUU GCUf AAG AGC cAU-Hp | 316 | AD-16221 | 2′-OMe + 2′F |
| A26550 | GgC UCU uAG cAA AGU cAA Guu-Hp | 315 | A26565 | CuU GAC UUU GCUf AAG AGC cAU-Hp | 316 | AD-16222 | 2′-OMe + 2′F |
| A26551 | GgC UCU uAG cAA AGU cAA GdTdT-Hp | 1 | A26565 | CuU GAC UUU GCUf AAG AGC cAU-Hp | 316 | AD-16223 | 2′-OMe + 2′F |
| A26552 | GgC UCU uAG cAA AGU cAA G-Hp | 302 | A26565 | CuU GAC UUU GCUf AAG AGC cAU-Hp | 316 | AD-16224 | 2′-OMe + 2′F |
| A26553 | GfgCf uCfu UfaGf cAfa AfgUf cAfa GfdTdT-Hp | 1 | A26565 | CuU GAC UUU GCUf AAG AGC cAU-Hp | 316 | AD-16225 | 2′-OMe + 2′F |
| A26554 | GgC uCu UaG cAa AgU cAa GdTdT-Hp | 1 | A26565 | CuU GAC UUU GCUf AAG AGC cAU-Hp | 316 | AD-16226 | 2′-OMe + 2′F |
| A26555 | GgC uCu UaG cAa AgU cAa G-Hp | 302 | A26565 | CuU GAC UUU GCUf AAG AGC cAU-Hp | 316 | AD-16227 | 2′-OMe + 2′F |
| A26547 | GGC UCU UAG CAA AGU CAA Guu | 315 | A26566 | CfuU GAC UUU GCU AAG AGC CfAU-Hp | 316 | AD-16228 | 2′-OMe + 2′F |
| A26548 | GGC UCU UAG CAA AGU CAA Guu-Hp | 315 | A26566 | CfuU GAC UUU GCU AAG AGC CfAU-Hp | 316 | AD-16229 | 2′-OMe + 2′F |
| A26549 | GgC UCU UAG CAA AGU CAA Guu-Hp | 315 | A26566 | CfuU GAC UUU GCU AAG AGC CfAU-Hp | 316 | AD-16230 | 2′-OMe + 2′F |
| A26550 | GgC UCU uAG cAA AGU cAA Guu-Hp | 315 | A26566 | CfuU GAC UUU GCU AAG AGC CfAU-Hp | 316 | AD-16231 | 2′-OMe + 2′F |
| A26551 | GgC UCU uAG cAA AGU cAA GdTdT-Hp | 1 | A26566 | CfuU GAC UUU GCU AAG AGC CfAU-Hp | 316 | AD-16232 | 2′-OMe + 2′F |
| A26552 | GgC UCU uAG cAA AGU cAA G-Hp | 302 | A26566 | CfuU GAC UUU GCU AAG AGC CfAU-Hp | 316 | AD-16233 | 2′-OMe + 2′F |
| A26553 | GfgCf uCfu UfaGf cAfa AfgUf cAfa GfdTdT-Hp | 1 | A26566 | CfuU GAC UUU GCU AAG AGC CfAU-Hp | 316 | AD-16234 | 2′-OMe + 2′F |
| A26554 | GgC uCu UaG cAa AgU cAa GdTdT-Hp | 1 | A26566 | CfuU GAC UUU GCU AAG AGC CfAU-Hp | 316 | AD-16235 | 2′-OMe + 2′F |
| A26555 | GgC uCu UaG cAa AgU cAa G-Hp | 302 | A26566 | CfuU GAC UUU GCU AAG AGC CfAU-Hp | 316 | AD-16236 | 2′-OMe + 2′F |
| A26547 | GGC UCU UAG CAA AGU CAA Guu | 315 | A26567 | CfUfU GAC UUU GCU AAG AGC CfAU-Hp | 316 | AD-16237 | 2′F |
| A26548 | GGC UCU UAG CAA AGU CAA Guu-Hp | 315 | A26567 | CfUfU GAC UUU GCU AAG AGC CfAU-Hp | 316 | AD-16238 | 2′F |
| A26549 | GgC UCU UAG CAA AGU CAA Guu-Hp | 315 | A26567 | CfUfU GAC UUU GCU AAG AGC CfAU-Hp | 316 | AD-16239 | 2′F |
| A26550 | GgC UCU uAG cAA AGU cAA Guu-Hp | 315 | A26567 | CfUfU GAC UUU GCU AAG AGC CfAU-Hp | 316 | AD-16240 | 2′F |
| A26551 | GgC UCU uAG cAA AGU cAA GdTdT-Hp | 1 | A26567 | CfUfU GAC UUU GCU AAG AGC CfAU-Hp | 316 | AD-16241 | 2′F |
| A26552 | GgC UCU uAG cAA AGU cAA G-Hp | 302 | A26567 | CfUfU GAC UUU GCU AAG AGC CfAU-Hp | 316 | AD-16242 | 2′F |
| A26553 | GfgCf uCfu UfaGf cAfa AfgUf cAfa GfdTdT-Hp | 1 | A26567 | CfUfU GAC UUU GCU AAG AGC CfAU-Hp | 316 | AD-16243 | 2′F |
| A26554 | GgC uCu UaG cAa AgU cAa GdTdT-Hp | 1 | A26567 | CfUfU GAC UUU GCU AAG AGC CfAU-Hp | 316 | AD-16244 | 2′F |
| A26555 | GgC uCu UaG cAa AgU cAa G-Hp | 302 | A26567 | CfUfU GAC UUU GCU AAG AGC CfAU-Hp | 316 | AD-16245 | 2′F |
| A26547 | GGC UCU UAG CAA AGU CAA Guu | 315 | A26568 | CfuU GAC UUU GCUf AAG AGC CfAU-Hp | 316 | AD-16246 | 2′-OMe + 2′F |
| A26548 | GGC UCU UAG CAA AGU CAA Guu-Hp | 315 | A26568 | CfuU GAC UUU GCUf AAG AGC CfAU-Hp | 316 | AD-16247 | 2′-OMe + 2′F |
| A26549 | GgC UCU UAG CAA AGU CAA Guu-Hp | 315 | A26568 | CfuU GAC UUU GCUf AAG AGC CfAU-Hp | 316 | AD-16248 | 2′-OMe + 2′F |
| A26550 | GgC UCU uAG cAA AGU cAA Guu-Hp | 315 | A26568 | CfuU GAC UUU GCUf AAG AGC CfAU-Hp | 316 | AD-16249 | 2′-OMe + 2′F |
| A26551 | GgC UCU uAG cAA AGU cAA GdTdT-Hp | 1 | A26568 | CfuU GAC UUU GCUf AAG AGC CfAU-Hp | 316 | AD-16250 | 2′-OMe + 2′F |
| A26552 | GgC UCU uAG cAA AGU cAA G-Hp | 302 | A26568 | CfuU GAC UUU GCUf AAG AGC CfAU-Hp | 316 | AD-16251 | 2′-OMe + 2′F |
| A26553 | GfgCf uCfu UfaGf cAfa AfgUf cAfa GfdTdT-Hp | 1 | A26568 | CfuU GAC UUU GCUf AAG AGC CfAU-Hp | 316 | AD-16252 | 2′-OMe + 2′F |
| A26554 | GgC uCu UaG cAa AgU cAa GdTdT-Hp | 1 | A26568 | CfuU GAC UUU GCUf AAG AGC CfAU-Hp | 316 | AD-16253 | 2′-OMe + 2′F |
| A26555 | GgC uCu UaG cAa AgU cAa G-Hp | 302 | A26568 | CfuU GAC UUU GCUf AAG AGC CfAU-Hp | 316 | AD-16254 | 2′-OMe + 2′F |
| A26547 | GGC UCU UAG CAA AGU CAA Guu | 315 | A26569 | CfUfU GAC UUU GCUf AAG AGC CfAU-Hp | 316 | AD-16255 | 2′F |
| A26548 | GGC UCU UAG CAA AGU CAA Guu-Hp | 315 | A26569 | CfUfU GAC UUU GCUf AAG AGC CfAU-Hp | 316 | AD-16256 | 2′F |
| A26549 | GgC UCU UAG CAA AGU CAA Guu-Hp | 315 | A26569 | CfUfU GAC UUU GCUf AAG AGC CfAU-Hp | 316 | AD-16257 | 2′F |
| A26550 | GgC UCU uAG cAA AGU cAA Guu-Hp | 315 | A26569 | CfUfU GAC UUU GCUf AAG AGC CfAU-Hp | 316 | AD-16258 | 2′F |
| A26551 | GgC UCU uAG cAA AGU cAA GdTdT-Hp | 1 | A26569 | CfUfU GAC UUU GCUf AAG AGC CfAU-Hp | 316 | AD-16259 | 2′F |
| A26552 | GgC UCU uAG cAA AGU cAA G-Hp | 302 | A26569 | CfUfU GAC UUU GCUf AAG AGC CfAU-Hp | 316 | AD-16260 | 2′F |
| A26553 | GfgCf uCfu UfaGf cAfa AfgUf cAfa GfdTdT-Hp | 1 | A26569 | CfUfU GAC UUU GCUf AAG AGC CfAU-Hp | 316 | AD-16261 | 2′F |
| A26554 | GgC uCu UaG cAa AgU cAa GdTdT-Hp | 1 | A26569 | CfUfU GAC UUU GCUf AAG AGC CfAU-Hp | 316 | AD-16262 | 2′F |
| A26555 | GgC uCu UaG cAa AgU cAa G-Hp | 302 | A26569 | CfUfU GAC UUU GCUf AAG AGC CfAU-Hp | 316 | AD-16263 | 2′F |
| A26547 | GGC UCU UAG CAA AGU CAA Guu | 315 | A26570 | CfuUf GAC UUU GCU AAG AGC CfAU-Hp | 316 | AD-16264 | 2′-OMe + 2′F |
| A26548 | GGC UCU UAG CAA AGU CAA Guu-Hp | 315 | A26570 | CfuUf GAC UUU GCU AAG AGC CfAU-Hp | 316 | AD-16265 | 2′-OMe + 2′F |
| A26549 | GgC UCU UAG CAA AGU CAA Guu-Hp | 315 | A26570 | CfuUf GAC UUU GCU AAG AGC CfAU-Hp | 316 | AD-16266 | 2′-OMe + 2′F |
| A26550 | GgC UCU uAG cAA AGU cAA Guu-Hp | 315 | A26570 | CfuUf GAC UUU GCU AAG AGC CfAU-Hp | 316 | AD-16267 | 2′-OMe + 2′F |
| A26551 | GgC UCU uAG cAA AGU cAA GdTdT-Hp | 1 | A26570 | CfuUf GAC UUU GCU AAG AGC CfAU-Hp | 316 | AD-16268 | 2′-OMe + 2′F |
| A26552 | GgC UCU uAG cAA AGU cAA G-Hp | 302 | A26570 | CfuUf GAC UUU GCU AAG AGC CfAU-Hp | 316 | AD-16269 | 2′-OMe + 2′F |
| A26553 | GfgCf uCfu UfaGf cAfa AfgUf cAfa GfdTdT-Hp | 1 | A26570 | CfuUf GAC UUU GCU AAG AGC CfAU-Hp | 316 | AD-16270 | 2′-OMe + 2′F |
| A26554 | GgC uCu UaG cAa AgU cAa GdTdT-Hp | 1 | A26570 | CfuUf GAC UUU GCU AAG AGC CfAU-Hp | 316 | AD-16271 | 2′-OMe + 2′F |
| A26555 | GgC uCu UaG cAa AgU cAa G-Hp | 302 | A26570 | CfuUf GAC UUU GCU AAG AGC CfAU-Hp | 316 | AD-16272 | 2′-OMe + 2′F |
| A26547 | GGC UCU UAG CAA AGU CAA Guu | 315 | A26571 | CfUfUf GAC UUU GCU AAG AGCf CfAU-Hp | 316 | AD-16273 | 2′F |
| A26548 | GGC UCU UAG CAA AGU CAA Guu-Hp | 315 | A26571 | CfUfUf GAC UUU GCU AAG AGCf CfAU-Hp | 316 | AD-16274 | 2′F |
| A26549 | GgC UCU UAG CAA AGU CAA Guu-Hp | 315 | A26571 | CfUfUf GAC UUU GCU AAG AGCf CfAU-Hp | 316 | AD-16275 | 2′F |
| A26550 | GgC UCU uAG cAA AGU cAA Guu-Hp | 315 | A26571 | CfUfUf GAC UUU GCU AAG AGCf CfAU-Hp | 316 | AD-16276 | 2′F |
| A26551 | GgC UCU uAG cAA AGU cAA GdTdT-Hp | 1 | A26571 | CfUfUf GAC UUU GCU AAG AGCf CfAU-Hp | 316 | AD-16277 | 2′F |
| A26552 | GgC UCU uAG cAA AGU cAA G-Hp | 302 | A26571 | CfUfUf GAC UUU GCU AAG AGCf CfAU-Hp | 316 | AD-16278 | 2′F |
| A26553 | GfgCf uCfu UfaGf cAfa AfgUf cAfa GfdTdT-Hp | 1 | A26571 | CfUfUf GAC UUU GCU AAG AGCf CfAU-Hp | 316 | AD-16279 | 2′F |
| A26554 | GgC uCu UaG cAa AgU cAa GdTdT-Hp | 1 | A26571 | CfUfUf GAC UUU GCU AAG AGCf CfAU-Hp | 316 | AD-16280 | 2′F |
| A26555 | GgC uCu UaG cAa AgU cAa G-Hp | 302 | A26571 | CfUfUf GAC UUU GCU AAG AGCf CfAU-Hp | 316 | AD-16281 | 2′F |
| A26547 | GGC UCU UAG CAA AGU CAA Guu | 315 | A26572 | CfUfUf GAC UUU GCUf AAG AGC CfAU-Hp | 316 | AD-16282 | 2′F |
| A26548 | GGC UCU UAG CAA AGU CAA Guu-Hp | 315 | A26572 | CfUfUf GAC UUU GCUf AAG AGC CfAU-Hp | 316 | AD-16283 | 2′F |
| A26549 | GgC UCU UAG CAA AGU CAA Guu-Hp | 315 | A26572 | CfUfUf GAC UUU GCUf AAG AGC CfAU-Hp | 316 | AD-16284 | 2′F |
| A26550 | GgC UCU uAG cAA AGU cAA Guu-Hp | 315 | A26572 | CfUfUf GAC UUU GCUf AAG AGC CfAU-Hp | 316 | AD-16285 | 2′F |
| A26551 | GgC UCU uAG cAA AGU cAA GdTdT-Hp | 1 | A26572 | CfUfUf GAC UUU GCUf AAG AGC CfAU-Hp | 316 | AD-16286 | 2′F |
| A26552 | GgC UCU uAG cAA AGU cAA G-Hp | 302 | A26572 | CfUfUf GAC UUU GCUf AAG AGC CfAU-Hp | 316 | AD-16287 | 2′F |
| A26553 | GfgCf uCfu UfaGf cAfa AfgUf cAfa GfdTdT-Hp | 1 | A26572 | CfUfUf GAC UUU GCUf AAG AGC CfAU-Hp | 316 | AD-16288 | 2′F |
| A26554 | GgC uCu UaG cAa AgU cAa GdTdT-Hp | 1 | A26572 | CfUfUf GAC UUU GCUf AAG AGC CfAU-Hp | 316 | AD-16289 | 2′F |
| A26555 | GgC uCu UaG cAa AgU cAa G-Hp | 302 | A26572 | CfUfUf GAC UUU GCUf AAG AGC CfAU-Hp | 316 | AD-16290 | 2′F |
| A26547 | GGC UCU UAG CAA AGU CAA Guu | 315 | A26573 | CfUfUf GAC UUU GCUf AAG AGCf CfAU-Hp | 316 | AD-16291 | 2′F |
| A26548 | GGC UCU UAG CAA AGU CAA Guu-Hp | 315 | A26573 | CfUfUf GAC UUU GCUf AAG AGCf CfAU-Hp | 316 | AD-16292 | 2′F |
| A26549 | GgC UCU UAG CAA AGU CAA Guu-Hp | 315 | A26573 | CfUfUf GAC UUU GCUf AAG AGCf CfAU-Hp | 316 | AD-16293 | 2′F |
| A26550 | GgC UCU uAG cAA AGU cAA Guu-Hp | 315 | A26573 | CfUfUf GAC UUU GCUf AAG AGCf CfAU-Hp | 316 | AD-16294 | 2′F |
| A26551 | GgC UCU uAG cAA AGU cAA GdTdT-Hp | 1 | A26573 | CfUfUf GAC UUU GCUf AAG AGCf CfAU-Hp | 316 | AD-16295 | 2′F |
| A26552 | GgC UCU uAG cAA AGU cAA G-Hp | 302 | A26573 | CfUfUf GAC UUU GCUf AAG AGCf CfAU-Hp | 316 | AD-16296 | 2′F |
| A26553 | GfgCf uCfu UfaGf cAfa AfgUf cAfa GfdTdT-Hp | 1 | A26573 | CfUfUf GAC UUU GCUf AAG AGCf CfAU-Hp | 316 | AD-16297 | 2′F |
| A26554 | GgC uCu UaG cAa AgU cAa GdTdT-Hp | 1 | A26573 | CfUfUf GAC UUU GCUf AAG AGCf CfAU-Hp | 316 | AD-16298 | 2′F |
| A26555 | GgC uCu UaG cAa AgU cAa G-Hp | 302 | A26573 | CfUfUf GAC UUU GCUf AAG AGCf CfAU-Hp | 316 | AD-16299 | 2′F |
| A26547 | GGC UCU UAG CAA AGU CAA Guu | 315 | A26574 | CfuUf GAC UUU GCUf AAG AGCf CfAU-Hp | 316 | AD-16300 | 2′-OMe + 2′F |
| A26548 | GGC UCU UAG CAA AGU CAA Guu-Hp | 315 | A26574 | CfuUf GAC UUU GCUf AAG AGCf CfAU-Hp | 316 | AD-16301 | 2′-OMe + 2′F |
| A26549 | GgC UCU UAG CAA AGU CAA Guu-Hp | 315 | A26574 | CfuUf GAC UUU GCUf AAG AGCf CfAU-Hp | 316 | AD-16302 | 2′-OMe + 2′F |
| A26550 | GgC UCU uAG cAA AGU cAA Guu-Hp | 315 | A26574 | CfuUf GAC UUU GCUf AAG AGCf CfAU-Hp | 316 | AD-16303 | 2′-OMe + 2′F |
| A26551 | GgC UCU uAG cAA AGU cAA GdTdT-Hp | 1 | A26574 | CfuUf GAC UUU GCUf AAG AGCf CfAU-Hp | 316 | AD-16304 | 2′-OMe + 2′F |
| A26552 | GgC UCU uAG cAA AGU cAA G-Hp | 302 | A26574 | CfuUf GAC UUU GCUf AAG AGCf CfAU-Hp | 316 | AD-16305 | 2′-OMe + 2′F |
| A26553 | GfgCf uCfu UfaGf cAfa AfgUf cAfa GfdTdT-Hp | 1 | A26574 | CfuUf GAC UUU GCUf AAG AGCf CfAU-Hp | 316 | AD-16306 | 2′-OMe + 2′F |
| A26554 | GgC uCu UaG cAa AgU cAa GdTdT-Hp | 1 | A26574 | CfuUf GAC UUU GCUf AAG AGCf CfAU-Hp | 316 | AD-16307 | 2′-OMe + 2′F |
| A26555 | GgC uCu UaG cAa AgU cAa G-Hp | 302 | A26574 | CfuUf GAC UUU GCUf AAG AGCf CfAU-Hp | 316 | AD-16308 | 2′-OMe + 2′F |
| A26547 | GGC UCU UAG CAA AGU CAA Guu | 315 | A26575 | cuUf GAC UUU GCUf AAG AGc cAU-Hp | 316 | AD-16309 | 2′-OMe + 2′F |
| A26548 | GGC UCU UAG CAA AGU CAA Guu-Hp | 315 | A26575 | cuUf GAC UUU GCUf AAG AGc cAU-Hp | 316 | AD-16310 | 2′-OMe + 2′F |
| A26549 | GgC UCU UAG CAA AGU CAA Guu-Hp | 315 | A26575 | cuUf GAC UUU GCUf AAG AGc cAU-Hp | 316 | AD-16311 | 2′-OMe + 2′F |
| A26550 | GgC UCU uAG cAA AGU cAA Guu-Hp | 315 | A26575 | cuUf GAC UUU GCUf AAG AGc cAU-Hp | 316 | AD-16312 | 2′-OMe + 2′F |
| A26551 | GgC UCU uAG cAA AGU cAA GdTdT-Hp | 1 | A26575 | cuUf GAC UUU GCUf AAG AGc cAU-Hp | 316 | AD-16313 | 2′-OMe + 2′F |
| A26552 | GgC UCU uAG cAA AGU cAA G-Hp | 302 | A26575 | cuUf GAC UUU GCUf AAG AGc cAU-Hp | 316 | AD-16314 | 2′-OMe + 2′F |
| A26553 | GfgCf uCfu UfaGf cAfa AfgUf cAfa GfdTdT-Hp | 1 | A26575 | cuUf GAC UUU GCUf AAG AGc cAU-Hp | 316 | AD-16315 | 2′-OMe + 2′F |
| A26554 | GgC uCu UaG cAa AgU cAa GdTdT-Hp | 1 | A26575 | cuUf GAC UUU GCUf AAG AGc cAU-Hp | 316 | AD-16316 | 2′-OMe + 2′F |
| A26555 | GgC uCu UaG cAa AgU cAa G-Hp | 302 | A26575 | cuUf GAC UUU GCUf AAG AGc cAU-Hp | 316 | AD-16317 | 2′-OMe + 2′F |
| A26553 | GfgCf uCfu UfaGf cAfa AfgUf cAfa GfdTdT-Hp | 1 | A26557 | CUfUf GACf UfUfUf GCUf AAG AGC CdTdT-Hp | 2 | AD-16318 | 2′F |
| A26553 | GfgCf uCfu UfaGf cAfa AfgUf cAfa GfdTdT-Hp | 1 | A26558 | CfUfUf GACf UfUfUf GCfUf AAG AGCf CfdTdT-Hp | 2 | AD-16319 | all Py 2′F |
| A26553 | GfgCf uCfu UfaGf cAfa AfgUf cAfa GfdTdT-Hp | 1 | A26559 | CUfU GAC UUfU GCUf AAG AGC CfdTdT-Hp | 2 | AD-16320 | 2′F |
| A26553 | GfgCf uCfu UfaGf cAfa AfgUf cAfa GfdTdT-Hp | 1 | A26560 | p-cUfu GfaCf uUfu GfcUf aAfg AfgCf cdTdT-Hp | 2 | AD-16321 | Altern 2F/OMe |
| A26554 | GgC uCu UaG cAa AgU cAa GdTdT-Hp | 1 | A26557 | CUfUf GACf UfUfUf GCUf AAG AGC CdTdT-Hp | 2 | AD-16322 | 2′F |
| A26554 | GgC uCu UaG cAa AgU cAa GdTdT-Hp | 1 | A26558 | CfUfUf GACf UfUfUf GCfUf AAG AGCf CfdTdT-Hp | 2 | AD-16323 | all Py 2′F |
| A26554 | GgC uCu UaG cAa AgU cAa GdTdT-Hp | 1 | A26559 | CUfU GAC UUfU GCUf AAG AGC CfdTdT-Hp | 2 | AD-16324 | 2′F |
| A26554 | GgC uCu UaG cAa AgU cAa GdTdT-Hp | 1 | A26560 | p-cUfu GfaCf uUfu GfcUf aAfg AfgCf cdTdT-Hp | 2 | AD-16325 | Altern 2F/OMe |
| A26555 | GgC uCu UaG cAa AgU cAa G-Hp | 302 | A26557 | CUfUf GACf UfUfUf GCUf AAG AGC CdTdT-Hp | 2 | AD-16326 | 2′F |
| A26555 | GgC uCu UaG cAa AgU cAa G-Hp | 302 | A26558 | CfUfUf GACf UfUfUf GCfUf AAG AGCf CfdTdT-Hp | 2 | AD-16327 | all Py 2′F |
| A26555 | GgC uCu UaG cAa AgU cAa G-Hp | 302 | A26559 | CUfU GAC UUfU GCUf AAG AGC CfdTdT-Hp | 2 | AD-16328 | 2′F |
| A26555 | GgC uCu UaG cAa AgU cAa G-Hp | 302 | A26560 | p-cUfu GfaCf uUfu GfcUf aAfg AfgCf cdTdT-Hp | 2 | AD-16329 | Altern 2F/OMe |
| A26551 | GgC UCU uAG cAA AGU cAA GdTdT-Hp | 1 | A26557 | CUfUf GACf UfUfUf GCUf AAG AGC CdTdT-Hp | 2 | AD-16330 | 2′F |
| A26551 | GgC UCU uAG cAA AGU cAA GdTdT-Hp | 1 | A26558 | CfUfUf GACf UfUfUf GCfUf AAG AGCf CfdTdT-Hp | 2 | AD-16331 | all Py 2′F |
| A26551 | GgC UCU uAG cAA AGU cAA GdTdT-Hp | 1 | A26559 | CUfU GAC UUfU GCUf AAG AGC CfdTdT-Hp | 2 | AD-16332 | 2′F |
| A26551 | GgC UCU uAG cAA AGU cAA GdTdT-Hp | 1 | A26560 | p-cUfu GfaCf uUfu GfcUf aAfg AfgCf cdTdT-Hp | 2 | AD-16333 | Altern 2F/OMe |
| A26550 | GgC UCU uAG cAA AGU cAA Guu-Hp | 315 | A26557 | CUfUf GACf UfUfUf GCUf AAG AGC CdTdT-Hp | 2 | AD-16334 | 2′F |
| A26550 | GgC UCU uAG cAA AGU cAA Guu-Hp | 315 | A26558 | CfUfUf GACf UfUfUf GCfUf AAG AGCf CfdTdT-Hp | 2 | AD-16335 | all Py 2′F |
| A26550 | GgC UCU uAG cAA AGU cAA Guu-Hp | 315 | A26559 | CUfU GAC UUfU GCUf AAG AGC CfdTdT-Hp | 2 | AD-16336 | 2′F |
| A26550 | GgC UCU uAG cAA AGU cAA Guu-Hp | 315 | A26560 | p-cUfu GfaCf uUfu GfcUf aAfg AfgCf cdTdT-Hp | 2 | AD-16337 | Altern 2F/OMe |
| A26549 | GgC UCU UAG CAA AGU CAA Guu-Hp | 315 | A26557 | CUfUf GACf UfUfUf GCUf AAG AGC CdTdT-Hp | 2 | AD-16338 | 2′F |
| A26549 | GgC UCU UAG CAA AGU CAA Guu-Hp | 315 | A26558 | CfUfUf GACf UfUfUf GCfUf AAG AGCf CfdTdT-Hp | 2 | AD-16339 | all Py 2′F |
| A26549 | GgC UCU UAG CAA AGU CAA Guu-Hp | 315 | A26559 | CUfU GAC UUfU GCUf AAG AGC CfdTdT-Hp | 2 | AD-16340 | 2′F |
| A26549 | GgC UCU UAG CAA AGU CAA Guu-Hp | 315 | A26560 | p-cUfu GfaCf uUfu GfcUf aAfg AfgCf cdTdT-Hp | 2 | AD-16341 | altern 2F/OMe |

Chemistry is denoted as follows
lower case is 2′ O-methyl (OMe) modification
HP: Exonuclease (Exo) protection = hydroxy pyrollidine (Hp) linker
Endonuclease (endo) light = UA/CA 2′ OMe(Chemistry 2); endo heavy = all pyrimidine (Py) as 2′-OMe (Chemistry 3);
2′-OMe, @ Pos 2 (Chemistry 4); TT-complem. @ 2′-OMe, PTO
2′Fluoro (F), 2′-OMe + 2′F, all pyrimidine (Py) 2′F, altern 2′F/OMe
p = 5′-phosphate

TABLE 8
in vitro activity of 2′OMe modified RSV siRNA.

| Duplex# | Average IC50 In-Vitro (PM) |
|---|---|
| RSV01 | 91 |
| AD-3520 | 142 |
| AD-3521 | 356 |
| AD-3522 | 416 |
| AD-3523 | 997 |
| AD-3524 | 136 |
| AD-3525 | 703 |
| AD-3526 | 129 |
| AD-3527 | 147 |
| AD-3528 | 54 |
| AD-3529 | 68 |
| AD-3530 | 36 |
| AD-3531 | 61 |
| AD-3532 | 78 |
| AD-3533 | 75 |
| AD-3534 | 72 |
| AD-3535 | 37 |
| AD-3581 | 24 |
| AD-3582 | 27 |
| AD-3583 | 72 |
| AD-3584 | 20 |
| AD-3585 | 14 |
| AD-3586 | 469 |
| AD-3587 | 99 |
| AD-3588 | 95 |
| AD-3589 | 270 |
| AD-3590 | 37 |
| AD-3591 | 25 |
| AD-3592 | 128 |
| AD-3593 | 21 |
| AD-3594 | 17 |
| AD-3595 | 56 |
| AD-3596 | 9 |
| AD-3597 | 19 |

IC50 of compounds with only 2′ OMe modification (lower case) no 2′Fluoro
Exo protection = phosphorothioate(s) or not protected and without uridine-adenine (UA) protected from endonuclease

TABLE 9
sequences and modifications of 2′OMe modified RSV siRNA.

| | | SEQ | | | SEQ | |
|---|---|---|---|---|---|---|
| | | ID | | | ID | |
| Ss ID# | sense strand (5′--3′) | NO: | As ID# | antisense strand (5′--3′) | NO: | Duplex # |
| 5718 | GGC UCU UAG CAA AGU CAA GdTdT | 1 | 5719 | CUU GAC UUU GCU AAG AGC CdTdT | 2 | RSV01 |
| A-30631 | GGcuCUUAGcAaAGucAAGdTsdT | 1 | A-30643 | CuUGACUuUGCUAAGAGCCdTsdT | 2 | AD-3520 |
| A-30625 | GGcuCUUAGcAaAGucAAGdTdT | 1 | A-30626 | CuUGACUuUGCUAAGAGCCdTdT | 2 | AD-3521 |
| A-30629 | GGcuCUUAGcAaAGucAaGdTsdT | 1 | A-30642 | CuUGACuUUGCUAAGAGCCdTsdT | 2 | AD-3522 |
| A-30623 | GGcuCUUAGcAaAGucAaGdTdT | 1 | A-30624 | CuUGACuUUGCUAAGAGCCdTdT | 2 | AD-3523 |
| A-30629 | GGcuCUUAGcAaAGucAaGdTsdT | 1 | A-30643 | CuUGACUuUGCUAAGAGCCdTsdT | 2 | AD-3524 |
| A-30623 | GGcuCUUAGcAaAGucAaGdTdT | 1 | A-30626 | CuUGACUuUGCUAAGAGCCdTdT | 2 | AD-3525 |
| A-30633 | GgCUCUuAGcAAAGUcAAGdTsdT | 1 | A-30646 | CUuGACuUUGCUAAGAGCcdTsdT | 2 | AD-3526 |
| A-30627 | GgCUCUuAGcAAAGUcAAGdTdT | 1 | A-30632 | CUuGACuUUGCUAAGAGCcdTdT | 2 | AD-3527 |
| A-30633 | GgCUCUuAGcAAAGUcAAGdTsdT | 1 | A-30647 | CUuGACUuUGCUAAGAGCcdTsdT | 2 | AD-3528 |
| A-30627 | GgCUCUuAGcAAAGUcAAGdTdT | 1 | A-30634 | CUuGACUuUGCUAAGAGCcdTdT | 2 | AD-3529 |
| A-30633 | GgCUCUuAGcAAAGUcAAGdTsdT | 1 | A-30654 | CUuGACUUuGCUAAGAGcCdTsdT | 2 | AD-3530 |
| A-30627 | GgCUCUuAGcAAAGUcAAGdTdT | 1 | A-30641 | CUuGACUUuGCUAAGAGcCdTdT | 2 | AD-3531 |
| A-30631 | GGcuCUUAGcAaAGucAAGdTsdT | 1 | A-30648 | CUuGACUUuGCUAAGAGCcdTsdT | 2 | AD-3532 |
| A-30625 | GGcuCUUAGcAaAGucAAGdTdT | 1 | A-30635 | CUuGACUUuGCUAAGAGCcdTdT | 2 | AD-3533 |
| A-30631 | GGcuCUUAGcAaAGucAAGdTsdT | 1 | A-30652 | CUuGACUuUGCUAAGAGcCdTsdT | 2 | AD-3534 |
| A-30625 | GGcuCUUAGcAaAGucAAGdTdT | 1 | A-30639 | CUuGACUuUGCUAAGAGcCdTdT | 2 | AD-3535 |
| A-30629 | GGcuCUUAGcAaAGucAaGdTsdT | 1 | A-30649 | CUuGACuUUGCuAAGAGCcdTsdT | 2 | AD-3581 |
| A-30631 | GGcuCUUAGcAaAGucAAGdTsdT | 1 | A-30649 | CUuGACuUUGCuAAGAGCcdTsdT | 2 | AD-3582 |
| A-30633 | GgCUCUuAGcAAAGUcAAGdTsdT | 1 | A-30649 | CUuGACuUUGCuAAGAGCcdTsdT | 2 | AD-3583 |
| A-30629 | GGcuCUUAGcAaAGucAaGdTsdT | 1 | A-30650 | CUuGACUUuGCuAAGAGCcdTsdT | 2 | AD-3584 |
| A-30631 | GGcuCUUAGcAaAGucAAGdTsdT | 1 | A-30650 | CUuGACUUuGCuAAGAGCcdTsdT | 2 | AD-3585 |
| A-30633 | GgCUCUuAGcAAAGUcAAGdTsdT | 1 | A-30650 | CUuGACUUuGCuAAGAGCcdTsdT | 2 | AD-3586 |
| A-30629 | GGcuCUUAGcAaAGucAaGdTsdT | 1 | A-30653 | CUuGACUuUGCuAAGAGCcdTsdT | 2 | AD-3587 |
| A-30631 | GGcuCUUAGcAaAGucAAGdTsdT | 1 | A-30653 | CUuGACUuUGCuAAGAGCcdTsdT | 2 | AD-3588 |
| A-30633 | GgCUCUuAGcAAAGUcAAGdTsdT | 1 | A-30653 | CUuGACUuUGCuAAGAGCcdTsdT | 2 | AD-3589 |
| A-30623 | GGcuCUUAGcAaAGucAaGdTdT | 1 | A-30636 | CUuGACuUUGCuAAGAGCcdTdT | 2 | AD-3590 |
| A-30625 | GGcuCUUAGcAaAGucAAGdTdT | 1 | A-30636 | CUuGACuUUGCuAAGAGCcdTdT | 2 | AD-3591 |
| A-30627 | GgCUCUuAGcAAAGUcAAGdTdT | 1 | A-30636 | CUuGACuUUGCuAAGAGCcdTdT | 2 | AD-3592 |
| A-30623 | GGcuCUUAGcAaAGucAaGdTdT | 1 | A-30637 | CUuGACUUuGCuAAGAGCcdTdT | 2 | AD-3593 |
| A-30625 | GGcuCUUAGcAaAGucAAGdTdT | 1 | A-30637 | CUuGACUUuGCuAAGAGCcdTdT | 2 | AD-3594 |
| A-30627 | GgCUCUuAGcAAAGUcAAGdTdT | 1 | A-30637 | CUuGACUUuGCuAAGAGCcdTdT | 2 | AD-3595 |
| A-30623 | GGcuCUUAGcAaAGucAaGdTdT | 1 | A-30640 | CUuGACUuUGCuAAGAGCcdTdT | 2 | AD-3596 |
| A-30625 | GGcuCUUAGcAaAGucAAGdTdT | 1 | A-30640 | CUuGACUuUGCuAAGAGCcdTdT | 2 | AD-3597 |

Chemistry: Only 2′ OMe modification (lower case) no F′
Exo protection = Phosothioste(s) or not protected and without UA protected from endonuclease

### Example 18

### In Vitro Antiviral Activity Against RSV A2 of Modified RSV siRNAs

Materials and Methods

Cell Culture: Vero Cells were maintained at 37° C., 5% carbon dioxide (CO₂) in DMEM (GIBCO Cat#11995-065) with 10% fetal bovine serum (FBS) (Omega Scientific Cat# FB02) 1% Antibiotics/Antimicotics (GIBCO Cat#15240-062).

Splitting Cells for Stock: Cells are normally 100% confluent before splitting. Wash cells with 3 ml of 0.25% Trypsin-EDTA. Trypsinize cells with 3 ml 0.25% Trypsin-EDTA and incubate at 37° C., 5% CO₂ until cells are no longer adherent to flask (approximately 2-5 minutes). Add 7 ml of DMEM 10% FBS 1% Antibiotics/Antimicotics and re-suspend thoroughly. Add appropriate aliquots to new flask containing 30 ml of fresh DMEM 10% FBS 1% Antibiotics/Antimicotics to obtain 100% confluence on desired day (cells split 1:3 for confluence on next day). Re-suspend and incubate at 37° C., 5% CO₂.

Splitting cells into 24-Well Plate: Cells are normally 100% confluent before splitting. Wash cells with 3 ml 0.25% Trypsin-EDTA. Trypsinize cells with 3 ml 0.25% Trypsin-EDTA and incubate at 37° C., 5% CO₂ until cells are no longer adherent (2-5 minutes). Add 7 ml of DMEM 10% FBS without Antibiotics/Antimicotics and re-suspend thoroughly. Add 14 ml of DMEM 10% FBS for every 1 ml of suspended cells and re-suspend (final volume should be 150 ml). Pipette 500 μl of suspended cells into each well of 24-well working plate and incubate for 24 hrs at 37° C., 5% CO₂ to have cells be 60%-80% confluent.

Transfection with transIT-TKO: Working Plate: Checked cells to be 60%-80% confluent. Aspirated media from 24-well plates and replaced with 200 μl of fresh DMEM 10% FBS. Cells were transfected with siRNA at a 4 fold decrease in final concentration ranging from 320 nM-0.08 nM. Dilution Plate In a separate 24-well plate 150 μl OPTI-MEM (GIBCO Cat#31985-070) was added to each well except for the first column, to which 200 μl was added. Added 7.68 μl siRNA to the first column to obtain a final concentration of 320 nM. 4 fold dilution was made by transferring 50 μl from the first column to the second and then from the second to the third and so on mixing gently each time. In a separate conical tube added 3.5 ul of transIT-TKO (Mirus Cat# MIR2150) to every 50 μl of OPTI-MEM for each well and incubated at room temperature for 5-10 minutes. After 10 minutes added 150 μl of the transIT-TKO/OPTI-MEM complex to each well of the working plate. Incubated at room temperature (RT) for 30 minutes to allow lipoplex to form. Mixed lipoplex gently and pipetted 95 ul of lipoplex to working plate (each well of dilution plate should have enough lipoplex to transfect 3 wells on working plate). Incubated at 37° C., 5% CO₂ for 24 hrs.

Transfection with Lipofectamine 2000: Working Plate: Checked cells to be 60%-80% confluent. Cells were transfected with siRNA at a 4 fold decrease in final concentration ranging from 320 nM-0.08 nM. Dilution Plate: In a separate 24-well plate added 150 μl OPTI-MEM (GIBCO Cat#31985-070) to each well except for the first column, to which 200 ul was added. Added 15.36 μl siRNA to the first column to obtain a final concentration of 320 nM. 4 fold dilution was made by transferring 50 μl from the first column to the second and then from the second to the third and so on mixing gently each time. In a separate conical tube added 3.0 μl of Lipofectamine2000 (Invitrogen Cat#11668-019) to every 50 μl of OPTI-MEM for each well and incubated at room temperature for 5-10 minutes. After 10 minutes added 150 ul of Lipofectamine2000/OPTI-MEM complex to each well of the working plate. Incubated at RT for 30 minutes to allow lipoplex to form. Mixed lipoplex gently and pipetted 95 μl of lipoplex to working plate (each well of dilution plate should have enough lipoplex to transfect 3 wells on working plate). Incubated at 37° C., 5% CO₂ for 24 hrs.

Infection with RSV A2: Thawed RSV A2 virus and placed on ice (titer 0.9×10̂6). Added 150 μl of virus for every 50 ml of DMEM 2% FBS 1% Antibiotics/Antimicotics. Mixed thoroughly and placed on ice. 24 hrs after transfection washed wells with 1.0 ml of Hanks Buffered Salt Solution (GIBCO Cat#14175-095). Add 200 μl of virus/DMEM to each well. Incubated at 37° C., 5% CO₂ for 1 hr. After 1 hr aspirated virus/media and overlayed with 1 ml of methylcellulose (Sigma Cat#125K0055) 2% FBS and 1% Antibiotics/Antimicotics. Incubated for 5 days at 37° C., 5% CO₂.

Methylcellulose Preparation: Ten grams of methylcellulose were mixed with 75 mL of boiling HBSS, followed by autoclaving for 20 minutes at 15 psi. The solution was cooled to 37° C., then diluted with 40 mL HBSS, 400 mL media, 5 mL antibiotics, and 10 mL FBS. After thorough mixing, the methylcellulose was cooled on ice for 20 minutes.

Plaque assay: 5 days after infection aspirated methylcellulose and fixed cells with ice-cold Acetone:Methanol (60:40) for 10-15 minutes and placed upside down overnight to let Acetone:Methanol evaporate. After 24 hrs blocked cells with 1× Powerblock (BioGenex Cat#HK085-5K) for 30 minutes at RT. Diluted Primary Antibody (131-2A-RSV F monoclonal antibody, Chemicon International Cat#MAB8599) 1:2000 in cold 0.1× Powerblock. Removed Powerblock from plate and added 250 ul of Primary Antibody. Incubated at 37° C., 5% CO₂ for 2 hrs. Washed cells twice for 10-15 minutes with 0.05% Tween (Sigma Cat#SL05303) 10% PBS (GIBCO Cat #70013-032). Diluted Secondary Antibody (Goat anti-mouse IgG whole molecule-Alkaline Phosphatase Conjugate, Sigma Cat#A9316) 1:1000 in cold 0.1× Powerblock. Added 250 ul of Secondary antibody to each well and incubated at 37° C., 5% CO₂ for 1 hr. Washed cells twice for 10 minutes with 0.05% Tween 10% PBS. Made Vector Black Staining Solution (Vector Laboratories Cat#Sk-5200) and added ˜300 μl of stain to each well for 15 min or until staining was distinct. Washed plates with deionized (DI) water and allowed to dry overnight. Counted Plaques.

Results

Table 10 shows the sequences and in vitro antiviral activity of modified RSV siRNA. Table 11 shows the sequences and in vitro antiviral activity of 2′OMe and exonuclease (exo) protected with hydroxy pyrollidine (hp) linker modified RSV siRNA. Table 12 shows the sequences and in vitro antiviral activity of 2′OMe and exonuclease (exo) protected modified RSV siRNA with or without phorothioate(s). Table 13 shows the sequences and in vitro antiviral activity of 2′OMe and exonuclease (exo) protected modified RSV siRNA with phorothioate(s) and with uridine-adenine (UA) protected from endonucleases.

TABLE 10
Sequences (Table 10a) and in vitro antiviral activity (Table 10b) of modified RSV siRNA

| | | SEQ | | | SEQ | |
|---|---|---|---|---|---|---|
| | Table 10a | ID | | | ID | |
| Ss ID # | sense strand (5′--3′) | NO: | AS ID # | antisense strand (5′--3′) | NO: | Duplex# |
| A17650 | GGC UCU UAG CAA AGU CAA GTsT | 1 | 5719 | CUU GAC UUU GCU AAG AGC CdTdT | 2 | AD-3136 |
| A17652 | GGc ucu uAG cAA AGu cAA GTsT | 1 | 5719 | CUU GAC UUU GCU AAG AGC CdTdT | 2 | AD-3137 |
| A17656 | GgC UCU UAG CAA AGU CAA GTsT | 1 | 5719 | CUU GAC UUU GCU AAG AGC CdTdT | 2 | AD-3138 |
| A17658 | GGC UCU UAG CAA AGU CAA Gusu | 315 | 5719 | CUU GAC UUU GCU AAG AGC CdTdT | 2 | AD-3139 |
| A17660 | GGC UCU UAG CAA AGU CAA GdTdT (ab) | 1 | 5719 | CUU GAC UUU GCU AAG AGC CdTdT | 2 | AD-3140 |
| 5718 | GGC UCU UAG CAA AGU CAA GdTdT | 1 | A17651 | CUU GAC UUU GCU AAG AGC CTsT | 2 | AD-3141 |
| 5718 | GGC UCU UAG CAA AGU CAA GdTdT | 1 | A17653 | CUU GAC UUU GCu AAG AGC CTsT | 2 | AD-3142 |
| 5718 | GGC UCU UAG CAA AGU CAA GdTdT | 1 | A17655 | Cuu GAC uuu GCu AAG AGC CTsT | 2 | AD-3143 |
| 5718 | GGC UCU UAG CAA AGU CAA GdTdT | 1 | A17657 | CuU GAC UUU GCU AAG AGC CTsT | 2 | AD-3144 |
| 5718 | GGC UCU UAG CAA AGU CAA GdTdT | 1 | A17659 | CUU GAC UUU GCU AAG AGC Casu | 316 | AD-3145 |
| 5718 | GGC UCU UAG CAA AGU CAA GdTdT | 1 | A17661 | CUU GAC UUU GCU AAG AGC CdTdT (ab) | 2 | AD-3146 |
| A17650 | GGC UCU UAG CAA AGU CAA GTsT | 1 | A17651 | CUU GAC UUU GCU AAG AGC CTsT | 2 | AD-3147 |
| A17652 | GGc ucu uAG cAA AGu cAA GTsT | 1 | A17653 | CUU GAC UUU GCu AAG AGC CTsT | 2 | AD-3148 |
| A17652 | GGc ucu uAG cAA AGu cAA GTsT | 1 | A17655 | Cuu GAC uuu GCu AAG AGC CTsT | 2 | AD-3149 |
| A17656 | GgC UCU UAG CAA AGU CAA GTsT | 1 | A17657 | CuU GAC UUU GCU AAG AGC CTsT | 2 | AD-3150 |
| A17658 | GGC UCU UAG CAA AGU CAA Gusu | 315 | A17659 | CUU GAC UUU GCU AAG AGC Casu | 316 | AD-3151 |
| A17660 | GGC UCU UAG CAA AGU CAA GdTdT (ab) | 1 | A17661 | CUU GAC UUU GCU AAG AGC CdTdT (ab) | 2 | AD-3152 |
| A12560 | GGc ucu uAG cAA Agu cAA GTT | 1 | 5719 | CUU GAC UUU GCU AAG AGC CdTdT | 2 | AD-3116 |
| A12561 | ggc ucu uag cAA AGU CAA GTT | 1 | 5720 | CUU GAC UUU GCU AAG AGC CdTdT | 2 | AD-3117 |
| A12562 | GGC UCU UAG caa agu caa gTT | 1 | 5721 | CUU GAC UUU GCU AAG AGC CdTdT | 2 | AD-3118 |
| A12563 | ggc uCU Uag caa AGU Caa gTT | 1 | 5722 | CUU GAC UUU GCU AAG AGC CdTdT | 2 | AD-3119 |
| 5718 | GGC UCU UAG CAA AGU CAA GdTdT | 1 | A12564 | cuu Gac uuu Gcu AAG Agc cTT | 2 | AD-3120 |
| 5718 | GGC UCU UAG CAA AGU CAA GdTdT | 1 | A12565 | cuu gac uuu gCU AAG AGC CTT | 2 | AD-3121 |
| 5718 | GGC UCU UAG CAA AGU CAA GdTdT | 1 | A12566 | CUU GAC UUU gcu aag agc cTT | 2 | AD-3122 |
| 5718 | GGC UCU UAG CAA AGU CAA GdTdT | 1 | A12567 | cuu gAC Uuu gcu AAG Agc cTT | 2 | AD-3123 |
| A23555 | GgC UCU uAG cAA AGU cAA GTsT | 1 | A17653 | CUU GAC UUU GCu AAG AGC CTsT | 2 | AD-3183 |
| A23555 | GgC UCU uAG cAA AGU cAA GTsT | 1 | A17657 | CuU GAC UUU GCU AAG AGC CTsT | 2 | AD-3184 |
| A23555 | GgC UCU uAG cAA AGU cAA GTsT | 1 | A23556 | CuU GAC UUU GCu AAG AGC CTsT | 2 | AD-3185 |
| A23555 | GgC UCU uAG cAA AGU cAA GTsT | 1 | A23558 | CuU GAC UUU GCu AAG AGC CdTdT (ab) | 2 | AD-3186 |
| A23557 | GgC UCU uAG cAA AGU cAA GdTdT (ab) | 1 | A23558 | CuU GAC UUU GCu AAG AGC CdTdT (ab) | 2 | AD-3187 |
| A23557 | GgC UCU uAG cAA AGU cAA GdTdT (ab) | 1 | A23556 | CuU GAC UUU GCu AAG AGC CTsT | 2 | AD-3188 |
| A23555 | GgC UCU uAG cAA AGU cAA GTsT | 1 | A23559 | p-CUU GAC UUU GCU AAG AGC CdTdT | 2 | AD-3189 |
| A23557 | GgC UCU uAG cAA AGU cAA GdTdT (ab) | 1 | A23559 | p-CUU GAC UUU GCU AAG AGC CdTdT | 2 | AD-3190 |
| 5718 | GGC UCU UAG CAA AGU CAA GdTdT | 1 | A23559 | p-CUU GAC UUU GCU AAG AGC CdTdT | 2 | AD-3191 |
| 5718 | GGC UCU UAG CAA AGU CAA GdTdT | 1 | 5719 | CUU GAC UUU GCU AAG AGC CdTdT | 2 | AD-3124 |
| scram | GGC UCU AAG CUA ACU GAA GdTdT | 291 | scram | CUU CACGUUA GCU UAG AGC CdTdT | 317 | AD-2153 |

| Table 10b | | IC50 |
|---|---|---|
| Duplex# | Activity % (80 nM) | in vitro |
| AD-3136 | 81 | |
| AD-3137 | 63 | 12.97 |
| AD-3138 | 79 | |
| AD-3139 | 80 | |
| AD-3140 | 75 | |
| AD-3141 | 79 | |
| AD-3142 | 74 | |
| AD-3143 | 47 | |
| AD-3144 | 77 | |
| AD-3145 | 73 | |
| AD-3146 | 69 | |
| AD-3147 | 71 | 1.97 |
| AD-3148 | 64 | 13.85 |
| AD-3149 | 9 | |
| AD-3150 | 71 | 3.59 |
| AD-3151 | 72 | 2.90 |
| AD-3152 | 69 | 5.80 |
| AD-3116 | 62 | |
| AD-3117 | 62 | |
| AD-3118 | 65 | |
| AD-3119 | 62 | |
| AD-3120 | 8 | >80 |
| AD-3121 | 9 | >80 |
| AD-3122 | 11 | >80 |
| AD-3123 | 9 | >80 |
| AD-3183 | 68 | 4.20 |
| AD-3184 | 70 | 4.00 |
| AD-3185 | 68 | 4.30 |
| AD-3186 | 69 | 4.50 |
| AD-3187 | 69 | 2.80 |
| AD-3188 | 69 | 4.50 |
| AD-3189 | 71 | 1.60 |
| AD-3190 | 71 | 1.17 |
| AD-3191 | 77 | 1.15 |
| AD-3124 | 80 | 0.92 |
| AD-2153 | 6 | |

Lower case is 2′OMe modification
Exo = s phosphothioate (Chemistry 1); exo (ab) = abasic support
endo light = UA/CA 2′ OMe(Chemistry 2); endo heavy = all Py as 2′-OMe (Chemistry 3)
heavy methylated = many modified nucleotides (nts) in a raw either from 5′ or 3′
2′-OMe, @ Pos 2 (Chemistry 4); TT —complem. @ 2′-OMe, PTO
p = 5′-phosphate

TABLE 11

Sequences (11a) and in vitro antiviral activity (11b) of 2′OMe and exonuclease
(exo) protected with hydroxy pyrollidine (hp) linker modified RSV siRNA

| | | SEQ | | | SEQ | |
|---|---|---|---|---|---|---|
| Table 11a | | ID | | | ID | Duplex |
| ss-ID # | sense strand (5′--3′) | NO: | as-ID # | antisense strand (5′--3′) | NO: | ID # |
| A26547 | GGC UCU UAG CAA AGU CAA Guu | 315 | A26563 | CUU GAC UUU GCU AAG AGC Cau-Hp | 316 | AD-16097 |
| A26547 | GGC UCU UAG CAA AGU CAA Guu | 315 | A26564 | CuU GAC UUU GCU AAG AGC cAU-Hp | 316 | AD-16098 |
| A26547 | GGC UCU UAG CAA AGU CAA Guu | 315 | A26576 | CUU GAc uUU gcU AAG Agc cAU-Hp | 316 | AD-16099 |
| A26547 | GGC UCU UAG CAA AGU CAA Guu | 315 | A26577 | CUU GAC UUU GCU AAG AGC Cau | 316 | AD-16100 |
| A26547 | GGC UCU UAG CAA AGU CAA Guu | 315 | A26578 | p-cuu GAC UUU GCU AAG AGC CdTdT | 2 | AD-16101 |
| A26547 | GGC UCU UAG CAA AGU CAA Guu | 315 | A26579 | p-cuu GAC UUU GCU AAG AGC C-hp | 305 | AD-16102 |
| A26547 | GGC UCU UAG CAA AGU CAA Guu | 315 | A26580 | p-cUU GAC UUU GCU AAG AGcc-hp | 305 | AD-16103 |
| A26547 | GGC UCU UAG CAA AGU CAA Guu | 315 | A26581 | p-cuU GAC UUU GCU AAG AGC c-hp | 305 | AD-16104 |
| A26547 | GGC UCU UAG CAA AGU CAA Guu | 315 | A26582 | cuu GAC UUU GCU AAG AGC CdTdT | 2 | AD-16105 |
| A26547 | GGC UCU UAG CAA AGU CAA Guu | 315 | A26583 | cuu GAC UUU GCU AAG AGC C-hp | 305 | AD-16106 |
| A26547 | GGC UCU UAG CAA AGU CAA Guu | 315 | A26584 | cUU GAC UUU GCU AAG AGcc-hp | 305 | AD-16107 |
| A26547 | GGC UCU UAG CAA AGU CAA Guu | 315 | A26585 | cuU GAC UUU GCU AAG AGC c-hp | 305 | AD-16108 |
| A26548 | GGC UCU UAG CAA AGU CAA Guu-Hp | 315 | A26563 | CUU GAC UUU GCU AAG AGC Cau-Hp | 316 | AD-16109 |
| A26548 | GGC UCU UAG CAA AGU CAA Guu-Hp | 315 | A26564 | CuU GAC UUU GCU AAG AGC cAU-Hp | 316 | AD-16110 |
| A26548 | GGC UCU UAG CAA AGU CAA Guu-Hp | 315 | A26576 | CUU GAc uUU gcU AAG Agc cAU-Hp | 316 | AD-16111 |
| A26548 | GGC UCU UAG CAA AGU CAA Guu-Hp | 315 | A26577 | CUU GAC UUU GCU AAG AGC Cau | 316 | AD-16112 |
| A26548 | GGC UCU UAG CAA AGU CAA Guu-Hp | 315 | A26578 | p-cuu GAC UUU GCU AAG AGC CdTdT | 2 | AD-16113 |
| A26548 | GGC UCU UAG CAA AGU CAA Guu-Hp | 315 | A26579 | p-cuu GAC UUU GCU AAG AGC C-hp | 305 | AD-16114 |
| A26548 | GGC UCU UAG CAA AGU CAA Guu-Hp | 315 | A26580 | p-cUU GAC UUU GCU AAG AGcc-hp | 305 | AD-16115 |
| A26548 | GGC UCU UAG CAA AGU CAA Guu-Hp | 315 | A26581 | p-cuU GAC UUU GCU AAG AGC c-hp | 305 | AD-16116 |
| A26548 | GGC UCU UAG CAA AGU CAA Guu-Hp | 315 | A26582 | cuu GAC UUU GCU AAG AGC CdTdT | 2 | AD-16117 |
| A26548 | GGC UCU UAG CAA AGU CAA Guu-Hp | 315 | A26583 | cuu GAC UUU GCU AAG AGC C-hp | 305 | AD-16118 |
| A26548 | GGC UCU UAG CAA AGU CAA Guu-Hp | 315 | A26584 | cUU GAC UUU GCU AAG AGcc-hp | 305 | AD-16119 |
| A26548 | GGC UCU UAG CAA AGU CAA Guu-Hp | 315 | A26585 | cuU GAC UUU GCU AAG AGC c-hp | 305 | AD-16120 |
| A26549 | GgC UCU UAG CAA AGU CAA Guu-Hp | 315 | A26563 | CUU GAC UUU GCU AAG AGC Cau-Hp | 316 | AD-16121 |
| A26549 | GgC UCU UAG CAA AGU CAA Guu-Hp | 315 | A26564 | CuU GAC UUU GCU AAG AGC cAU-Hp | 316 | AD-16122 |
| A26549 | GgC UCU UAG CAA AGU CAA Guu-Hp | 315 | A26576 | CUU GAc uUU gcU AAG Agc cAU-Hp | 316 | AD-16123 |
| A26549 | GgC UCU UAG CAA AGU CAA Guu-Hp | 315 | A26577 | CUU GAC UUU GCU AAG AGC Cau | 316 | AD-16124 |
| A26549 | GgC UCU UAG CAA AGU CAA Guu-Hp | 315 | A26578 | p-cuu GAC UUU GCU AAG AGC CdTdT | 2 | AD-16125 |
| A26549 | GgC UCU UAG CAA AGU CAA Guu-Hp | 315 | A26579 | p-cuu GAC UUU GCU AAG AGC C-hp | 305 | AD-16126 |
| A26549 | GgC UCU UAG CAA AGU CAA Guu-Hp | 315 | A26580 | p-cUU GAC UUU GCU AAG AGcc-hp | 305 | AD-16127 |
| A26549 | GgC UCU UAG CAA AGU CAA Guu-Hp | 315 | A26581 | p-cuU GAC UUU GCU AAG AGC c-hp | 305 | AD-16128 |
| A26549 | GgC UCU UAG CAA AGU CAA Guu-Hp | 315 | A26582 | cuu GAC UUU GCU AAG AGC CdTdT | 2 | AD-16129 |
| A26549 | GgC UCU UAG CAA AGU CAA Guu-Hp | 315 | A26583 | cuu GAC UUU GCU AAG AGC C-hp | 305 | AD-16130 |
| A26549 | GgC UCU UAG CAA AGU CAA Guu-Hp | 315 | A26584 | cUU GAC UUU GCU AAG AGcc-hp | 305 | AD-16131 |
| A26549 | GgC UCU UAG CAA AGU CAA Guu-Hp | 315 | A26585 | cuU GAC UUU GCU AAG AGC c-hp | 305 | AD-16132 |
| A26550 | GgC UCU uAG cAA AGU cAA Guu-Hp | 315 | A26563 | CUU GAC UUU GCU AAG AGC Cau-Hp | 316 | AD-16133 |
| A26550 | GgC UCU uAG cAA AGU cAA Guu-Hp | 315 | A26564 | CuU GAC UUU GCU AAG AGC cAU-Hp | 316 | AD-16134 |
| A26550 | GgC UCU uAG cAA AGU cAA Guu-Hp | 315 | A26576 | CUU GAc uUU gcU AAG Agc cAU-Hp | 316 | AD-16135 |
| A26550 | GgC UCU uAG cAA AGU cAA Guu-Hp | 315 | A26577 | CUU GAC UUU GCU AAG AGC Cau | 316 | AD-16136 |
| A26550 | GgC UCU uAG cAA AGU cAA Guu-Hp | 315 | A26578 | p-cuu GAC UUU GCU AAG AGC CdTdT | 2 | AD-16137 |
| A26550 | GgC UCU uAG cAA AGU cAA Guu-Hp | 315 | A26579 | p-cuu GAC UUU GCU AAG AGC C-hp | 305 | AD-16138 |
| A26550 | GgC UCU uAG cAA AGU cAA Guu-Hp | 315 | A26580 | p-cUU GAC UUU GCU AAG AGcc-hp | 305 | AD-16139 |
| A26550 | GgC UCU uAG cAA AGU cAA Guu-Hp | 315 | A26581 | p-cuU GAC UUU GCU AAG AGC c-hp | 305 | AD-16140 |
| A26550 | GgC UCU uAG cAA AGU cAA Guu-Hp | 315 | A26582 | cuu GAC UUU GCU AAG AGC CdTdT | 2 | AD-16141 |
| A26550 | GgC UCU uAG cAA AGU cAA Guu-Hp | 315 | A26583 | cuu GAC UUU GCU AAG AGC C-hp | 305 | AD-16142 |
| A26550 | GgC UCU uAG cAA AGU cAA Guu-Hp | 315 | A26584 | cUU GAC UUU GCU AAG AGcc-hp | 305 | AD-16143 |
| A26550 | GgC UCU uAG cAA AGU cAA Guu-Hp | 315 | A26585 | cuU GAC UUU GCU AAG AGC c-hp | 305 | AD-16144 |
| A26551 | GgC UCU uAG cAA AGU cAA GdTdT-Hp | 1 | A26563 | CUU GAC UUU GCU AAG AGC Cau-Hp | 316 | AD-16145 |
| A26551 | GgC UCU uAG cAA AGU cAA GdTdT-Hp | 1 | A26564 | CuU GAC UUU GCU AAG AGC cAU-Hp | 316 | AD-16146 |
| A26551 | GgC UCU uAG cAA AGU cAA GdTdT-Hp | 1 | A26576 | CUU GAc uUU gcU AAG Agc cAU-Hp | 316 | AD-16147 |
| A26551 | GgC UCU uAG cAA AGU cAA GdTdT-Hp | 1 | A26577 | CUU GAC UUU GCU AAG AGC Cau | 316 | AD-16148 |
| A26551 | GgC UCU uAG cAA AGU cAA GdTdT-Hp | 1 | A26578 | p-cuu GAC UUU GCU AAG AGC CdTdT | 2 | AD-16149 |
| A26551 | GgC UCU uAG cAA AGU cAA GdTdT-Hp | 1 | A26579 | p-cuu GAC UUU GCU AAG AGC C-hp | 305 | AD-16150 |
| A26551 | GgC UCU uAG cAA AGU cAA GdTdT-Hp | 1 | A26580 | p-cUU GAC UUU GCU AAG AGcc-hp | 305 | AD-16151 |
| A26551 | GgC UCU uAG cAA AGU cAA GdTdT-Hp | 1 | A26581 | p-cuU GAC UUU GCU AAG AGC c-hp | 305 | AD-16152 |
| A26551 | GgC UCU uAG cAA AGU cAA GdTdT-Hp | 1 | A26582 | cuu GAC UUU GCU AAG AGC CdTdT | 2 | AD-16153 |
| A26551 | GgC UCU uAG cAA AGU cAA GdTdT-Hp | 1 | A26583 | cuu GAC UUU GCU AAG AGC C-hp | 305 | AD-16154 |
| A26551 | GgC UCU uAG cAA AGU cAA GdTdT-Hp | 1 | A26584 | cUU GAC UUU GCU AAG AGcc-hp | 305 | AD-16155 |
| A26551 | GgC UCU uAG cAA AGU cAA GdTdT-Hp | 1 | A26585 | cuU GAC UUU GCU AAG AGC c-hp | 305 | AD-16156 |
| A26552 | GgC UCU uAG cAA AGU cAA G-Hp | 302 | A26563 | CUU GAC UUU GCU AAG AGC Cau-Hp | 316 | AD-16157 |
| A26552 | GgC UCU uAG cAA AGU cAA G-Hp | 302 | A26564 | CuU GAC UUU GCU AAG AGC cAU-Hp | 316 | AD-16158 |
| A26552 | GgC UCU uAG cAA AGU cAA G-Hp | 302 | A26576 | CUU GAc uUU gcU AAG Agc cAU-Hp | 316 | AD-16159 |
| A26552 | GgC UCU uAG cAA AGU cAA G-Hp | 302 | A26577 | CUU GAC UUU GCU AAG AGC Cau | 316 | AD-16160 |
| A26552 | GgC UCU uAG cAA AGU cAA G-Hp | 302 | A26578 | p-cuu GAC UUU GCU AAG AGC CdTdT | 2 | AD-16161 |
| A26552 | GgC UCU uAG cAA AGU cAA G-Hp | 302 | A26579 | p-cuu GAC UUU GCU AAG AGC C-hp | 305 | AD-16162 |
| A26552 | GgC UCU uAG cAA AGU cAA G-Hp | 302 | A26580 | p-cUU GAC UUU GCU AAG AGcc-hp | 305 | AD-16163 |
| A26552 | GgC UCU uAG cAA AGU cAA G-Hp | 302 | A26581 | p-cuU GAC UUU GCU AAG AGC c-hp | 305 | AD-16164 |
| A26552 | GgC UCU uAG cAA AGU cAA G-Hp | 302 | A26582 | cuu GAC UUU GCU AAG AGC CdTdT | 2 | AD-16165 |
| A26552 | GgC UCU uAG cAA AGU cAA G-Hp | 302 | A26583 | cuu GAC UUU GCU AAG AGC C-hp | 305 | AD-16166 |
| A26552 | GgC UCU uAG cAA AGU cAA G-Hp | 302 | A26584 | cUU GAC UUU GCU AAG AGcc-hp | 305 | AD-16167 |
| A26552 | GgC UCU uAG cAA AGU cAA G-Hp | 302 | A26585 | cuU GAC UUU GCU AAG AGC c-hp | 305 | AD-16168 |
| A26554 | GgC uCu UaG cAa AgU cAa GdTdT-Hp | 1 | A26561 | p-cUu GaC uUu GcU aAg AgC cdTdT-Hp | 2 | AD-16169 |
| A26554 | GgC uCu UaG cAa AgU cAa GdTdT-Hp | 1 | A26562 | p-cUu GaC uUu GcU aAg AgC c-Hp | 305 | AD-16170 |
| A26555 | GgC uCu UaG cAa AgU cAa G-Hp | 302 | A26561 | p-cUu GaC uUu GcU aAg AgC cdTdT-Hp | 2 | AD-16171 |
| A26555 | GgC uCu UaG cAa AgU cAa G-Hp | 302 | A26562 | p-cUu GaC uUu GcU aAg AgC c-Hp | 305 | AD-16172 |
| A26831 | GgC UCU uAG cAA AGU cAA GdTdT-HP | 1 | A26844 | CuU GAC uUU GCU AAG AGC CdTdT-HP | 2 | AD-16441 |
| A26831 | GgC UCU uAG cAA AGU cAA GdTdT-HP | 1 | A26845 | CuU GAC UuU GCU AAG AGC CdTdT-HP | 2 | AD-16442 |
| A26831 | GgC UCU uAG cAA AGU cAA GdTdT-HP | 1 | A26846 | CuU GAC UUu GCU AAG AGC CdTdT-HP | 2 | AD-16443 |
| A26831 | GgC UCU uAG cAA AGU cAA GdTdT-HP | 1 | A26847 | CuU GAC uUu GCU AAG AGC CdTdT-HP | 2 | AD-16444 |
| A26831 | GgC UCU uAG cAA AGU cAA GdTdT-HP | 1 | A26848 | CuU GAC uUU GCu AAG AGC CdTdT-HP | 2 | AD-16445 |
| A26831 | GgC UCU uAG cAA AGU cAA GdTdT-HP | 1 | A26849 | CuU GAC UuU GCu AAG AGC CdTdT-HP | 2 | AD-16446 |
| A26831 | GgC UCU uAG cAA AGU cAA GdTdT-HP | 1 | A26850 | CuU GAC UUu GCu AAG AGC CdTdT-HP | 2 | AD-16447 |
| A26831 | GgC UCU uAG cAA AGU cAA GdTdT-HP | 1 | A26851 | CuU GAC uUu GCu AAG AGC CdTdT-HP | 2 | AD-16448 |
| A26831 | GgC UCU uAG cAA AGU cAA GdTdT-HP | 1 | A26867 | Cuu GAC uUu GCu AAG AGc cdTdT-HP | 2 | AD-16464 |
| A26831 | GgC UCU uAG cAA AGU cAA GdTdT-HP | 1 | A26868 | Cuu GAC UUU GCu AAG AGC cdTdT-HP | 2 | AD-16465 |
| A26831 | GgC UCU uAG cAA AGU cAA GdTdT-HP | 1 | A26869 | Cuu GAC UUU GCu AAG AGc cdTdT-HP | 2 | AD-16466 |
| A26831 | GgC UCU uAG cAA AGU cAA GdTdT-HP | 1 | A26870 | CUu GAC uUU GCU AAG AGC cdTdT-HP | 2 | AD-16467 |
| A26831 | GgC UCU uAG cAA AGU cAA GdTdT-HP | 1 | A26871 | CUu GAC UuU GCU AAG AGC cdTdT-HP | 2 | AD-16468 |
| A26831 | GgC UCU uAG cAA AGU cAA GdTdT-HP | 1 | A26872 | CUu GAC UUu GCU AAG AGC cdTdT-HP | 2 | AD-16469 |
| A26831 | GgC UCU uAG cAA AGU cAA GdTdT-HP | 1 | A26873 | CUu GAC uUU GCu AAG AGC cdTdT-HP | 2 | AD-16470 |
| A26831 | GgC UCU uAG cAA AGU cAA GdTdT-HP | 1 | A26874 | CUu GAC UuU GCu AAG AGC cdTdT-HP | 2 | AD-16471 |
| A26831 | GgC UCU uAG cAA AGU cAA GdTdT-HP | 1 | A26875 | CUu GAC UUu GCu AAG AGC cdTdT-HP | 2 | AD-16472 |
| A26831 | GgC UCU uAG cAA AGU cAA GdTdT-HP | 1 | A26876 | CUu GAC uUU GCU AAG AGc CdTdT-HP | 2 | AD-16473 |
| A26831 | GgC UCU uAG cAA AGU cAA GdTdT-HP | 1 | A26877 | CUu GAC UuU GCU AAG AGc CdTdT-HP | 2 | AD-16474 |
| A26831 | GgC UCU uAG cAA AGU cAA GdTdT-HP | 1 | A26878 | CUu GAC UUu GCU AAG AGc CdTdT-HP | 2 | AD-16475 |
| A26832 | GGc uCU UAG cAa AGu cAA GdTdT-HP | 1 | A26836 | cuU GAC uUU GCU AAG AGC CdTdT-HP | 2 | AD-16476 |
| A26832 | GGc uCU UAG cAa AGu cAA GdTdT-HP | 1 | A26837 | cuU GAC UuU GCU AAG AGC CdTdT-HP | 2 | AD-16477 |
| A26832 | GGc uCU UAG cAa AGu cAA GdTdT-HP | 1 | A26838 | cuU GAC UUu GCU AAG AGC CdTdT-HP | 2 | AD-16478 |
| A26832 | GGc uCU UAG cAa AGu cAA GdTdT-HP | 1 | A26839 | cuU GAC uUu GCU AAG AGC CdTdT-HP | 2 | AD-16479 |
| A26832 | GGc uCU UAG cAa AGu cAA GdTdT-HP | 1 | A26840 | cuU GAC uUU GCu AAG AGC CdTdT-HP | 2 | AD-16480 |
| A26832 | GGc uCU UAG cAa AGu cAA GdTdT-HP | 1 | A26841 | cuU GAC UuU GCu AAG AGC CdTdT-HP | 2 | AD-16481 |
| A26832 | GGc uCU UAG cAa AGu cAA GdTdT-HP | 1 | A26842 | cuU GAC UUu GCu AAG AGC CdTdT-HP | 2 | AD-16482 |
| A26832 | GGc uCU UAG cAa AGu cAA GdTdT-HP | 1 | A26843 | cuU GAC uUu GCu AAG AGC CdTdT-HP | 2 | AD-16483 |
| A26832 | GGc uCU UAG cAa AGu cAA GdTdT-HP | 1 | A26844 | CuU GAC uUU GCU AAG AGC CdTdT-HP | 2 | AD-16484 |
| A26832 | GGc uCU UAG cAa AGu cAA GdTdT-HP | 1 | A26845 | CuU GAC UuU GCU AAG AGC CdTdT-HP | 2 | AD-16485 |
| A26832 | GGc uCU UAG cAa AGu cAA GdTdT-HP | 1 | A26846 | CuU GAC UUu GCU AAG AGC CdTdT-HP | 2 | AD-16486 |
| A26832 | GGc uCU UAG cAa AGu cAA GdTdT-HP | 1 | A26847 | CuU GAC uUu GCU AAG AGC CdTdT-HP | 2 | AD-16487 |
| A26832 | GGc uCU UAG cAa AGu cAA GdTdT-HP | 1 | A26848 | CuU GAC uUU GCu AAG AGC CdTdT-HP | 2 | AD-16488 |
| A26832 | GGc uCU UAG cAa AGu cAA GdTdT-HP | 1 | A26849 | CuU GAC UuU GCu AAG AGC CdTdT-HP | 2 | AD-16489 |
| A26832 | GGc uCU UAG cAa AGu cAA GdTdT-HP | 1 | A26850 | CuU GAC UUu GCu AAG AGC CdTdT-HP | 2 | AD-16490 |
| A26832 | GGc uCU UAG cAa AGu cAA GdTdT-HP | 1 | A26851 | CuU GAC uUu GCu AAG AGC CdTdT-HP | 2 | AD-16491 |
| A26832 | GGc uCU UAG cAa AGu cAA GdTdT-HP | 1 | A26852 | Cuu GAC uUU GCU AAG AGC cdTdT-HP | 2 | AD-16492 |
| A26832 | GGc uCU UAG cAa AGu cAA GdTdT-HP | 1 | A26853 | Cuu GAC UuU GCU AAG AGC cdTdT-HP | 2 | AD-16493 |
| A26832 | GGc uCU UAG cAa AGu cAA GdTdT-HP | 1 | A26869 | Cuu GAC UUU GCu AAG AGc cdTdT-HP | 2 | AD-16509 |
| A26832 | GGc uCU UAG cAa AGu cAA GdTdT-HP | 1 | A26870 | CUu GAC uUU GCU AAG AGC cdTdT-HP | 2 | AD-16510 |
| A26832 | GGc uCU UAG cAa AGu cAA GdTdT-HP | 1 | A26871 | CUu GAC UuU GCU AAG AGC cdTdT-HP | 2 | AD-16511 |
| A26832 | GGc uCU UAG cAa AGu cAA GdTdT-HP | 1 | A26872 | CUu GAC UUu GCU AAG AGC cdTdT-HP | 2 | AD-16512 |
| A26832 | GGc uCU UAG cAa AGu cAA GdTdT-HP | 1 | A26873 | CUu GAC uUU GCu AAG AGC cdTdT-HP | 2 | AD-16513 |
| A26832 | GGc uCU UAG cAa AGu cAA GdTdT-HP | 1 | A26874 | CUu GAC UuU GCu AAG AGC cdTdT-HP | 2 | AD-16514 |
| A26832 | GGc uCU UAG cAa AGu cAA GdTdT-HP | 1 | A26875 | CUu GAC UUu GCu AAG AGC cdTdT-HP | 2 | AD-16515 |
| A26832 | GGc uCU UAG cAa AGu cAA GdTdT-HP | 1 | A26876 | CUu GAC uUU GCU AAG AGc CdTdT-HP | 2 | AD-16516 |
| A26832 | GGc uCU UAG cAa AGu cAA GdTdT-HP | 1 | A26877 | CUu GAC UuU GCU AAG AGc CdTdT-HP | 2 | AD-16517 |
| A26832 | GGc uCU UAG cAa AGu cAA GdTdT-HP | 1 | A26878 | CUu GAC UUu GCU AAG AGc CdTdT-HP | 2 | AD-16518 |
| A26833 | GGc uCU UAG cAa AGu cAa GdTdT-HP | 1 | A26836 | cuU GAC uUU GCU AAG AGC CdTdT-HP | 2 | AD-16519 |
| A26833 | GGc uCU UAG cAa AGu cAa GdTdT-HP | 1 | A26837 | cuU GAC UuU GCU AAG AGC CdTdT-HP | 2 | AD-16520 |
| A26833 | GGc uCU UAG cAa AGu cAa GdTdT-HP | 1 | A26838 | cuU GAC UUu GCU AAG AGC CdTdT-HP | 2 | AD-16521 |
| A26833 | GGc uCU UAG cAa AGu cAa GdTdT-HP | 1 | A26839 | cuU GAC uUu GCU AAG AGC CdTdT-HP | 2 | AD-16522 |
| A26833 | GGc uCU UAG cAa AGu cAa GdTdT-HP | 1 | A26840 | cuU GAC uUU GCu AAG AGC CdTdT-HP | 2 | AD-16523 |
| A26833 | GGc uCU UAG cAa AGu cAa GdTdT-HP | 1 | A26841 | cuU GAC UuU GCu AAG AGC CdTdT-HP | 2 | AD-16524 |
| A26833 | GGc uCU UAG cAa AGu cAa GdTdT-HP | 1 | A26842 | cuU GAC UUu GCu AAG AGC CdTdT-HP | 2 | AD-16525 |
| A26833 | GGc uCU UAG cAa AGu cAa GdTdT-HP | 1 | A26843 | cuU GAC uUu GCu AAG AGC CdTdT-HP | 2 | AD-16526 |
| A26833 | GGc uCU UAG cAa AGu cAa GdTdT-HP | 1 | A26844 | CuU GAC uUU GCU AAG AGC CdTdT-HP | 2 | AD-16527 |
| A26833 | GGc uCU UAG cAa AGu cAa GdTdT-HP | 1 | A26845 | CuU GAC UuU GCU AAG AGC CdTdT-HP | 2 | AD-16528 |
| A26833 | GGc uCU UAG cAa AGu cAa GdTdT-HP | 1 | A26846 | CuU GAC UUu GCU AAG AGC CdTdT-HP | 2 | AD-16529 |
| A26833 | GGc uCU UAG cAa AGu cAa GdTdT-HP | 1 | A26847 | CuU GAC uUu GCU AAG AGC CdTdT-HP | 2 | AD-16530 |
| A26833 | GGc uCU UAG cAa AGu cAa GdTdT-HP | 1 | A26848 | CuU GAC uUU GCu AAG AGC CdTdT-HP | 2 | AD-16531 |
| A26833 | GGc uCU UAG cAa AGu cAa GdTdT-HP | 1 | A26849 | CuU GAC UuU GCu AAG AGC CdTdT-HP | 2 | AD-16532 |
| A26833 | GGc uCU UAG cAa AGu cAa GdTdT-HP | 1 | A26850 | CuU GAC UUu GCu AAG AGC CdTdT-HP | 2 | AD-16533 |
| A26833 | GGc uCU UAG cAa AGu cAa GdTdT-HP | 1 | A26866 | Cuu GAC UUu GCu AAG AGc cdTdT-HP | 2 | AD-16549 |
| A26833 | GGc uCU UAG cAa AGu cAa GdTdT-HP | 1 | A26867 | Cuu GAC uUu GCu AAG AGc cdTdT-HP | 2 | AD-16550 |
| A26833 | GGc uCU UAG cAa AGu cAa GdTdT-HP | 1 | A26868 | Cuu GAC UUU GCu AAG AGC cdTdT-HP | 2 | AD-16551 |
| A26833 | GGc uCU UAG cAa AGu cAa GdTdT-HP | 1 | A26869 | Cuu GAC UUU GCu AAG AGc cdTdT-HP | 2 | AD-16552 |
| A26833 | GGc uCU UAG cAa AGu cAa GdTdT-HP | 1 | A26870 | CUu GAC uUU GCU AAG AGC cdTdT-HP | 2 | AD-16553 |
| A26833 | GGc uCU UAG cAa AGu cAa GdTdT-HP | 1 | A26871 | CUu GAC UuU GCU AAG AGC cdTdT-HP | 2 | AD-16554 |
| A26833 | GGc uCU UAG cAa AGu cAa GdTdT-HP | 1 | A26872 | CUu GAC UUu GCU AAG AGC cdTdT-HP | 2 | AD-16555 |
| A26833 | GGc uCU UAG cAa AGu cAa GdTdT-HP | 1 | A26873 | CUu GAC uUU GCu AAG AGC cdTdT-HP | 2 | AD-16556 |
| A26833 | GGc uCU UAG cAa AGu cAa GdTdT-HP | 1 | A26874 | CUu GAC UuU GCu AAG AGC cdTdT-HP | 2 | AD-16557 |
| A26833 | GGc uCU UAG cAa AGu cAa GdTdT-HP | 1 | A26875 | CUu GAC UUu GCu AAG AGC cdTdT-HP | 2 | AD-16558 |
| A26833 | GGc uCU UAG cAa AGu cAa GdTdT-HP | 1 | A26876 | CUu GAC uUU GCU AAG AGc CdTdT-HP | 2 | AD-16559 |
| A26833 | GGc uCU UAG cAa AGu cAa GdTdT-HP | 1 | A26877 | CUu GAC UuU GCU AAG AGc CdTdT-HP | 2 | AD-16560 |
| A26833 | GGc uCU UAG cAa AGu cAa GdTdT-HP | 1 | A26878 | CUu GAC UUu GCU AAG AGc CdTdT-HP | 2 | AD-16561 |
| A26834 | GGc uCU UAG cAa AGu cAA Guu-HP | 315 | A26844 | CuU GAC uUU GCU AAG AGC CdTdT-HP | 2 | AD-16570 |
| A26834 | GGc uCU UAG cAa AGu cAA Guu-HP | 315 | A26845 | CuU GAC UuU GCU AAG AGC CdTdT-HP | 2 | AD-16571 |
| A26835 | GGc uCU UAG cAa AGu cAa Guu-HP | 315 | A26836 | cuU GAC uUU GCU AAG AGC CdTdT-HP | 2 | AD-16605 |
| A26835 | GGc uCU UAG cAa AGu cAa Guu-HP | 315 | A26837 | cuU GAC UuU GCU AAG AGC CdTdT-HP | 2 | AD-16606 |
| A26835 | GGc uCU UAG cAa AGu cAa Guu-HP | 315 | A26838 | cuU GAC UUu GCU AAG AGC CdTdT-HP | 2 | AD-16607 |
| A26835 | GGc uCU UAG cAa AGu cAa Guu-HP | 315 | A26844 | CuU GAC uUU GCU AAG AGC CdTdT-HP | 2 | AD-16613 |
| A26835 | GGc uCU UAG cAa AGu cAa Guu-HP | 315 | A26845 | CuU GAC UuU GCU AAG AGC CdTdT-HP | 2 | AD-16614 |

| Table 11b | | | |
|---|---|---|---|
| Duplex ID # | 20 nM-Activity % | IC50 | Notes |
| AD-16097 | 64 | | |
| AD-16098 | 62 | | |
| AD-16099 | 63 | 1 | (3-2-2) 2′-OMe |
| AD-16100 | 63 | | |
| AD-16101 | 29 | | |
| AD-16102 | 33 | | |
| AD-16103 | 61 | >20 | |
| AD-16104 | 41 | | |
| AD-16105 | 43 | | |
| AD-16106 | 39 | | |
| AD-16107 | 41 | | |
| AD-16108 | 16 | | |
| AD-16109 | 58 | | |
| AD-16110 | 64 | | |
| AD-16111 | 60 | 1 | (3-2-2) 2′-OMe |
| AD-16112 | 55 | | |
| AD-16113 | 20 | >80 | |
| AD-16114 | 56 | 80 | |
| AD-16115 | 27 | | |
| AD-16116 | 22 | | |
| AD-16117 | −2 | | |
| AD-16118 | 16 | | |
| AD-16119 | 39 | | |
| AD-16120 | 38 | | |
| AD-16121 | 54 | | |
| AD-16122 | 52 | 6 | |
| AD-16123 | 55 | | (3-2-2) 2′-OMe |
| AD-16124 | 52 | | |
| AD-16125 | 35 | | |
| AD-16126 | 12 | | |
| AD-16127 | 48 | 12 | |
| AD-16128 | 13 | | |
| AD-16129 | 7 | | |
| AD-16130 | 6 | | |
| AD-16131 | 47 | | |
| AD-16132 | 30 | | |
| AD-16133 | 52 | 8 | |
| AD-16134 | 43 | | |
| AD-16135 | 62 | 15 | (3-2-2) 2′-OMe |
| AD-16136 | 50 | | |
| AD-16137 | 8 | | |
| AD-16138 | 31 | | |
| AD-16139 | 21 | | |
| AD-16140 | 11 | | |
| AD-16141 | 25 | | |
| AD-16142 | 11 | | |
| AD-16143 | 40 | | |
| AD-16144 | 34 | | |
| AD-16145 | 50 | | |
| AD-16146 | 41 | | |
| AD-16147 | 50 | 10 | (3-2-2) 2′-OMe |
| AD-16148 | 55 | | |
| AD-16149 | 20 | | |
| AD-16150 | 11 | | |
| AD-16151 | 33 | | |
| AD-16152 | 36 | | |
| AD-16153 | 47 | | |
| AD-16154 | 31 | | |
| AD-16155 | 38 | | |
| AD-16156 | 22 | | |
| AD-16157 | 41 | | |
| AD-16158 | 40 | | |
| AD-16159 | 45 | 11 | (3-2-2) 2′-OMe |
| AD-16160 | 59 | 3 | |
| AD-16161 | 26 | | |
| AD-16162 | 22 | | |
| AD-16163 | 28 | | |
| AD-16164 | 34 | | |
| AD-16165 | 31 | | |
| AD-16166 | 25 | | |
| AD-16167 | 19 | >80 | |
| AD-16168 | 0 | | |
| AD-16169 | 44 | 80 | alt 2′OH/OMe |
| AD-16170 | 35 | <80 | alt 2′OH/OMe |
| AD-16171 | 40 | 80-20 | alt 2′OH/OMe |
| AD-16172 | 37 | >80 | alt 2′OH/OMe |
| AD-16441 | 29 | | |
| AD-16442 | 32 | | |
| AD-16443 | 32 | | |
| AD-16444 | 44 | | |
| AD-16445 | 45 | | |
| AD-16446 | 44 | | |
| AD-16447 | 51 | | |
| AD-16448 | 54 | | |
| AD-16464 | 42 | | |
| AD-16465 | 8 | | |
| AD-16466 | 18 | | |
| AD-16467 | 83 | 1 | |
| AD-16468 | 83 | 1 | |
| AD-16469 | 70 | >1.2 | |
| AD-16470 | 69 | >1.2 | |
| AD-16471 | 85 | >1.2 | |
| AD-16472 | 78 | >1.2 | |
| AD-16473 | 87 | <1.2 | |
| AD-16474 | 82 | >1.2 | |
| AD-16475 | 75 | 1 | |
| AD-16476 | 49 | | |
| AD-16477 | 50 | | |
| AD-16478 | 38 | | |
| AD-16479 | 49 | | |
| AD-16480 | 38 | | |
| AD-16481 | 49 | | |
| AD-16482 | 49 | | |
| AD-16483 | 38 | | |
| AD-16484 | 80 | | |
| AD-16485 | 79 | | |
| AD-16486 | 70 | >1.2 | |
| AD-16487 | 75 | >1.2 | |
| AD-16488 | 68 | | |
| AD-16489 | 74 | | |
| AD-16490 | 65 | | |
| AD-16491 | 65 | | |
| AD-16492 | 42 | | |
| AD-16493 | 56 | | |
| AD-16509 | 45 | | |
| AD-16510 | 84 | <1.2 | |
| AD-16511 | 85 | <1.2 | |
| AD-16512 | 85 | <1.2 | |
| AD-16513 | 83 | 1 | |
| AD-16514 | 86 | >1.2 | |
| AD-16515 | 85 | >1.2 | |
| AD-16516 | 84 | <1.2 | |
| AD-16517 | 87 | 1 | |
| AD-16518 | 81 | <1.2 | |
| AD-16519 | 50 | | |
| AD-16520 | 50 | | |
| AD-16521 | 39 | | |
| AD-16522 | 36 | | |
| AD-16523 | 80 | | |
| AD-16524 | 79 | | |
| AD-16525 | 50 | | |
| AD-16526 | 51 | | |
| AD-16527 | 80 | | |
| AD-16528 | 79 | | |
| AD-16529 | 78 | >1.2 | |
| AD-16530 | 77 | >1.2 | |
| AD-16531 | 77 | | |
| AD-16532 | 85 | | |
| AD-16533 | 73 | | |
| AD-16549 | 45 | | |
| AD-16550 | 51 | | |
| AD-16551 | 10 | | |
| AD-16552 | 55 | | |
| AD-16553 | 84 | 1 | |
| AD-16554 | 83 | 1 | |
| AD-16555 | 85 | <1.2 | |
| AD-16556 | 85 | <1.2 | |
| AD-16557 | 85 | <1.2 | |
| AD-16558 | 85 | <1.2 | |
| AD-16559 | 87 | <1.2 | |
| AD-16560 | 85 | 1 | |
| AD-16561 | 79 | >1.2 | |
| AD-16570 | 80 | | |
| AD-16571 | 79 | | |
| AD-16605 | 44 | | |
| AD-16606 | 46 | | |
| AD-16607 | 43 | | |
| AD-16613 | 80 | | |
| AD-16614 | 79 | | |

2′OMe and exo protected with hp linker; some strands are 19 nts only

TABLE 12
Sequences (Table 12a) and in vitro antiviral activity (Table 12b) of 2′OMe and
exonuclease (exo) protected modified RSV siRNA with or without phorothioate(s)

| | | SEQ | | | SEQ | |
|---|---|---|---|---|---|---|
| Table 12a | | ID | | | ID | Duplex |
| SS ID # | sense strand (5′--3′) | NO: | AS ID # | antisense strand (5′--3′) | NO: | ID # |
| 5718 | GGC UCU UAG CAA AGU CAA | 1 | 5719 | CUU GAC UUU GCU AAG AGC CdTdT | 2 | RSV01 |
| | GdTdT | | | | | |
| A-30631 | GGcuCUUAGcAaAGucAAGdTsdT | 1 | A-30642 | CuUGACuUUGCUAAGAGCCdTsdT | 2 | AD-3518 |
| A-30625 | GGcuCUUAGcAaAGucAAGdTdT | 1 | A-30624 | CuUGACuUUGCUAAGAGCCdTdT | 2 | AD-3519 |
| A-30631 | GGcuCUUAGcAaAGucAAGdTsdT | 1 | A-30643 | CuUGACUuUGCUAAGAGCCdTsdT | 2 | AD-3520 |
| A-30625 | GGcuCUUAGcAaAGucAAGdTdT | 1 | A-30626 | CuUGACUuUGCUAAGAGCCdTdT | 2 | AD-3521 |
| A-30629 | GGcuCUUAGcAaAGucAaGdTsdT | 1 | A-30642 | CuUGACuUUGCUAAGAGCCdTsdT | 2 | AD-3522 |
| A-30623 | GGcuCUUAGcAaAGucAaGdTdT | 1 | A-30624 | CuUGACuUUGCUAAGAGCCdTdT | 2 | AD-3523 |
| A-30629 | GGcuCUUAGcAaAGucAaGdTsdT | 1 | A-30643 | CuUGACUuUGCUAAGAGCCdTsdT | 2 | AD-3524 |
| A-30623 | GGcuCUUAGcAaAGucAaGdTdT | 1 | A-30626 | CuUGACUuUGCUAAGAGCCdTdT | 2 | AD-3525 |
| A-30633 | GgCUCUuAGcAAAGUcAAGdTsdT | 1 | A-30646 | CUuGACuUUGCUAAGAGCcdTsdT | 2 | AD-3526 |
| A-30627 | GgCUCUuAGcAAAGUcAAGdTdT | 1 | A-30632 | CUuGACuUUGCUAAGAGCcdTdT | 2 | AD-3527 |
| A-30633 | GgCUCUuAGcAAAGUcAAGdTsdT | 1 | A-30647 | CUuGACUuUGCUAAGAGCcdTsdT | 2 | AD-3528 |
| A-30627 | GgCUCUuAGcAAAGUcAAGdTdT | 1 | A-30634 | CUuGACUuUGCUAAGAGCcdTdT | 2 | AD-3529 |
| A-30633 | GgCUCUuAGcAAAGUcAAGdTsdT | 1 | A-30654 | CUuGACUUuGCUAAGAGcCdTsdT | 2 | AD-3530 |
| A-30627 | GgCUCUuAGcAAAGUcAAGdTdT | 1 | A-30641 | CUuGACUUuGCUAAGAGcCdTdT | 2 | AD-3531 |
| A-30631 | GGcuCUUAGcAaAGucAAGdTsdT | 1 | A-30648 | CUuGACUUuGCUAAGAGCcdTsdT | 2 | AD-3532 |
| A-30625 | GGcuCUUAGcAaAGucAAGdTdT | 1 | A-30635 | CUuGACUUuGCUAAGAGCcdTdT | 2 | AD-3533 |
| A-30631 | GGcuCUUAGcAaAGucAAGdTsdT | 1 | A-30652 | CUuGACUuUGCUAAGAGcCdTsdT | 2 | AD-3534 |
| A-30625 | GGcuCUUAGcAaAGucAAGdTdT | 1 | A-30639 | CUuGACUuUGCUAAGAGcCdTdT | 2 | AD-3535 |
| A-30633 | GgCUCUuAGcAAAGUcAAGdTsdT | 1 | A-30644 | CuUGACUUuGCUAAGAGCCdTsdT | 2 | AD-3536 |
| A-30627 | GgCUCUuAGcAAAGUcAAGdTdT | 1 | A-30628 | CuUGACUUuGCUAAGAGCCdTdT | 2 | AD-3537 |
| A-30633 | GgCUCUuAGcAAAGUcAAGdTsdT | 1 | A-30645 | CuUGACuUuGCUAAGAGCCdTsdT | 2 | AD-3538 |
| A-30627 | GgCUCUuAGcAAAGUcAAGdTdT | 1 | A-30630 | CuUGACuUuGCUAAGAGCCdTdT | 2 | AD-3539 |

| Table 12b | Average IC50 In- |
|---|---|
| Duplex ID # | Vitro |
| RSV01 | 1 |
| AD-3518 | 2 |
| AD-3519 | 9 |
| AD-3520 | 1 |
| AD-3521 | 9 |
| AD-3522 | 3 |
| AD-3523 | 7 |
| AD-3524 | 2 |
| AD-3525 | 7 |
| AD-3526 | 4 |
| AD-3527 | 12 |
| AD-3528 | 2 |
| AD-3529 | 12 |
| AD-3530 | 3 |
| AD-3531 | 6 |
| AD-3532 | 1 |
| AD-3533 | 3 |
| AD-3534 | 1 |
| AD-3535 | 1 |
| AD-3536 | 50 |
| AD-3537 | 80 |
| AD-3538 | 80 |
| AD-3539 | 80 |

TABLE 13

Sequences (Table 13a) and in vitro antiviral activity (Table 13b) of 2′OMe and
exonuclease (exo) protected modified RSV siRNA with phorothioate(s) and with
uridine-adenine (UA) protected from endonucleases

| | SEQ | | | SEQ | | |
|---|---|---|---|---|---|---|
| Table 13a | ID | | | ID | | DUPLEX |
| SS ID # | NO: | sense strand (5′--3′) | AS ID # | NO: | antisense strand (5′--3′) | ID # |
| 5718 | 1 | GGC UCU UAG CAA AGU CAA GdTdT | 5719 | 2 | CUU GAC UUU GCU AAG AGC | RSV01 |
| | | | | | CdTdT | |
| A-30629 | 1 | GGcuCUUAGcAaAGucAaGdTsdT | A-30649 | 2 | CUuGACuUUGCuAAGAGCcdTsdT | AD-3581 |
| A-30631 | 1 | GGcuCUUAGcAaAGucAAGdTsdT | A-30649 | 2 | CUuGACuUUGCuAAGAGCcdTsdT | AD-3582 |
| A-30633 | 1 | GgCUCUuAGcAAAGUcAAGdTsdT | A-30649 | 2 | CUuGACuUUGCuAAGAGCcdTsdT | AD-3583 |
| A-30629 | 1 | GGcuCUUAGcAaAGucAaGdTsdT | A-30650 | 2 | CUuGACUUuGCuAAGAGCcdTsdT | AD-3584 |
| A-30631 | 1 | GGcuCUUAGcAaAGucAAGdTsdT | A-30650 | 2 | CUuGACUUuGCuAAGAGCcdTsdT | AD-3585 |
| A-30633 | 1 | GgCUCUuAGcAAAGUcAAGdTsdT | A-30650 | 2 | CUuGACUUuGCuAAGAGCcdTsdT | AD-3586 |
| A-30629 | 1 | GGcuCUUAGcAaAGucAaGdTsdT | A-30653 | 2 | CUuGACUuUGCuAAGAGCcdTsdT | AD-3587 |
| A-30631 | 1 | GGcuCUUAGcAaAGucAAGdTsdT | A-30653 | 2 | CUuGACUuUGCuAAGAGCcdTsdT | AD-3588 |
| A-30633 | 1 | GgCUCUuAGcAAAGUcAAGdTsdT | A-30653 | 2 | CUuGACUuUGCuAAGAGCcdTsdT | AD-3589 |
| A-30623 | 1 | GGcuCUUAGcAaAGucAaGdTdT | A-30636 | 2 | CUuGACuUUGCuAAGAGCcdTdT | AD-3590 |
| A-30625 | 1 | GGcuCUUAGcAaAGucAAGdTdT | A-30636 | 2 | CUuGACuUUGCuAAGAGCcdTdT | AD-3591 |
| A-30627 | 1 | GgCUCUuAGcAAAGUcAAGdTdT | A-30636 | 2 | CUuGACuUUGCuAAGAGCcdTdT | AD-3592 |
| A-30623 | 1 | GGcuCUUAGcAaAGucAaGdTdT | A-30637 | 2 | CUuGACUUuGCuAAGAGCcdTdT | AD-3593 |
| A-30625 | 1 | GGcuCUUAGcAaAGucAAGdTdT | A-30637 | 2 | CUuGACUUuGCuAAGAGCcdTdT | AD-3594 |
| A-30627 | 1 | GgCUCUuAGcAAAGUcAAGdTdT | A-30637 | 2 | CUuGACUUuGCuAAGAGCcdTdT | AD-3595 |
| A-30623 | 1 | GGcuCUUAGcAaAGucAaGdTdT | A-30640 | 2 | CUuGACUuUGCuAAGAGCcdTdT | AD-3596 |
| A-30625 | 1 | GGcuCUUAGcAaAGucAAGdTdT | A-30640 | 2 | CUuGACUuUGCuAAGAGCcdTdT | AD-3597 |
| A-30627 | 1 | GgCUCUuAGcAAAGUcAAGdTdT | A-30640 | 2 | CUuGACUuUGCuAAGAGCcdTdT | AD-3598 |

| Table 13b | Average IC50 In- |
|---|---|
| DUPLEX ID # | Vitro |
| RSV01 | 1.0 |
| AD-3581 | 2 |
| AD-3582 | 1 |
| AD-3583 | 6 |
| AD-3584 | 1 |
| AD-3585 | 1 |
| AD-3586 | 2 |
| AD-3587 | 1 |
| AD-3588 | 1 |
| AD-3589 | 2 |
| AD-3590 | 1 |
| AD-3591 | 1 |
| AD-3592 | 5 |
| AD-3593 | 3 |
| AD-3594 | 1 |
| AD-3595 | 9 |
| AD-3596 | 1 |
| AD-3597 | 1 |
| AD-3598 | 4 |

### Example 19

### In Vivo Antiviral Activity of Modified RSV siRNAs

Materials and Methods

For the prophylaxis model, Balb/c mice were anesthetized by intraperitoneal (i.p.) administration of 2,2,2-tribromoethanol (Avertin) and instilled intranasally (i.n.) with siRNA in a total volume of 50 μl of PBS. At 4 hours post siRNA instillation, the mice were anesthetized and infected i.n. with 1×10⁶ plaque forming units (PFU) of RSV/A2 in 50 μl. Prior to removal of lungs at day 4 post-infection, anesthetized mice were exsanguinated by severing the right caudal artery. Lung tissue was collected in 1 ml ice cold phosphate-buffered saline (PBS; GIBCO Invitrogen). RSV titers from lungs were measured by immunostaining plaque assay. Lungs were homogenized with a hand-held Tissumiser homogenizer (Fisher Scientific, Pittsburgh, Pa.) and lung homogenates were placed on ice for 5-10 minutes to allow debris to settle. Clarified lung lysates were serially diluted 10-fold in serum-free DMEM, added to 95% confluent Vero E6 cells cultured in D-MEM in 24-well plates (BD Falcon, San Jose, Calif.). Infected cells incubated at 37° C., 10% CO2 for one and half hour, lysate aspirated from the cells and overlaid with Methylcellulose and incubated for 5 days and plaque assay performed as below.

Plaque assay: 5 days post infection aspirated methylcellulose and fixed cells with ice cold Acetone:Methanol (60:40) for 10-15 minutes and placed upside down overnight to let Acetone:Methanol evaporate. After 24 hrs blocked cells with 1× Powerblock (BioGenex Cat#HK085-5K) for 30 minutes at RT. Diluted Primary Antibody (131-2A-RSV F monoclonal antibody, Chemicon International Cat#MAB8599) 1:2000 in cold 0.1× Powerblock. Removed Powerblock from plate and added 250 ul of Primary Antibody. Incubated at 37° C., 5% CO₂ for 2 hrs. Washed cells twice for 10-15 minutes with 0.05% Tween (Sigma Cat#SL05303) 10% PBS (GIBCO Cat #70013-032). Diluted Secondary Antibody (Goat anti-mouse IgG whole molecule-Alkaline Phosphatase Conjugate, Sigma Cat#A9316) 1:1000 in cold 0.1× Powerblock. Added 250 ul of Secondary antibody to each well and incubated at 37° C., 5% CO₂ for 1 hr. Washed cells twice for 10 minutes with 0.05% Tween 10% phosphate buffered saline (PBS). Made Vector Black Staining Solution (Vector Laboratories Cat#Sk-5200) and added ˜300 ul of stain to each well for 15 min or until staining was distinct. Washed plates with DI water and allowed to dry overnight. Counted Plaques.

Results

Suppression of the RSV A2 viral titer (Log−pfus/g lung) in the prophylactic mouse model compared to phosphate buffered saline (PBS) (5-5.5 Log−pfus/g lung) is shown in Table 14. Table 14 shows the in vivo antiviral activity of modified RSV siRNA. Table 15 shows the sequences of modified RSV siRNA.

TABLE 14
in vivo antiviral activity of modified RSV siRNA

| | | Viral Titer | Viral titer |
|---|---|---|---|
| Compound | | Reduction to PBS | reduction to PBS |
| ID# | Dose/mouse | (Log) | (Fold) |
| ALN-3532 | 25 ug | 0.8 | 6 |
| | 50 ug | 2.0 | 100 |
| | 100 ug | 3.1 | 1259 |
| ALN-3534 | 25 ug | 0.6 | 4 |
| | 50 ug | 1.8 | 63 |
| | 100 ug | 3.6 | 3981 |
| ALN-3586 | 25 ug | 1.0 | 10 |
| | 50 ug | 1.8 | 63 |
| | 100 ug | 3.4 | 2512 |
| ALN-3587 | 25 ug | 1.2 | 16 |
| | 50 ug | 1.8 | 63 |
| | 100 ug | 2.7 | 501 |
| ALN-3588 | 25 ug | 0.8 | 6 |
| | 50 ug | 1.6 | 40 |
| | 100 ug | 2.8 | 631 |
| ALN-3589 | 25 ug | 0.8 | 6 |
| | 50 ug | 2.0 | 100 |
| | 100 ug | 2.3 | 200 |
| ALN-16484 | 25 ug | 1.3 | 20 |
| | 50 ug | 2.8 | 631 |
| | 100 ug | 3.9 | 7943 |
| ALN-16485 | 25 ug | 0.4 | 3 |
| | 50 ug | 2.2 | 158 |
| | 100 ug | 3.3 | 1995 |
| ALN-16527 | 25 ug | 1.4 | 25 |
| | 50 ug | 2.4 | 250 |
| | 100 ug | 5.0 | 100000 |
| ALN-16528 | 25 ug | 1.4 | 25 |
| | 50 ug | 2.6 | 398 |
| | 100 ug | 3.1 | 1259 |
| ALN-16570 | 25 ug | 1.2 | 16 |
| | 50 ug | 2.4 | 250 |
| | 100 ug | 3.6 | 3981 |
| ALN-16571 | 25 ug | 1.3 | 20 |
| | 50 ug | 1.6 | 40 |
| | 100 ug | 3.4 | 2512 |
| ALN-16613 | 25 ug | 1.3 | 20 |
| | 50 ug | 2.3 | 200 |
| | 100 ug | 3.9 | 7943 |
| ALN-16614 | 25 ug | 1.0 | 10 |
| | 50 ug | 2.7 | 501 |
| | 100 ug | 3.0 | 1000 |
| ALN-3185 | 25 ug | 1.5 | 32 |
| | 50 ug | 2.1 | 126 |
| | 100 ug | 3.1 | 1259 |
| ALN-3220 | 25 ug | 1.6 | 40 |
| | 50 ug | 2.6 | 398 |
| | 100 ug | 3.8 | 6310 |
| ALN-3221 | 25 ug | 1.5 | 32 |
| | 50 ug | 2.6 | 398 |
| | 100 ug | 3.3 | 1995 |
| ALN-3148 | 25 ug | 1.1 | 13 |
| | 50 ug | 2.7 | 501 |
| | 100 ug | 5.2 | 158489 |
| ALN-3150 | 25 ug | 2.0 | 100 |
| | 50 ug | 2.2 | 159 |
| | 100 ug | 3.4 | 2512 |
| ALN-3151 | 25 ug | 2.2 | 159 |
| | 50 ug | 3.2 | 1585 |
| | 100 ug | 3.3 | 1995 |

TABLE 15
Sequences of modified RSV siRNA

| | | | | | | |
|---|---|---|---|---|---|---|
| | SEQ ID | | | SEQ ID | | Duplex |
| SS ID # | NO: | sense strand (5′--3′) | AS ID # | NO: | antisense strand (5′--3′) | ID # |

| | | | | | | |
|---|---|---|---|---|---|---|
| A17652 | 1 | GGc ucu uAG cAA AGu cAA GTsT | A17653 | 2 | CUU GAC UUU GCu AAG AGC CTsT | AD-3148 |
| A17656 | 1 | GgC UCU UAG CAA AGU CAA GTsT | A17657 | 2 | CuU GAC UUU GCU AAG AGC CTsT | AD-3150 |
| A17658 | 315 | GGC UCU UAG CAA AGU CAA Gusu | A17659 | 316 | CUU GAC UUU GCU AAG AGC Casu | AD-3151 |
| 5718 | 1 | GGC UCU UAG CAA AGU CAA GdTdT | A12564 | 2 | cuu Gac uuu Gcu AAG Agc cTT | AD-3120 |
| 5718 | 1 | GGC UCU UAG CAA AGU CAA GdTdT | A12565 | 2 | cuu gac uuu gCU AAG AGC CTT | AD-3121 |
| A23555 | 1 | GgC UCU uAG cAA AGU cAA GTsT | A23556 | 2 | CuU GAC UUU GCu AAG AGC CTsT | AD-3185 |
| A26832 | 1 | GGc uCU UAG cAa AGu cAA GdTdT-HP | A26844 | 2 | CuU GAC uUU GCU AAG AGC CdTdT-HP | AD-16484 |
| A26832 | 1 | GGc uCU UAG cAa AGu cAA GdTdT-HP | A26845 | 2 | CuU GAC UuU GCU AAG AGC CdTdT-HP | AD-16485 |
| A26833 | 1 | GGc uCU UAG cAa AGu cAa GdTdT-HP | A26844 | 2 | CuU GAC uUU GCU AAG AGC CdTdT-HP | AD-16527 |
| A26833 | 1 | GGc uCU UAG cAa AGu cAa GdTdT-HP | A26845 | 2 | CuU GAC UuU GCU AAG AGC CdTdT-HP | AD-16528 |
| A26833 | 1 | GGc uCU UAG cAa AGu cAa GdTdT-HP | A26846 | 2 | CuU GAC UUu GCU AAG AGC CdTdT-HP | AD-16529 |
| A26834 | 315 | GGc uCU UAG cAa AGu cAA Guu-HP | A26844 | 2 | CuU GAC uUU GCU AAG AGC CdTdT-HP | AD-16570 |
| A26834 | 315 | GGc uCU UAG cAa AGu cAA Guu-HP | A26845 | 2 | CuU GAC UuU GCU AAG AGC CdTdT-HP | AD-16571 |
| A26835 | 315 | GGc uCU UAG cAa AGu cAa Guu-HP | A26844 | 2 | CuU GAC uUU GCU AAG AGC CdTdT-HP | AD-16613 |
| A26835 | 315 | GGc uCU UAG cAa AGu cAa Guu-HP | A26845 | 2 | CuU GAC UuU GCU AAG AGC CdTdT-HP | AD-16614 |
| A-30631 | 1 | GGcuCUUAGcAaAGucAAGdTsdT | A-30648 | 2 | CUuGACUUuGCUAAGAGCcdTsdT | AD-3532 |
| A-30631 | 1 | GGcuCUUAGcAaAGucAAGdTsdT | A-30652 | 2 | CUuGACUuUGCUAAGAGcCdTsdT | AD-3534 |
| A-30633 | 1 | GgCUCUuAGcAAAGUcAAGdTsdT | A-30650 | 2 | CUuGACUUuGCuAAGAGCcdTsdT | AD-3586 |
| A-30629 | 1 | GGcuCUUAGcAaAGucAaGdTsdT | A-30653 | 2 | CUuGACUuUGCuAAGAGCcdTsdT | AD-3587 |
| A-30631 | 1 | GGcuCUUAGcAaAGucAAGdTsdT | A-30653 | 2 | CUuGACUuUGCuAAGAGCcdTsdT | AD-3588 |
| A-30633 | 1 | GgCUCUuAGcAAAGUcAAGdTsdT | A-30653 | 2 | CUuGACUuUGCuAAGAGCcdTsdT | AD-3589 |

lower case is 2′ OMe modification
Exo = s phosphothioate (Chemistry 1)
endo light = UA/CA 2′ OMe(Chemistry 2); endo heavy = all Py as 2′-OMe (Chemistry 3)
heavy methylated = many modified nts in a raw either from 5′ or 3′
2′-OMe, @ Pos 2 (Chemistry 4); TT-complem. @ 2′-OMe, PTO

### Example 20

### RNAi-Specific Activity of RSV-Targeted siRNAs

Materials and Methods

Animals. Six- to eight-week old, pathogen-free female BALB/c mice were purchased from Harlan Sprague-Dawley Laboratories (Indianapolis, Ind.). The mice were housed in microisolator cages and fed sterilized water and food ad libitum.

Virus preparation, cell lines and viral titering. Vero E6 cells were maintained in tissue culture medium (TCM) consisting of Dulbecco's Modified Eagle Medium (D-MEM, GIBCO Invitrogen, Carlsbad, Calif.) supplemented with 10% fetal bovine serum (Hyclone, Logan, Utah). RSV/A2 and RSV/B1 were prepared in Vero E6 cells. Briefly, confluent Vero E6 cells (American Type Culture Collection, Manassas, Va.) in serum-free D-MEM, were infected with RSV at a multiplicity of infection (MOI) of 0.1. The virus was adsorbed for 1 h at 37° C. after which TCM was added. Infected cells were incubated for 72-96 h at 37° C. until >90% cytopathic effect (CPE) was observed by light microscopy. Infected cells were harvested by removal of the medium and replacement with a minimal volume of serum-free D-MEM followed by three freeze-thaw cycles at −70 and 4° C., respectively. The contents were collected and centrifuged at 4000×g for 20 min at 4° C. to remove cell debris, and the titer was determined by immunostaining plaque assay as previously described. Briefly, Vero E6 cells are infected with serial dilutions of stock RSV, adsorbed for 1 h at 37° C., then overlayed with 2% methylcellulose media (DMEM, supplemented with 2% fetal bovine serum, 1% antibiotic/antimycotic solution, 2% methylcellulose). After 5 days at 37° C./5% CO₂, plates are removed and cells are fixed with ice-cold Acetone:Methanol (60:40). Cells are blocked with Powerblock, universal blocking reagent (Biogenix, San Ramon, Calif.), incubated with Anti-RSV F protein monoclonal antibody 131-2A dilute 1:200 (Millipore-Chemicon,), followed by Goat anti-mouse IgG whole molecule alkaline phosphatase secondary antibody. The reaction was developed with Alkaline phosphatase substrate kit (Vector Black, Vector Laboratories, Burlingame, Calif.) and plaques were visualized and counted using a light microscope. For RSV primary isolate cultures, samples were obtained from Dr. John DeVincenzo from the University of Tennessee, Memphis, Tenn. RSV isolates were obtained from RSV infected children diagnosed by either a conventional direct fluorescent antibody (DFA) method or by a rapid antigen detection method in the Le Bonheur Children's Medical Center Virology Laboratory in Memphis, Tenn. Nasal secretions were collected by aspiration, grown and passaged in HEp-2 cells and harvested at 90% cytopathic effect. Individual aliquots of supernatant containing RSV were then subjected to nucleic acid extraction using QiAmp Viral RNA mini kit, according to the manufacturer's protocol (Qiagen, Valencia, Calif.). RSV isolates were also obtained from Mark Van Ranst from the University of Leuven, Leuven, Belgium and Larry Anderson from the Centers for Disease Control and Prevention, Atlanta, Ga.

RSV-specific siRNA selection. Using one of the National Center for Biotechnology Information (NCBI) databases a Basic Local Alignment Search Tool (BLAST), was performed. In this analysis, a sequence comparison algorithm is used to search sequence databases for optimal local alignments to a query (2). In this case, the query is the 19 nt sequence comprising the sense or antisense strand of ALN-RSV01, excluding the dTdT overhang. The database, Reference Sequence (RefSeq), provides a comprehensive, integrated, non-redundant set of sequences, including genomic DNA, transcript (RNA), and protein products, and is updated weekly. Only siRNAs that showed no significant homology to any sequence from the RefSeq database were selected for synthesis and further study.

In vitro RSV inhibition assay. Vero cells, in 24-well plates, were grown in a 5% CO₂ humidified incubator at 37° C. in DMEM supplemented with 10% fetal bovine serum (Life Technologies-Invitrogen, Carlsbad, Calif.), 100 units/ml penicillin, and 100 g/ml streptomycin (BioChrom, Cambridge, UK) to 80% confluence. siRNAs were diluted to the indicated concentrations in 50 μl Opti-MEM Reduced Serum Medium (Invitrogen). Separately, 3 μl Lipofectamine 2000 (Invitrogen) was diluted in 50 μl Opti-MEM mixed and incubated for 5 minutes at room temperature. siRNA and lipofectamine mixtures were combined, incubated for 20-25 minutes at room temperature, then added to cells and incubated at 37° C. overnight. The mixture was then removed from cells and 200-400 plaque forming units of RSV/A2 was incubated with cells for 1 hour at 37° C. The infected cells were covered with methylcellulose media and incubated for 5 days at 37° C. and plaques visualized by immunostaining plaque assay as described.

In vivo screening of RSV-specific siRNAs. For the prophylaxis model, BALB/c mice were anesthetized by intraperitoneal (i.p.) administration of 2,2,2-tribromoethanol (Avertin) and instilled intranasally (i.n.) with siRNA in a total volume of 50 μl of PBS. At 4 hours post siRNA instillation, the mice were anesthetized and infected i.n. with 1×10⁶ PFU of RSV/A2 in 50 μl. Prior to removal of lungs at day 4 post-infection, anesthetized mice were exsanguinated by severing the right caudal artery. Lung tissue was collected in 1 ml ice cold phosphate-buffered saline (PBS; GIBCO Invitrogen). RSV titers from lungs were measured by immunostaining plaque assay. Lungs were homogenized with a hand-held Tissumiser homogenizer (Fisher Scientific, Pittsburgh, Pa.) and lung homogenates were placed on ice for 5-10 minutes to allow debris to settle. Clarified lung lysates were serially diluted 10-fold in serum-free D-MEM, added to 95% confluent Vero E6 cells cultured in D-MEM in 24-well plates (BD Falcon, San Jose, Calif.), and plaque assays were performed as described above. For the treatment model, BALB/c mice were anesthetized as above and instilled i.n. with 1×10⁶ PFU of RSV/A2 in 50 μl. At one, two, three or four days post viral infection, mice were reanesthetized and instilled i.n. with siRNA in 50 μl and then viral concentrations were measured in the lungs on day 5 post infection, as described above.

siRNA generation. RNA oligonucleotides were synthesized using commercially available 5′-O-(4,4′-dimethoxytrityl)'3′O-(2-cyanoethyl-N,N-diisopropyl) phosphoramidite monomers of uridine (U), 4-N-benzyoylcytidine (C^{Bz}), 6-N-benzoyladenosine (A^{bz}) and 2-N-isobutyrlguanosine (G^{iBu}) with 2′-O-t-butyldimethylsilyl protected phosphoramidites according to standard solid phase oligonucleotide synthesis protocols (13). After cleavage and de-protection, RNA oligonucleotides were purified by anion-exchange high-performance liquid chromatography and characterized by ES mass spectrometry and . To generate siRNAs from RNA single strands, equimolar amounts of complementary sense and antisense strands were mixed and annealed, and siRNAs were further characterized by CGE.

PBMC assay. To examine the ability of siRNAs to stimulate interferon alpha (IFNα) or tumor necrosis factor alpha (TNFα), human peripheral blood mononuclear cells (hPBMCs) were isolated from concentrated fractions of leukocytes (buffy coats) obtained from the Blood Bank Suhl, Institute for Transfusion Medicine, Germany. Buffy coats were diluted 1:1 in PBS, added to a tube of Histopaque (Sigma, St. Louis, Mo.) and centrifuged for 20 minutes at 2200 rpm to allow fractionation. White blood cells were collected, washed in PBS, followed by centrifugation. Cells were resuspended in RPMI 1640 culture medium (Invitrogen) supplemented with 10% fetal calf serum, IL-3 (10 ng/ml) (Sigma) and phytohemagglutinin-P (PHA-P) (5 μg/ml) (Sigma) for IFNα assay, or with no additive for TNFα assay at a concentration of 1×10⁶ cells/ml, seeded onto 96-well plates and incubated at 37° C., 5% CO₂. Control oligonucleotides siRNA AL-DP-5048 duplex: 5′-GUCAUCACACUGAAUACCAAU-3′(SEQ ID NO: 287) and 3′-CACAGUAGUGUGACUUAUGGUUA-5′ (SEQ ID NO: 288); siRNA AL-DP-7296 duplex: 5′-CUACACAAAUCAGCGAUUUCCAUGU-3′(SEQ ID NO: 289) and 3′-GAUGUGUUUAGUCGCUAAAGGUACA-5′ (SEQ ID NO: 290); siRNA AL-DP-1730 duplex: 5′-CGAUUAUAUUACAGGAUGAdTsdT-3′ (SEQ ID NO: 249) and 3′-dTsdTGCUAAUAUAAUGUCCUACU-5′ (SEQ ID NO: 268); and siRNA AL-DP-2153 duplex: 5′-GGCUCUAAGCUAACUGAAGdTdT-3′(SEQ ID NO: 291) and 3′-dTdTCCGAGAUUCGAUUGACUUC-5′(SEQ ID NO: 292). Cells in culture were combined with either 500 nM oligonucleotide, pre-diluted in OptiMEM (Invitrogen), or 133 nM oligonucleotide pre-diluted in OptiMEM and Geneporter, GP2 transfection reagent (Genlantis, San Diego, Calif.) for IFN assay or N-[1-(2,3-Dioleoyloxy)propyl]-N,N,N-trimethylammonium methylsulfate (DOTAP) (Roche, Switzerland) for TNFα assay and incubated at 37° C. for 24 hrs. IFNα and TNFα were measured using the Bender MedSystems (Vienna, Austria) instant ELISA kit according to manufacturer's instruction.

In vitro and In vivo RACE. Total RNA was purified from either in vitro transfected Vero E6 cells or from lungs harvested at day 5 post-infection as described above, using Tryzol (Invitrogen), followed by DNase treatment and final processing using RNeasy, according to manufacturer instructions (Qiagen). Five to ten microliters of RNA preparation from pooled samples was ligated to GeneRacer adaptor (5′-CGACUGGAGCACGAGGACACUGACAUGGACUGAAGGAGUAGAAA-3′ (SEQ ID NO: 293)) without prior treatment. Ligated RNA was reverse transcribed using a gene specific primer (cDNA primer: 5′-CTCAAAGCTCTACATCATTATC-3′(SEQ ID NO: 294)). To detect RNAi specific cleavage products, two rounds of consecutive PCR were performed using primers complimentary to the RNA adaptor and RSV A2 N gene mRNA (GR 5′ primer: 5′-CGACTGGAGCACGAGGACACTGA-3′(SEQ ID NO: 295) and Rev Primer: 5′-CCACTCCATTTGCTTTTACATGATATCC-3′ (SEQ ID NO: 296)) for the first round, followed by a second round of nested PCR (GRN 5′ primer: GGACACTGACATGGACTGAAGGAGTA-3′(SEQ ID NO: 297) and Rev N Primer: 5′-GCTTTTACATGATATCCCGCATCTCTGAG-3′ (SEQ ID NO: 298). Amplified products were resolved by agarose gel electrophoresis and visualized by ethidium bromide staining. Specific cleavage products migrating at the correct size were excised, cloned into a sequencing vector and sequenced by standard method.

Sequence Analysis of Clinical Isolates for ALN-RSV01 Target Site Conservation. Amplification of the RSV N gene fragment containing the ALN-RSV01 recognition site was performed using two-step RT-PCR. Briefly, RNA was reverse transcribed using random hexamers and Superscript III reverse transcriptase (Invitrogen) at 42° C. for 1 hr to generate a cDNA library. A 1200 nucleotide gene specific fragment was amplified using the RSV N forward primer: 5′-AGAAAACTTGATGAAAGACA-3′ (SEQ ID NO: 285) and the RSV N reverse primer: 5′-ACCATAGGCATTCATAAA-3′ (SEQ ID NO: 286) for 35 cycles at 55° C. for 30 sec followed by 68° C. for 1 min using Platinum Taq polymerase (Invitrogen). PCR products were analyzed by 1% agarose gel electorphoresis. As a control, a laboratory strain of RSV A long was subjected to the identical procedures for analysis. PCR products were purified using QIAquick PCR purification kit (Qiagen) according to the manufacturer's protocol and sequenced using standard protocols (Agencourt Bioscience, Beverly, Mass.). For each clone, forward and reverse sequence was obtained. Sequences were analyzed and aligned via Clustal W and ContigExpress using Vector NTI software (Invitrogen).

RSV viral genotyping. Genotyping of all 21 isolates received from Mark Van Ranst were performed as described previously (78). Genotyping of the remaining 78 (57 from Dr. John DeVincenzo, University of Tennessee, 13 from Dr. Larry Anderson, Centers for Disease Control and Prevention, and 8 from Dr. Jeffrey Kahn, Yale University) isolates was performed by Dr. Jeffrey Kahn's Laboratory, Department of Pediatrics, Yale University, New Haven, Conn. Analysis of the RSV G gene was performed by first generating cDNA using random hexamers and M-MuLV reverse transcriptase (New England Biolabs, Beverly, Mass.) at 37° C. for 1 hr, followed by PCR amplification using G gene specific primers GTmF: 5′-CCGCGGGTTCTGGCAATGATAATCTCAAC-3′ (SEQ ID NO: 299) and subgroup specific G gene specific primers RSV A-GAR2: 5′-GCCGCGTGTATAATTCATAAACCTTGGTAG-3′ (SEQ ID NO: 300) or RSV B-GBR: 5′-GGGGCCCCGCGGCCGCGCATTAATAGCAAGAGTTAGGAAG-3′ (SEQ ID NO: 301) by denaturing at 95° C. for 15 min, followed by 40 cycles of 95° C. for 1 min, 60° C. for 1 min and 72° C. for 1 min, followed by a single 10 min extension at 72° C. using HotStar Taq DNA polymerase (Qiagen). PCR products were analyzed by 2% agarose gel electorphoresis. If an appropriate size PCR product was identified (RSV A: 1200 nt or RSV B: 900 nt), the product was purified using QIAquick Extraction Kit (Qiagen) according to the manufacturer's protocol. Purified PCR products were analyzed by agarose gel electrophoresis and sequenced on a 3730 XL DNA Analyzer (Applied Biosystems, Foster City, Calif.) at the Yale University School of Medicine W. M. Keck Foundation Biotechnology Resource Laboratory.

Nucleotide sequences were aligned manually and alignment confirmed using Clustal W. RSV A and RSV B isolates were distinguished by comparing G gene nucleotide sequences and laboratory standards. Phylogenetic analysis for RSV A isolates was performed using an aligned 417 nt segment of the G gene corresponding to nucleotide position 5010 to 5426 (GenBank Accession # M74568). Phylogenetic analysis of RSV B isolates was performed using an aligned 288 nt segment of the G gene corresponding to nucleotide positions 5036 to 5323 (GenBank Accession #AF013254). Bootstrap datasets containing 100 aligned permuted nucleotide sequence sets were produced using SEQBOOT in the PHYLIP 3.65 software package. A nucleotide distance matrix was computed assuming a transition:transversion ratio of 2 and gamma distribution of site-specific mutation rates with an alpha of 2 using DNADIST (PHYLIP). The neighbor joining method was then used to analyze the distance matrix with NEIGHBOR (PHYLIP). CONSENSE (PHYLIP) was used to produce an extended majority rule phylogenetic tree and the trees were drawn using Treeview 1.6.6. Bootstrap values and isolate clustering were used to identify specific RSV genotypes.

Results

Bioinformatic analysis of RSV genome and selection of ALN-RSV01. The three proteins contained within the nucleocapsid (nucleoprotein (N), phosphoprotein (P), and polymerase (L)) are required for various steps within the replication cycle of RSV (Collins, P. et al., (1996) Respiratory Syncytial Virus, p. 1313-1351, Fields' Virology), and are among the most highly conserved regions of the RSV genome (Sullender, W M (2000) Clin Microbiol Rev 13:1-15; Sullender et al., (1993) J Clin Microbiol 31:1224-31). To select appropriate siRNAs targeting these three mRNAs of the RSV genome, GenBank sequences AF03506 (RSV/A2), AF0132254 (RSV/A long), AY911262 (RSV/B1), and D00736 (RSV/18537) were aligned using the Clustal W algorithm to identify conserved 19mers amongst all RSV sequences analyzed. To determine uniqueness of each 19 mer across the human genome, a Basic Local Alignment Search Tool (BLAST) analysis was performed against the Reference Sequence (RefSeq) database. Only siRNAs with homology of 16 nucleotides or fewer to any gene in the human genome were selected for further analysis.

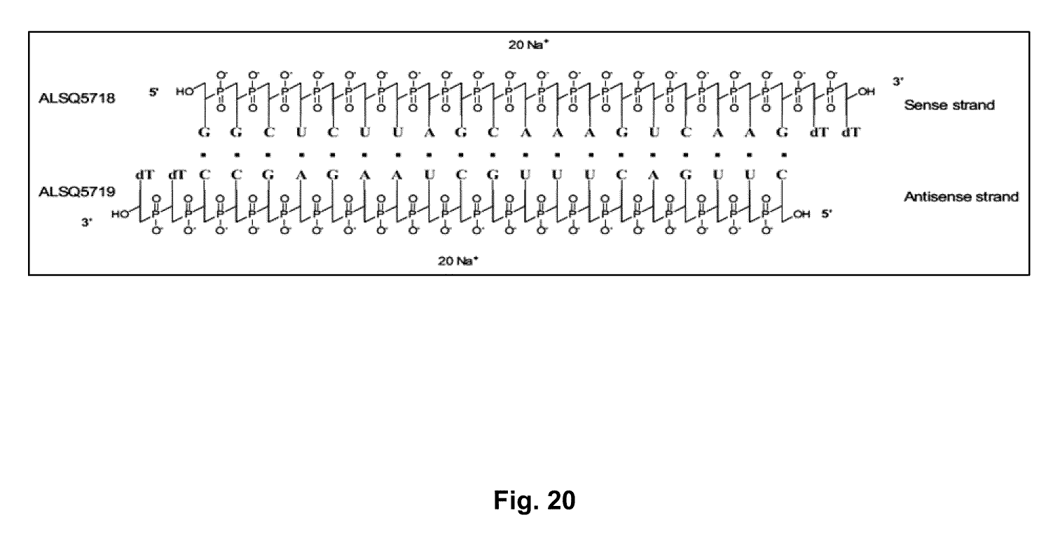

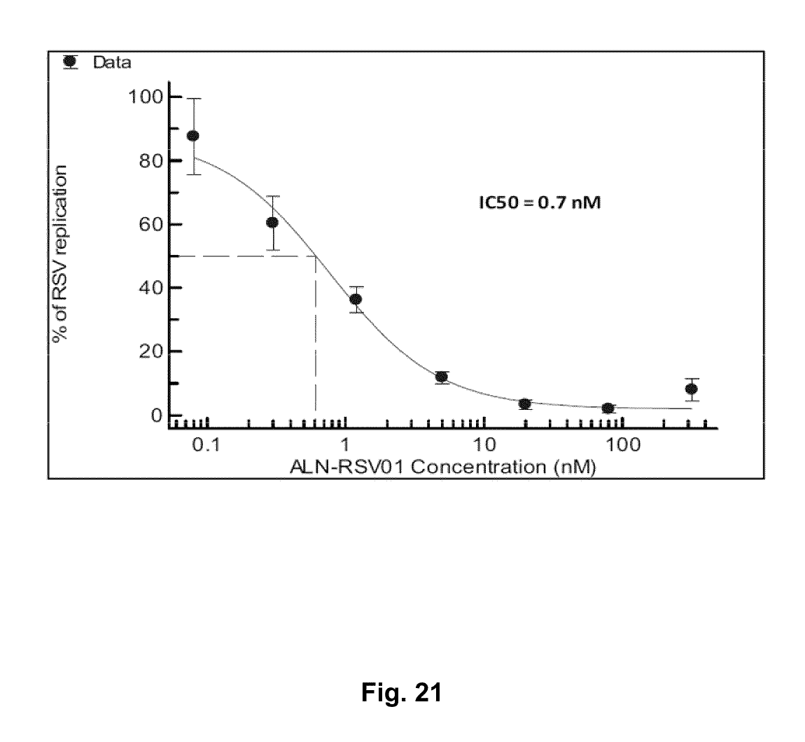

Seventy siRNAs targeting the RSV N, P, and L genes were analyzed in a plaque inhibition assay and 19 exhibited >80% inhibition of plaque formation versus a PBS control at siRNA concentrations of 20 nM (data not shown). Of these 19 siRNAs, the siRNA designated “ALN-RSV01” (FIG. 20) that targets the N gene, consistently demonstrated the highest anti-viral activity. Indeed, ALN-RSV01 showed an IC50 of 0.7 nM in the RSV plaque inhibition assay (FIG. 21).

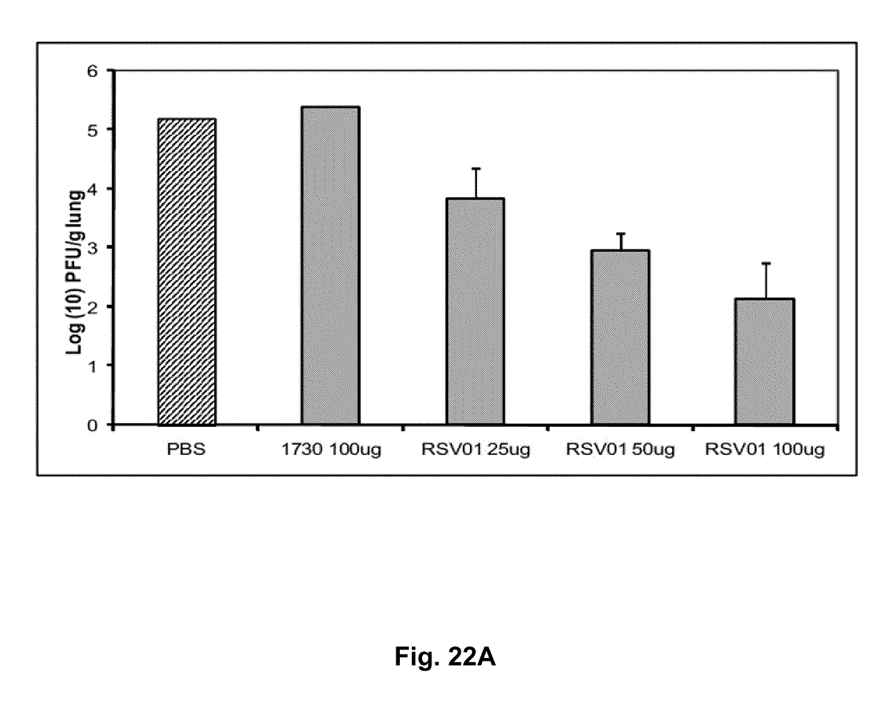

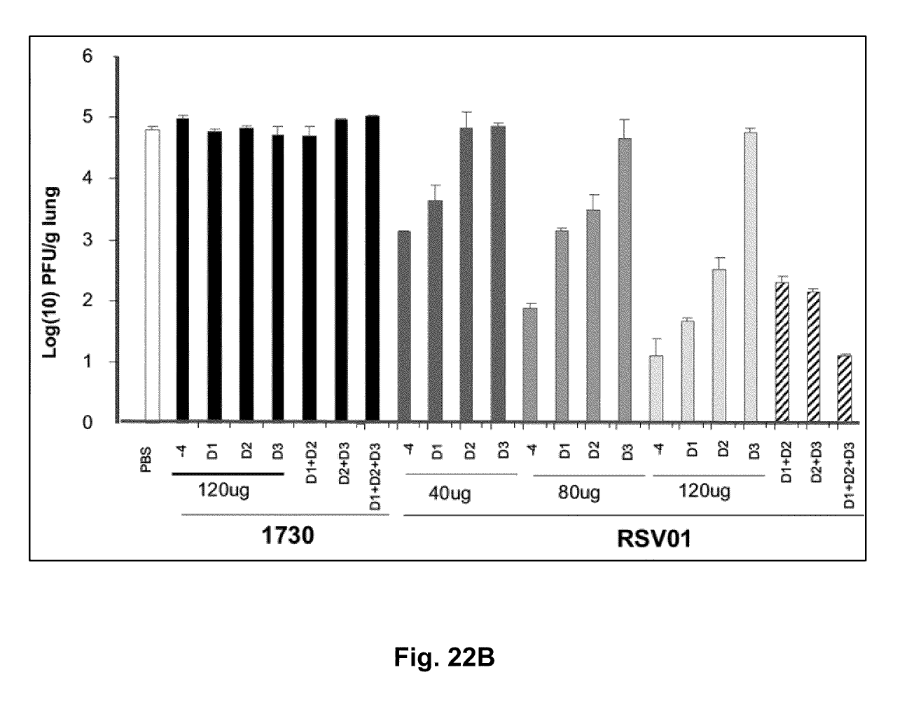

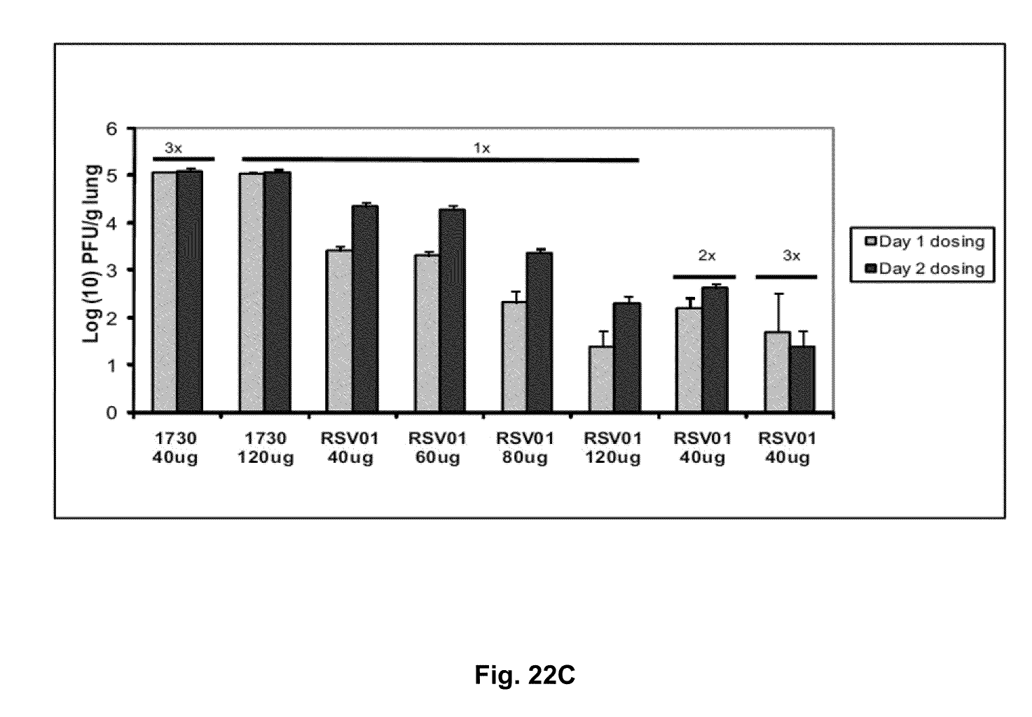

In vivo studies of ALN-RSV01. The BALB/c mouse is a well-established model for RSV infection, and was thus chosen as the in vivo system for evaluation of anti-viral efficacy of ALN-RSV01. Initially in a prophylaxis model, siRNA was administered intranasally (i.n.) to mice four hours prior to infection with 10⁶ pfu of RSV/A2. There was dose dependent inhibition of RSV/A2 replication in the lungs of mice, with a 100 g dose of ALN-RSV01 reducing titers between 2.5 to 3.0 log₁₀ pfu/g lung as compared to either PBS controls or a non-specific siRNA (FIG. 22A). Fifty and 25 g doses yielded reductions of approximately 2.0 and 1.25 log₁₀ pfu/g, respectively (FIG. 22A).

To evaluate the efficiency of viral inhibition in a treatment paradigm, ALN-RSV01 was delivered i.n., in single or multiple daily doses at 1, 2 and/or 3 days post infection. When delivered as a single dose, the most efficacious silencing by ALN-RSV01 occurred in prophylactic (−4 h) dosing in a dose-dependent fashion. As compared to the mismatch control AL-DP-1730, administration of 120 g of ALN-RSV01 as a single prophylactic dose resulted in maximal viral inhibition, decreasing lung concentrations down to background levels in this assay. When ALN-RSV01 was administered in a treatment regime as a single dose following viral inoculation, anti-viral efficacy was maintained in a dose-dependent manner but found to decrease as a function of time of dosing post viral infection (FIG. 22B). Indeed, by Day 3 post infection, single doses as high as 120 g did not yield any significant viral inhibition. However, when multiple 40 g doses of ALN-RSV01 were delivered daily on days 1, 2, and 3, the efficiency of silencing was maintained and viral titers were again reduced to background levels (FIG. 22B).

To further explore alternative dosing paradigms that could be employed in future clinical studies, additional multi-dose regimens were evaluated. To this end, RSV-infected mice were treated over a 12 hour period with ALN-RSV01 either in a 2× per day or 3× per day dose regimen. Interestingly, this multiple daily dose regimen of the RSV-specific siRNA (40 g 3×/day) was found to be as efficacious as a single 120 g dose (FIG. 22C). In aggregate, these data show that a multi-dose treatment regimen of ALN-RSV01 can provide maximal anti-viral efficacy in a fashion readily applicable to human clinical studies in relevant patient populations.

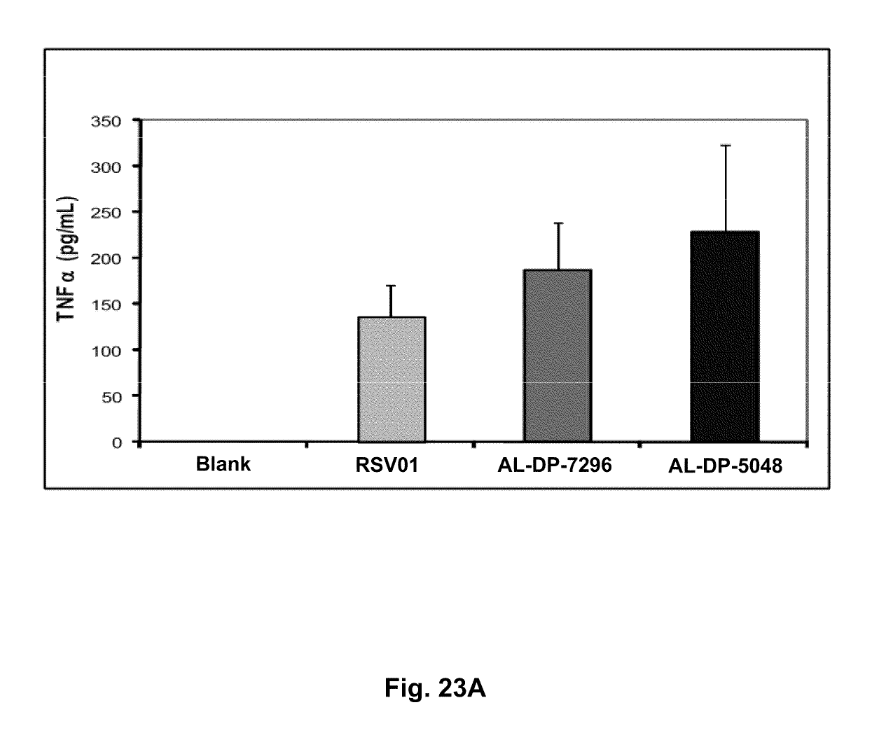

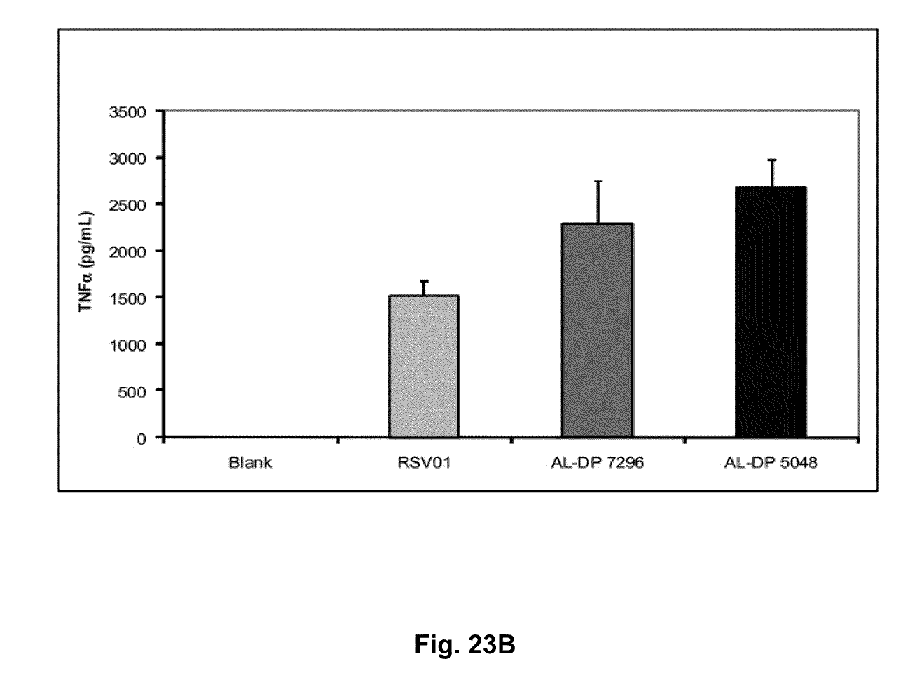

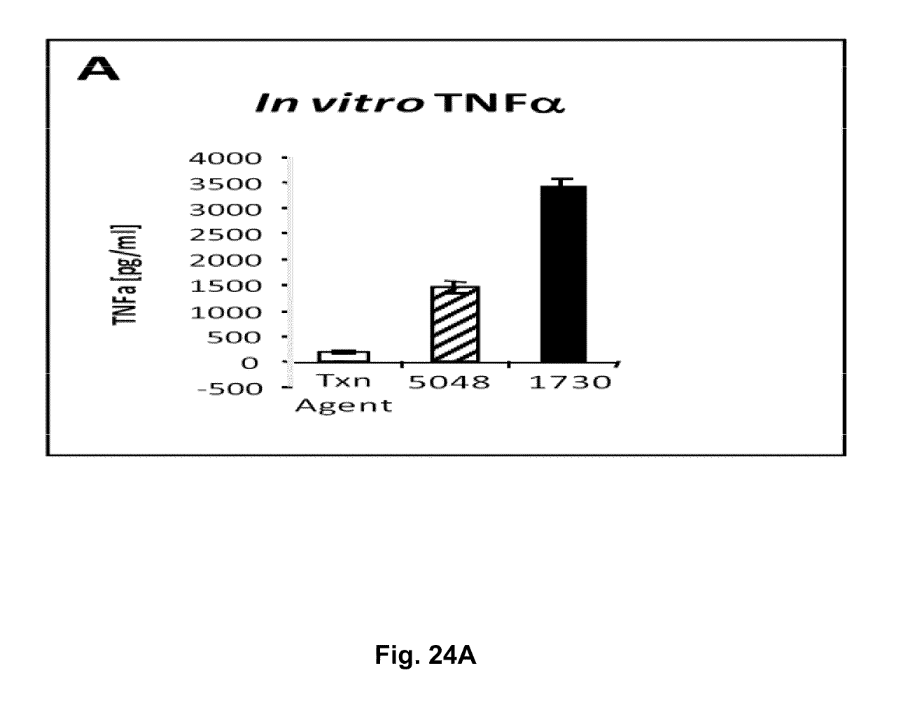

ALN-RSV01 and cytokine induction. Many nucleic acids, including double-stranded RNA (dsRNA), single-stranded RNAs (ssRNA) and siRNAs have been shown to stimulate the innate immune response through a variety of RNA binding receptors (Robbins et al. (2007) Mol Ther 15:1663-9.). This stimulation can be monitored in vitro in a peripheral blood mononuclear cell (PBMC) assay (Sioud, M. (2005) J Mol Biol 348:1079-90.). While an immunostimulatory property of an siRNA could act synergistically with an RNAi-mediated mechanism for the treatment of a viral infection, such a feature might also confound interpretation of results related to an siRNA treatment strategy. Accordingly, ALN-RSV01 was evaluated for its ability to stimulate IFNα and TNFα in vitro by incubating with freshly purified peripheral blood mononuclear cells (PBMCs) as previously described (Hornung, V., M. Guenthner-Biller, C. Bourquin, A. Ablasser, M. Schlee, S. Uematsu, A. Noronha, M. Manoharan, S. Akira, A. de Fougerolles, S. Endres, and G. Hartmann. 2005. Sequence-specific potent induction of IFN-alpha by short interfering RNA in plasmacytoid dendritic cells through TLR7. Nat Med 11:263-70.). High concentrations of ALN-RSV01 were used in these assays (133 nM), exceeding the IC₅₀ for anti-viral effect by over 100-fold. After 24 hours, only modest levels of both IFNα and TNFα were detected by ELISA, with an average of approximately 147 pg/ml of IFNα (FIG. 23A) and 1500 pg/ml of TNFα (FIG. 23B) induced, as compared to media alone controls

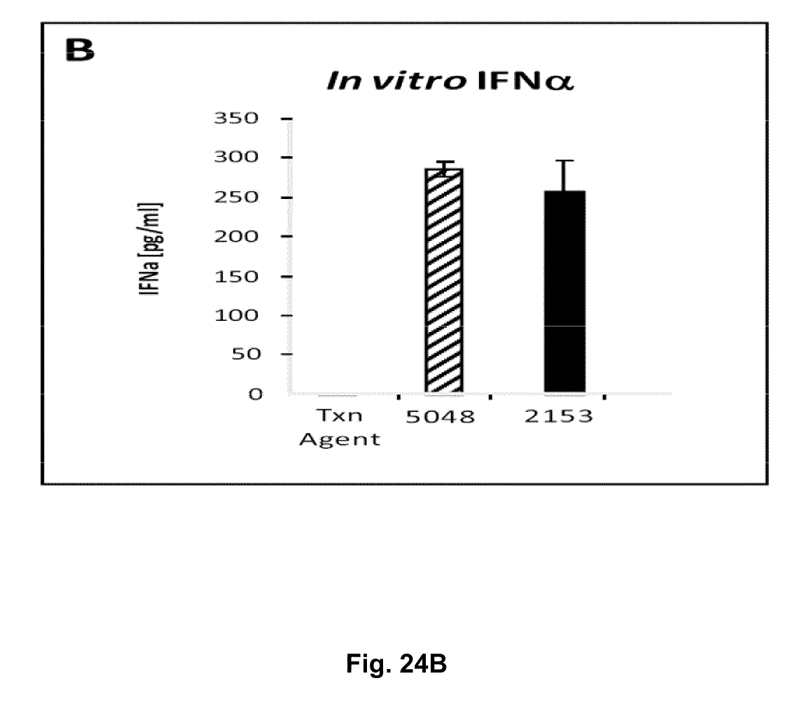

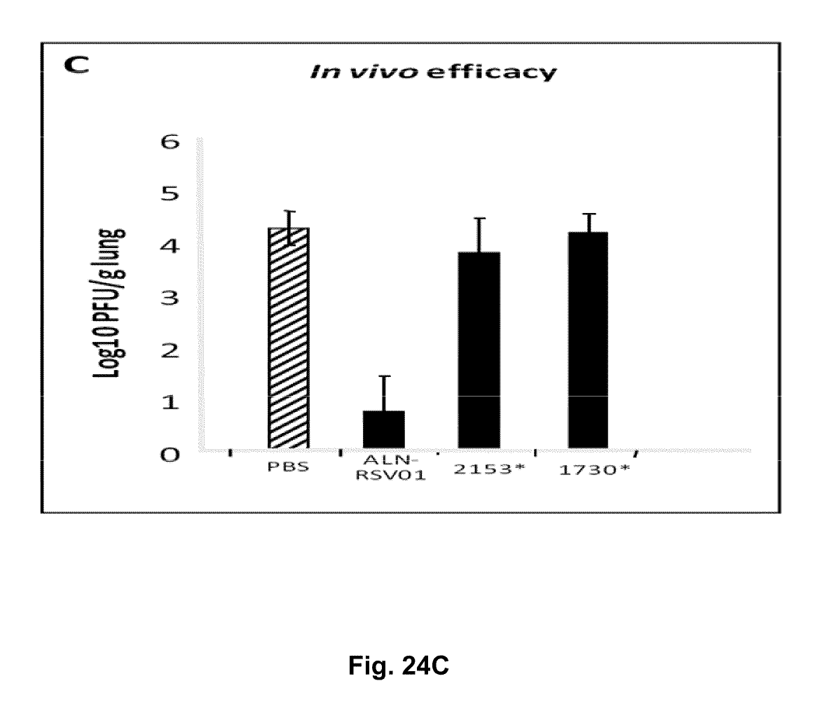

To verify that the antiviral activity of ALN-RSV01 was not influenced by this modest induction of cytokines, two non-RSV specific siRNAs previously shown to more significantly induce either IFNα or TNF [symbol] were assayed in our in vivo BALB/c mouse model. Neither AL-DP-1730, a TNFα inducer (FIG. 24A) nor AL-DP-2153, a IFNα inducer (FIG. 24B) inhibited RSV/A2 when administered intranasally (100 μg) into mice, as compared to the strong inhibition observed when delivering ALN-RSV01 (FIG. 24C). Importantly, even 10 fold higher doses of AL-DP-1730 had no effect on RSV levels when delivered prophylactically, 4 hrs prior to infection (data not shown). These data support the conclusion that ALN-RSV01 antiviral effects are mediated via an RNAi mechanism and not via induction of innate immunity.

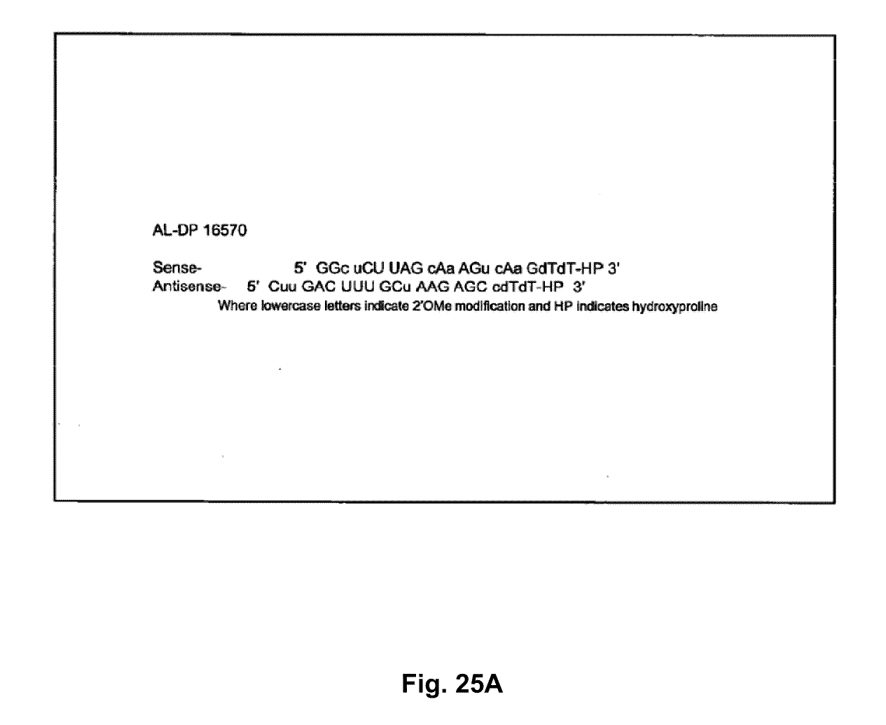

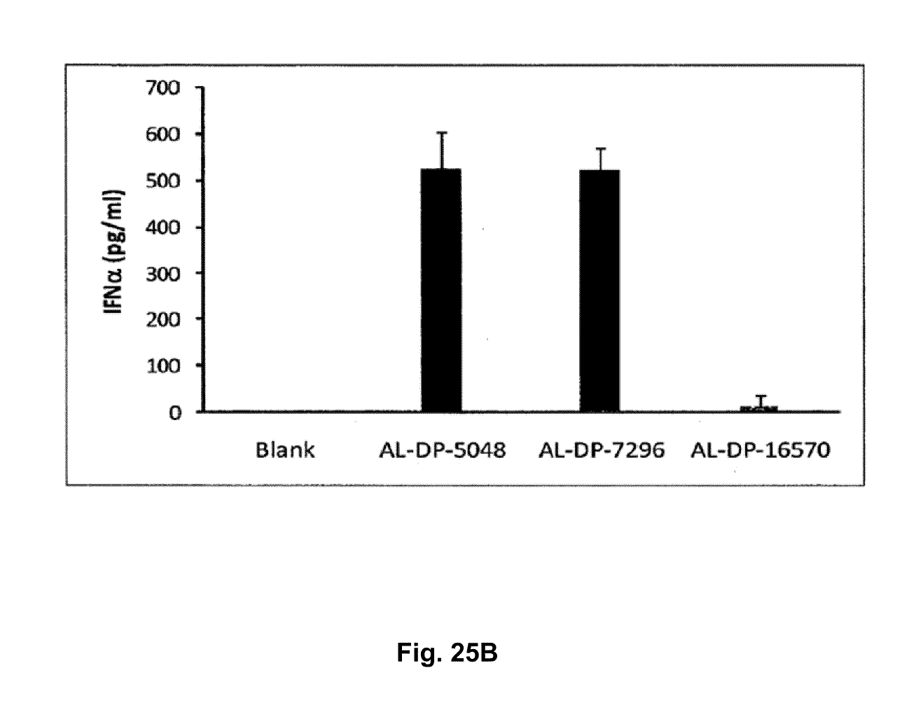

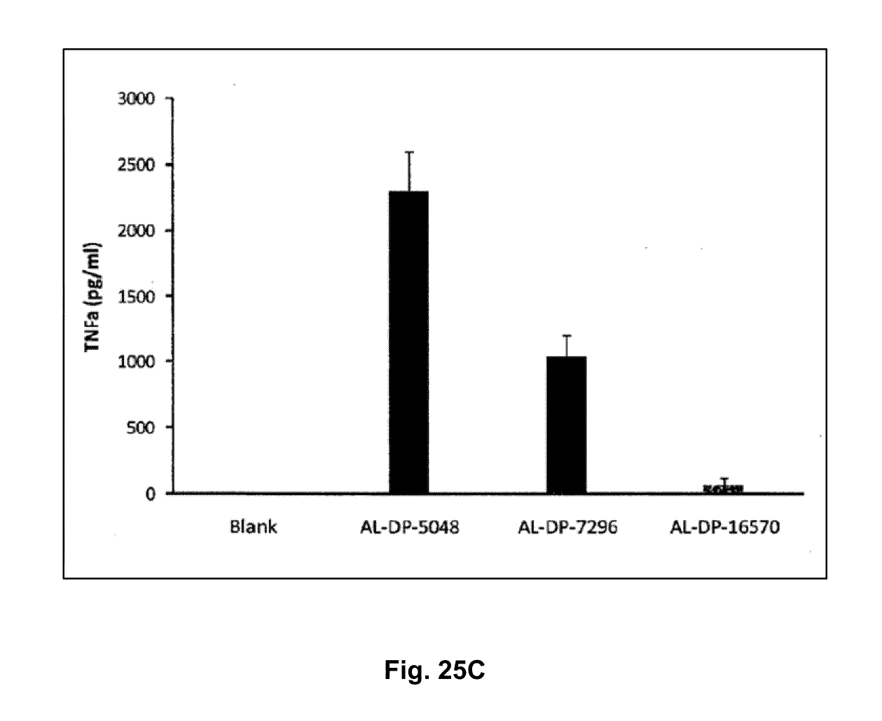

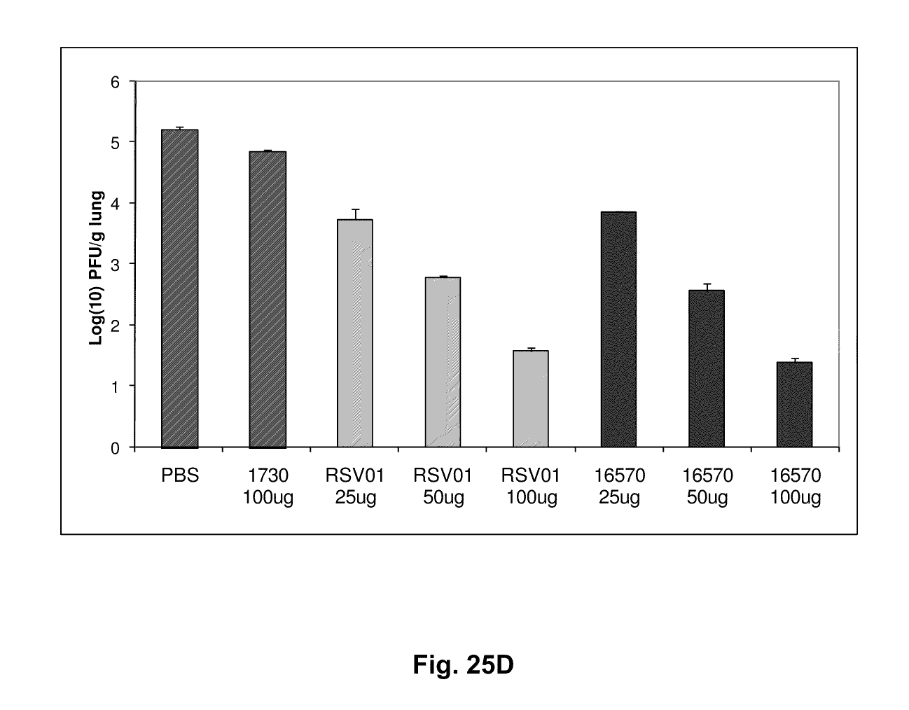

To further evaluate the role of immune activation on the anti-viral efficacy of ALN-RSV01, an immune-silent form of this siRNA, AL-DP-16570 was synthesized containing 2′-O-Me modifications as illustrated in FIG. 25A. AL-DP-16570 showed potent anti-viral activity in vitro with an IC₅₀ of ˜1 nM (data not shown), comparable to that measured for ALN-RSV01. Further, in PBMC assays, high concentrations of AL-DP-16570 (133 nM) showed no significant induction of either IFNα or TNFα (FIGS. 25B and 25C). AL-DP-16570 was then tested for in vivo anti-viral efficacy in the mouse model. As compared with a non-specific siRNA control, the chemically-modified, immune-silent, RSV-specific AL-DP-16570 showed potent anti-viral efficacy, equivalent to the parent sequence ALN-RSV01 (FIG. 25D).

In vitro and in vivo RACE Analysis of ALN-RSV01 cleavage product. To definitively confirm an RNAi-mediated mechanism of action for ALN-RSV01, a 5′ Rapid Amplification of cDNA Ends (RACE) assay was used. This assay allows the potential capture and sequence analysis of the specific RNAi cleavage product mRNA intermediate following ALN-RSV01 treatment both in in vitro and in vivo; the RISC cleavage of a specific mRNA transcript occurring exactly 10 nucleotides from the 5′-end of the siRNA antisense strand.

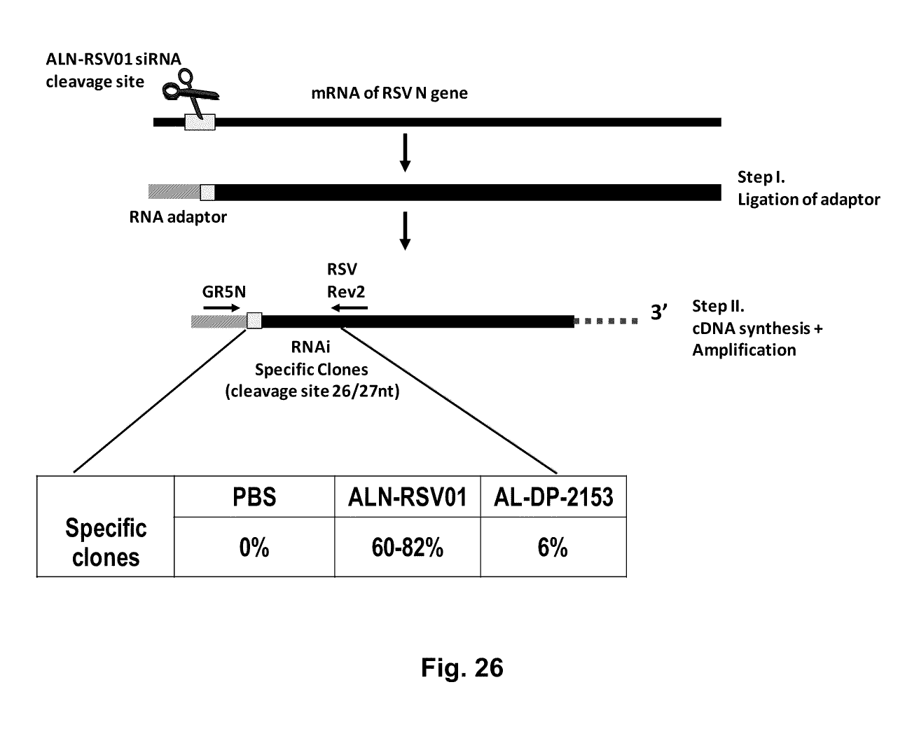

Following siRNA transfection (200 nM) into Vero cells and subsequent infection with RSV/A2, a specific cleavage fragment could be detected only in the samples treated with ALN-RSV01 as compared to either PBS or a non-specific siRNA (AL-DP-2153) control (data not shown). In these experiments, 92% of the sequenced clones resulted from site-specific cleavage (between positions 26/27 of RSV/A2 N mRNA) (data not shown).When analyzed in vivo, 60-82% of clones isolated from lung tissue of ALN-RSV01 treated, RSV-infected mice demonstrated site-specific cleavage of the N-gene transcript between positions 26/27 (FIG. 26). Only animals treated with ALN-RSV01, in contradistinction with those treated with PBS or mismatch controls, yielded significant numbers of clones whose sequence was confirmed as the predicted cleavage site (FIG. 26).

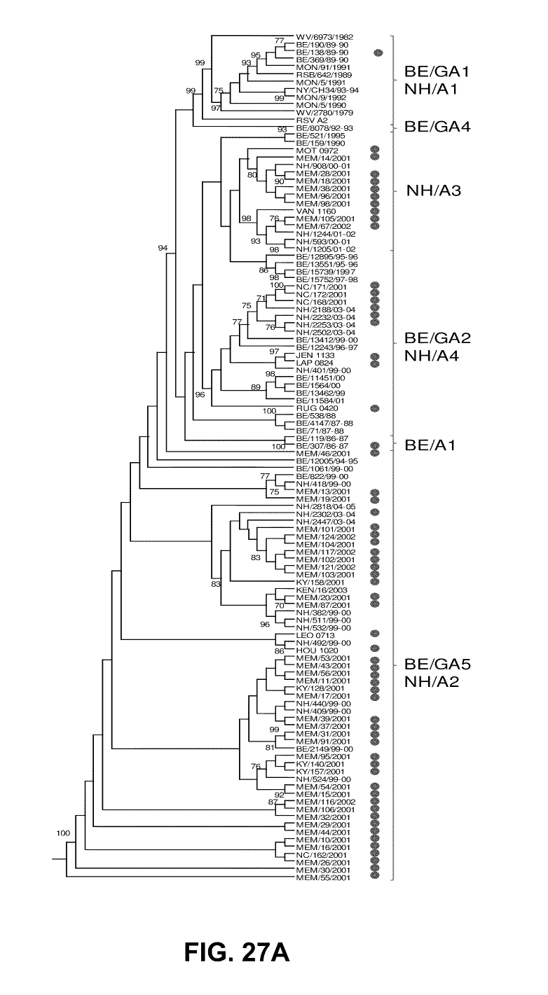

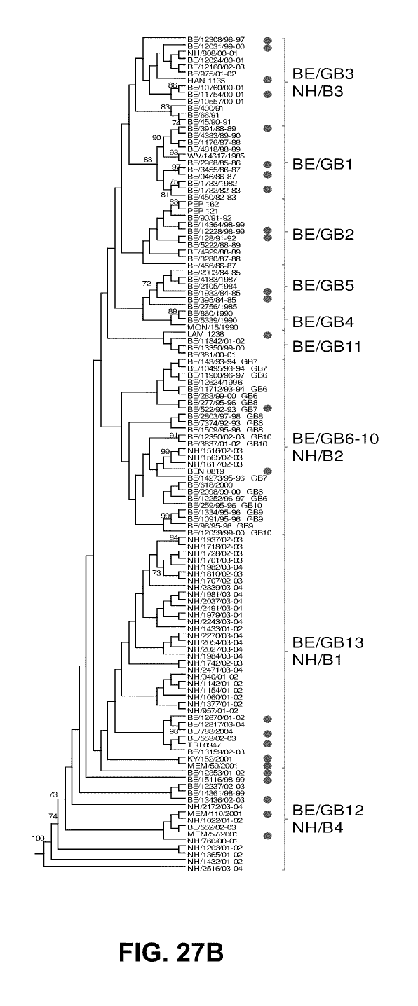

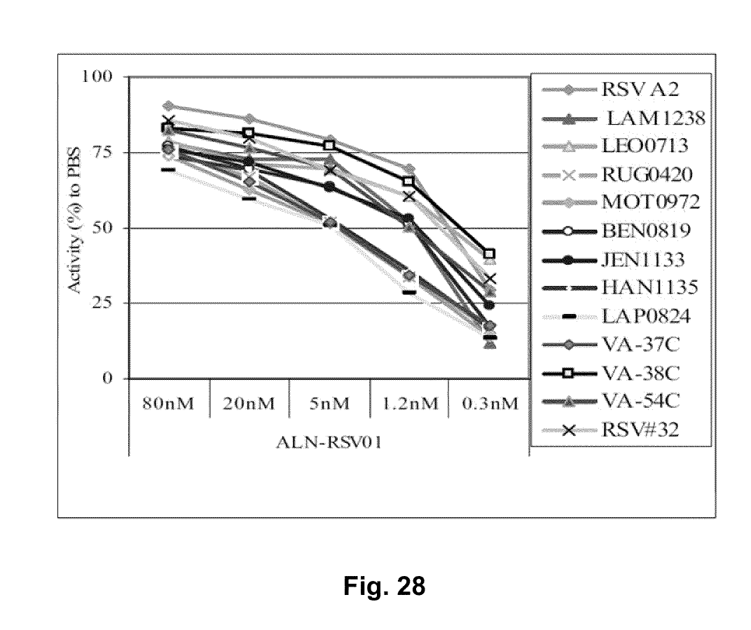

ALN-RSV01 inhibition of RSV primary isolates. The relationship between clinical disease and molecular epidemiology of RSV is poorly understood, as several different genotypes cocirculate during most seasons, and dominating genotypes can vary from year to year. It is therefore crucial that any prospective anti-viral agent targets the broadest possible array of identified genotypes. Based on the mechanism of RNAi, it is predicted that sequence identity between an siRNA and its target, implies functional silencing. For this reason, a series of primary isolates (genotype analysis, FIGS. 27A and 27B) taken from nasal washes of children with confirmed RSV disease, were sequenced across the ALN-RSV01 recognition element. Of the RSV primary isolates sequenced, 94% (89/95) showed absolute conservation across the ALN-RSV01 target site. The six isolates that were not 100% conserved each had a single base alteration within the ALN-RSV01 target site. Four had C-U mutations at position 4 with respect to the 5′ end of the antisense strand of ALN-RSV01, one had A-G mutation at position 7 and one had G-A mutation at position 1. A subset of these 95 isolates was tested in the in vitro viral inhibition assay including one isolate with a mismatch at position 4 and another with a mismatch at position 7 (Table 16). Of these, 12/12 (100%) exhibited ˜70% inhibition at 80 nM ALN-RSV01 as compared to PBS control, and all had similar dose response curves for ALN-RSV01 inhibition (FIG. 28).

TABLE 16

| Name | Target site | Seq id no: |
|---|---|---|
| ALN-RSV01 | GGCUCUUAGCAAAGUCAAG | 302 |
| RSV A2 | GGCUCUUAGCAAAGUCAAG | 302 |
| LAM 1238 | GGCUCUUAGCAAAGUCAAG | 302 |
| LEO0713 | GGCUCUUAGCAAAGUCAAG | 302 |
| RUG0420 | GGCUCUUAGCAAAGUCAAG | 302 |
| MOT0972 | GGCUCUUAGCAAAGUCAAG | 302 |
| BEN0819 | GGCUCUUAGCAAAGUCAAG | 302 |
| JEN 1133 | GGCUCUUAGCAAAGUCAAG | 302 |
| HAN 1135 | GGCUCUUAGCAAAGUUAAG | 303 |
| LAP 0824 | GGCUCUUAGCAAGGUCAAG | 304 |
| VA-37C | GGCUCUUAGCAAAGUCAAG | 302 |
| VA-38C | GGCUCUUAGCAAAGUCAAG | 302 |
| VA-54C | GGCUCUUAGCAAAGUCAAG | 302 |
| RSV#32 | GGCUCUUAGCAAAGUCAAG | 302 |

### Example 21

### Cytokine Activation in PBMCs of Modified RSV Targeting siRNAs

Candidate siRNAs targeting RSV were screened for cytokine stimulation in an in vitro human PBMC assay.

To examine the ability of siRNAs to stimulate interferon alpha (IFNα) or tumor necrosis factor alpha (TNFα), human peripheral blood mononuclear cells (hPBMCs) were isolated from concentrated fractions of leukocytes (buffy coats) obtained from the Blood Bank Suhl, Institute for Transfusion Medicine, Germany. Buffy coats were diluted 1:1 in PBS, added to a tube of Histopaque (Sigma, St. Louis, Mo.) and centrifuged for 20 minutes at 2200 rpm to allow fractionation. White blood cells were collected, washed in PBS, followed by centrifugation. Cells were resuspended in RPMI 1640 culture medium (Invitrogen) supplemented with 10% fetal calf serum, IL-3 (10 ng/ml) (Sigma) and phytohemagglutinin-P (PHA-P) (5 μg/ml) (Sigma) for IFNα assay, or with no additive for TNFα assay at a concentration of 1×10⁶ cells/ml, seeded onto 96-well plates and incubated at 37° C., 5% CO₂. Control oligonucleotides were the following siRNAs:

Positive control AL-DP-5048 duplex:

| | |
|---|---|
| 5′-GUCAUCACACUGAAUACCAAU-3′ | (SEQ ID NO: 287) |
| 3′-CACAGUAGUGUGACUUAUGGUUA-5′; | (SEQ ID NO: 288) |

Negative control AL-DP-7296 duplex:

| | |
|---|---|
| 5′-CUACACAAAUCAGCGAUUUCCAUGU-3′ | (SEQ ID NO: 289) |
| 3′-GAUGUGUUUAGUCGCUAAAGGUACA-5′ | (SEQ ID NO: 290) |

Cells in culture were combined with either 500 nM oligonucleotide, pre-diluted in OptiMEM (Invitrogen), or 133 nM oligonucleotide pre-diluted in OptiMEM and Geneporter, GP2 transfection reagent (Genlantis, San Diego, Calif.) for IFNα assay or N-[1-(2,3-Dioleoyloxy)propyl]-N,N,N-trimethylammonium methylsulfate (DOTAP) (Roche, Switzerland) for TNFα assay and incubated at 37° C. for 24 hrs. IFNα and TNFα were measured using the Bender MedSystems (Vienna, Austria) instant ELISA kit according to manufacturer's instruction.

Results are shown in the following table. Data are represented in % relative to the positive control AD-5048.

TABLE 17

| siRNA duplex | IFNα | % TNFα |
|---|---|---|
| AD_5048 | 100 | 100 |
| Blank | 0 | 0 |
| AD_7296 | >80 <170 | >15 <450 |
| AD_RSV01 | >30 <115 | >15 <220 |
| AD_16484 | <5 | <5 |
| AD_16485 | <5 | <5 |
| AD_16527 | 0 | <5 |
| AD_16528 | <5 | <5 |
| AD_16570 | <5 | <5 |
| AD_16571 | 0 | <10 |
| AD_16613 | <10 | <5 |
| AD_16614 | <5 | <5 |
| AD_16467 | <5 | <20 |
| AD_16468 | 0 | <15 |
| AD_16469 | <5 | <5 |
| AD_16470 | 0 | <5 |
| AD_16471 | 0 | <5 |
| AD_16472 | <10 | <5 |
| AD_16473 | <5 | <5 |
| AD_16474 | 0 | <10 |
| AD_16475 | 0 | <15 |
| AD_16486 | 0 | <10 |
| AD_16487 | <5 | <10 |
| AD_16510 | <5 | <5 |
| AD_16511 | 0 | <5 |
| AD_16512 | 0 | <10 |
| AD_16513 | 0 | <5 |
| AD_16514 | <5 | <5 |
| AD_16515 | 0 | <15 |
| AD_16516 | 0 | <5 |
| AD_16517 | 0 | <5 |
| AD_16518 | 0 | <10 |
| AD_16529 | 0 | <5 |
| AD_16530 | <5 | <5 |
| AD_16553 | 0 | <10 |
| AD_16554 | 0 | <5 |
| AD_16555 | 0 | <10 |
| AD_16556 | 0 | <10 |
| AD_16557 | 0 | 0 |
| AD_16558 | 0 | 0 |
| AD_16559 | 0 | <5 |
| AD_16560 | 0 | 0 |
| AD_16561 | 0 | <5 |
| AD_3518 | <20 | <10 |
| AD_3519 | <30 | <15 |
| AD_3520 | <10 | <15 |
| AD_3521 | <20 | <10 |
| AD_3522 | <20 | <10 |
| AD_3523 | <25 | <10 |
| AD_3524 | <25 | <5 |
| AD_3525 | <20 | <5 |
| AD_3526 | <15 | <5 |
| AD_3527 | <60 | <5 |
| AD_3528 | <60 | <10 |
| AD_3529 | <60 | <15 |
| AD_3530 | <25 | <20 |
| AD_3531 | <25 | <30 |
| AD_3532 | <10 | <20 |
| AD_3533 | <55 | <30 |
| AD_3534 | <35 | <30 |
| AD_3524 | <25 | <5 |
| AD_3536 | <5 | <20 |
| AD_3537 | <20 | <20 |
| AD_3538 | <60 | <25 |
| AD_3539 | <50 | <25 |
| AD_3581 | <10 | <10 |
| AD_3582 | <5 | <15 |
| AD_3583 | <1 | <15 |
| AD_3584 | <5 | <10 |
| AD_3585 | <5 | <15 |
| AD_3586 | <5 | <10 |
| AD_3587 | <5 | <10 |
| AD_3588 | <5 | <10 |
| AD_3589 | <10 | <25 |
| AD_3590 | <10 | <20 |
| AD_3591 | <15 | <20 |
| AD_3592 | <20 | <30 |
| AD_3593 | <5 | <25 |
| AD_3594 | <20 | <10 |
| AD_3595 | <20 | <15 |
| AD_3596 | <5 | <15 |
| AD_3597 | <20 | <10 |
| AD_3598 | <20 | <20 |
| AD_3581 | <5 | 0 |
| AD_3582 | 0 | <10 |
| AD_3583 | 0 | 0 |
| AD_3584 | 0 | <5 |
| AD_3585 | <5 | <5 |
| AD_3586 | <6 | <5 |
| AD_3587 | <7 | <5 |
| AD_3588 | <8 | <5 |
| AD_3589 | <9 | <5 |
| AD_3590 | <10 | <5 |
| AD_3591 | <11 | <5 |
| AD_3592 | <12 | <10 |
| AD_3593 | <13 | <5 |
| AD_3594 | <14 | <5 |
| AD_3595 | <15 | <10 |
| AD_3596 | <15 | <15 |
| AD_3597 | 0 | <5 |
| AD_3598 | <10 | <10 |

### Example 22

### Chemical Modification Can Alter Immunostimulatory Profile of siRNAs

Immunostimulatory effects are sequence, concentration (and structure) dependent. It has been shown that modifications at the 2′ position of ribose eliminate TLR-mediated innate immune stimulation by siRNAs in vitro and in vivo. It has also been shown that innate immune stimulation through RIG-I pathway can also be eliminated by 2′ modifications (Sioud et al., Eur J Immunol 36:1222 (2006); Judge et al., Mol Ther 13:494 (2006); Cekaite et al., JMB 365:90 (2007); Sioud et al., BBRC 361:122 (2007); Robbins et al., Mol Ther 15:1663 (2007); Hornung et al., Science 314:994 (2006)).

Using the TNF-α in vitro assay described herein, the effect of chemically modified siRNA on cytokine activation was compared to the effect of unmodified siRNA. Two modified siRNAs were used: AD-3532 and AD-3534. The unmodified version was ALN-RSV01 (AD-2017).

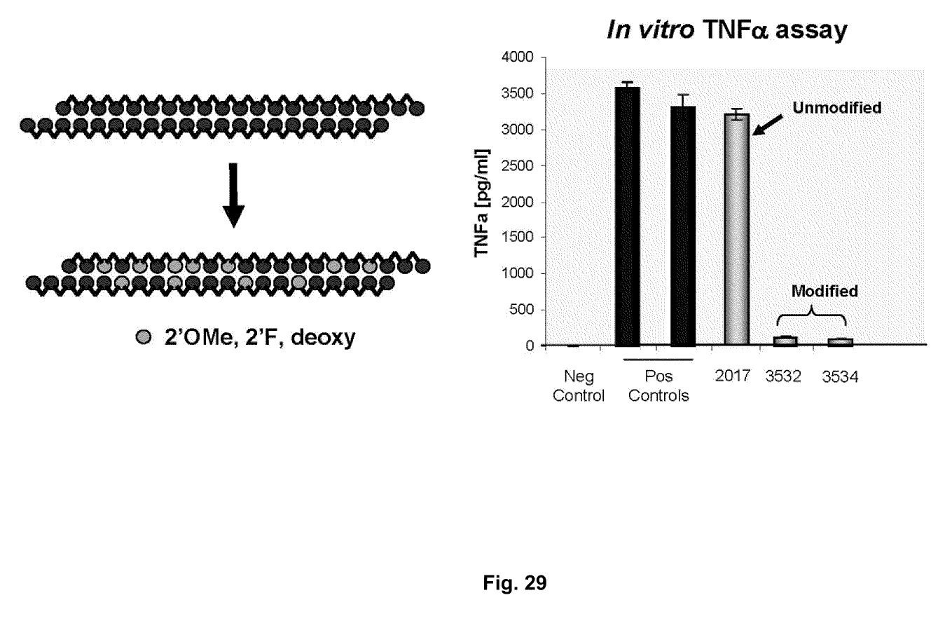

The results are shown in FIG. 29. Modified siRNAs demonstrated a greatly decreased immunostimulatory profile when compared to the unmodified version of the duplex.

### Example 23

### Attenuation of Immuno-Stimulation Activity by Modified ALN-RSV01 Molecules

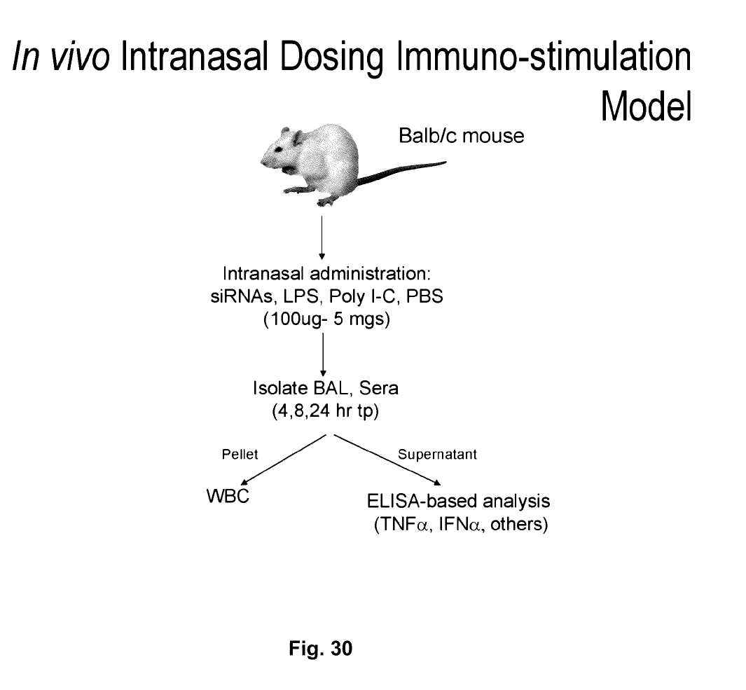

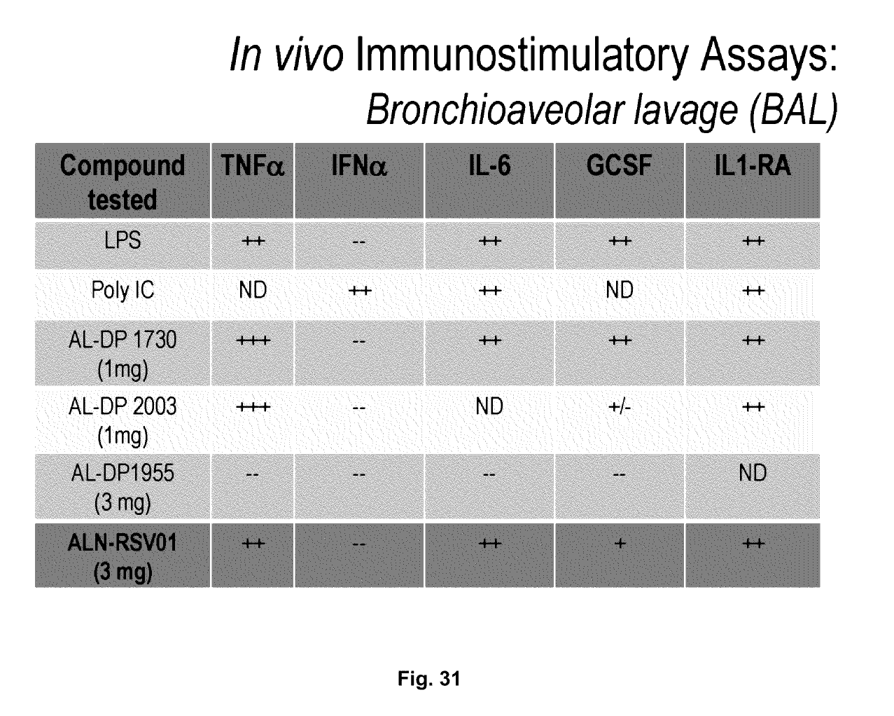

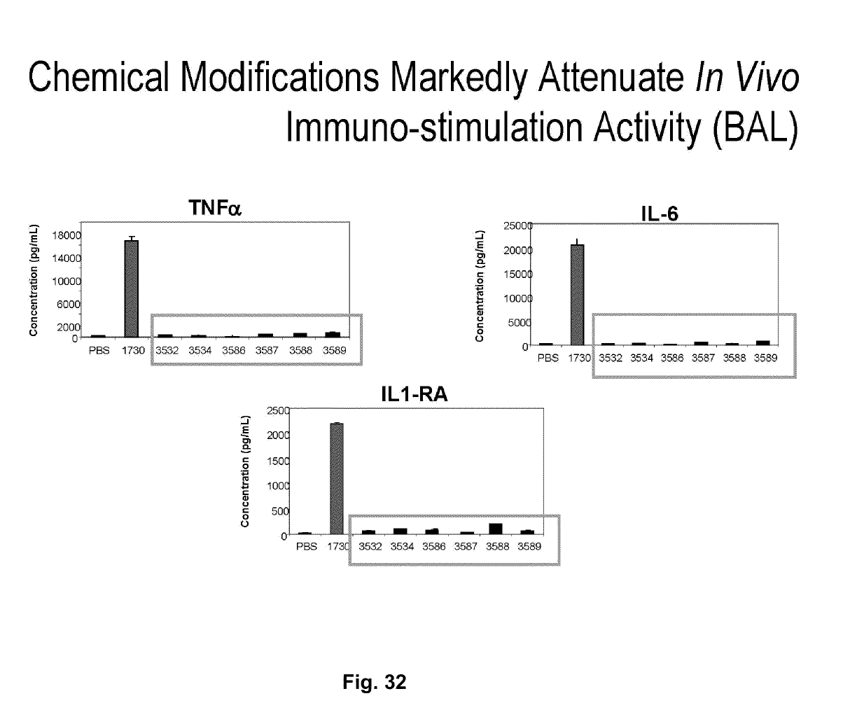

Immuno-stimulation by modified ALN-RSV01 sequences was studied using an in vivo intranasal dosing model. Briefly, the method entailed intranasally administering an indicated dose of siRNA (or control) to mice then, following an incubation period, obtaining a serum sample or a sample of the epithelial lining fluid using bronchoalveolar lavage (BAL). See, e.g., FIG. 30. An in vitro assay, e.g., a standardized ELISA test, is then used to measure the expression of one or more cytokines as described herein. Results for ALN-RSV01 and various controls are depicted in FIG. 31. FIG. 32 shows that immuno-stimulation by siRNAs is markedly attenuated when modified siRNAs are used. Compared to the control siRNA 1730, the observed concentrations of TNF-alpha, IL-6 and IL1-RA are substantially reduced, by one or more orders of magnitude in some instances. Thus, in certain embodiments, the invention provides siRNA compositions for inhibiting the expression of RSV genes, wherein said compositions comprise chemically modified nucleotide sequences and wherein said modifications markedly attenuate in vivo immunostimulation activity relative to unmodified compositions.

### Example 24

### Chemically Modified ALN-RSV01 siRNA Sequences Exhibit Significantly Reduced Immunostimulatory Activity Over the Parental Unmodified RSV01 siRNA Sequence in Human PBMC In Vitro

Material and Methods

PBMC Assay: To examine the ability of siRNAs to stimulate innate immune activation, human peripheral blood mononuclear cells (PBMCs) were isolated from freshly collected whole blood obtained from healthy donors (Research Blood Components, Inc., Boston, Mass.). Blood was diluted 1:1 in PBS, and centrifuged over Lymphocyte Separation Medium (MP Biologicals) for 30 minutes at 400×g to allow fractionation. PBMCs were collected, washed in PBS, followed by centrifugation. Cells were resuspended in RPMI 1640 Glutamax tissue culture medium (Invitrogen) supplemented with 10% fetal calf serum and Antibiotic-Antimycotic (AA; Invitrogen) at a concentration of 1×10⁶ cells/ml, seeded at 1×10⁵ cells/100 μl/well onto 96-well plates and incubated at 37° C., 5% CO₂. Control oligonucleotides were the following siRNAs (N and n represent adenosine (A), guanosine (G), cytidine (C), uracil (U) or deoxythymidine (dT); N=2′-OH and n=2′-OMe modification; s=phosphorothioate linkage):

| Positive control AL-DP 5048 duplex: | |
|---|---|
| (SEQ ID NO: 287) | |
| Sense: | 5′-GUCAUCACACUGAAUACCAAU-3′ |
| (SEQ ID NO: 288) | |
| Antisense: | 5′-AUUGGUAUUCAGUGUGAUGACAC-3′ |
| Positive control AL-DP-1730 duplex: | |
| (SEQ ID NO: 249) | |
| Sense: | 5′-CGAUUAUAUUACAGGAUGAdTsdT-3′ |
| (SEQ ID NO: 268) | |
| Antisense: | 5′-UCAUCCUGUAAUAUAAUCGdTsdT-3′ |
| Negative control AL-DP-1955 duplex: | |
| (SEQ ID NO: 318) | |
| Sense: | 5′-cuuAcGcuGAGuAcuucGAdTsdT-3′ |
| (SEQ ID NO: 319) | |
| Antisense: | 5′-UCGAAGuACUcAGCGuAAGdTsdT-3′ |

Three 2′-O-Me modified siRNA duplexes were used: AL-DP-3532, AL-DP-3586, and AL-DP-3587.

| 2′-O-Me modified AL-DP-3532 duplex: | |
|---|---|
| (SEQ ID NO: 320) | |
| Sense: | 5′-GGcuCUUAGcAaAGucAAGdTsdT-3′ |
| (SEQ ID NO: 323) | |
| Antisense: | 5′-CUuGACUUuGCUAAGAGCcdTsdT-3′ |
| 2′-O-Me modified AL-DP-3586 duplex: | |
| (SEQ ID NO: 321) | |
| Sense: | 5′-GgCUCUuAGcAAAGUcAAGdTsdT-3′ |
| (SEQ ID NO: 325) | |
| Antisense: | 5′-CUuGACUUuGCuAAGAGCcdTsdT-3′ |
| 2′-O-Me modified AL-DP-3587 duplex: | |
| (SEQ ID NO: 322) | |
| Sense: | 5′-GGcuCUUAGcAaAGucAaGdTsdT-3′ |
| (SEQ ID NO: 326) | |
| Antisense: | 5′-CUuGACUuUGCuAAGAGCcdTsdT-3′ |
| Unmodified AL-DP-2017 (ALN-RSV01) duplex: | |
| (SEQ ID NO: 1) | |
| Sense: | 5′-GGCUCUUAGCAAAGUCAAGdTdT-3′ |
| (SEQ ID NO: 2) | |
| Antisense: | 5′-CUUGACUUUGCUAAGAGCCdTdT-3′ |

Cells in culture were combined with various concentrations of siRNA duplexes pre-diluted in OptiMEM Reduced Serum Medium (Invitrogen) and complexed with N-[1-(2,3-Dioleoyloxy)propyl]-N,N,N-trimethylammonium methylsulfate (DOTAP) transfection reagent (Roche Applied Science) incubated at 37° C. for 24 hrs. siRNA/DOTAP complexes were prepared and incubated as specified by the reagent manufacturer's instructions. siRNAs were used at final concentrations of 133 nM -2.5 nM. DOTAP was used at constant final concentration of 8 μg/ml. Supernatants were harvested and stored at −80° C. until analyzed for cytokine concentrations. PBMC were processed directly for total RNA isolation.

IFN-α was measured using the Bender MedSystems (Vienna, Austria) instant ELISA kit according to manufacturer's instruction. TNF-α, IL-6, IP-10, IFN-γ, G-CSF, and IL-1ra were measured using a human cytokine multiplex kit (BioRad Human Cytokine Group 1 6-plex Express Assay) on the Biorad Bio-Plex 200 Luminex instrument, and the data were analyzed using Bio-Plex Manager Software, version 5.0. The required agents were purchased from BioRad (Hercules, Calif.).

Total RNA isolation using MagMAX-96 Total RNA Isolation Kit (Applied Biosystem, Foster City Calif., cat #: AM1830). Plates containing cells were centrifuged at 150×g for 5 min, residual culture supernatant carefully removed so as not to disrupt cell pellets, and 70 μl of Lysis/Binding solution/well was added. The Lysis/Binding solution containing the cells was mixed for 1 minute at 850 rpm using an Eppendorf Thermomixer (the mixing speed was the same throughout the process). Twenty micro liters of magnetic beads were added into cell-lysate and mixed for 5 minutes. Magnetic beads were captured using magnetic stand and the supernatant was removed without disturbing the beads. After removing supernatant, magnetic beads were washed with 150 μl Wash Solution 1 (isopropanol added) and mixed for 1 minute. Beads were capture again and supernatant removed. Beads were then washed with 150 μl Wash Solution 2 (Ethanol added), captured and supernatant was removed. Fifty microliters of DNase mixture (MagMax turbo DNase Buffer and Turbo DNase) was then added to the beads and they were mixed for 10 to 15 minutes. After mixing, 100 μl of RNA Rebinding Solution was added and mixed for 3 minutes. Supernatant was removed and magnetic beads were washed again with 150 μl Wash Solution 2 and mixed for 1 minute and supernatant was removed completely. The magnetic beads were mixed for 2 minutes to dry before RNA it was eluted with 35 μl of water.

cDNA synthesis using ABI High capacity cDNA reverse transcription kit (Applied Biosystems, Foster City, Calif., Cat #4368813): A master mix of 2 μl 10× Buffer, 0.8 μl 25×dNTPs, 2 μl Random primers, 1 μl Reverse Transcriptase, 1 μl RNase inhibitor and 3.2 μl of H2O per reaction were added into 10 μl total RNA. cDNA was generated using a Bio-Rad C-1000 or S-1000 thermal cycler (Hercules, Calif.) through the following steps: 25° C. 10 min, 37° C. 120 min, 85° C. 5 sec, 4° C. hold.

Quantification of interferon (IFN)-inducible genes by real time polymerase chain reaction(PCR): Two microliters of cDNA was added to a master mix of 0.5 μl 18S TaqMan Probe (Applied Biosystems Cat #4319413E), 5 μl LightCycler 480 Probes Master (Roche Cat #04 887 301 001), 2 μl Nuclease-free water (Qiagen Material#1039480) and 0.5 μl of one of the following TaqMan probes: human IFI27 (Applied Biosystems Cat# Hs00271467_m1), human IFI27 (Applied Biosystems Cat# Hs00271467_m1), human IFN-γ (Applied Biosystems Cat# Hs00174143_m1), human IFIT1 (Applied Biosystems Cat# Hs01675197_m1), human IFIT2 (Applied Biosystems Cat# Hs00533665_m1), human IP-10 (Applied Biosystems Cat# Hs00171042_m1), human IL-6 (Applied Biosystems Cat# Hs00174131_m1), humanOAS3 (Applied Biosystems Cat# Hs00196324_m1), and human RSAD2/Viperin (Applied Biosystems Cat# Hs00369813_m1). Ten microliters per well of master mix was added to a LightCycler Multiwell plate 384, white (Roche Cat #04 729 749 001). Real time PCR was done in a LightCycler 480 II system (Roche) using the Dual Hydrolysis Probe assay from LightCycler 480 Software version 1.5.0.39. All reactions were performed in duplicate. Real time data were analyzed using the ΔΔCt method and normalized to assays performed from cells treated with assay medium alone.

Results

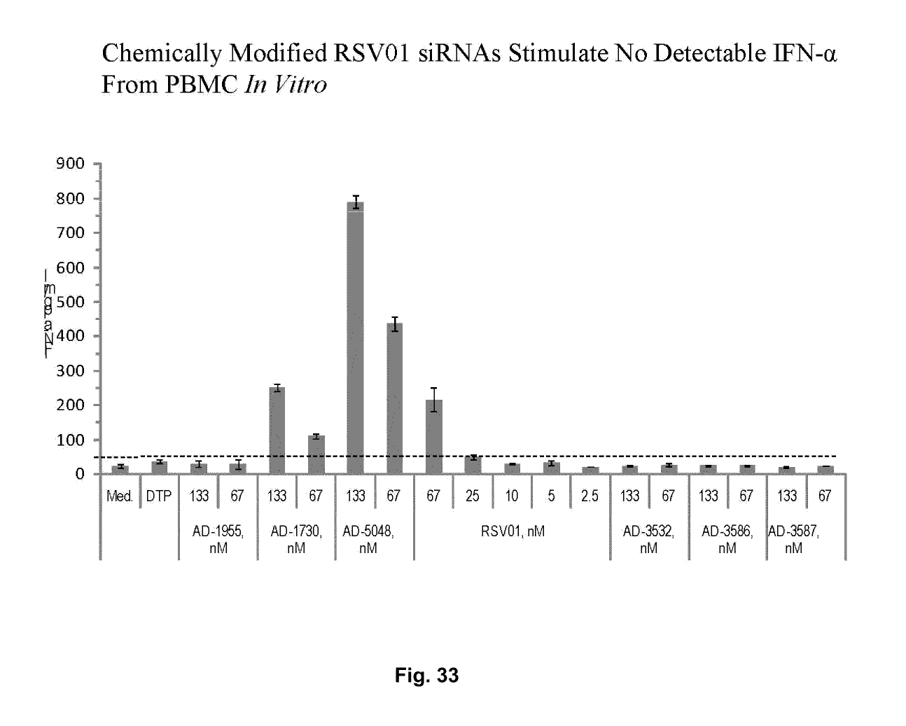

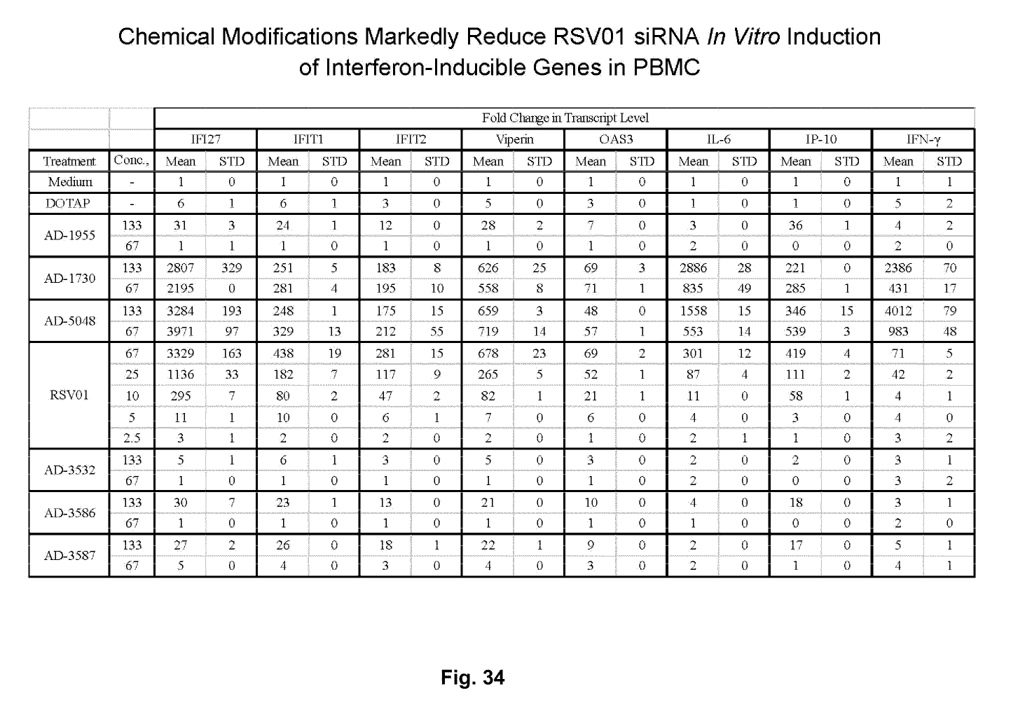

Using the in vitro PBMC assay described herein, the effect of modified ALN-RSV01 siRNA on cytokine activation was compared with the effect of unmodified ALN-RSV01 using both ELISA and cytokine multiplex analysis. Three modified ALN-RSV01 siRNAs with selectively introduced 2′-O-Me modifications were used: AD-3532, AD-3586, and AD-3587. The unmodified version was ALN-RSV01 siRNA. PBMC were treated with various doses of the modified siRNAs, ALN-RSV01 (AD-2017), and control siRNAs, after which the expression of a panel of seven innate immune cytokines (IFN-α, TNF-α, IL-6, IP-10, G-CSF, IFN-γ, and IL-ra) was measured. Data are shown in FIG. 33 and FIG. 34. Results show that the immuno-stimulation by the ALN-RSV01 siRNA sequence is significantly reduced by the addition of 2-O-Me chemical modifications. All three modified ALN-RSV01 siRNAs stimulated no detectable IFN-α whereas the parental AD-2017 siRNA sequence evoked moderate IFN-α production (FIG. 33). Compared to the parental AD-2017 siRNA sequence, the observed concentrations of TNF-α, IL-6 and IP-10 are reduced overall by a magnitude or greater for all three modified siRNA sequences (FIG. 34; <LLOQ; cytokine levels below the lower level of detection. Limits of detection for the cytokines measured: TNF-α, 7-25879 pg/ml; IL-6, 2-8002 pg/ml; IP-10, 38-26230 pg/ml; IFN-γ, 19-26280 pg/ml; G-CSF, 1-5447 pg/ml; IL-ra, 5-79814 pg/ml). Strikingly, the levels of TNF-α, IL-6 and IP-10 induced by the modified ALN-RSV01 sequences were either undetectable or were equal to those observed for the chemical modified, immunosilent control AD-1955. The modest G-CSF and IL-1ra responses induced by the ALN-RSV01 siRNA were fully abrogated by chemical modification. No specific IFN-γ induction was detected in response to treatment with either the parental RSV)1 siRNA or the modified ALN-RSV01 siRNAs. The capacity of the donor PBMC to produce all six cytokines in response to immuno-stimulatory siRNA was confirmed by the robust responses evoked by the positive controls AD-1730 and AD-5048. These data demonstrate that chemical modification of the parental ALN-RSV01 siRNA duplex effectively abrogates the capacity of this sequence to induce multiple cytokines from in vitro PBMC.

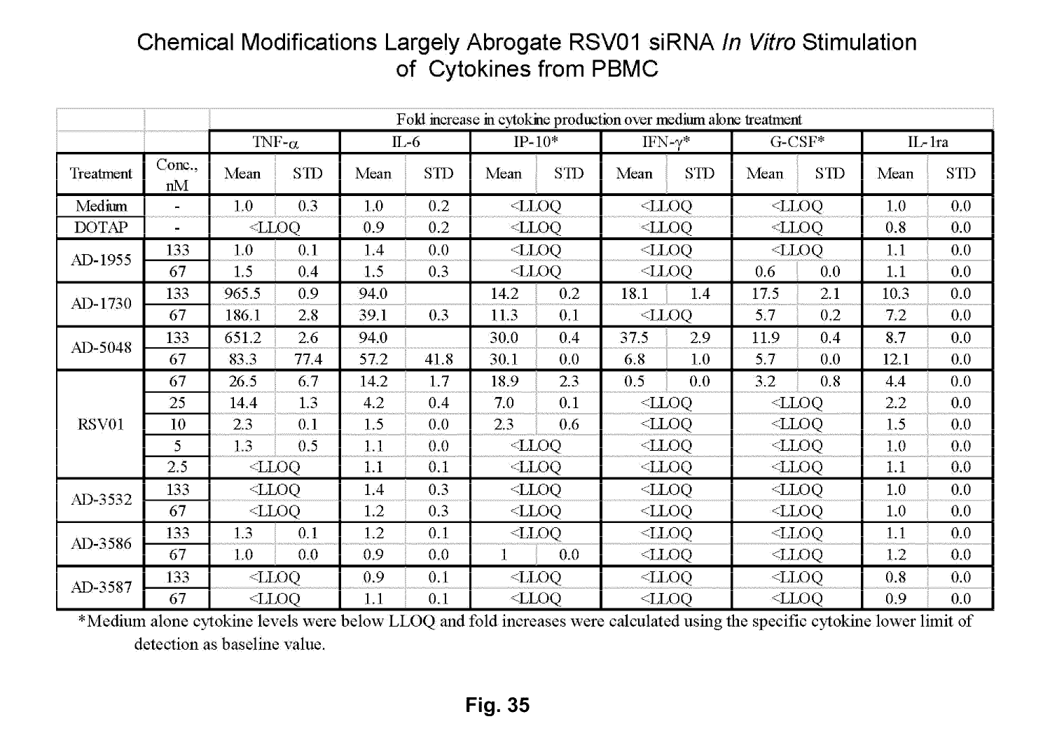

Using the cDNA synthesis and real-time PCR methods described herein, the effect of modified ALN-RSV01 immune activation compared to unmodified ALN-RSV01 immune activation was further assessed by measuring the induction of IFN-inducible mRNA transcripts. IFN-inducible mRNA transcripts have been demonstrated to serve as readouts for measuring the activity of TLR ligands in primary immune cells in vitro (Sims, P. et al., in: Toll-Like Receptors: Methods and Protocols, Methods Molec. Bio., 517, Humana Press, NY, N.Y., 415-440 (C. E. McCoy and L. A. J. O'Neill, eds.)) and therapeutic siRNAs in vivo (Judge, A. et al., (2009) J. Clin. Invest. 119:1-13). In related experiments, a panel of IFN-inducible genes was screened for differential induction in PBMC cultures by siRNAs AD-5048 and AD-1730 (positive controls), AD-1955 (immunosilent control), medium alone and DOTAP alone. This analysis identified the IFI27, IFIT1, IFIT2, viperin, OAS3, IL-6, IFN-γ, and IP-10 mRNAs as being highly induced in a specific and reproducible manner. PBMC were treated with various doses of the modified siRNAs, the AD-2017, and control siRNAs. Cells were harvested 24 hours post transfection and subjected to real-time PCR analysis to detect expression of the above-described mRNAs. Data are shown in FIG. 35.

When mRNA levels are compared as a function of treatment dose, the modified siRNAs are 13 to 26-fold less immunostimulatory than the parental ALN-RSV01 siRNA for seven of the eight mRNAs measured. The modified siRNAs showed a five-fold decrease in IFN-γ mRNA induction compared to the unmodified AD-2017. These gene expression data demonstrate the modified ALN-RSV01 siRNAs are measured to be on the order of 5-26 fold less immunostimulatory than the parental unmodified ALN-RSV01 siRNA, depending on the specific IFN-inducible transcript measured.

### Example 25

### In Vivo Antiviral Activity of Modified RSV Candidates in Multi-Dose Treatment/Prophylaxis Model

In this Example, the in vivo model is run as previously described (Example 19) except that RSV specific-siRNAs are delivered in several dosing paradigms. Some groups are dosed as single prophylactic dose of 40, 80, or 120 μg at 4 hr prior to viral infection. In other groups, siRNAs are delivered prophylactically as 2×40 μg doses, one dose at 1 day prior to infection and the other dose at 4 hours prior to infection. In still other groups, siRNAs are delivered prophylactically and in treatment as 3×40 μg doses, one dose at 4 hr prior to viral infection, a second dose at 1 day prior to infection, and a third dose at day 1 post infection.

All RSV-specific siRNAs are highly active, resulting in an almost 3 log reduction in viral titers, with levels of inhibition indistinguishable amongst ALN-RSV01, AD-3532 and AD-3587, as shown in Table 18, below.

TABLE 18

| | Viral titer | viral titer |
|---|---|---|
| | Log(10) PFU/g | reduction fold to |
| Treatment | lung | PBS |
| PBS | 5.1 | 1 |
| 1730 100ug | 4.9 | |
| ALN-RSV01 40ug 1x | 4.3 | 6.3 |
| ALN-RSV01 40ug 2x | 3.7 | 25 |
| ALN-RSV01 40ug 3x | 2.4 | 500 |
| ALN-RSV01 80ug 1x | 3.7 | 25 |
| ALN-RSV01 120ug 1x | 2.2 | 785 |
| 3532 40ug 1x | 4.7 | 2.5 |
| 3532 40ug 2x | 3.8 | 20 |
| 3532 40ug 3x | 2.7 | 250 |
| 3532 80ug 1x | 3.7 | 25 |
| 3532 120ug 1x | 2.9 | 160 |
| 3587 40ug 1x | 4.3 | 6.3 |
| 3587 40ug 2x | 3.6 | 31.5 |
| 3587 40ug 3x | 2.7 | 250 |
| 3587 80ug 1x | 3.9 | 16 |
| 3587 120ug 1x | 2.7 | 250 |

### Example 26

### In Vivo Antiviral Activity of Modified RSV Candidates in Split-Dose Treatment Model

For this Example, the in vivo model was run as previously described (e.g., Example 19) except that RSV specific-siRNAs were delivered in a treatment dosing paradigm with doses delivered on day 2. Some groups were dosed as single treatment dose of 40, 80, or 120 ug at day 2 post viral infection. In other groups, siRNAs were delivered BID (bi-daily) as 2×40 ug doses, 12 hours apart, day 2 post viral infection. In still other groups, siRNAs were delivered TID (tri-daily) as 3×40 ug doses, 6 hours apart, on day 2 post viral infection.

The results show that all RSV specific siRNAs are highly active, resulting in as much as ˜600 reduction in viral titers, with levels of inhibition indistinguishable amongst ALN-RSV01, AD-3532 and AD-3587, as shown in Table 19.

TABLE 19

| | Viral titer | Viral titer |
|---|---|---|
| | Log(10) PFU/g | reduction fold |
| Treatment | lung | to PBS |
| PBS | 5.4 | 1 |
| 1730 120 ug | 6.5 | |
| ALN-RSV01 40 ug | 4.7 | 5 |
| ALN-RSV01 40 ug BID | 3.8 | 40 |
| ALN-RSV01 40 ug TID | 2.8 | 400 |
| ALN-RSV01 80 ug | 3.7 | 50 |
| ALN-RSV01 120 ug | 2.7 | 500 |
| AD-3532 40 ug | 4.6 | 6.2 |
| AD-3532 40 ug BID | 3.8 | 40 |
| AD-3532 40 ug TID | 2.7 | 500 |
| AD-3532 80 ug | 3.7 | 50 |
| AD-3532 120 ug | 2.6 | 630 |
| AD-3587 40 ug | 4.5 | 8 |
| AD-3587 40 ug BID | 3.8 | 40 |
| AD-3587 40 ug TID | 2.7 | 500 |
| AD-3587 80 ug | 3.7 | 50 |
| AD-3587 120 ug | 2.8 | 400 |

### Example 27

### Stability of Modified ALN-RSV01 Duplexes

In this Example, six modified ALN-RSV01 duplexes were tested for their stability in different biological matrixes. The results and experimental details are summarized in this Example.

The six modified ALN-RSV01 duplexes differ in the number of 2′-O-Methyl-base modifications in the sense and antisense strand, but otherwise share the same sequence of nucleotides. Table 20 represents the combinations of 4 different sense strands with 4 different antisense strands.

TABLE 20

| | | | | | | |
|---|---|---|---|---|---|---|
| | sense strand | | | | | |
| | (5′--3′) (Core sequences | | | antisense strand | | |
| | (i.e. lacking the | | | (5′--3′) (Core sequences | | |
| | 3′ dTdsT nucleotides) | | | (i.e. acking the 3′ dTdsT | | |
| | disclosed as SEQ ID NOS | | | nucleotides) disclosed as | | |
| | 302, 327, 327, 328, 329, 327 | SEQ | Anti- | SEQ ID NOS 305, 330-333, | SEQ | |
| Sense | and 328, respectively, | ID | sense | 333 and 333, respectively, | ID | Duplex |
| ID# | in order of appearance) | NO: | ID # | in order of appearance) | NO: | ID# |

| | | | | | | |
|---|---|---|---|---|---|---|
| A-5718 | GGC UCU UAG CAA AGU CAA GdTdT | 1 | A-5719 | CUU GAC UUU GCU AAG AGC CdTdT | 2 | ALN-RSV01 |
| A-30631 | GGcuCUUAGcAaAGucAAGdTsdT | 320 | A-30648 | CUuGACUUuGCUAAGAGCcdTsdT | 323 | AD-3532 |
| A-30631 | GGcuCUUAGcAaAGucAAGdTsdT | 320 | A-30652 | CUuGACUuUGCUAAGAGcCdTsdT | 324 | AD-3534 |
| A-30633 | GgCUCUuAGcAAAGUcAAGdTsdT | 321 | A-30650 | CUuGACUUuGCuAAGAGCcdTsdT | 325 | AD-3586 |
| A-30629 | GGcuCUUAGcAaAGucAaGdTsdT | 322 | A-30653 | CUuGACUuUGCuAAGAGCcdTsdT | 326 | AD-3587 |
| A-30631 | GGcuCUUAGcAaAGucAAGdTsdT | 320 | A-30653 | CUuGACUuUGCuAAGAGCcdTsdT | 326 | AD-3588 |
| A-30633 | GgCUCUuAGcAAAGUcAAGdTsdT | 321 | A-30653 | CUuGACUuUGCuAAGAGCcdTsdT | 326 | AD-3589 |

Nomenclature:
Capital letters = RNA base,
small letters = 2′-O-Methyl base,
dT = DNA Thymidine,
s = Phosphorothioate linkage.

The stability of the six modified ALN-RSV01 dsRNAs were examined in the following different media: Human serum, Nasal wash from RSV sick volunteers (frozen samples), in fresh rat and fresh cynomolgus monkeys (NHP) bronchial areola lavage (BAL) fluid. In all cases, the same analytical procedure for unmodified and moderately modified sequences (e.g. 2′-O-Methyl-modification) was used, as described below.

Materials and Methods

The following abbreviations are used in this Example: NaOH—Sodium Hydroxide; NaBr—Sodium Bromide; ACN—Acetonitrile ; HCl—Hydrogen Chloride; PBS—Phosphate Buffered Saline; DI water—Deionized water; PCR—Polymerase Chain Reaction; IEX—Ion exchange chromatography, NHP—Non human primate, BAL—bronchial alveolar lavage.

The siRNA was incubated in 90% of a suitable media (e.g., human serum) at a temperature of 37° C. for specific time periods. The reaction was stopped or quenched using ProteinaseK in lysis buffer which degrades any nucleases present and therefore prevents further degradation of the RNA. The samples were filtered to remove debris and particles which could harm the subsequent HPLC setup. The filtered samples were separated chromatographically by ion exchange chromatography on a Dionex DNAPac PA-200 column using denaturing conditions. The duplex was intentionally denatured into its corresponding single strands using elevated temperature and high pH. The separation was based on anion exchange mechanism and the physicochemical differences of the sequences. The secondary structures of RNA were minimized at elevated temperature and high pH. For stability evaluation the area of the intact remaining RNA single strands areas was compared to the T=0 time point area (set to 100%) of the corresponding strands. The T=0 time point underwent the same processing steps and was exposed to the same reagents and dilutions as any other time points evaluated, but was processed immediately after combining all components without further time loss. The identity of the sense or antisense strand was confirmed by injecting the appropriate single strand of known sequence and using the same method and comparing the retention times. As an external control for possible unspecific degradation a PBS sample of the siRNA incubated also at 37° C. for 24 h was used. No internal standards were used. All time points were prepared independently in separate vials in duplicate.

Reagents: Reagents were purchased or provided as follows: Human serum (Bioreclamation, #HMSRM); Mouse serum (Sigma, #M5905); BAL rat (in house manufactured); BAL cynomolgus monkey (Charles River corporation, Wilmington, Mass.); 10×PBS Buffer (Gibco, #70013-032); Proteinase K (20 mg/ml) (Invitrogen, #25330-049); Epicentre cell and tissue lysis buffer (Epicentre #MTC096H); Eppendorf water, RNase, DNase, endotoxin free, (Eppendorf #1039480); 10×PBS Buffer (Gibco, #70013-032); Proteinase K (20 mg/ml) (Invitrogen, #25330-049). All solvents and chemicals were at least of ACS quality if not higher in particular was used Acetonitrile, HPLC grade (Burdick & Jackson, #10071743); Sodium Hydroxide (VWR, #VW3247-1); Sodium Bromide (J. T. Baker, #3836-05); Hydrochloric Acid (BDH Aristar, #BDH3026). De-ionized water, generated in house of MilliQ quality was used throughout all steps.

Equipment: As an IEX HPLC, a Dionex Ultimate 3000 HPLC was used with the following setup: an SRD-3600 Solvent rack and degasser, a DGP-3600A Dual-gradient analytical pump, a WPS-3000SL Analytical in-line split loop autosampler, a TCC-3100 1×2P-10P Thermostatted column compartment with 1×2 position 10-port switching valve equipped with 2× system capillary kits to run two columns in tandem, and a VWD-3x00 Variable wavelength detector. The setup was controlled by the Chromeleon software, version 6.8. A Dionex HPLC and an analytical column, DNAPac PA-200, 4×250, P/N 063000, was used for the separation of the six duplexes. To ensure proper temperature control of the stability reaction, an Eppendorf Mastercycler Gradient PCR Machine or Eppendorf Thermomixer with microplate adapter adjusted to 37° C. or 65° C. was used for the ProteinaseK step. As reaction vessels, Axygen PCR tubes (0.2 ml; VWR, #10011-764) covered with Axygen PCR 8-strip flat caps (VWR, #89080-526) were used to avoid unnecessary evaporation. For filtering the stopped reaction the Captiva 96-well 0.2 μm Polypropylene filter plate (Varian, #A5960002) and a Sorvall Legend RT plus tabletop centrifuge equipped with a Sorvall HIGH Plate rotor for Legend RT plus centrifuge was used. The samples were filtered into a Agilent Technologies, 96-Well Plate, Polypropylene deep well plate (VWR, #HP5042-1385) and the plate was sealed with tape, Sealing Film, (USA Scientific, #2923-5010), and then analyzed by IEX chromatography.

Solutions, matrices, and buffer preparation. The six siRNA duplex were adjusted to 50 μM siRNA solution in 1×PBS, and the appropriate sense and antisense strands were separately adjusted to 5 μM in water or 1×PBS. Serum and other matrixes were used straight, undiluted from aliquoted frozen stock to avoid freeze thaw cycles. The following serums were used for testing: Human serum (Bioreclamation, #HMSRM); Mouse serum (Sigma, #M5905). The bronchial aveola lavage (BAL) was generated fresh and used the same day. Rat BAL was produced in house from Sprague Dawley rats of mixed gender. About 4 ml of BAL from each rat was produced and kept separately. For the NHP BAL, male cynomolgus monkey macaques of about 4 kg bodyweight were used. A total of approximately 50 ml BAL per NHP from three cyno monkeys in three aliquots of about 15 ml each from Charles River animal facility in Wilmington, Mass. was delivered and used fresh the same day. The three aliquots represented three consecutive washes to generate the BAL. From each NHP only the first wash with the most cell debris was used, representing the most aggressive medium possible. The other two aliquots were kept as back-up. Further Buffer Solutions were prepared, including a Stop Solution (for one plate 200 μL of proteinase K (20 mg/mL), 2.5 mL of Epicentre tissue and cell lysis buffer, and 3.5 mL of water (RNase, DNase, endotoxin free) is combined and mixed) and a system blank for IEX (25% Lysis buffer solution; mix 250 μL of Epicentre tissue and cell lysis solution with 750 uL of 1×PBS).

IEX-HPLC Conditions and buffers. Buffer A for the IEX HPLC mobile phase was 20 mM NaOH, 20 mM NaBr in 10% ACN, adjusted to pH 12. Buffer B was 20 mM NaOH, 1 M NaBr, in 10% CAN, also adjusted to pH 12 with NaOH. Buffer C was 1 mM HCl in 90% ACN. All buffers were filtered through 0.2 micron nylon membrane filters, stored ambient in sealed, triple rinsed glass Schott bottles and made fresh every two weeks. The IEX-HPLC conditions were as follows: column temperature 35° C.; flow rate 1.0 ml minute; UV-detection at 260 nm; 50 μl injections. The gradient developed as written: 0-1 min 95% A, 5% B; 1-2 min 50% A, 50% B, 2-17 min 40% A, 60% B; 17.5-19.5 min 100% C. The total run time was 27 minutes.

Incubation conditions. 40 μL of appropriate serum was combined with 4 μL of a 50 μM siRNA duplex in a appropriate labeled PCR tube. The tubes were closed immediately to avoid evaporation. All samples were prepared in duplicate and incubate for the indicated times at 37° C. shaking at 1200 rpm for the first 20 minutes. Full range of time points were used: 0, 0.5, 1, 2, 6, 16, and 24 hours. All reactions were started at the designated time prior to the 24 hour time point and stopped at the same time. The 24 hour samples were prepared first and placed at 37° C. 18 hours later, the 6 hour samples were prepared and placed at 37° C. For the 16 hour time point, samples were incubated for 16 hours as above and then placed at −80° C. When the zero time point was reached, the 16 hour sample was warmed to 37° C. and quenched as described below. For the zero hour time point, 60 μL of stop solution was added to a new PCR tube, then 40 μL of serum followed by 4 μL of a 50 mM siRNA solution. As the PBS control sample for unspecific degradation, 40 μL of 1×PBS with 4 μL of a 50 μM siRNA solution in a PCR tube was combined and incubated for 24 hours at 37° C., shaking at 1200 rpm for the first 20 minutes.

Reaction quenching at each time point was achieved by adding 62 μL of stop solution to the sample tube. All samples and controls were incubated for 20 min. at 52° C., shaking at 1200 rpm. After completion the samples were spun at 1400 rpm in a centrifuge for 20 sec to quickly remove any liquid remaining on the lids. Then all samples were transferred to the 0.2 μm 96 well filter plate for filtration. The incubation tubes were washed with 100 μL of Eppendorf water. This wash was added to the filter plate along with the sample and the plate spun at 1400 rpm for 10 minutes into an Agilent 96-well plate. The plate was then covered with sealing film for analysis on the HPLC. Two System Blanks of 50 μL were injected at the beginning of the run. The samples were then analyzed in the following order: three system suitability samples of the sense or antisense strand, one sample of the other strand that was not used for system suitability, then the incubated samples starting with 0 hour and continuing to 24 hours followed by the PBS incubation. Duplicates were run sequentially. If there was more than one duplex, each set of duplex samples was analyzed consecutively.

The single stranded antisense or sense RNA served as the system suitability standard with a required relative standard deviation (RSD) of the retention times for the main peak of less than 5%. They also were used to identify the retention times of the single strands during the siRNA analyses.

Data reporting and calculations: The integration was performed using the Chromeleon Software. The peak areas for the sense and antisense strands are recorded. Microsoft Excel was used for statistical calculations and graph plotting. The percent strand remaining was calculated by dividing the time point area for each strand by the value of the area for the zero time point (t=0) and multiplying by 100%, as shown below (i.e., Percent strand remaining=Area_{(time point)}/Area_{(zero time point)}*100%). Half life values for the sense and antisense strand were calculated using Microsoft Excel XLfit or Graphpad Prism.

Results

Stability of modified ALN-RSV01 siRNAs in human serum. The six modified ALN-RSV01 siRNAs were evaluated for their stability over 24 hours in human serum following the procedure described in the section before. Two different lots of human serum from different vendors (Bioreclamation and Sigma Aldrich) were used separately to minimize the chance of variance due to serum effects.

A summary of the individual strand half lives (hours) plus standard deviation for the six modified ALN-RSV01 molecules in either of two human serum lots is shown in Table 21, below:

TABLE 21

| | AS | ± | S | ± | AS | ± | S | ± | AS | ± | S | ± |
|---|---|---|---|---|---|---|---|---|---|---|---|---|
| | AD-3532 | | | | AD-3534 | | | | AD-3586 | | | |
| Bioreclamation | 30.32 | 1.52 | 37.12 | 1.86 | 37.02 | 1.85 | 42.77 | 2.14 | 51.03 | 2.55 | 69.38 | 3.47 |
| T½ (Hours) | | | | | | | | | | | | |
| Sigma T½ | 24.32 | 1.22 | 31.83 | 1.59 | 26.58 | 1.33 | 32.99 | 1.65 | 36.01 | 1.80 | 53.65 | 2.68 |
| (Hours) | | | | | | | | | | | | |
| | AD-3587 | | | | AD-3588 | | | | AD-3589 | | | |
| Bioreclamation | 57.47 | 2.87 | 62.63 | 3.13 | 48.55 | 2.43 | 62.72 | 3.14 | 59.04 | 2.95 | 78.69 | 3.93 |
| T½ (Hours) | | | | | | | | | | | | |
| Sigma T½ | 44.60 | 2.23 | 48.77 | 2.44 | 43.79 | 2.19 | 58.80 | 2.94 | 46.39 | 2.32 | 68.19 | 3.41 |
| (Hours) | | | | | | | | | | | | |

The data showed that all duplexes have greater than 24 hour half lives in human serum. The duplexes AD-3532 and AD-3534 have a lower stability by nearly a factor of 2 than the remaining four duplexes, i.e., AD-3586, AD-3687, AD-3588 and AD-3589. In contrast, ALN-RSV01 has a half-life of less than 5 minutes in serum

Stability of modified ALN-RSV01 siRNAS in nasal wash of RSV-infected, sick volunteers. The six modified RSV01 candidates were evaluated for their stability over 24 hours in previously obtained, stored frozen aliquots of nasal washes from RSV-infected, sick volunteers. Following the procedure described above, the nasal wash aliquots were thawed and used immediately afterwards in the assay.

The tabulated half lives (hours) in the nasal wash samples, including standard deviation, for the sense and antisense strands of the six modified ALN-RSV01 siRNAs candidates is shown in Table 22.

TABLE 22

| | AD-3532 | ± | AD-3534 | ± | AD-3586 | ± |
|---|---|---|---|---|---|---|
| Antisense | 12.50 | 0.63 | 11.20 | 0.56 | 16.40 | 0.82 |
| Sense | 14.80 | 0.74 | 12.20 | 0.61 | 18.90 | 0.95 |
| | AD-3587 | ± | AD-3588 | ± | AD-3589 | ± |
| Antisense | 12.50 | 0.63 | 18.00 | 0.90 | 16.00 | 0.80 |
| Sense | 22.20 | 1.11 | 21.40 | 1.07 | 18.00 | 0.90 |

The data showed that all duplexes have 12-18 hour lower half lives in the nasal washes relative to human serum. The duplexes AD-3532 and AD-3534 have a lower stability by nearly a factor of two then the other duplexes (AD-3586, AD03687, AD-3588 and AD-3589), similar to what was found in the serum.

Stability of modified ALN-RSV01 siRNAs in fresh rat and fresh non-human primate (NHP) BAL. In the first rat assay, pooled fresh Rat BAL from nine different rats was used. All six candidates where tested in duplicate and at time points ½, 1, 4, 8 hours. The data showed that within error, all six siRNAs showed similar minor degradation after 8 hours in the range of 70% for the sense strand and 85% for the antisense strand. Half lives could not be calculated due to the minimum degradation occurred. The second rat BAL study was executed with individualized rat BAL from three rats. Only the three duplexes AD-3532, AD-3587 and AD-3589 were tested in duplicate, in three “Lots” of Rat BAL. The time points evaluated were ½, 1, 4, and 8 hours. All three siRNAs showed similar minor degradation. Half lives could not be calculated due to the minimum amount of degradation observed.

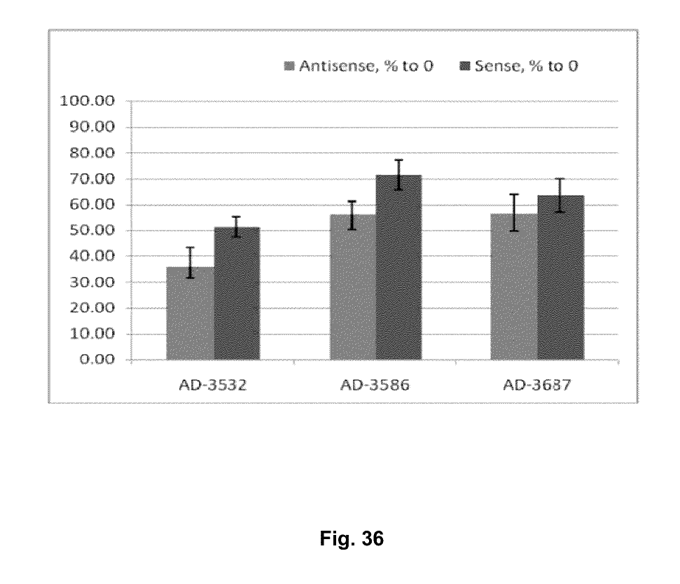

The stability of three duplexes (AD-3532, AD-3586 and AD-3587) in individualized fresh NHP BAL from three cynomolgus monkeys was also tested. All three duplexes where tested, in duplicate, at time points ½, 1, 4, and 8 hours. The results observed from each of the three animal samples were averaged and shown in FIG. 36.

As shown in FIG. 36, more degradation is obtained in NHP BAL after 8 hours compared to the data obtained with rat BAL. The data was also variable from animal to animal, most likely due to the relatively non-homogeneous nature of the NHP BAL. Even taking into account the variability of the data, it is possible to differentiate between the three duplexes AD-3532, AD-3586, and AD-3587. AD-3532 appears to be the less stable of the three in the NHP BAL assay, whereas AD-3586 and AD-3587 appear to seem to have similar stability this assay.

In summary, the stability data shows a clear tendency across the duplexes AD-3532, AD-3587 and AD-3589 or 3586. In all matrices tested, the duplex AD-3532 was less stable than AD-3587, AD-3589 or 3586, respectively, whereas duplexes AD-3586, AD-3587 and AD-3589 appear to have the same stability.

1. A modified double-stranded ribonucleic acid (dsRNA) for inhibiting expression of a Respiratory Syncytial Virus (RSV) gene, wherein said dsRNA comprises a modified antisense strand, the antisense strand comprising a region complementary to a part of the N gene of RSV, wherein said region of complementarity comprises 15 or more contiguous nucleotides of the sequence CUUGACUUUGCUAAGAGCC (SEQ ID NO: 305), wherein at least three nucleotides in said antisense sequence are modified.

2. The dsRNA of claim 1, wherein the sense strand comprises a modified nucleotide sequence selected from the group consisting of A-30629 (SEQ ID NO: 322), A-30631 (SEQ ID NO: 320) and A-30633 (SEQ ID NO: 321).

3. The dsRNA of claim 1, wherein the sense strand comprises 15 or more contiguous nucleotides of GGCUCUUAGCAAAGUCAAG (SEQ ID NO: 302), and wherein at least three nucleotides in said sense strand are modified.

4. (canceled)

5. The dsRNA of claim 2, wherein the sense strand consists of the modified nucleotide sequence A-30629 (SEQ ID NO: 322).

6. The dsRNA of claim 2, wherein the sense strand consists of the modified nucleotide sequence A-30631 (SEQ ID NO: 320).

7. (canceled)

8. The dsRNA of claim 1, wherein the antisense strand consists of the modified nucleotide sequence A-30653 (SEQ ID NO: 326).

9. The dsRNA of claim 1, wherein the antisense strand consists of the modified nucleotide sequence A-30648 (SEQ ID NO:323).

10. (canceled)

11. (canceled)

12. (canceled)

13. (canceled)

14. The dsRNA of claim 1, wherein the sense strand consists of the modified nucleotide sequence A-30629 (SEQ ID NO: 322) and the antisense strand consists of the modified nucleotide sequence A-30653 (SEQ ID NO: 326).

15. The dsRNA of claim 1, wherein the sense strand consists of the modified nucleotide sequence 30631 (SEQ ID NO: 320) and the antisense strand consists of the modified nucleotide sequence A-30648 (SEQ ID NO:323).

16. The dsRNA of claim 1, wherein the region of complementarity is 19 nucleotides in length.

17. (canceled)

18. The dsRNA of claim 1 wherein each strand of the dsRNA is 19, 20, 21, 22, 23, or 24 nucleotides in length.

19. The dsRNA of claim 1 wherein each strand is 21 nucleotides in length.

20. (canceled)

21. (canceled)

22. (canceled)

23. The dsRNA of claim 1, wherein each strand comprises between three and six 2′-O-methyl modified pyrimidine nucleotides.

24. The dsRNA of claims 1, wherein said antisense strand comprises three 2′-O-methyl modified pyrimidine nucleotides.

25. The dsRNA of claims 1, wherein said antisense strand comprises three 2′-O-methyl modified pyrimidine nucleotides and said sense strand comprises four to six 2′-O-methyl modified pyrimidine nucleotides.

26. The dsRNA of claims 1, wherein each strand comprises a dTdT overhang.

27. The dsRNA of claim 26, wherein said dTdT overhang comprises a phosphorothioate linkage.

28. The dsRNA of claims 1, wherein said antisense strand comprises three 2′-O-methyl modified nucleotides and said sense strand comprises four to six 2′-O-methyl modified nucleotides, and wherein each strand comprises a modified or unmodified dTdT overhang.

29. The dsRNA of claim 28, wherein said dTdT overhang on each strand comprises a phosphorothioate linkage.

30. The dsRNA of claims 1, wherein the dsRNA is formulated for intranasal or intrapulmonary delivery.

31. The dsRNA of claim 30, wherein the dsRNA is formulated in a buffered saline solution.

32. The dsRNA of claim 30, wherein the dsRNA is formulated in a phosphate buffered saline solution.

33. (canceled)

34. (canceled)

35. The dsRNA of claim 30, wherein administration of the dsRNA in vivo results in reduced immunostimulation relative to ALN-RSV01, as measured by TNF-α, IL-6 and IL1-RA ELISA assays on epithelial lining fluid obtained by bronchoalveolar lavage.

36. The dsRNA of claim 30, wherein the dsRNA is non-immunostimulatory.

37. The dsRNA of claim 30, wherein administration of the dsRNA does not result in immunostimulatory activity in human peripheral blood mononuclear cells (PBMCs) as measured by IFN-alpha ELISA assays.

38. The dsRNA of claim 30, wherein administration of the dsRNA results in reduced immunostimulatory activity by at least an order of magnitude in human peripheral blood mononuclear cells (PBMCs) relative to ALN-RSV01, as measured by real-time PCR measurements of TNF-α, IL-6 and IP-10 mRNA.

39. (canceled)

40. (canceled)

41. The dsRNA of claim 30, wherein administration of the dsRNA results in a 5 fold decrease in IFN-γ mRNA induction in human peripheral blood mononuclear cells (PBMCs) relative to ALN-RSV01, as measured by real-time PCR.

42. (canceled)

43. (canceled)

44. (canceled)

45. The dsRNA of claim 30, wherein said sense or antisense strand of said dsRNA has a half-life of at least 24 hours in human serum.

46. The dsRNA of claim 30, wherein said sense or antisense strand of said dsRNA has a half-life of at least 48 hours in human serum.

47. The dsRNA of claim 30, wherein said sense or antisense strand of said dsRNA has a half-life of at least 10 hours in human nasal washes.

48. (canceled)

49. (canceled)

50. (canceled)

51. A cell containing the modified dsRNA of claim 1.

52. (canceled)

53. (canceled)

54. A pharmaceutical composition for reducing viral titer or retarding viral proliferation in a cell of a subject, wherein said composition comprises the modified dsRNA of any of claim 1 and a pharmaceutically acceptable carrier.

55. A method of inhibiting RSV replication in a cell of a subject, the method comprising:
(a) contacting the cell with the modified dsRNA of claim 1; and
(b) maintaining the cell produced in step (a) for a time sufficient to obtain degradation of an mRNA transcript of an RSV gene, thereby inhibiting replication of the virus in the cell.

56.-81. (canceled)
